(19) United States
(12) Patent Application Publication
(10) Pub. No.: US 2024/0335516 A1
(43) Pub. Date: Oct. 10, 2024

(54) COMPOSITIONS AND METHODS FOR THE TREATMENT OF SPINAL MUSCULAR ATROPHY (SMA)

(71) Applicant: Beam Therapeutics Inc., Cambridge, MA (US)

(72) Inventors: Shaunna Berkovitch, Cambridge, MA (US); Jason Michael Gehrke, Cambridge, MA (US); Holly Rees, Cambridge, MA (US); Conrad Rinaldi, Cambridge, MA (US); Tanggis Bohnuud, Cambridge, MA (US)

(73) Assignee: Beam Therapeutics Inc., Cambridge, MA (US)

(21) Appl. No.: 18/744,175

(22) Filed: Jun. 14, 2024

Related U.S. Application Data
(63) Continuation of application No. PCT/US2022/081825, filed on Dec. 16, 2022.
(60) Provisional application No. 63/291,290, filed on Dec. 17, 2021.

(51) Int. Cl. A61K 38/50; A61K 31/7105; A61K 38/46; C07K 14/47; C12N 9/22; C12N 9/78; C12N 15/11; C12N 15/90

(52) U.S. Cl. CPC A61K 38/50; A61K 31/7105; A61K 38/465; C07K 14/4702; C12N 9/22; C12N 9/78; C12N 15/11; C12N 15/907; C12Y 305/04004; C12Y 305/04005; C12N 2310/20

(57) ABSTRACT

The invention of the disclosure features compositions and methods for treating spinal muscular atrophy (SMA) by introducing alterations to a survival of motor neuron 2 (SMN2) polynucleotide(s) in a cell. In particular embodiments, the invention provides a base editor system (e.g., a fusion protein or complex comprising a programmable DNA binding protein, a nucleobase editor, and gRNA) for modifying an SMN2 polynucleotide(s), where the alteration is associated with increased expression of the SMN2 polypeptide(s) encoded by the polynucleotide(s).

### CROSS REFERENCE TO RELATED APPLICATIONS

This application is a continuation under under 35 U.S.C. § 111(a) of International Application No. PCT/US2022/081825, filed Dec. 16, 2022, which claims priority to and benefit of U.S. Provisional Application No. 63/291,290, filed Dec. 17, 2021, the entire contents of which is hereby incorporated herein by reference in its entirety.

### SEQUENCE LISTING

This application contains a Sequence Listing which has been submitted electronically in XML format and is hereby incorporated by reference in its entirety. The Sequence Listing XML file, created on Jul. 1, 2024, is named 180802_046103US_SL.xml and is 1,247,242 bytes in size.

### BACKGROUND OF THE INVENTION

Spinal muscular atrophy (SMA) is a genetic disorder characterized by weakness and wasting in skeletal muscles, which is associated with the homozygous deletion and/or mutation of alleles of the survival of motor neuron 1 (SMN1) gene located on chromosome 5q13. It is characterized by degeneration of the alpha motor neurons in the spinal cord, which causes proximal, symmetrical limb and trunk muscle weakness that progresses to paralysis. SMA affects 1 per 8,000 to 10,000 newborns worldwide and approximately 1 to 2 per 100,000 persons. Outcomes in the natural course of the disease vary from death within a few weeks after birth to normal life expectancy. If left untreated, the many children diagnosed with SMA do not reach the age of 4, with a primary cause of death being recurrent respiratory problems. Long-term survival in severe cases of SMA is uncommon.

Thus, there is a need for improved compositions and methods for the treatment of SMA.

### SUMMARY OF THE INVENTION

As described below, the invention of the present disclosure features compositions and methods for treating spinal muscular atrophy (SMA) by introducing alterations to a survival of motor neuron 2 (SMN2) polynucleotide in a cell. In particular embodiments, the invention provides a base editor system (e.g., a fusion protein or complex containing a programmable DNA binding protein, a nucleobase editor, and a guide polynucleotide) for modifying an SMN2 polynucleotide, where the alteration is associated with increased expression of the SMN2 polypeptide(s) encoded by the polynucleotide.

In one aspect, the invention features a method of editing a nucleobase of a survival of motor neuron 2 (SMN2) polynucleotide. The method involves contacting the SMN2 polynucleotide with a guide polynucleotide and a base editor containing a fusion protein or a protein complex containing a nucleic acid programmable DNA binding protein (napDNAbp) domain and a deaminase domain, or a polynucleotide encoding the base editor. The guide polynucleotide targets the base editor to effect an alteration of the nucleobase of the SMN2 polynucleotide.

In one aspect, the invention features a method of editing a nucleobase of a survival of motor neuron 2 (SMN2) polynucleotide. The method involves contacting the SMN2 polynucleotide with one or more guide polynucleotides and a base editor containing a fusion protein or a protein complex containing a nucleic acid programmable DNA binding protein (napDNAbp) domain and a deaminase domain. The one or more guide polynucleotides target the base editor to effect an alteration of the nucleobase of the SMN2 polynucleotide.

In another aspect, the invention features a method of treating spinal muscular atrophy (SMA) in a subject in need thereof. The method involves contacting a cell of the subject with a base editor containing a fusion protein or protein complex containing a nucleic acid programmable DNA binding protein (napDNAbp) domain and a deaminase domain, or a polynucleotide encoding the base editor, and a guide polynucleotide, or a polynucleotide encoding the guide polynucleotide, that targets the base editor to effect an alteration of a survival of motor neuron 2 (SMN2) polynucleotide, thereby treating SMA in the subject.

In another aspect, the invention features a modified cell containing an alteration in a nucleobase of a survival of motor neuron 2 (SMN2) polynucleotide. The alteration increases expression and/or activity of the encoded SMN2 polypeptide as compared to a control cell without the alteration.

In another aspect, the invention features a base editor system containing a fusion protein or a polynucleotide encoding the fusion protein. The fusion protein contains a nucleic acid programmable DNA binding protein (napDNAbp) domain, a deaminase domain, and a guide polynucleotide containing a spacer sequence selected from Table 2A or Table 2C.

In another aspect, the invention features a polynucleotide encoding the base editor system of any one of the above aspects, or a component thereof.

In another aspect, the invention features a pharmaceutical composition containing an effective amount of a base editor system containing a fusion protein or a polynucleotide encoding the fusion protein. The fusion protein contains a nucleic acid programmable DNA binding protein (napDNAbp) domain, a deaminase domain, and a guide polynucleotide containing a spacer selected from Table 2A or Table 2C.

In another aspect, the invention features a guide polynucleotide containing a sequence listed in Tables 2A-2C.

In another aspect, the invention features cell produced by the method of any of the above aspects.

In another aspect, the invention features a kit containing a base editor system containing a fusion protein or a polynucleotide encoding the fusion protein. The fusion protein contains a nucleic acid programmable DNA binding protein (napDNAbp) domain, a deaminase domain, and a guide polynucleotide containing a spacer selected from Table 2A or Table 2C.

In any of the above aspects, or embodiments thereof, the polynucleotide is in a cell, and where the cell is in vivo or in vitro. In any of the above aspects, or embodiments thereof, the cell is a mammalian cell. In any of the above aspects, or embodiments thereof, the cell is a rodent, non-human primate, or human cell.

In another aspect, the invention features a vector containing a polynucleotide of any of the above aspects.

In any of the above aspects, or embodiments thereof, the cell is contacted with two or more guide polynucleotides. In any of the above aspects, or embodiments thereof, the guide polynucleotide contains a spacer selected from those listed in Table 2A or Table 2C. In any of the above aspects, or embodiments thereof, the guide polynucleotide contains a nucleic acid sequence containing at least 10 contiguous nucleotides of a spacer nucleic acid sequence listed in Table 2A or Table 2C.

In any of the above aspects, or embodiments thereof, the deaminase domain is an adenosine deaminase domain or a cytidine deaminase domain. In embodiments, the adenosine deaminase domain converts a target A•T to G•C in the SMN2 polynucleotide. In embodiments, the cytidine deaminase domain converts a target C•G to T•A in the SMN2 polynucleotide.

In any of the above aspects, or embodiments thereof, alteration of the nucleobase is associated with an increase in full-length polynucleotides encoding the SMN2 polypeptide transcribed from the SMN2 polynucleotide. In any of the above aspects, or embodiments thereof, alteration of a nucleobase in the SMN2 polynucleotide results in an increase in the number of transcripts transcribed from the SMN2 polynucleotide that include Exon 7. In any of the above aspects, or embodiments thereof, the altered nucleobase in the SMN2 polynucleotide is associated with an alteration in splicing. In any of the above aspects, or embodiments thereof, the alteration is associated with an increase in levels of a survival motor neuron (SMN) polypeptide in a cell. In any of the above aspects, or embodiments thereof, the alteration is selected from one or more of a C10 alteration in Intron 7 of the SMN2 polynucleotide, a C7 alteration in Intron 7 of the SMN2 polynucleotide, a C11 alteration in Intron 7 of the SMN2 polynucleotide, a T6C alteration in Exon 7 of the SMN2 polynucleotide, and a A54G alteration in Exon 7 of the SMN2 polynucleotide.

In any of the above aspects, or embodiments thereof, the napDNAbp domain contains a Cas9, Cas12a/Cpf1, Cas12b/C2c1, Cas12c/C2c3, Cas12d/CasY, Cas12e/CasX, Cas12g, Cas12h, Cas12i, or Cas12j/CasΦ polynucleotide or a portion thereof. In any of the above aspects, or embodiments thereof, the napDNAbp domain contains a Cas9 polynucleotide or a portion thereof. In any of the above aspects, or embodiments thereof, the napDNAbp domain contains a dead Cas9 (dCas9) or a Cas9 nickase (nCas9). In any of the above aspects, or embodiments thereof, the napDNAbp domain is a modified Staphylococcus aureus Cas9 (SaCas9), Streptococcus thermophilus 1 Cas9 (St1Cas9), a modified Streptococcus pyogenes Cas9 (SpCas9), or variants thereof. In any of the above aspects, or embodiments thereof, the napDNAbp domain contains a variant of SpCas9 having an altered protospacer-adjacent motif (PAM) specificity.

In any of the above aspects, or embodiments thereof, the cytidine deaminase domain is an APOBEC deaminase domain or a derivative thereof.

In any of the above aspects, or embodiments thereof, the adenosine deaminase domain is TadA deaminase domain. In embodiments, the adenosine deaminase domain is a TadA*8 variant. In embodiments, the adenosine deaminase domain is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In any of the above aspects, or embodiments thereof, the guide polynucleotide contains a nucleic acid sequence containing at least 10 contiguous nucleotides of a spacer nucleic acid sequence listed in Table 2A or Table 2C. In any of the above aspects, or embodiments thereof, guide polynucleotide contains a spacer sequence listed in Table 2A or Table 2C. In any of the above aspects, or embodiments thereof, the guide polynucleotide contains a nucleic acid analog. In any of the above aspects, or embodiments thereof, the guide polynucleotide contains one or more of a 2′-OMe and a phosphorothioate.

In any of the above aspects, or embodiments thereof, the napDNAbp domain further contains one or more uracil glycosylase inhibitors (UGIs). In any of the above aspects, or embodiments thereof, the napDNAbp domain further contains one or more nuclear localization sequences (NLS).

In any of the above aspects, or embodiments thereof, expression and/or function is increased by at least 1, 2, 3, 4, 5, 6, 7, 8, 9, or 10-fold as compared to a control cell without the alteration. In any of the above aspects, or embodiments thereof, the alteration is associated with an increase in the number of full-length transcripts encoding the SMN2 polypeptide transcribed from the SMN2 polynucleotide.

In any of the above aspects, or embodiments thereof, the cell is a neuron.

In any of the above aspects, or embodiments thereof, the fusion protein further contains one or more uracil glycosylase inhibitors (UGIs). In any of the above aspects, or embodiments thereof, the fusion protein further contains one or more nuclear localization sequences (NLS).

In any of the above aspects, or embodiments thereof, the napDNAbp is a nuclease inactive or nickase variant.

In any of the above aspects, or embodiments thereof, the deaminase domain is capable of deaminating cytidine or adenine in DNA. In any of the above aspects, or embodiments thereof, the deaminase domain is a cytidine deaminase domain. In any of the above aspects, or embodiments thereof, the cytidine deaminase domain is an APOBEC deaminase domain or a derivative thereof.

In any of the above aspects, or embodiments thereof, the deaminase domain is an adenosine deaminase domain.

In any of the above aspects, or embodiments thereof, the deaminase is a monomer or heterodimer.

In any of the above aspects, or embodiments thereof, the kit further contains written instructions for the use of the kit in the treatment of spinal muscular atrophy.

In any of the above aspects, or embodiments thereof, the cell is a neuron. In any of the above aspects, or embodiments thereof, the neuron is a motor neuron. In any of the above aspects, or embodiments thereof, the cell is a fibroblast cell.

In any of the above aspects, or embodiments thereof, the guide polynucleotide contains the nucleotide sequence ACUCCUUAAUUUAAGGAAUG (SEQ ID NO: 562; gRNA1962); GACAAAAUCAAAAAGAAGGA (SEQ ID No: 514; gRNA1973); UUAAGGAGUAAGUCUGCCAG (SEQ ID NO: 626; gRNA2349); UGCUCACAUUCCUUAAAUUA (SEQ ID NO: 574; gRNA1974); GCAGACUUACUCCUUAAUUUA (SEQ ID NO: 576; gRNA1976); or UCACAUUCCUUAAAUUAAGGA (SEQ ID NO: 592; gRNA1992). In any of the above aspects, or embodiments thereof, the guide polynucleotide contains a scaffold containing the nucleotide sequence:

SpCas9 scaffold
(SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU;
or
SaCas9 scaffold
(SEQ ID NO: 425)
GUUUUAGUACUCUGUAAUGAAAAUUACAGAAUCUACUAAAACAAGGCAA
AAUGCCGUGUUUAUCUCGUCAACUUGUUGGCGAGAUUUU.
In any of the above aspects, or embodiments
thereof, the guide polynucleotide contains the
sequence
(SEQ ID NO: 503; gRNA1962)
ACUCCUUAAUUUAAGGAAUGGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 514; gRNA1973)
GACAAAAUCAAAAAGAAGGAGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 630; gRNA2349)
UUAAGGAGUAAGUCUGCCAGGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 515; gRNA1974)
UGCUCACAUUCCUUAAAUUAGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 517; gRNA1976)
GCAGACUUACUCCUUAAUUUAGUUUUAGUACUCUGUAAUGAAAAUUACA
GAAUCUACUAAAACAAGGCAAAAUGCCGUGUUUAUCUCGUCAACUUGUU
GGCGAGAUUUU;
or
(SEQ ID NO: 533; gRNA1992)
UCACAUUCCUUAAAUUAAGGAGUUUUAGUACUCUGUAAUGAAAAUUACA
GAAUCUACUAAAACAAGGCAAAAUGCCGUGUUUAUCUCGUCAACUUGUU
GGCGAGAUUUU .

In any of the above aspects, or embodiments thereof, the guide polynucleotide contains one or more modified nucleotides. In any of the above aspects, or embodiments thereof, the one or more modified nucleotides are at the 5′ terminus and/or the 3′ terminus of the guide polynucleotide. In any of the above aspects, or embodiments thereof, the one or more modified nucleotides are 2′-O-methyl-3′-phosphorothioate nucleotides.

In any of the above aspects, or embodiments thereof, at least 45% of the transcripts transcribed from the altered SMN2 polynucleotide include Exon 7. In any of the above aspects, or embodiments thereof, the levels of the SMN polypeptide in the cell are increased by at least about 50% relative to levels prior to being contacted with the base editor or relative to a reference level. In any of the above aspects, or embodiments thereof, the levels of the SMN polypeptide in the cell are increased by at least about 70% relative to levels prior to being contacted with the base editor or relative to a reference level.

In any of the above aspects, or embodiments thereof, the base editor contains the deaminase domain inserted at an internal location of the napDNAbp protein.

In any of the above aspects, or embodiments thereof, the method involves locally or systemically administering to the subject the base editor, or the polynucleotide encoding the base editor, and the guide polynucleotide, or the polynucleotide encoding the guide polynucleotide. In any of the above aspects, or embodiments thereof, the local administration involves intracisternal magna injection, intracerebroventricular injection, or intra-thecal injection.

In any of the above aspects, or embodiments thereof, contacting the cell involves administering a polynucleotide to the subject, wherein the polynucleotide encodes the base editor, or a component thereof, and/or the guide polynucleotide.

In any of the above aspects, or embodiments thereof, the polynucleotide is administered to the subject using a vector containing the polynucleotide.

In any of the above aspects, or embodiments thereof, the vector is a lipid nanoparticle. In any of the above aspects, or embodiments thereof, the vector is a viral vector. In any of the above aspects, or embodiments thereof, the vector is an adeno-associated virus (AAV) vector. In any of the above aspects, or embodiments thereof, the AAV vector is an AAV9 vector or an AAV-PHP.B vector.

In any of the above aspects, or embodiments thereof, the method further involves: (i) contacting the cell with a first polynucleotide encoding a fusion protein containing an N-terminal fragment of the base editor fused to a split intein-N, and (ii) contacting the cell with a second polynucleotide encoding a fusion protein containing the remaining C-terminal fragment of the base editor fused to a split intein-C. In any of the above aspects, or embodiments thereof, the base editor system contains (i) a first polynucleotide encoding a fusion protein containing an N-terminal fragment of the base editor fused to a split intein-N, and (ii) a second polynucleotide encoding a fusion protein containing the remaining C-terminal fragment of the base editor fused to a split intein-C.

In any of the above aspects, or embodiments thereof, the C-terminal amino acid of the N-terminal fragment of the base editor is positioned within the napDNAbp domain of the base editor. In any of the above aspects, or embodiments thereof, the C-terminal amino acid of the N-terminal fragment of the base editor corresponds to position 309 or 576 of the napDNAbp and the N-terminal amino acid of the C-terminal fragment of the base editor corresponds to position 310 or 577 of the napDNAbp, wherein the napDNAbp amino acid position is referenced to the following sequence: SpCas9

(SEQ ID NO: 197)
MDKKYSIGLDIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD.

In any of the above aspects, or embodiments thereof, the split intein-N and split intein-C are components of a split intein selected from one or more of a Cfa, Cfa(GEP), Gp41.1, Gp41.8, IMPDH.1, NrdJ.1, Npu. In any of the above aspects, or embodiments thereof, the split intein-N and/or split intein-C contains a sequence selected from those listed in any one of Tables 20A-20C (SEQ ID NOs: 389-424), or fragments thereof functional as a split intein-N or split intein-C.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “survival of motor neuron 1 (SMN1) polypeptide” is meant a protein or fragment thereof, having at least about 85% amino acid sequence identity to a polypeptide sequence provided at NCBI Reference Sequence No. NP_000335.1 and functioning in small nuclear ribonucleoprotein (snRNP) biogenesis. An exemplary SMN1 polypeptide amino acid sequence from Homo Sapiens is provided below (NCBI Reference Sequence No. NP_000335.1):

(SEQ ID NO: 426)
MAMSSGGSGGGVPEQEDSVLFRRGTGQSDDSDIWDDTALIKAYDKAVAS
FKHALKNGDICETSGKPKTTPKRKPAKKNKSQKKNTAASLQQWKVGDKC
SAIWSEDGCIYPATIASIDFKRETCVVVYTGYGNREEQNLSDLLSPICE
VANNIEQNAQENENESQVSTDESENSRSPGNKSDNIKPKSAPWNSFLPP
PPPMPGPRLGPGKPGLKFNGPPPPPPPPPPHLLSCWLPPFPSGPPIIPP
PPPICPDSLDDADALGSMLISWYMSGYHTGYYMGFRQNQKEGRCSHSL
N.

By “SMN1 polynucleotide” is meant a nucleic acid molecule encoding an SMN1 polypeptide, as well as the introns, exons, and regulatory sequences associated with its expression, or fragments thereof. In embodiments, an SMN1 polynucleotide is the genomic sequence, mRNA, or gene that expresses an SMN1 polypeptide. An exemplary SMN1 nucleotide sequence from Homo Sapiens is provided below, where plain text represents untranslated regions, bold text represents introns, and italicized text represents coding regions (Ensemble Gene Accession No. ENSG00000172062). Splice junctions in the SMN1 polynucleotide sequence provided below are listed in Table 1.

(SEQ ID NO: 427)
AGAAGCCCCGGGCGGCGGAAGTCGTCACTCTTAAGAAGGGACGGGGCCCCACGCTGCGCACCCG
CGGGTTTGCTATGGCGATGAGCAGCGGCGGCAGTGGTGGCGGCGTCCCGGAGCAGGAGGATTCC
GTGCTGTTCCGGCGCGGCACAGGCCAGGTGAGGTCGCAGCCAGTGCAGTCTCCCTATTAGCGCT
CTCAGCACCCTTCTTCCGGCCCAACTCTCCTTCCGCAGCCTCGGGACAGCATCAAGTCGATCCG
CTCACTGGAGTTGTGGTCCGCGTTTTTCTACGTCTTTTCCCACTCCGTTCCCTGCGAACCACAT
CCGCAAGCTCCTTCCTCGAGCAGTTTGGGCTCCTTGATAGCGTTGAGTGGAGGCCCTGCCGCGA
CTTGGCAGTAGCTTATTTTGTTCACTCCTCTCTGGCTGGTGTGGGGGAGGTGGGGGCATTAGGC
CAGGGTGAAGCAGGGGAACCACTTAGGAGTCTGTTAAGATGATCTGAACTTCAGAACAAGATGT
TATTAACAGAGTGAAAGTATTTGGATTCTGGGTATATTTTGAAATCGGAGGCAACAGGTTTTTC
AGATAGATTCGATAACGGAGGTTATCCTGAATAGTTGAAAAGATAAAGTTGCCTTTTGCTGAGG
TGGGAAAGAGAAGATTGCCAGTAGAGCAGGTTTCTCAGGAGTTCAGTCTTGGGCATAGCATGGT
AGGGGTGAATTTGGCTGGAGTGAGTTGGAGAGTAGGAGAAGAGAAATCCAAGGCAACATTTGAC
CAGCCTGGGCAACATAGTGTGACTCCGAGTCTGCAAAAATTAGACGGGTGTTGTGGTGCGCGTC
TGTGGTCTCAGCTACCTGGAAGGTTCAGGCCTTGGAAGGCTCAGGGAGGTGGAGGCTGCAGTGA
TCTGTGATTGCGCCTCTGCACTCCAGCCTGGGCGACAGAGCCAGACCCTGTCTTAAAACAAAAT
AAACGGCCGGGCGCGGTGGCTCAAGCCTGTAATCCCAGCACTTTGGGAGGCCGAGGCGGCCGGA
TCACAAGGTCAGGAGATCGAGACCATCCTGGCTAACACGGTGAAACCCCGTCTCTACTACAAAT
ACAAAAAATTAGCCGGGCGTGGTGACGGGCGCCTGTAGTCCCAGCTACTCGGGAGGCTGAGGCA
GGAGAATGTCATGAAGCCGGGAGGCGGAGCTTGCAGTGAGCCGAGATCGCGCCACTGCACTCCA
GCCTGGGCGATAGAGCAAGACTCCGTCTCAAATAAATAAATAAATAAATAAATAAATAATAAAA
ACATCGGTAGGCATATTTCAAGGAATTCTATTTAAAAAAAATTTTTTTAGAGACAAGTTCGCTC
TCTGTGGCCCAGGCTGGAGTACAGTGGCATGATCCTAGCCCATGGCAGCGTTGATCTCTTGGCC
TCAAGCGACCCTCCTTTGGAGTCGCTGGGCCTAAAGGAGTGAGCCACCACGAAATTTTATTATA
AATGGAGGGTAGAGAAATTGGGCAATAAATGGAGGGGGAAGTGAGTTAAGAGGAATTTTAATTA
TGTGTGTGTGGTTTTAAAAGAGGGGGGTCTTGCTCTGTTGCCCAGGCTGCTGGGGTGCCAGTGG
CGCAATCATGAATCACTACAGCCTTGGACTCCTGGCCTCAAGCTATCCTCCCACCTCTGCCTCC
CAAAGTACTGGGATTACTAGTGTGAGCCACTGCACTAAGATAGGAGCAACATGTTTCAGCATGT
TTGTGGGTTGATAGGAAAGATGAGAATGGGAAAGTTGATGTCGGAAAGAAGACAATGGCTAGAG
CAATGTCCTAGAGTAGGTAAGAAGGGATGGATTTGGCCTTTGTTGGAAACATTAGCGGTTCTTT
TGGTGACAGCTATATAGTTAACACATCTATGATACGTGAATGGGCAGATAGGATGGCAGGAGAT
TTTGAAAGTTCTCTTGATTCTTACTGTTCTCTTAGTGAAAGAAGCAAGGTTATCAGCTAGAAGC
TGGGATGGGAGAGGAAAGAGAAGATGGGAAGTAGATAGTTCTTTAGAAGAGTGGGCAAGGGTTG
GACTAGGGAAGTTTAGTGGAAATATTGCTAGGCAACATAAAGAGCCTACTTGAGATTCGTGGTC
ATGAGTTGAAGGAGACCAGACAGCAAGATTGTGTATGAGGGCACCCACAGAGTAAATGGAGAGT
TGAAATTAATGCAGTTGTGATTTTACCACGTGGATATGAAGAAGTGAGGGGGAGAAGTACAAAG
GAGTTCTCTTAATGATTGACCATGGAATTTAAGCTGGCTAAGAAAGGAAGTGAGAGGCCGGGCG
CGGTGGCTCACGCCTGTAATCCCAGCACTTTGGGAGACTGAGGTGGGTGGATTACCTGAGGTCA
GGAGTTTGAGACCAACCTGGCCGATATGGCGAAACCCCATCTCTAATAAAAATACAGAAAAATT
AGCCGGGAATGGTGGCAGGTGCCTGTAATCCCAGCTACTCAAGAGGCTGTGGCAGGAGTATCCC
TTGGACCCAGGAGGTGGAGGTTGCAGTGAGCCGAGATCACGCCACTGTACTCCAGCCTGGACGA
TATAGTGAGACTTCACCTCAAAAAAAAAAAAAAAAGAAAGGAAGTGAGGATTTTAAGACCCTGA
GAGACAGTTTAAAAAGTGGGAGGATCGGCCGGGCGCTGTGGCTGACACCTGTAATCCCAGCACT
TTGGGAGGCCGAGTTGGGCAGATCACAAGGTCAGGAGTTCGAGACCAGCCTGGCCAATATGGTG
AAACCTTGTCTCTACTAAAAATACAAAAATTAGCCGGGCATGGTGTCACGTGTCTATAATCCCA
GCTACTCGGGAGGCTGAGGCAGAAAAATTGCTTGAACCTGGGAGGCAGAGGTTGCAGACAGCTG
AGATCACTCCATTGCACTCCAGCCTGGGCAACAAGAGCAAAACTTTGTCTTTAAAAAAAAAAAA
AAAAAAAGAATACAAAAATTAGCCGGGCGTGGTGGCGCGTGCCTATAATCCCAGCTACTTGGGA
GGCTGAGGCAGGAGAATCAGTTGAACACGGGAGGCGAGGTTTGCAGTGAGCCGAGATTGCGCCA
CTGCACTCCAGCCTGGGCGACAGAGCAGGACTCCTCTTGGAAAAAAAAAATTAGCTGGGCATGG
TGGCAGGTGCCTGTAGTCTCAGCTACTAGGGAGGCTGAGGCAGGAAAATCACTTGAACCCGGGA
TGTGGAGTTTGCAGTGACCCGAGATCGTGCCACTGTACTCCATCCTGGGCGACAAAATGAGACT
CTGCCTCAAAAAAAAAAAAAAAAAAAAAAAAAGTGGGAGGATCAATGTACTGCCAGTCCTAATG
AAGTGGAATGATTGTCCCCATCAAATCACTAGTAGGAGTAAGTTGCAGAGCCTAGAAGGTGATG
GTTAAGAGAGTGGGATTCTTGAAACTGCATTTATGGAGAGGTTGTGGTTATTGGTTATAATAAA
TAAATACAGTTGAAGTGAGTGAGTAGCTGAGATTTGGGGATGTATCAGTTCATTCTTACACTGC
TACAAAGACATACCTGAGACCAGGTATTTATAAAGATAAGAGGTTTAATCAGCTCACAGTTCTG
CTGCCTGTACAGGCTTCTCTTGTGGAGGCCTAAGGAAACTTACAGTCATGGTGGAAGGTGAAGG
GGAAACAAGCACAGTCTTCACATGGCCAGCAGGAGAGAGAGAGAAGGGGGAAGTGCTACATACT
TTAAAACAACCAGATCTTGTGAGAACGCTTATCAGGAAACAGCACTTGGGGATGGTGCTAAATC
ATTAGAAATCACCCCCATGATCCAGTCGCCTCCTACCATGCCCACCTCCAACACTGGGGATCAC
AATTCAGCATGAGATTTGGGTAGGAACACAGAGCTGCACCACATCAGAGGATGTACAAGATTGT
GGTGGAGAGGAGTTTAGAGACCTGCAAATATAGGGTAATTGAAGGGATCATCTACATGGATATT
TAAATCACCAAAAATTATGACAGGAGTAGTGTTGGAGAGAGAACTGCGATGTAAACATTAAGGA
ATGAGGAAGAGTGACTCGGTAGGCTGTAGGTGACTGCAATAGGAAACGATAATAGACTGTGAGT
CTGGTGACAAGATTTTCCTTCTTTCTTTTTTTCCCCCCCCCCGAGACAGGGCCTCTTTTTGTTG
CCCAGGTGGGAGTGCAGTGGCGCGATCACGGCTCACTACAACCTCCTCCCAAGCTCAAGGGATT
CTCCCACTTCAGCCTCTCAAGTAGCTGGAACTACAGGTGCTGACCACCATGCCTGGCTACTTTT
TGTCAGGATTTTCAAGGCTGGGAATTTTGAGAGGGGAATGGAGGAGAATAATCTGAAAGTGCAA
GTAAGGAGCAGGGAAGATTTCTTTTTTCTTTTTTTTTTTTTTTTTTGAGTCGGAGTCTGGCTCA
GTCGCCCAGGCTGGAGTGCAGTGGCGAGATCTCCGCTCACTGCAAGCTCCGCCTCCCGTGTTCA
CGCCATTCTCCTCCTTCAGCCTCCCGAGTAGCTGGGACTACAGGCGCCCGCCACCACGCCCAGC
TAATTGTTTTTTTGTATTTTTAGTAGAGACGGGGTTTCACCGTGTTAGCCAGGATGGTCTCAAT
CTCCTGACTTTGTGATCCGCCCACCCCGGCCTCCCAAAGCGCTTGGGATTACAGGCGTGAGCCA
CCGCGCCAGCCAGAGCAGGGAAGATTTCTTCCCCACATCTCCAGTAGGTACAGTGATATGAAGT
GTGTGGAGGAGAAAAGAGGAAACATCTATCATTTGAGATGGCTGCGAAAGGAAAAGGCATCCTC
AGGGAGCTAGATTTTACTTAGAGCAAGAAATGAAGGGATGATTCAGAGGTTAAAAGAGTGGATT
TTATGAATTACTCAAGGGAGCACAGTGGAAGTTTCAGGAAGTGGTAGGAGAAGGTAGAAGATGG
CAGGGTGTTGGGAATAATTTGAGAAATCTGAGCTACTGGAAATGACTGAGAATCAGATATAAAG
GCAGTCCTGGTGGTCCGTTCTGGCTGCCGTTGCTGTGTAACGAATCTGCCAAAACTTAGTGGCT
TGAAACAACAAAGAACATTTTATTATCTCTCATTGTTTCTGTGGGTTAGGAATTTGTGAGAGCC
GTGCTGGGCAGTTTTCGTGCGGCTGTCTCGTGGTTGCACCTACATAGTTGCTAGAGCTACAGTA
GCTGGGGACTGAGCAGCTAGGGATTGGCAGGCTATCTCTTTTTTTCATGTAGTCTCATGAAGAT
TTCTTTATGTGGTTTCAATGTGTGGGCTGGTTTGGATTTCCTTATAGCATGGTGGCCTCAGTTG
GATTGCTGTTTTGTGATCCTTTTCATCCCTCCTTGTCCTGTCCCCAGACAACCACTGATCTACT
TTCTGTCACCATAGATTAGCCTGCATTTTTAAGAATTTTTATAAACGTGGAATGATAGAGTACC
TTTTTTGTCACGTTTCTTTTATTTATCATAGCTATTTTGATTTTCATCCATTTTATTGCTGAGT
AGTATCCCATTGCATGTATATACTATACTGTATTCATTCGCTTGCTTGTGAACATTTGGGCTTT
TTCCAGTTTGGGACTGTTAACAAGTAGAGCCACTATGAATATTAGTGTATAAGACTTCATATAG
CCAAGGCTGGCAGATCGCTTGAGCCCAGGAGTTTGAGACCAGCCTGGGAAACATGGTGAAACCT
CTATTTTTATTTTAAAATCAAAAATTAAAAATTTTCTATAAAAAATTTTAAAGAAGACTTTGTA
TAGACATACGCTTTCATTTTTCTTGAGTGAATACTTAGGTCTCAGGGTAGATGTATTTTAAGTC
TTTAAGGAGCTGTCAAACTCTTCCTCAAAGTGGTGGTTGTACCATGTTACTTTTTAATATAACA
GAGATTAATTGAGCAAAGAAAAATTCAAAAGTTGGACAGCCCCCACAACTAAATAGGTTCAGAA
CAGCTCCCCCATTTTGCATTTTGACCAGCAATGTATGAAAGTTCCATTTGCTCAGTGTCCCTGC
AAACACCTGGTATGGTCAGTCTTTTTAATTTTAGGCATTATAATAGATATAGTGGCTTCTTGTG
ATTTTAATTAGCATTTCCTAATGACCAGTGCTGCTGTTGATCATTTCATGAGTGTATTTGCCAT
CCGTATATCTTTTTTGGTGAAGTGTCTATTCAAATCATTTGGGTTTTTTTTTTTTTTGTTTTTT
TTTTTTGGAGACAGTGTCTCACTCTGTCACCCAGGCTGTTGTGCAGTGGTGCAATCACACAGCC
TACTGCAGCCTCCACCTCCTGCGCTCAGTCTTCTTGTCTCAGCCTTCTGAGTAGCTGAAATTAC
GAGCACACGCCACAATGCCTGGCTAATTTTTTAAAATTTTGTAGAAACAAGGTCTCATTATGTT
GCCTGGGCTTGTCGTGAACTCCTGGGCTCAAGCAATCTTCCTGCCTCAGCCTCCCAAAGATTGG
GATTGCAAGTATGAGCCACTGCACCCGGCCAACTTACCCATCTTTTAATTGAATTTTTTTGTTG
TTGAGGTTTGAGAGTTCTTCATGTTTGCTGGGTACAATATCTTTATCAGATAGGTAACTTGCAT
GTATTTTCTCCCGGTTTACACTTTGGTTTTTCATTTTGTTAACAACGTCTTTTTAAGAACAGAA
AATCTTAATTTTGCTGAAATCTAATTTTTCAGTTTTTTCTTTGATGGTTTTGAGAGAGGAGGTA
AAAAAAGACTAGGTAAGCCGATAGTTAGACAGAGTCCTCGGTAGAACTTCCCTTCTAACAAAAA
GCAGCCCAAGAAATCACTTCTCTTCTAACAAGGAGCAGCCTGGAAGATCGGGCTGTAAACATGT
ATAAGGAAGCAGCTCTGGCACAGAGGGGGAGCTTCCTGGGTAATCAGCAAGCTTCACATACGTA
AGGTGGGTATGTGAAGTAAACACAGTATGTGAAGTAAACACAGTGGACCTTAGTACATACTCAG
ATAAGGAAGCTGGAAGCTTGCATGTTGTGAGTTGTTGGGGTTGCCTGCAGCTGCACGGAGAGAA
AGGGGTACCTGGGGCCAGGCATGTCCACCATGGTGGCTCCACCTCCCCTTATTTAGCACATGCA
CAATAGGAAAGAGATAAGCAATGTGGAGTAGCTCAGGCCAAGGACCTGCCTGCATAATAAAAGG
TTGGGGTGGGGGATGCCAGAGATTCACGCTCTGTGCAGATGGCAACACCTGGTCCTAACTGGTT
TTTTGCTCCCTATGTGTAGATAAGCTACCCCCTTCCCATTAGCTCATTTATAAAAATGCTTGCA
TTTCACTGTGGAATGGGAACTCTTTTCAGGACCTCTCTCTGCAGGAGAGAGCTAGTCTCTTTCT
TTTGCCTATTAAACTTCTGCTCTAGCCTCACACCCTTGGTGTGTCAGCGTCCTTGATTTCCTCA
GCGTGAGACCAAGAACCTCGGGTGCCACCCCAGGCAACAAGGCCATTTCAGTTTGTTCTTTTGT
TATAGGCAATCCATGATCACAGATTTTTCTCTCTTTTTTTTTTTTACACAGTTTAGAGTTTTAG
TTTTACACTTAGGTCTGTAATCCATTTTGTATTAATTCTTATATGTGGCTCAGTGTAGGTGGAA
ATTTGGTTTGTTTTTGCATAAGGATTTCCAATAGTTTTACCACCATTTCTTGAAACTACTATGC
TTTCTCTATTAAACCACATTTGTAACTTTAGTTAAAATCAGTCACATATATCACAGGGCTATTT
CTGACTCTCAATTCTGTTACATTGTCTATTAGTGTATATTGATGTCAGTACTACACTTTTAATT
ACTATTGCTTCAGGGTATGTCTTGTAAACCAAAAATAAAATTATAGGCCCCCCCCGCCCCTGCA
CAACCAACTGAATGGACCCATCCTCTCAGCCAAGGGCATTCCAAAATTAACCTGAAAAACTAGT
TCAAGCCATGATGGGAAGGGGGAGTTGGACATGTCTCATCACACCCTACTACCTTTTGGAATTA
CTGATAGAACAGACTCTTAAAGTCTGAAAAGAAACATTTACAACCTACCCTCTCTGAAGCCTGC
TACCTGGGAGCTTCATCTGCATGATAAAACCTTGGTCTCCACAACCCCTTATGGTAACCCAAAC
ATTCCTTTCTGTTGATAATAACTCTTTCAACTAGTTGCCAATTAGAAAATCTTTAAATCTTCCT
ATGACCTAGAAACCTCCCTACCCCCACTTTGAGTTGTCCTGCCTTTCCTGACAGAACTCATGTA
CATCTTACATATATTGATTGATGCCTCATGTCTCCCTAAAATGTATAAAACAAAGCTGTACCCC
ACCACCTTGGGGACATGTCATCAGGACCTCCTGTGGCTGTGTCATAGGAGCGTCTTTAACTTTG
GCAAAATAAACTTTCTAAATTGATTGAAACCTGTCTTAGCTACTTCTGGTTTACAGTCTTAAAG
TTAGATAATGTAAATTGTCCAGCTTTGGTTTATTTTTGTCCTTAGTAGTTCCATATAAATTTTA
GAATCAGCTTTTCAATTTAATACACTACTTTCCTCTTAGATCCACAATTAAATATATTTGATGC
TAACAATTCTGTTTTATGTTTTTCGTTTTTTTTTTTTGAGACAAGAGTTTCGCTCTTGTTGCCC
AGGCTGGAGTGCAGTGGCGCGATCTTGGCTCACCACAACCTCCACCTCCCAGGTTCAAGCAATT
CTTCTGCCTCAGCCTCCCGAGTAGCTGGGATTACAGGCATGCGCCACCACGCCCGGCTAATTTT
GTATTTTTAGTAGAGACGGGGTTTCACCATGTTGATCAGGCTGGTCTTGAACTCCTGACCTCAG
GTGATCCACCCACCTCGGCCTCCCAAAGTGTTGGGATTACAGGCGTGAACCACCATGCCTGGCC
AGTTCTGTTATTTTTAAAACCCAAGTTTCCCTGGTCATATCTTGGTTGGATGAAGCGTATTTTC
AATAGATTACCCTGGAAAGGCTAGTGAGTACGGTATTCTTCTACATTTTAGACTTTTCTTAGTC
TTGCTACTTCAAGGACAGCTAGGCTGCATATAAAATTCTTGGCTCATACTTTTTCCCCATAAAT
TTCTATGAGAAAGTCTAATGATAACTGATTTTCTTTATTTTGTAACTTAGTCTTTTTGCTTAGA
GGCTCTCTGAGGATGGGAGGGGGTTCTTCCTCCCATCCCTAGGAATTTTTCTTTTTTTTAAATT
CCTAATCACTAGACCACCAGGAAGATTGTTTGTTTTGTTTTGTTTTTATTCTTCAGGGACCCCA
TTTATACATACGTTAAATAAATACTGTTTGCCAATGTATCAACCATTTTGCTTCTTATTTATTT
TTGTTCCTTTGGTTCTTTTTCATGGCTTTGCTTTGGTGCTCCTTAGATTTTCAGTCAGATGTAT
TTGTCCTTGGGTACCTTGTAATCAGTATTACCTTTTCTTCTGTCGCTTTGTTTTCTGTTCGTTT
TGAAATTACTTGTTTCCTGGTCTGGCAATAACAGTTGAGATATGAGGAGTTTGAGCTGCCATCT
GTCTATGTATCTTGCTTTAAGACTGCACTCTTCTATTGATATCACTGGCCTTGATTTTGTGATT
TCTTTATTTCTTCAGGACCACCCTTCATTTTCTACTGTTTGCTTCCTTTTTTTTTGAGATGGAG
TCTCACTCTGTCACTCAGGCTGGAGTGCAGTGATCTTGGCTCATTGCAACCTCTGCCTCCCGGG
TTCCAGCAATTCTCCTGCCTCAGCCTCCCAAGTATCTGGGACTACAGGTGTGCACCACCATGCC
CGGCTAAGTTTTGTATTTTTAATAGAGACGGGGTTTTGCCACATTGGCAGGCTGGTCTCAAACT
CCTGATGTCAAGTGATCCACCCACCCCACCCACCTCTGCATCCCAAAGTGCTGGGATTACAGGA
ATGAGCTGCCGTGCCCAGCCTCCCCCCTACCCCCCTTTTTTTCTTTCGAGACAGAGATTATAGG
TGTGAGCCACTGGACCCAGCCTGTTTTTATTCCTTTTACCAAATCTCCAAGGAATATCTTCCCT
TCCAAGTGCGAATGTAACCTTAAGTCAGTTAACCTCTTTGTGATTACTTTTCTTATCTGCAAAG
TGACTTAATGATCTTAAGTACTTTTTTTTTTTGAGACAGGGTCTCACTGTCACCCTGGCTGGAG
TGCAGTGGCACGATCTCTGATCTCCACTCACTGCAATCTCCTCTTCCCTGGTTCAAGCGGCCCT
CCCACCTTAGCCTTCTGGGTAGCTGGGACTACAGATGTGAACCACCACGCCCAGCTAATTTTTG
TACTTTTTGTAGAGATGGGGTTTTGCCATGTTGCCCAGGCTGGGATTATTAAGTACTTTTTATC
ATACAGCAAGATTGACATTTTATATTGGAATACATTTGTCTCTATATAACGGAGATTAACAGGA
AAATGACAAGCCTGGGTGCGGTGGCTCATGCCTGTAATCCCAGCACTTTGGGAGGCTGAGGTGG
GAGGATCACTTGAGGTCAGGAGTTCGAGACCAGTTTTGCCAAGATGATGAAAGCCCATGTCTAC
TAAAAATACAAAAATTAGCCCAGCTTGATGGTGGGCGCCTATAATCCCAGCTATTTGAGAGACT
GAGGCAGGAGAATCACTTGAACCTGGGCGGCAGAGGTTGCAGTGAGCCGAGATCATGCCACTGC
ACTCCAGCCTGGGTGGCATAGCGAGACTCTTGTCTCAAGAGAAAACAAAACAAAACAAAAAAAA
AACAGGAAAATGACAAAAAGTAATATTACAACTCAGTGAATTTTATAACAAACTTTTTTGGAAT
TCATTGACTAATACTATACCAAATCCAAAATACTCTCTAGTATACCAAATCCAACTCTACCCTA
TAGTATAAATTGGATTCTATTTGGACTTGTCTCACTAATCCCTCATACAGTGTGTTTTATTTTT
TATTGAAGTAAAAAAATTTGTCATTTTAACCATTTTTAAGTATATAGTTCAGTAATATTAAGTA
TGTTCATGTTGTTGCGCAATAGATCTTCGGAAGTTTTTCGTCTTGCAACCTGAAACTCTACCCA
TTAGCAAATTCCCATTTCTCCTTACACTTAGCCCTTGGTAATCATCATTCTTTTTTTTTTTTTT
TTGAGATGGAGTTTTACTCTTGTTGCCCAGGCTGGAGTGCAATGGTGCAATCTCGACTCACCAC
AACCTCCGCCTCCCAGGTTCAAGCAATTCTACCTCAGCCTCCCGAGTAGCTGGGATTACAGTCA
TGCACCACCACGCCCGGCTAATTTTGTATTTTTAGTAGAGAAGGGGTTTCTCCATGTTGAGGCT
GGTCTCGAACTCCTGACCTCAGGTGATCTGCCCACCTCGGCCTCCCAAAGTGCTGGGATTACAG
GCGTGAGCCACTGCGCCTGGCCCATTCTTTCTAATTCTATAAATTTGACTACTTAGTTACCTTA
CATAAATAAATTCTTATAGTTAGTGTTATTTTTGCTTCCATGCCTTTTTTGTTGTTGTTCATGC
TCTTACTTGGAATGCGTTCTATTTTGTCTACCTATGCACATCCTGTTGGGTTTTTTTTTTTTTT
GGGGGTTTTTTTTGTTTTTTTTTGTTTTTTTTTCCCAGACAAGGTCTCAATTTGTTACCCAGGC
TGGAGTGCAGCGGCGCCATCTCCACTCACTGCATCCTCAACTTCCTGGGCCCAGGTGATCCTCT
CGCCTCAGCCCCTGCAGGTAGCTGGGACTATAGGCATGTGCCACCATGCCCAGCTAAATTTGGT
TTTTTTGTTTGTTTGTTTTTGAGACAGAGTCTCACTCTGTCACCCAGGCTGGAGTGCAGTGGCA
CAATCTCAGCTCACTGCAATCTCTGCCGCCCGGGTTCAAGTGATTCTCCTGCCTCAGCCTCCCA
AGCAGCTGGGATTACAGGTGACTGCCACCACGCCAGCTAAGTTTTGTAGTTTTAGTAGAGATGG
GGTTTCACCTTGTTGGCCATGCTGGTCTCGAACTCCTGACCTCGTGATCTGCCTGCTTCTGCCT
CCCAAAGTGCTGGAATTACAGGCATGAGCCACCACGCCCGGCCAGAATTTTTGTATTTTTAGTA
GACACAAGGTTCTTACCCTGTTGCCTAGGCTGGTCTGGAAGTCCTGGACTCAAGCAATTCACCT
GCCTTGGCCTCCCAAAATGCTGGGATTACAAGCCACCATGCCCGGCCTAAATCCTGTTGTTTTG
TTTTGTTTTATTTTGTTTTGTTTTGTTTTGTTTGTTTTTTGAGACAGAGTCTCGCTATGTCTCT
CAGGCTGTAGTGCAGTGGCGCGATCTTGGCTCACTGCCACCTCTGCCTCCCAGGTTCAAGTGAT
TCTCCTGCCTCAGCCTCCCAAGTAGCTGGGATTACAGGCATGTGCTACTATGTCCGGCTAATTT
TTGTATTTTTAGTAGAGACAGGGTTTCACCATGTTGGCCAGGCTGGTCTCGAACTCCTGACCTC
GTGATCCACCCACCTCGGCCACCCAAAGTGCTGGGATTACAGGCGTGAGTGGTTTTTATTTCTT
AGGCCGGTTTCCTCCATATGATCTTGCAGTAGACATTAATTTCTTTCCTTTTTAATTAAAATAC
TGTTTGTATTTCACATTTTGATGTTTGTTAAGATTTGTTTTATATTGTTTTTTGTTTTGTCTTG
TGTGATAGTCTTAAATCCCTAGTTAGATAATAACTGGAGAGTACCATGTTTCTATATATCTCTC
AGTGACTTGCACAGTGCTAGCAGATAGTGCTAAAAAATTATTTATTATTATTATTATTTTGTTA
TTGTTGTTGTTGTTGTTAGACAGGGTCTTCCTCTGTCACCCAGGCTAGAGGGCAATGGGATGAT
CATAGCTTACTGCAGCCTCCAACAACTGGGCTCATGTAATTCTCCTGCCTCAGCTTCCCAAGTA
GCTGGGATTACAGGCATGAGCCACCATGTCTGGACAAAAATATTTCCAGGTGCAGTGGCTCATG
CCTGTAATTCCCACACTTGGGAGGCCGAGCGAGGCTGGAGGATCACTTGAGCCTAGGAGTTCAA
GACCAGCTTGGCTAAGATGGCGAGACCCCGTCCCTACAAAAAATTTTAAAAACTAGCCAGGCAT
GGTGGCATGCACCTATATTCCCAACTACTCAGTGGGCTGAGGTGGGAGGGTCATTTGAACACAG
GAATTTGAGGGGAGAAAAAAAGAAGAGAGAAAGAGAAGTGAAGGAAGGAAGAAAGGAAGGAGGG
AGGGAGAGAAGAAAGAAACGAAAGAAAGGAAAAGAAAAGGAAGGAAAGAAAATTGGTACCAGGA
AAGCAGGAAAGGGAAATGGAAGTAAAAAAATAATAATAATAATAAAATGAAAATTGGTTAGTCA
CTATTAACAATTTGTATCCTTATAATCTGGAAACATTATAATTTCAAAAGAAAAAATATTCTTT
GGATCATAGGTTCTGAGGTCAGAACAGCATTCCCGTAGTCTAGATGAAGTCAAGTTTTATCTGA
TCTTAATTGAAATAAATATAGCTGGCCTTGAACAAATCTACTCATGGTATGTGGATAGGAATTA
AATTGTAGGGGCATTCACTTGATGGCATTCATTCTTAGAACATTTACCTATGTCTAGCTTTTGG
AGTAAAGTCACATAACCTCTAACCAGGTAAGTTTCCTGTGGCTTTATTTAGGATTTTAAATACT
CATTTTCAGTGTAATTTTGTTATGTGTGGATTAAGATGACTCTTGGTACTAACATACATTTTCT
GATTAAACCTATCTGAACATGAGTTGTTTTTATTTCTTACCCTTTCCAGAGCGATGATTCTGAC
ATTTGGGATGATACAGCACTGATAAAAGCATATGATAAAGCTGTGGCTTCATTTAAGGTATGAA
ATGCTTGCTTAGTCGTTTTCTTATTTTCTCGTTATTCATTTGGAAAGGAATTGATAACATACGA
TAAAGTGTTAAAGTACATGTTATTCAGTTTTCATTTTGAAGATTAGATGGTAGTATGAGTTAGT
TAAATCAGGTGATATCCTCCTTTAGAAGTTGATAGCCTATATATGTCATCCTTTGTGGAGGCAA
TTTAAATAAAATTTAAAACATTTATTCCTGGCTGGGTATGGTGGCTCACTCCTGTAATCCCAGC
ACTTTGAGAGGCTGAGGCGGGTGGATCACCTGAGGTCAGGAGTTTGAGACCAGCCTGGCCAACA
TGGTGAAACCCCGTCTTTACTAAAAATACAAAAATTAGCCAAGCATGGTGGCACGTGCCTGTAA
TCCCAGCTGCTTGGGACACTGAGGCAGGAGAATTGCTTGAACCTGGGGGGCAGAGGTTGCAATG
ATTGCACCACTGCACTCCAGCCTGGGCGATAGAGTGAGACTCCATCTCAGAAAACGAACAAACA
ATGTATTCCTTTTAGTATTTTTACATTGTATCAAACTATGGAAGTCCTCTAATTGAGATTAATA
AGAAAAAGACAATCTGAATTATAATTTTAAACATTTAACAAGCATGTAGTAAAATAATGATGAA
GATAAATAGCATTAGTACAGCAATTAATATTTGTAGCATGCTGACAGTGCTCTGTGTGCGTTTC
ATATATTAAATTACTCTAATCATCCCAAATCCTGTAAGTTGGGTATCAATTCAAGTGTTCCTAT
TGGGTAGGAATATACAGTTCTTTTAGGAAATGTAGTATGGTTCTGTGTCTCAAACAGGACACTT
ACACAGTTGGCCAACATCATCACCTTCTCCATTCTCTGAGATGTTTAGTCTTACTGAGCACTAA
ATATGGGTCATCAATAGTCCAGACTACCTTGAGCAAACAATAGTCCAGACTACCTTGAGCAAAC
AGAGCATATACTCATACAGTGTATAAAGAGCACCAAGCATACAGATTTCATGTCTTTCTCATAG
TTACTCTTGTAACATGAGCTAAAGATCAGACCTCTATGTCACCTTTGTAACTGATTTCTAGATT
TTTTTTTTTTTTTGAGATGGGGTCTTGCCCTGTCACCCAGGCTGGAGTGTAGTGGCGTGATCAT
GCCTCATTGGAGCCTTCAACTCATGAGCTCAAACAATCCTCCTACCTCAGCTTCCTGAGTAGTT
GGGACCACAGGTGTGTGCCACCACACCCAGCTCATTTTTGTATTCTTTGTAGAGATGCAGTCTC
ACCCTGTTGCCCACGCTGGCCTGGAACTCCTGAGCTCAAAAGATCCCTCCGCCTTGACCTTCCA
AAGTGCTGGGATTACAAGCATGAACCACTGCACCCGGCCTAGATTTTTAAATGTGCTTTCCAGT
ATACACTGAAACTAGAAGTCGACTAAAGAATTACCAAGAGAATTCTATAAAATAGAGATTGAAA
TGGGGCTCGATGTGGGATGGGTTGGTGATATTGCAGGGAGAAGTAATCTGAGTAAAGGAGGAAA
AGAACTGATTTGGGAAAACGATAGTTTTAGTAGTGAGTTTGAGTATGAATTAAGTTGAGATTGA
ATTTGAATTAAGTTGAGGTTGAATATGAATTAAGTTGAGGTTGAGTTTGAGGTATGAATTAAGA
TGTGAAATTGATCATTGGAAATGTTAGATTGAGAAAAGTCACAGCTGGATTAATAGCTTCAGAA
GTGTGTTTGCAGACAGTTGCAACTAAAGTAATAAGAATAGATGGCCTTGGCCGGGCGCGGTGGC
TCACGCCTGTAATCCCAGTACTTTGGGAGGCTGAGGCGAGCAAATCACGAGGTCAGGAGTTCAA
GACCAGCCTGGCCCACATGGTGAAACCCCGTCTTTATTAAAAATACAAAAATTAGCTGTGCACA
GTGGTGCACGCCTGTAATCCCAGCTACTCGGGAGGCTGAGACAGGAGAATCGCTTGAACCTGGG
AGGTGGAGGTTGCAGTGAGCTGAGATCAGTGTGACTGCACTCCAGCCCGGTGACAGAGTGAGAC
TCTGTGTAAAAAAATAAAATAAATAAAATAATGGCCGTAAGCAAGTAAAGAAGGATGGCCAGCT
CTTATTGGGAATGCCTAAATCTAAGGCTTGATCAGAAGTAATGAAACCGTTGGGGCCCTACATT
GCTATGACATCCAAAGGGCCATGAATATCAGGAAGAAAGATAATTAACAGGGTCTAATGTTACA
GAGAGGTTGAGAGCAAGGAGATTTGATTAAAAGGGTCTTTAGAGCTGATGTCAGGTGTATGATG
CCTTTAAGAGCAGTTTTTATAGTGCAGGGGGTGGTCAAAAGAGAAAATAGGTGCTTTCTGAGGT
GACGGAGCCTTGAGACTAGCTTATAGTAGTAACTGGGTTATGTCGTGACTTTTATTCTGTGCAC
CACCCTGTAACATGTACATTTTTATTCCTATTTTCGTAGCATGCTCTAAAGAATGGTGACATTT
GTGAAACTTCGGGTAAACCAAAAACCACACCTAAAAGAAAACCTGCTAAGAAGAATAAAAGCCA
AAAGAAGAATACTGCAGCTTCCTTACAACAGGTTATTTTAAAATGTTGAGATTTAACTTCAAAG
GATGTCTCATTAGTCCTTATTTAATAGTGTAAAATGTCTTTAACTTAAGTGATTAGTACAGTGT
TTCTATTGACATATACTTATACAACTTCAAAAACAACTATTAAATTTTCTGTTATTTAGGAACA
TGCATATTAGTCATGAAAGTATAAAGAATTAGATGGGAATGATAAATGCTAAAATCAGGACATG
TGTTCCATTTGTGAATGGAAGGCAGGGAGAAGGTGCCGTTTGGAAGGAGTACCCAAGAGCCGTA
AGCTGAATTGGCAGTGTTTTACATCTTAAGCTGAGAGATAGATTTTTTTTTCCCCTTTTTCTTT
AAAAACTCTAAAACTGTTAATTCCAAGGAACCCAGAAGTCTAGGTAGATTATTTCTGCTAGTTA
AAAGCAGTAGTCCTGAAAGCTGAATATTTTGGTGTCTTTTGAGCCAACTTTAGTTTCATCATTA
CCAAGGGGGAAGAGAGCTAACAGTTGATGAGCACTTGCTCTAGGCCAGTCCAGAGTGCTGGGCA
CCATACGCATTTTATCTCCCTCCCGCTATTCACAACAAATATGGGAGGTAGTTTATATTATAGC
CATCTAATAAGATGGGGAAACTAAGACTCAAAGAGATTCAGAAACTTGTCCATGATTATAAATG
TAAGAGAGTTGGAATTCAGATTTATGTATTTAGACCCCAAGCCTTTCTCATTACATCATTTTGC
CTTCCAAATCTCTACCCTCTATCCTTCACCTCCCCACTGATCAAAACGAGATGATAGTTTGCCC
TCTTCAAAAGAAATGTGTGCATGTATATATCTTTGATTTCTTTTGTAGTGGAAAGTTGGGGACA
AATGTTCTGCCATTTGGTCAGAAGACGGTTGCATTTACCCAGCTACCATTGCTTCAATTGATTT
TAAGAGAGAAACCTGTGTTGTGGTTTACACTGGATATGGAAATAGAGAGGAGCAAAATCTGTCC
GATCTACTTTCCCCAATCTGTGAAGTAGCTAATAATATAGAACAAAATGCTCAAGAGGTAAGGA
TACAAAAAAAAAAAAATTCAATTTCTGGAAGCAGAGACTAGATGAGAAACTGTTAAACAGTATA
CACAGTTGTCAGTTTGATCCACCGAGGCATTAATTTTTTCTTAATCACACCCTTATAACAAAAA
CCTGCATATTTTTTCTTTTTAAAGAATGAAAATGAAAGCCAAGTTTCAACAGATGAAAGTGAGA
ACTCCAGGTCTCCTGGAAATAAATCAGATAACATCAAGCCCAAATCTGCTCCATGGAACTCTTT
TCTCCCTCCACCACCCCCCATGCCAGGGCCAAGACTGGGACCAGGAAAGGTAAACCTTCTATGA
AAGTTTTCCAGAAAATAGTTAATGTCGGGACATTTAACCTCTCTGTTAACTAATTTGTAGCTCT
CCCATGAAACTTTTGTAGCTTAAATACACAAGAATTTTTTGAAAAGGAAATAAGATAATGATGC
AAAATAGTTAATTTTTTAAAAAAATGTTAGACACTGCAGTGGATGCAACAAAATACTTTATATG
AAAGATTTATCCAGTTAACTTTTGTGGAGTATTAGGTATTAGACTAATAATTAGCACACTTACT
TAAGTTAGAAAGTATAATAATGCGCCGGACGCGGTAGCTCACGCCTGTAATCCCAGCACTTTGG
GAGGCCAAGGTGGGCGGATCACAAGGTCAGGAGATCGAGACCATCCTGGCTAACACGGTGAAAC
CCCATCTCTACTGAAAATACAAAAAAATTTGCCGGGCGTGATGGCGGGCACCTGTAGTCCCAGC
TACTCGGGAGGCTGAGGCAGGAGGATGGTGTGAACCCCGGAGGCAGAGCTTGCAGTGAGTCAAG
ATCGTGCCACTGCACTCCAACCTGGGCGACAGAATGAGACTCCATCTCAAACAAAAAAACAAAA
CAAAACAAAAAAAAGTGTAATAATAATTTATCATTAGCTGGATGATATGCTGTTGTTTCCCATG
TCACCTGTATAAGATATGTAAAATAAGAACACATTATTTACATCTAATATAGATAAAATCCTGA
GGCGCTCTCAGATTGTTTTGTAGAGTTCAAATGTAAATATTGTTTTCATTTATGGTCCTTTTGG
TTATAAGTAACAGAAATCAACTCTAAAAAGATTTTTATTATAGGTTAGATTATGTCATGGAACC
TTAAGGCTTGTCCCTTTCTAGTTCTTTTGTGTAAAGCGGTGATTTCTTCCATGGAGGGAATGGT
ATTTAGGCAATTTTTTTTTTTTTTCGAGATGGAGTCTTGCTCTGTCGCTCAGGCTGGAGTGCAG
TGGCACCATTTCAGCTCACTGCAACTTCCACCTCCTGGGTTCAAGTGATTCTCCTGCTTCAGCC
TCCCAAGTAGCTGAGATTACAGGCACCCGCCACCACACCCGGCTTATTTTGTATTTTTAGTAGA
GATGGGGTTTCACCATGTTGGCCAGGCTGGTCTTGAACTCCTGACCTCAAGTGATCTCCCCACC
TTGGCCTTCCAAAGTGCTAGGATTACAGGCGCCTAGCCTAGGCAGTCATTTTCAAAAAACAAGC
ATGACTCACCAAAAGTTTTAAGATTTTCTGTGATAATGTTCTTATTGAGGCTTACATTATATTA
CAGTTTCTTGAATCTAAAATGATGTACCCTCTTAGGATATATACATCATGCTTCATTGGTCTCA
GGGGGCTGATTTTTATAAGGAGAGATTTGCTAGTTTTCACAATATGTCCTCTAAGTTGGCATGT
ATAGCTAAACAGGCTTTCATAAAAATATACAATTTAGTTAATGAAATTTGGGATATAGTCTTTT
ATGATTGAAATAATTTTGCTAAATAGACTGTCTCTGATTTATTAGGTAATCACCACTCTTATTT
TGTTTTACTTCCTTAATGTCTACATAGAAAGGAAATGAGAAAAATCCAGAGGTTGTCATTTGAC
TTATGAGTCTGTTTGACTTCAGGATTTGGTACATGAAATTTCACTTAATCTTTTTGATATGTAT
AAAACAAATATTCTGGGTAATTATTTTTATCCTTTTGGTTTTGAGTCCTTTTTATTCCTATCAT
ATTGAAATTGGTAAGTTAATTTTCCTTTGAAATATTCCTTATAGCCAGGTCTAAAATTCAATGG
CCCACCACCGCCACCGCCACCACCACCACCCCACTTACTATCATGCTGGCTGCCTCCATTTCCT
TCTGGACCACCAGTAAGTAAAAAAGAGTATAGGTTAGATTTTGCTTTCACATACAATTTGATAA
TTAGCAGAATAGAGGATTGTAAAATGTCATTGTAGAACATCCCTTGGGCCAGATTCTAATGGGT
AGAAATTTGAACTAAACCTCTGGGTTTTGTTTGTTTTTAATGCCTTTCTGTTACCCAGATGCAG
TGCTCTTGTAGTCCCAAGTCTAAGCTCTAGGTTGCCTTCTTTCCTGGCAGAAGTTGGTGTCTAT
GCCATAAGGAGGTAGTTCCTGTTAGAAGGGATTTAATTATACCTTATATAAGGAATTAGTGTTT
GCCCTTCTAGGTATAGTTGGATGTTAGCTTCTGATGTAAACTGGATTTCTTTTTCTTTCTCTCT
CTTTTTTTTTTTTTGTTTTGGAGGCAGAGTTTTGCCCTTGTACCCCAGGCTGGAGTGCAGTGGT
GTGATCTCAGCTCACAGCAACCTCCGCCTCCTGGGTTCAAGCAATTCTGCCTCGGCCTCCCAAG
TAGCTGGGATTACAGGCGACTGCCACCACACCCGGCTAATTTTTGTTTTATTAGTAGAGATGGG
GTTTCACCATGTTGGCCAGACTGATCTTGAACTCCTGACCTCAGGTGATCCACCCGCCTTGGCC
TCCCAAAGCGCTGGGATTACAGGCGTGAGCTGCCGCACCCAGCTGTAAACTGGATTTCTAATGG
TAGATTTTTAGGTATTAACAATAGATAAAAAGATACTTTTTGGCATACTGTGTATTGGGATGGG
GTTAGAACAGGTGTTCTACCCAAGACATTTACTTAAAATCGCCCTCGAAATGCTATGTGAGCTG
TGTGTGTGTGTGTGTGTGTGTGTGTGTATTAAGGAAAAGCATGAAAGTATTTATGCTTGATTTT
TTTTTTTTACTCATAGCTTCATAGTGGAACAGATACATAGTCTAAATCAAAATGTTTAAACTTT
TTATGTCACTTGCTGTCTTTTCGTCCTCGTTAAATTTAATTTTGTTGGTCTTTTGTTGTTATTG
GTTGGTTTTCTCCAAATGCTAGCTATGTTAAGAAATTTAAGGCCAGGTACAGTGGCTCATGCCT
GTAATCCCGGCATTTTAGAAGGCTGAGGCAGGAGGATCACTTGAGCTCAGGAGTTTGAGACCAG
TCTGGGCAACATAGCAAGACCTCGTCTTTGTTTAGGGGAAAAAAAAGAAATTTAAGTAGGAGAT
TATATAAGCAAAAATACAATTAATTTCCAGCATTCACTATATAATATAAATCTCCAGACTTTAC
TTTTTTGTTTACTGGATATAAACAATATCTTTTTCTGTCTCCAGATAATTCCCCCACCACCTCC
CATATGTCCAGATTCTCTTGATGATGCTGATGCTTTGGGAAGTATGTTAATTTCATGGTACATG
AGTGGCTATCATACTGGCTATTATATGGTAAGTAATCACTCAGCATCTTTTCCTGACAATTTTT
TTGTAGTTATGTGACTTTGTTTTGTAAATTTATAAAATACTACTTGCTTCTCTCTTTATATTAC
TAAAAAATAAAAATAAAAAAATACAACTGTCTGAGGCTTAAATTACTCTTGCATTGTCCCTAAG
TATAATTTTAGTTAATTTTAAAAAGCTTTCATGCTATTGTTAGATTATTTTGATTATACACTTT
TGAATTGAAATTATACTTTTTCTAAATAATGTTTTAATCTCTGATTTGAAATTGATTGTAGGGA
ATGGAAAAGATGGGATAATTTTTCATAAATGAAAAATGAAATTCTTTTTTTTTTTTTTTTTTTT
TTGAGACGGAGTCTTGCTCTGTTGCCCAGGCTGGAGTGCAATGGCGTGATCTTGGCTCACAGCA
AGCTCTGCCTTCTGGATTCACGCCATTCTCCTGCCTCAGCCTCAGAGGTAGCTGGGACTACAGG
TGCCTGCCACCACGCCTGTCTAATTTTTTGTATTTTTTTGTAAAGACAGGGTTTCACTGTGTTA
GCCAGGATGGTCTCAATCTCCTGACCCCGTGATCCACCCGCCTCGGCCTTCCAAGAGAAATGAA
ATTTTTTTAATGCACAAAGATCTGGGGTAATGTGTACCACATTGAACCTTGGGGAGTATGGCTT
CAAACTTGTCACTTTATACGTTAGTCTCCTACGGACATGTTCTATTGTATTTTAGTCAGAACAT
TTAAAATTATTTTATTTTATTTTATTTTTTTTTTTTTTTTGAGACGGAGTCTCGCTCTGTCACC
CAGGCTGGAGTACAGTGGCGCAGTCTCGGCTCACTGCAAGCTCCGCCTCCCGGGTTCACGCCAT
TCTCCTGCCTCAGCCTCTCCGAGTAGCTGGGACTACAGGCGCCCGCCACCACGCCCGGCTAATT
TTTTTTTATTTTTAGTAGAGACGGGGTTTCACCGTGGTCTCAATCTCCTGACCTCGTGATCCAC
CCGCCTCGGCCTCCCAAAGTGCTGGGATTACAAGCGTGAGCCACCGCGCCCGGCCTAAAATTAT
TTTTAAAAGTAAGCTCTTGTGCCCTGCTAAAATTATGATGTGATATTGTAGGCACTTGTATTTT
TAGTAAATTAATATAGAAGAAACAACTGACTTAAAGGTGTATGTTTTTAAATGTATCATCTGTG
TGTGCCCCCATTAATATTCTTATTTAAAAGTTAAGGCCAGACATGGTGGCTTACAACTGTAATC
CCAACAGTTTGTGAGGCCGAGGCAGGCAGATCACTTGAGGTCAGGAGTTTGAGACCAGCCTGGC
CAACATGATGAAACCTTGTCTCTACTAAAAATACCAAAAAAAATTTAGCCAGGCATGGTGGCAC
ATGCCTGTAATCCGAGCTACTTGGGAGGCTGTGGCAGGAAAATTGCTTTAATCTGGGAGGCAGA
GGTTGCAGTGAGTTGAGATTGTGCCACTGCACTCCACCCTTGGTGACAGAGTGAGATTCCATCT
CAAAAAAAGAAAAAGGCCTGGCACGGTGGCTCACACCTATAATCCCAGTACTTTGGGAGGTAGA
GGCAGGTGGATCACTTGAGGTTAGGAGTTCAGGACCAGCCTGGCCAACATGGTGACTACTCCAT
TTCTACTAAATACACAAAACTTAGCCCAGTGGCGGGCAGTTGTAATCCCAGCTACTTGAGAGGT
TGAGGCAGGAGAATCACTTGAACCTGGGAGGCAGAGGTTGCAGTGAGCCGAGATCACACCGCTG
CACTCTAGCCTGGCCAACAGAGTGAGAATTTGCGGAGGGAAAAAAAAGTCACGCTTCAGTTGTT
GTAGTATAACCTTGGTATATTGTATGTATCATGAATTCCTCATTTTAATGACCAAAAAGTAATA
AATCAACAGCTTGTAATTTGTTTTGAGATCAGTTATCTGACTGTAACACTGTAGGCTTTTGTGT
TTTTTAAATTATGAAATATTTGAAAAAAATACATAATGTATATATAAAGTATTGGTATAATTTA
TGTTCTAAATAACTTTCTTGAGAAATAATTCACATGGTGTGCAGTTTACCTTTGAAAGTATACA
AGTTGGCTGGGCACAATGGCTCACGCCTGTAATCCCAGCACTTTGGGAGGCCAAGGCAGGTGGA
TCACGAGGTCAGGAGATCGAGACCATCCTGGCTAACATGGTGAAACCCCGTCTCTACTAAAAGT
ACAAAAACAAATTAGCCGGGCATGTTGGCGGGCACCTTTTGTCCCAGCTGCTCGGGAGGCTGAG
GCAGGAGAGTGGCGTGAACCCAGGAGGTGGAGCTTGCAGTGAGCCGAGATTGTGCCAGTGCACT
CCAGCCTGGGCGACAGAGCGAGACTCTGTCTCAAAAAATAAAATAAAAAAGAAAGTATACAAGT
CAGTGGTTTTGGTTTTCAGTTATGCAACCATCACTACAATTTAAGAACATTTTCATCACCCCAA
AAAGAAACCCTGTTACCTTCATTTTCCCCAGCCCTAGGCAGTCAGTACACTTTCTGTCTCTATG
AATTTGTCTATTTTAGATATTATATATAAACGGAATTATACGATATGTGGTCTTTTGTGTCTGG
CTTCTTTCACTTAGCATGCTATTTTCAAGATTCATCCATGCTGTAGAATGCACCAGTACTGCAT
TCCTTCTTATTGCTGAATATTCTGTTGTTTGGTTATATCACATTTTATCCATTCATCAGTTCAT
GGACATTTAGGTTGTTTTTATTTTTGGGCTATAATGAATAATGTTGCTATGAACATTCGTTTGT
GTTCTTTTTGTTTTTTTGGTTTTTTGGGTTTTTTTTGTTTTGTTTTTGTTTTTGAGACAGTCTT
GCTCTGTCTCCTAAGCTGGAGTGCAGTGGCATGATCTTGGCTTACTGCAAGCTCTGCCTCCCGG
GTTCACACCATTCTCCTGCCTCAGCCCGACAAGTAGCTGGGACTACAGGCGTGTGCCACCATGC
ACGGCTAATTTTTTGTATTTTTAGTAGAGATGGGGTTTCACCGTGTTAGCCAGGATGGTCTCGA
TCTCCTGACCTCGTGATCTGCCTGCCTAGGCCTCCCAAAGTGCTGGGATTACAGGCGTGAGCCA
CTGCACCTGGCCTTAAGTGTTTTTAATACGTCATTGCCTTAAGCTAACAATTCTTAACCTTTGT
TCTACTGAAGCCACGTGGTTGAGATAGGCTCTGAGTCTAGCTTTTAACCTCTATCTTTTTGTCT
TAGAAATCTAAGCAGAATGCAAATGACTAAGAATAATGTTGTTGAAATAACATAAAATAGGTTA
TAACTTTGATACTCATTAGTAACAAATCTTTCAATACATCTTACGGTCTGTTAGGTGTAGATTA
GTAATGAAGTGGGAAGCCACTGCAAGCTAGTATACATGTAGGGAAAGATAGAAAGCATTGAAGC
CAGAAGAGAGACAGAGGACATTTGGGCTAGATCTGACAAGAAAAACAAATGTTTTAGTATTAAT
TTTTGACTTTAAATTTTTTTTTTATTTAGTGAATACTGGTGTTTAATGGTCTCATTTTAATAAG
TATGACACAGGTAGTTTAAGGTCATATATTTTATTTGATGAAAATAAGGTATAGGCCGGGCACG
GTGGCTCACACCTGTAATCCCAGCACTTTGGGAGGCCGAGGCAGGCGGATCACCTGAGGTCGGG
AGTTAGAGACTAGCCTCAACATGGAGAAACCCCGTCTCTACTAAAAAAAATACAAAATTAGGCG
GGCGTGGTGGTGCATGCCTGTAATCCCAGCTACTCAGGAGGCTGAGGCAGGAGAATTGCTTGAA
CCTGGGAGGTGGAGGTTGCGGTGAGCCGAGATCACCTCATTGCACTCCAGCCTGGGCAATAAGA
GCAAAACTCCATCTCAAAAAAAAAAAAATAAGGTATAAGCGGGCTCAGGAACATCATTGGACAT
ACTGAAAGAAGAAAAATCAGCTGGGCGCAGTGGCTCACGCCGGTAATCCCAACACTTTGGGAGG
CCAAGGCGGGTGAATCACCTGAAGTCGGGAGTTCCAGATCAGCCTGACCAACATGGAGAAACCC
TGTCTCTACTAAAAATACAAAACTAGCCGGGCATGGTGGCGCATGCCTGTAATCCCAGCTACTT
GGGAGGCTGAGGCAGGAGAGTTGCTTGAACTGAGAAGGCGGAGGTTGCGGTGAGCCAAGATTGC
ACCATTGCACTCCAGCCTGGGCAACAAGAGCGAAACTCCGTCTCAAAAAAAAAAGGAAGAAAAA
TATTTTTTTAAATTAATTAGTTTATTTATTTTTTAAGATGGAGTTTTGCCCTGTCGCCCAGGCT
GGGGTGCAATGGTGCAATCTCGGCTCACTGCAACCTCCGCCTCCTGGGTTCAAGTGATTCTCCT
GCCTCAGCTTCCCGAGTAGCTGTGATTACAGCCATATGCCACCACGCCCAGCCAGTTTTGTGTT
TTGTTTTGTTTTTTGTTTTTTTTTTTTGAGAGGGTGTCTTGCTCTGTCCCCCAAGCTGGAGTGC
AGCGGCGCGATCTTGGCTCACTGCAAGCTCTGCCTCCCAGGTTCACACCATTCTCTTGCCTCAG
CCTCCCGAGTAGCTGGGACTACAGGTGCCCGCCACCACACCCGGCTAATTTTTTTGTGTTTTTA
GTAGAGATGGGGTTTCACTGTGTTAGCCAGGATGGTCTCGATCTCCTGACCTTTTGATCCACCC
GCCTCAGCCTCCCCAAGTGCTGGGATTATAGGCGTGAGCCACTGTGCCCGGCCTAGTCTTGTAT
TTTTAGTAGAGTCGGGGTTTCTCCATGTTGGTCAGGCTGTTCTCCAAATCCGACCTCAGGTGAT
CCGCCCGCCTTGGCCTCCAAAAGTGCAAGGCATTACAGGCATGAGCCACTGTGACCGGCAATGT
TTTTAAATTTTTTAAATTTAAATTTTATTTTTTAGAGACCAGGTCTCACTCTATTGCTCAGGCT
GGAGTGCAAGGGCACATTCACAGCTCACTGCAGCCTTGACCTCCAGGGCTCAAGCAGTCCTCTC
ACCTCAGTTTCCCGAGTAGCTGGGACTACAGTGATAATGCCACTGCACCTGGCTAATTTTTATT
TTTATTTATTTATTTTTTTTTGAGACAGAGTCTTGCTCTGTCACCCAGGCTGGAGTGCAGTGGT
GTAAATCTCAGCTCACTGCAGCCTCCGCCTCCTGGGTTCAAGTGATTCTCCTGCCTCAGCCTCC
CAAGTAGCTGGGATTAGAGGTCCCCACCACCATGCCTGGCTAATTTTTTGTACTTTCAGTAGAA
ATGGGGTTTTGCCATGTTGGCCAGGCTGTTCTCGAACTCCTGAGCTCAGGTGATCCAACTGTCT
CGGCCTCCCAAAGTGCTGGGATTACAGGCGTGAGCCACTGTGCCTAGCCTGAGCCACCACGCCG
GCCTAATTTTTAAATTTTTTGTAGAGACAGGGTCTCATTATGTTGCCCAGGGTGGTGTCAAGCT
CCAGGTCTCAAGTGATCCCCCTACCTCCGCCTCCCAAAGTTGTGGGATTGTAGGCATGAGCCAC
TGCAAGAAAACCTTAACTGCAGCCTAATAATTGTTTTCTTTGGGATAACTTTTAAAGTACATTA
AAAGACTATCAACTTAATTTCTGATCATATTTTGTTGAATAAAATAAGTAAAATGTCTTGTGAA
ACAAAATGCTTTTTAACATCCATATAAAGCTATCTATATATAGCTATCTATGTCTATATAGCTA
TTTTTTTTAACTTCCTTTATTTTCCTTACAGGGTTTCAGACAAAATCAAAAAGAAGGAAGGTGC
TCACATTCCTTAAATTAAGGAGTAAGTCTGCCAGCATTATGAAAGTGAATCTTACTTTTGTAAA
ACTTTATGGTTTGTGGAAAACAAATGTTTTTGAACATTTAAAAAGTTCAGATGTTAAAAAGTTG
AAAGGTTAATGTAAAACAATCAATATTAAAGAATTTTGATGCCAAAACTATTAGATAAAAGGTT
AATCTACATCCCTACTAGAATTCTCATACTTAACTGGTTGGTTATGTGGAAGAAACATACTTTC
ACAATAAAGAGCTTTAGGATATGATGCCATTTTATATCACTAGTAGGCAGACCAGCAGACTTTT
TTTTATTGTGATATGGGATAACCTAGGCATACTGCACTGTACACTCTGACATATGAAGTGCTCT
AGTCAAGTTTAACTGGTGTCCACAGAGGACATGGTTTAACTGGAATTCGTCAAGCCTCTGGTTC
TAATTTCTCATTTGCAGGAAATGCTGGCATAGAGCAGCACTAAATGACACCACTAAAGAAACGA
TCAGACAGATCTGGAATGTGAAGCGTTATAGAAGATAACTGGCCTCATTTCTTCAAAATATCAA
GTGTTGGGAAAGAAAAAAGGAAGTGGAATGGGTAACTCTTCTTGATTAAAAGTTATGTAATAAC
CAAATGCAATGTGAAATATTTTACTGGACTCTATTTTGAAAAACCATCTGTAAAAGACTGGGGT
GGGGGTGGGAGGCCAGCACGGTGGTGAGGCAGTTGAGAAAATTTGAATGTGGATTAGATTTTGA
ATGATATTGGATAATTATTGGTAATTTTATGAGCTGTGAGAAGGGTGTTGTAGTTTATAAAAGA
CTGTCTTAATTTGCATACTTAAGCATTTAGGAATGAAGTGTTAGAGTGTCTTAAAATGTTTCAA
ATGGTTTAACAAAATGTATGTGAGGCGTATGTGGCAAAATGTTACAGAATCTAACTGGTGGACA
TGGCTGTTCATTGTACTGTTTTTTTCTATCTTCTATATGTTTAAAAGTATATAATAAAAATATT
TAATTTTTTTTTAAATTAGCTGTATCTGTGATTGTATTTCTTTTTTGCATATTATTTTGCCCTT
TGGCCCATATTTTGATATGGATGCCACCATAGCATTTTGTGTATGTGCATGTGTATTCCCACTT
AATGTCACATTTTTCATGTCTTTACATATTCTTATTTTTGTTTGTTTTTGAGACAGAGTCTCGC
TCTGCTGCCCACGCTGGAGTGCAGTGGTGCAATCTCAGCTCACTGCAACCTGTGCTATCCGGGT
TCAAGCGGTTCTCGTGCCTCAGCCACGTGAGTAGTTGGGATTACAGGCATGTGGCACCATGCCC
CACTAAGTTTTGTATTTTTAGTAGAGATGGAGTTTCACCATGTTGGCCAGGCTGGTCTCAAACT
CCTGCCCTCAAGTGATTCGACCACCCTGGCCTCCCAAAGTGCTGGGATTACAGCCGTGAGCCAC
CGCACACGGCCTCTCTATTTATTTCTATACATAGCTTTTCACATTATATTATGTTTATATATTG
TTTATATCTGTATTTCCTCTTTCATTAGAGAAAAGGTAGTACATCTTATTCTTCATGGTGTCTA
CAATATCTGGCAGTTTTTGGAAGTCAAGCGTGAGCTTAGAGCATAGACTGGTGGGATTGTCAAA
GAAGAGGGCAACTGGAAGAGAACTGTCAGTTATTTTTGGATCAGTCTTTAATTCATCATGACGG
GTTAGGCATTAGTTGTATTTCTTGCTAATTTTGAAGAAGACTTATTAACAAATCCTACATTAGG
TAAATGGTTTTGAAAGTTGAGTTAATCATAATGGTGTTTGACCTAGGACTATTTTTAGGCCCTA
TTTATCTTAATATCGAATAATGAAGCAGCTTCCCCCTTAGATATAGACAGAAAACATCAAAGCC
ACCACACTACCTGGCTGGATTTATCCTAGTAATAAAATCAAAACTGAGC.

TABLE 1
SMN1 and SMN2 splice junctions

| Exon | | 3′3′SJ | | 5′ SJ |
|---|---|---|---|---|
| No. | 3′ Splice Junction | SEQ ID | 5′ Splice Junction | SEQ ID |
| | (3′3′SJ) | NO | (5′SJ) | NO |
| 1 | — | — | CAG/gtgaggtcgcagcc | 436 |
| | | | a | |
| 2a | tcttaccctttccag/ | 428 | AAG/gtatgaaatgcttg | 437 |
| | AGC | | n | |
| 2b | ttcctattttcgtag/ | 429 | CAG/gttattttaaaatg | 438 |
| | CAT | | t | |
| 3 | tgatttcttttgtag/ | 430 | GAG/gtaaggatacaaaa | 439 |
| | GTG | | a | |
| 4 | tttttctttttaaag/ | 431 | AAG/gtaaaccttctatg | 440 |
| | AAT | | a | |
| 5 | aaatattccttatag/ | 432 | CCA/gtaagtaaaaaaga | 441 |
| | CCA | | g | |
| 6 | tttttctgtctccag/ | 433 | ATG/gtaagtaatcactc | 442 |
| | ATA | | a | |
| 7 | ttattttccttacag/ | 434 | gga/gtaagtctgccagc | 443 |
| | GGT | | a | |
| 8 | atttctcatttgcag/ | 435 | — | — |
| | gaa | | | |

Table 1 provides SMN1 and SMN2 splice junctions (see, DiDonato, et al., “Complete nucleotide sequence, genomic organization, and promoter analysis of the murine survival motor neuron gene (Smn),” Mammalian Genome, 10:638-641 (1999), the disclosure of which is incorporated herein in its entirety for all purposes). In the table, coding sequences are identified by capitalized letters and the end of the intron is indicated by a “/” adjacent to a three nucleotide-long non-intron sequence (e.g., a untranslated region and/or a coding exon sequence) to the right of a 3′ Splice Junction and to the left of a 5′ Splice Junction.

By “survival of motor neuron 2 (SMN2) polypeptide” or “survival motor neuron (SMN) polypeptide” is meant a protein or fragment thereof, having at least about 85% amino acid sequence identity to a polypeptide sequence provided at GenBank Accession No. AAH15308.1 and having small nuclear ribonucleoprotein (snRNP) biogenesis activity. An exemplary SMN1 polypeptide amino acid sequence from Homo Sapiens is provided below (GenBank Accession No. AAH15308.1):

(SEQ ID NO: 444)
MAMSSGGSGGGVPEQEDSVLFRRGTGQSDDSDIWDDTALIKAYDKAVAS
FKHALKNGDICETSGKPKTTPKRKPAKKNKSQKKNTAASLQQWKVGDKC
SAIWSEDGCIYPATIASIDFKRETCVVVYTGYGNREEQNLSDLLSPICE
VANNIEQNAQENENESQVSTDESENSRSPGNKSDNIKPKSAPWNSFLPP
PPPMPGPRLGPGKPGLKFNGPPPPPPPPPPHLLSCWLPPFPSGPPIIPP
PPPICPDSLDDADALGSMLISWYMSGYHTGYYMGFRQNQKEGRCSHSL
N.

In embodiments, the SMN1 polypeptide sequence is identical or virtually identical to the SMN2 polypeptide sequence and both of the polypeptide sequences are referred to as “survival motor neuron (SMN) polypeptide sequences.”

By “SMN2 polynucleotide” or “survival motor neuron (SMN) protein” is meant a nucleic acid molecule encoding an SMN2 polypeptide, as well as the introns, exons, and regulatory sequences associated with its expression, or fragments thereof. In embodiments, an SMN2 polynucleotide is the genomic sequence, mRNA, or gene that encodes an SMN1 polypeptide. An exemplary SMN2 nucleotide sequence from Homo Sapiens is provided below, where plain text represents untranslated regions, bold text represents introns, and italicized text represents coding regions (Ensemble Gene Accession No. ENSG00000205571). Splice junctions in the SMN2 polynucleotide sequence provided below are listed in Table 1.

(SEQ ID NO: 445)
ACTCTTAAGAAGGGACGGGGCCCCACGCTGCGCACCCGCGGGTTTGCTATGGCGATGAGCAGCG
GCGGCAGTGGTGGCGGCGTCCCGGAGCAGGAGGATTCCGTGCTGTTCCGGCGCGGCACAGGCCA
GGTGAGGTCGCAGCCAGTGCAGTCTCCCTATTAGCGCTCTCAGCACCCTTCTTCCGGCCCAACT
CTCCTTCCGCAGCCTCGGGACAGCATCAAGTCGATCCGCTCACTGGAGTTGTGGTCCGCGTTTT
TCTACGTCTTTTCCCACTCCGTTCCCTGCGAACCACATCCGCAAGCTCCTTCCTCGAGCAGTTT
GGGCTCCTTGATAGCGTTGAGTGGAGGCCCTGCCGCGACTTGGCAGTAGCTTATTTTGTTCACT
CCTCTCTGGCTGGTGTGGGGGAGGTGGGGGCATTAGGCCAGGGTGAAGCAGGGGAACCACTTAG
GAGTCTGTTAAGATGATCTGAACTTCAGAACAAGATGTTATTAACAGAGTGAAAGTATTTGGAT
TCTGGGTATATTTTGAAATCGGAGGCAACAGGTTTTTCAGATAGATTCGATAACGGAGGTTATC
CTGAATAGTTGAAAAGATAAAGTTGCCTTTTGCTGAGGTGGGAAAGAGAAGATTGCCAGTAGAG
CAGGTTTCTCAGGAGTTCAGTCTTGGGCATAGCATGGTAGGGGTGAATTTGGCTGGAGTGAGTT
GGAGAGTAGGAGAAGAGAAATCCAAGGCAACATTTGACCAGCCTGGGCAACATAGTGTGACTCC
GAGTCTGCAAAAATTAGACGGGTGTTGTGGTGCGCGTCTGTGGTCTCAGCTACCTGGAAGGTTC
AGGCCTTGGAAGGCTCAGGGAGGTGGAGGCTGCAGTGATCTGTGATTGCGCCTCTGCACTCCAG
CCTGGGCGACAGAGCCAGACCCTGTCTTAAAACAAAATAAACGGCCGGGCGCGGTGGCTCAAGC
CTGTAATCCCAGCACTTTGGGAGGCCGAGGCGGCCGGATCACAAGGTCAGGAGATCGAGACCAT
CCTGGCTAACACGGTGAAACCCCGTCTCTACTACAAATACAAAAAATTAGCCGGGCGTGGTGAC
GGGCGCCTGTAGTCCCAGCTACTCGGGAGGCTGAGGCAGGAGAATGTCATGAAGCCGGGAGGCG
GAGCTTGCAGTGAGCCGAGATCGCGCCACTGCACTCCAGCCTGGGCGATAGAGCAAGACTCCGT
CTCAAATAAATAAATAAATAAATAAATAAATAATAAAAACATCGGTAGGCATATTTCAAGGAAT
TCTATTTAAAAAAAATTTTTTTAGAGACAAGTTCGCTCTCTGTGGCCCAGGCTGGAGTACAGTG
GCATGATCCTAGCCCATGGCAGCGTTGATCTCTTGGCCTCAAGCGACCCTCCTTTGGAGTCGCT
GGGCCTAAAGGAGTGAGCCACCACGAAATTTTATTATAAATGGAGGGTAGAGAAATTGGGCAAT
AAATGGAGGGGGAAGTGAGTTAAGAGGAATTTTAATTATGTGTGTGTGGTTTTAAAAGAGGGGG
GTCTTGCTCTGTTGCCCAGGCTGCTGGGGTGCCAGTGGCGCAATCATGAATCACTACAGCCTTG
GACTCCTGGCCTCAAGCTATCCTCCCACCTCTGCCTCCCAAAGTACTGGGATTACTAGTGTGAG
CCACTGCACTAAGATAGGAGCAACATGTTTCAGCATGTTTGTGGGTTGATAGGAAAGATGAGAA
TGGGAAAGTTGATGTCGGAAAGAAGACAATGGCTAGAGCAATGTCCTAGAGTAGGTAAGAAGGG
ATGGATTTGGCCTTTGTTGGAAACATTAGCGGTTCTTTTGGTGACAGCTATATAGTTAACACAT
CTATGATACGTGAATGGGCAGATAGGATGGCAGGAGATTTTGAAAGTTCTCTTGATTCTTACTG
TTCTCTTAGTGAAAGAAGCAAGGTTATCAGCTAGAAGCTGGGATGGGAGAGGAAAGAGAAGATG
GGAAGTAGATAGTTCTTTAGAAGAGTGGGCAAGGGTTGGACTAGGGAAGTTTAGTGGAAATATT
GCTAGGCAACATAAAGAGCCTACTTGAGATTCGTGGTCATGAGTTGAAGGAGACCAGACAGCAA
GATTGTGTATGAGGGCACCCACAGAGTAAATGGAGAGTTGAAATTAATGCAGTTGTGATTTTAC
CACGTGGATATGAAGAAGTGAGGGGGAGAAGTACAAAGGAGTTCTCTTAATGATTGACCATGGA
ATTTAAGCTGGCTAAGAAAGGAAGTGAGAGGCCGGGCGCGGTGGCTCACGCCTGTAATCCCAGC
ACTTTGGGAGACTGAGGTGGGTGGATTACCTGAGGTCAGGAGTTTGAGACCAACCTGGCCGATA
TGGCGAAACCCCATCTCTAATAAAAATACAGAAAAATTAGCCGGGAATGGTGGCAGGTGCCTGT
AATCCCAGCTACTCAAGAGGCTGTGGCAGGAGTATCCCTTGGACCCAGGAGGTGGAGGTTGCAG
TGAGCCGAGATCACGCCACTGTACTCCAGCCTGGACGATATAGTGAGACTTCACCTCAAAAAAA
AAAAAAAAGAAAGGAAGTGAGGATTTTAAGACCCTGAGAGACAGTTTAAAAAGTGGGAGGATCG
GCCGGGCGCTGTGGCTGACACCTGTAATCCCAGCACTTTGGGAGGCCGAGTTGGGCAGATCACA
AGGTCAGGAGTTCGAGACCAGCCTGGCCAATATGGTGAAACCTTGTCTCTACTAAAAATACAAA
AATTAGCCGGGCATGGTGTCACGTGTCTATAATCCCAGCTACTCGGGAGGCTGAGGCAGAAAAA
TTGCTTGAACCTGGGAGGCAGAGGTTGCAGACAGCTGAGATCACTCCATTGCACTCCAGCCTGG
GCAACAAGAGCAAAACTTTGTCTTTAAAAAAAAAAAAAAAAAAAGAATACAAAAATTAGCCGGG
CGTGGTGGCGCGTGCCTATAATCCCAGCTACTTGGGAGGCTGAGGCAGGAGAATCAGTTGAACA
CGGGAGGCGAGGTTTGCAGTGAGCCGAGATTGCGCCACTGCACTCCAGCCTGGGCGACAGAGCA
GGACTCCTCTTGGAAAAAAAAAATTAGCTGGGCATGGTGGCAGGTGCCTGTAGTCTCAGCTACT
AGGGAGGCTGAGGCAGGAAAATCACTTGAACCCGGGATGTGGAGTTTGCAGTGACCCGAGATCG
TGCCACTGTACTCCATCCTGGGCGACAAAATGAGACTCTGCCTCAAAAAAAAAAAAAAAAAAAA
GTGGGAGGATCAATGTACTGCCAGTCCTAATGAAGTGGAATGATTGTCCCCATCAAATCACTAG
TAGGAGTAAGTTGCAGAGCCTAGAAGGTGATGGTTAAGAGAGTGGGATTCTTGAAACTGCATTT
ATGGAGAGGTTGTGGTTATTGGTTATAATAAATAAATACAGTTGAAGTGAGTGAGTAGCTGAGA
TTTGGGGATGTATCAGTTCATTCTTACACTGCTACAAAGACATACCTGAGACCAGGTATTTATA
AAGATAAGAGGTTTAATCAGCTCACAGTTCTGCTGCCTGTACAGGCTTCTCTTGTGGAGGCCTA
AGGAAACTTACAGTCATGGTGGAAGGTGAAGGGGAAACAAGCACAGTCTTCACATGGCCAGCAG
GAGAGAGAGAGAAGGGGGAAGTGCTACATACTTTAAAACAACCAGATCTTGTGAGAACGCTTAT
CAGGAAACAGCACTTGGGGATGGTGCTAAATCATTAGAAATCACCCCCATGATCCAGTCGCCTC
CTACCATGCCCACCTCCAACACTGGGGATCACAATTCAGCATGAGATTTGGGTAGGAACACAGA
GCTGCACCACATCAGAGGATGTACAAGATTGTGGTGGAGAGGAGTTTAGAGACCTGCAAATATA
GGGTAATTGAAGGGATCATCTACATGGATATTTAAATCACCAAAAATTATGACAGGAGTAGTGT
TGGAGAGAGAACTGCGATGTAAACATTAAGGAATGAGGAAGAGTGACTCGGTAGGCTGTAGGTG
ACTGCAATAGGAAACGATAATAGACTGTGAGTCTGGTGACAAGATTTTCCTTCTTTCTTTTTTT
CCCCCCCCCCGAGACAGGGCCTCTTTTTGTTGCCCAGGTGGGAGTGCAGTGGCGCGATCACGGC
TCACTACAACCTCCTCCCAAGCTCAAGGGATTCTCCCACTTCAGCCTCTCAAGTAGCTGGAACT
ACAGGTGCTGACCACCATGCCTGGCTACTTTTTGTCAGGATTTTCAAGGCTGGGAATTTTGAGA
GGGGAATGGAGGAGAATAATCTGAAAGTGCAAGTAAGGAGCAGGGAAGATTTCTTTTTTCTTTT
TTTTTTTTTTTTTTGAGTCGGAGTCTGGCTCAGTCGCCCAGGCTGGAGTGCAGTGGCGAGATCT
CCGCTCACTGCAAGCTCCGCCTCCCGTGTTCACGCCATTCTCCTCCTTCAGCCTCCCGAGTAGC
TGGGACTACAGGCGCCCGCCACCACGCCCAGCTAATTGTTTTTTTGTATTTTTAGTAGAGACGG
GGTTTCACCGTGTTAGCCAGGATGGTCTCAATCTCCTGACTTTGTGATCCGCCCACCCCGGCCT
CCCAAAGCGCTTGGGATTACAGGCGTGAGCCACCGCGCCAGCCAGAGCAGGGAAGATTTCTTCC
CCACATCTCCAGTAGGTACAGTGATATGAAGTGTGTGGAGGAGAAAAGAGGAAACATCTATCAT
TTGAGATGGCTGCGAAAGGAAAAGGCATCCTCAGGGAGCTAGATTTTACTTAGAGCAAGAAATG
AAGGGATGATTCAGAGGTTAAAAGAGTGGATTTTATGAATTACTCAAGGGAGCACAGTGGAAGT
TTCAGGAAGTGGTAGGAGAAGGTAGAAGATGGCAGGGTGTTGGGAATAATTTGAGAAATCTGAG
CTACTGGAAATGACTGAGAATCAGATATAAAGGCAGTCCTGGTGGTCCGTTCTGGCTGCCGTTG
CTGTGTAACGAATCTGCCAAAACTTAGTGGCTTGAAACAACAAAGAACATTTTATTATCTCTCA
TTGTTTCTGTGGGTTAGGAATTTGTGAGAGCCGTGCTGGGCAGTTTTCGTGCGGCTGTCTCGTG
GTTGCACCTACATAGTTGCTAGAGCTACAGTAGCTGGGGACTGAGCAGCTAGGGATTGGCAGGC
TATCTCTTTTTTTCATGTAGTCTCATGAAGATTTCTTTATGTGGTTTCAATGTGTGGGCTGGTT
TGGATTTCCTTATAGCATGGTGGCCTCAGTTGGATTGCTGTTTTGTGATCCTTTTCATCCCTCC
TTGTCCTGTCCCCAGACAACCACTGATCTACTTTCTGTCACCATAGATTAGCCTGCATTTTTAA
GAATTTTTATAAACGTGGAATGATAGAGTACCTTTTTTGTCACGTTTCTTTTATTTATCATAGC
TATTTTGATTTTCATCCATTTTATTGCTGAGTAGTATCCCATTGCATGTATATACTATACTGTA
TTCATTCGCTTGCTTGTGAACATTTGGGCTTTTTCCAGTTTGGGACTGTTAACAAGTAGAGCCA
CTATGAATATTAGTGTATAAGACTTCATATAGCCAAGGCTGGCAGATCGCTTGAGCCCAGGAGT
TTGAGACCAGCCTGGGAAACATGGTGAAACCTCTATTTTTATTTTAAAATCAAAAATTAAAAAT
TTTCTATAAAAAATTTTAAAGAAGACTTTGTATAGACATACGCTTTCATTTTTCTTGAGTGAAT
ACTTAGGTCTCAGGGTAGATGTATTTTAAGTCTTTAAGGAGCTGTCAAACTCTTCCTCAAAGTG
GTGGTTGTACCATGTTACTTTTTAATATAACAGAGATTAATTGAGCAAAGAAAAATTCAAAAGT
TGGACAGCCCCCACAACTAAATAGGTTCAGAACAGCTCCCCCATTTTGCATTTTGACCAGCAAT
GTATGAAAGTTCCATTTGCTCAGTGTCCCTGCAAACACCTGGTATGGTCAGTCTTTTTAATTTT
AGGCATTATAATAGATATAGTGGCTTCTTGTGATTTTAATTAGCATTTCCTAATGACCAGTGCT
GCTGTTGATCATTTCATGAGTGTATTTGCCATCCGTATATCTTTTTTGGTGAAGTGTCTATTCA
AATCATTTGGGTTTTTTTTTTTTTTGTTTTTTTTTTTTGGAGACAGTGTCTCACTCTGTCACCC
AGGCTGTTGTGCAGTGGTGCAATCACACAGCCTACTGCAGCCTCCACCTCCTGCGCTCAGTCTT
CTTGTCTCAGCCTTCTGAGTAGCTGAAATTACGAGCACACGCCACAATGCCTGGCTAATTTTTT
AAAATTTTGTAGAAACAAGGTCTCATTATGTTGCCTGGGCTTGTCGTGAACTCCTGGGCTCAAG
CAATCTTCCTGCCTCAGCCTCCCAAAGATTGGGATTGCAAGTATGAGCCACTGCACCCGGCCAA
CTTACCCATCTTTTAATTGAATTTTTTTGTTGTTGAGGTTTGAGAGTTCTTCATGTTTGCTGGG
TACAATATCTTTATCAGATAGGTAACTTGCATGTATTTTCTCCCGGTTTACACTTTGGTTTTTC
ATTTTGTTAACAACGTCTTTTTAAGAACAGAAAATCTTAATTTTGCTGAAATCTAATTTTTCAG
TTTTTTCTTTGATGGTTTTGAGAGAGGAGGTAAAAAAAGACTAGGTAAGCCGATAGTTAGACAG
AGTCCTCGGTAGAACTTCCCTTCTAACAAAAAGCAGCCCAAGAAATCACTTCTCTTCTAACAAG
GAGCAGCCTGGAAGATCGGGCTGTAAACATGTATAAGGAAGCAGCTCTGGCACAGAGGGGGAGC
TTCCTGGGTAATCAGCAAGCTTCACATACGTAAGGTGGGTATGTGAAGTAAACACAGTATGTGA
AGTAAACACAGTGGACCTTAGTACATACTCAGATAAGGAAGCTGGAAGCTTGCATGTTGTGAGT
TGTTGGGGTTGCCTGCAGCTGCACGGAGAGAAAGGGGTACCTGGGGCCAGGCATGTCCACCATG
GTGGCTCCACCTCCCCTTATTTAGCACATGCACAATAGGAAAGAGATAAGCAATGTGGAGTAGC
TCAGGCCAAGGACCTGCCTGCATAATAAAAGGTTGGGGTGGGGGATGCCAGAGATTCACGCTCT
GTGCAGATGGCAACACCTGGTCCTAACTGGTTTTTTGCTCCCTATGTGTAGATAAGCTACCCCC
TTCCCATTAGCTCATTTATAAAAATGCTTGCATTTCACTGTGGAATGGGAACTCTTTTCAGGAC
CTCTCTCTGCAGGAGAGAGCTAGTCTCTTTCTTTTGCCTATTAAACTTCTGCTCTAGCCTCACA
CCCTTGGTGTGTCAGCGTCCTTGATTTCCTCAGCGTGAGACCAAGAACCTCGGGTGCCACCCCA
GGCAACAAGGCCATTTCAGTTTGTTCTTTTGTTATAGGCAATCCATGATCACAGATTTTTCTCT
CTTTTTTTTTTTTACACAGTTTAGAGTTTTAGTTTTACACTTAGGTCTGTAATCCATTTTGTAT
TAATTCTTATATGTGGCTCAGTGTAGGTGGAAATTTGGTTTGTTTTTGCATAAGGATTTCCAAT
AGTTTTACCACCATTTCTTGAAACTACTATGCTTTCTCTATTAAACCACATTTGTAACTTTAGT
TAAAATCAGTCACATATATCACAGGGCTATTTCTGACTCTCAATTCTGTTACATTGTCTATTAG
TGTATATTGATGTCAGTACTACACTTTTAATTACTATTGCTTCAGGGTATGTCTTGTAAACCAA
AAATAAAATTATAGGCCCCCCCCGCCCCTGCACAACCAACTGAATGGACCCATCCTCTCAGCCA
AGGGCATTCCAAAATTAACCTGAAAAACTAGTTCAAGCCATGATGGGAAGGGGGAGTTGGACAT
GTCTCATCACACCCTACTACCTTTTGGAATTACTGATAGAACAGACTCTTAAAGTCTGAAAAGA
AACATTTACAACCTACCCTCTCTGAAGCCTGCTACCTGGGAGCTTCATCTGCATGATAAAACCT
TGGTCTCCACAACCCCTTATGGTAACCCAAACATTCCTTTCTGTTGATAATAACTCTTTCAACT
AGTTGCCAATTAGAAAATCTTTAAATCTTCCTATGACCTAGAAACCTCCCTACCCCCACTTTGA
GTTGTCCTGCCTTTCCTGACAGAACTCATGTACATCTTACATATATTGATTGATGCCTCATGTC
TCCCTAAAATGTATAAAACAAAGCTGTACCCCACCACCTTGGGGACATGTCATCAGGACCTCCT
GTGGCTGTGTCATAGGAGCGTCTTTAACTTTGGCAAAATAAACTTTCTAAATTGATTGAAACCT
GTCTTAGCTACTTCTGGTTTACAGTCTTAAAGTTAGATAATGTAAATTGTCCAGCTTTGGTTTA
TTTTTGTCCTTAGTAGTTCCATATAAATTTTAGAATCAGCTTTTCAATTTAATACACTACTTTC
CTCTTAGATCCACAATTAAATATATTTGATGCTAACAATTCTGTTTTATGTTTTTCGTTTTTTT
TTTTTGAGACAAGAGTTTCGCTCTTGTTGCCCAGGCTGGAGTGCAGTGGCGCGATCTTGGCTCA
CCACAACCTCCACCTCCCAGGTTCAAGCAATTCTTCTGCCTCAGCCTCCCGAGTAGCTGGGATT
ACAGGCATGCGCCACCACGCCCGGCTAATTTTGTATTTTTAGTAGAGACGGGGTTTCACCATGT
TGATCAGGCTGGTCTTGAACTCCTGACCTCAGGTGATCCACCCACCTCGGCCTCCCAAAGTGTT
GGGATTACAGGCGTGAACCACCATGCCTGGCCAGTTCTGTTATTTTTAAAACCCAAGTTTCCCT
GGTCATATCTTGGTTGGATGAAGCGTATTTTCAATAGATTACCCTGGAAAGGCTAGTGAGTACG
GTATTCTTCTACATTTTAGACTTTTCTTAGTCTTGCTACTTCAAGGACAGCTAGGCTGCATATA
AAATTCTTGGCTCATACTTTTTCCCCATAAATTTCTATGAGAAAGTCTAATGATAACTGATTTT
CTTTATTTTGTAACTTAGTCTTTTTGCTTAGAGGCTCTCTGAGGATGGGAGGGGGTTCTTCCTC
CCATCCCTAGGAATTTTTCTTTTTTTTAAATTCCTAATCACTAGACCACCAGGAAGATTGTTTG
TTTTGTTTTGTTTTTATTCTTCAGGGACCCCATTTATACATACGTTAAATAAATACTGTTTGCC
AATGTATCAACCATTTTGCTTCTTATTTATTTTTGTTCCTTTGGTTCTTTTTCATGGCTTTGCT
TTGGTGCTCCTTAGATTTTCAGTCAGATGTATTTGTCCTTGGGTACCTTGTAATCAGTATTACC
TTTTCTTCTGTCGCTTTGTTTTCTGTTCGTTTTGAAATTACTTGTTTCCTGGTCTGGCAATAAC
AGTTGAGATATGAGGAGTTTGAGCTGCCATCTGTCTATGTATCTTGCTTTAAGACTGCACTCTT
CTATTGATATCACTGGCCTTGATTTTGTGATTTCTTTATTTCTTCAGGACCACCCTTCATTTTC
TACTGTTTGCTTCCTTTTTTTTTGAGATGGAGTCTCACTCTGTCACTCAGGCTGGAGTGCAGTG
ATCTTGGCTCATTGCAACCTCTGCCTCCCGGGTTCCAGCAATTCTCCTGCCTCAGCCTCCCAAG
TATCTGGGACTACAGGTGTGCACCACCATGCCCGGCTAAGTTTTGTATTTTTAATAGAGACGGG
GTTTTGCCACATTGGCAGGCTGGTCTCAAACTCCTGATGTCAAGTGATCCACCCACCCCACCCA
CCTCTGCATCCCAAAGTGCTGGGATTACAGGAATGAGCTGCCGTGCCCAGCCTCCCCCCTACCC
CCCTTTTTTTCTTTCGAGACAGAGATTATAGGTGTGAGCCACTGGACCCAGCCTGTTTTTATTC
CTTTTACCAAATCTCCAAGGAATATCTTCCCTTCCAAGTGCGAATGTAACCTTAAGTCAGTTAA
CCTCTTTGTGATTACTTTTCTTATCTGCAAAGTGACTTAATGATCTTAAGTACTTTTTTTTTTT
GAGACAGGGTCTCACTGTCACCCTGGCTGGAGTGCAGTGGCACGATCTCTGATCTCCACTCACT
GCAATCTCCTCTTCCCTGGTTCAAGCGGCCCTCCCACCTTAGCCTTCTGGGTAGCTGGGACTAC
AGATGTGAACCACCACGCCCAGCTAATTTTTGTACTTTTTGTAGAGATGGGGTTTTGCCATGTT
GCCCAGGCTGGGATTATTAAGTACTTTTTATCATACAGCAAGATTGACATTTTATATTGGAATA
CATTTGTCTCTATATAACGGAGATTAACAGGAAAATGACAAGCCTGGGTGCGGTGGCTCATGCC
TGTAATCCCAGCACTTTGGGAGGCTGAGGTGGGAGGATCACTTGAGGTCAGGAGTTCGAGACCA
GTTTTGCCAAGATGATGAAAGCCCATGTCTACTAAAAATACAAAAATTAGCCCAGCTTGATGGT
GGGCGCCTATAATCCCAGCTATTTGAGAGACTGAGGCAGGAGAATCACTTGAACCTGGGCAGCA
GAGGTTGCAGTGAGCCGAGATCATGCCACTGCACTCCAGCCTGGGTGGCATAGCGAGACTCTTG
TCTCAAGAGAAAACAAAACAAAACAAAAAAAAAACAGGAAAATGACAAAAAGTAATATTACAAC
TCAGTGAATTTTATAACAAACTTTTTTGGAATTCATTGACTAATACTATACCAAATCCAAAATA
CTCTCTAGTATACCAAATCCAACTCTACCCTATAGTATAAATTGGATTCTATTTGGACTTGTCT
CACTAATCCCTCATACAGTGTGTTTTATTTTTTATTGAAGTAAAAAAATTTGTCATTTTAACCA
TTTTTAAGTATATAGTTCAGTAATATTAAGTATGTTCATGTTGTTGCGCAATAGATCTTCGGAA
GTTTTTCGTCTTGCAACCTGAAACTCTACCCATTAGCAAATTCCCATTTCTCCTTACACTTAGC
CCTTGGTAATCATCATTCTTTTTTTTTTTTTTTTGAGATGGAGTTTTACTCTTGTTGCCCAGGC
TGGAGTGCAATGGTGCAATCTCGACTCACCACAACCTCCGCCTCCCAGGTTCAAGCAATTCTAC
CTCAGCCTCCCGAGTAGCTGGGATTACAGTCATGCACCACCACGCCCGGCTAATTTTGTATTTT
TAGTAGAGAAGGGGTTTCTCCATGTTGAGGCTGGTCTCGAACTCCTGACCTCAGGTGATCTGCC
CACCTCGGCCTCCCAAAGTGCTGGGATTACAGGCGTGAGCCACTGCGCCTGGCCCATTCTTTCT
AATTCTATAAATTTGACTACTTAGTTACCTTACATAAATAAATTCTTATAGTTAGTGTTATTTT
TGCTTCCATGCCTTTTTTGTTGTTGTTCATGCTCTTACTTGGAATGCGTTCTATTTTGTCTACC
TATGCACATCCTGTTGGGTTTTTTTTTTTTTTGGGGGTTTTTTTTGTTTTTTTTTGTTTTTTTT
TCCCAGACAAGGTCTCAATTTGTTACCCAGGCTGGAGTGCAGCGGCGCCATCTCCACTCACTGC
ATCCTCAACTTCCTGGGCCCAGGTGATCCTCTCGCCTCAGCCCCTGCAGGTAGCTGGGACTATA
GGCATGTGCCACCATGCCCAGCTAAATTTGGTTTTTTTGTTTGTTTGTTTTTGAGACAGAGTCT
CACTCTGTCACCCAGGCTGGAGTGCAGTGGCACAATCTCAGCTCACTGCAATCTCTGCCGCCCG
GGTTCAAGTGATTCTCCTGCCTCAGCCTCCCAAGCAGCTGGGATTACAGGTGACTGCCACCACG
CCAGCTAAGTTTTGTAGTTTTAGTAGAGATGGGGTTTCACCTTGTTGGCCATGCTGGTCTCGAA
CTCCTGACCTCGTGATCTGCCTGCTTCTGCCTCCCAAAGTGCTGGAATTACAGGCATGAGCCAC
CACGCCCGGCCAGAATTTTTGTATTTTTAGTAGACACAAGGTTCTTACCCTGTTGCCTAGGCTG
GTCTGGAAGTCCTGGACTCAAGCAATTCACCTGCCTTGGCCTCCCAAAATGCTGGGATTACAAG
CCACCATGCCCGGCCTAAATCCTGTTGTTTTGTTTTGTTTTATTTTGTTTTGTTTTGTTTTGTT
TGTTTTTTGAGACAGAGTCTCGCTATGTCTCTCAGGCTGTAGTGCAGTGGCGCGATCTTGGCTC
ACTGCCACCTCTGCCTCCCAGGTTCAAGTGATTCTCCTGCCTCAGCCTCCCAAGTAGCTGGGAT
TACAGGCATGTGCTACTATGTCCGGCTAATTTTTGTATTTTTAGTAGAGACAGGGTTTCACCAT
GTTGGCCAGGCTGGTCTCGAACTCCTGACCTCGTGATCCACCCACCTCGGCCACCCAAAGTGCT
GGGATTACAGGCGTGAGTGGTTTTTATTTCTTAGGCCGGTTTCCTCCATATGATCTTGCAGTAG
ACATTAATTTCTTTCCTTTTTAATTAAAATACTGTTTGTATTTCACATTTTGATGTTTGTTAAG
ATTTGTTTTATATTGTTTTTTGTTTTGTCTTGTGTGATAGTCTTAAATCCCTAGTTAGATAATA
ACTGGAGAGTACCATGTTTCTATATATCTCTCAGTGACTTGCACAGTGCTAGCAGATAGTGCTA
AAAAATTATTTATTATTATTATTATTTTGTTATTGTTGTTGTTGTTGTTAGACAGGGTCTTCCT
CTGTCACCCAGGCTAGAGGGCAATGGGATGATCATAGCTTACTGCAGCCTCCAACAACTGGGCT
CATGTAATTCTCCTGCCTCAGCTTCCCAAGTAGCTGGGATTACAGGCATGAGCCACCATGTCTG
GACAAAAATATTTCCAGGTGCAGTGGCTCATGCCTGTAATTCCCACACTTGGGAGGCCGAGCGA
GGCTGGAGGATCACTTGAGCCTAGGAGTTCAAGACCAGCTTGGCTAAGATGGCGAGACCCCGTC
CCTACAAAAAATTTTAAAAACTAGCCAGGCATGGTGGCATGCACCTATATTCCCAACTACTCAG
TGGGCTGAGGTGGGAGGGTCATTTGAACACAGGAATTTGAGGGGAGAAAAAAAGAAGAGAGAAA
GAGAAGTGAAGGAAGGAAGAAAGGAAGGAGGGAGGGAGAGAAGAAAGAAACGAAAGAAAGGAAA
AGAAAAGGAAGGAAAGAAAATTGGTACCAGGAAAGCAGGAAAGGGAAATGGAAGTAAAAAAATA
ATAATAATAATAAAATGAAAATTGGTTAGTCACTATTAACAATTTGTATCCTTATAATCTGGAA
ACATTATAATTTCAAAAGAAAAAATATTCTTTGGATCATAGGTTCTGAGGTCAGAACAGCATTC
CCGTAGTCTAGATGAAGTCAAGTTTTATCTGATCTTAATTGAAATAAATATAGCTGGCCTTGAA
CAAATCTACTCATGGTATGTGGATAGGAATTAAATTGTAGGGGCATTCACTTGATGGCATTCAT
TCTTAGAACATTTACCTATGTCTAGCTTTTGGAGTAAAGTCACATAACCTCTAACCAGGTAAGT
TTCCTGTGGCTTTATTTAGGATTTTAAATACTCATTTTCAGTGTAATTTTGTTATGTGTGGATT
AAGATGACTCTTGGTACTAACATACATTTTCTGATTAAACCTATCTGAACATGAGTTGTTTTTA
TTTCTTACCCTTTCCAGAGCGATGATTCTGACATTTGGGATGATACAGCACTGATAAAAGCATA
TGATAAAGCTGTGGCTTCATTTAAGGTATGAAATGCTTGCTTAGTCGTTTTCTTATTTTCTCGT
TATTCATTTGGAAAGGAATTGATAACATACGATAAAGTGTTAAAGTACATGTTATTCAGTTTTC
ATTTTGAAGATTAGATGGTAGTATGAGTTAGTTAAATCAGGTGATATCCTCCTTTAGAAGTTGA
TAGCCTATATATGTCATCCTTTGTGGAGGCAATTTAAATAAAATTTAAAACATTTATTCCTGGC
TGGGTATGGTGGCTCACTCCTGTAATCCCAGCACTTTGAGAGGCTGAGGCGGGTGGATCACCTG
AGGTCAGGAGTTTGAGACCAGCCTGGCCAACATGGTGAAACCCCGTCTTTACTAAAAATACAAA
AATTAGCCAAGCATGGTGGCACGTGCCTGTAATCCCAGCTGCTTGGGACACTGAGGCAGGAGAA
TTGCTTGAACCTGGGGGGCAGAGGTTGCAATGATTGCACCACTGCACTCCAGCCTGGGCGATAG
AGTGAGACTCCATCTCAGAAAACGAACAAACAATGTATTCCTTTTAGTATTTTTACATTGTATC
AAACTATGGAAGTCCTCTAATTGAGATTAATAAGAAAAAGACAATCTGAATTATAATTTTAAAC
ATTTAACAAGCATGTAGTAAAATAATGATGAAGATAAATAGCATTAGTACAGCAATTAATATTT
GTAGCATGCTGACAGTGCTCTGTGTGCGTTTCATATATTAAATTACTCTAATCATCCCAAATCC
TGTAAGTTGGGTATCAATTCAAGTGTTCCTATTGGGTAGGAATATACAGTTCTTTTAGGAAATG
TAGTATGGTTCTGTGTCTCAAACAGGACACTTACACAGTTGGCCAACATCATCACCTTCTCCAT
TCTCTGAGATGTTTAGTCTTACTGAGCACTAAATATGGGTCATCAATAGTCCAGACTACCTTGA
GCAAACAATAGTCCAGACTACCTTGAGCAAACAGAGCATATACTCATACAGTGTATAAAGAGCA
CCAAGCATACAGATTTCATGTCTTTCTCATAGTTACTCTTGTAACATGAGCTAAAGATCAGACC
TCTATGTCACCTTTGTAACTGATTTCTAGATTTTTTTTTTTTTTTGAGATGGGGTCTTGCCCTG
TCACCCAGGCTGGAGTGTAGTGGCGTGATCATGCCTCATTGGAGCCTTCAACTCATGAGCTCAA
ACAATCCTCCTACCTCAGCTTCCTGAGTAGTTGGGACCACAGGTGTGTGCCACCACACCCAGCT
CATTTTTGTATTCTTTGTAGAGATGCAGTCTCACCCTGTTGCCCACGCTGGCCTGGAACTCCTG
AGCTCAAAAGATCCCTCCGCCTTGACCTTCCAAAGTGCTGGGATTACAAGCATGAACCACTGCA
CCCGGCCTAGATTTTTAAATGTGCTTTCCAGTATACACTGAAACTAGAAGTCGACTAAAGAATT
ACCAAGAGAATTCTATAAAATAGAGATTGAAATGGGGCTCGATGTGGGATGGGTTGGTGATATT
GCAGGGAGAAGTAATCTGAGTAAAGGAGGAAAAGAACTGATTTGGGAAAACGATAGTTTTAGTA
GTGAGTTTGAGTATGAATTAAGTTGAGATTGAATTTGAATTAAGTTGAGGTTGAATATGAATTA
AGTTGAGGTTGAGTTTGAGGTATGAATTAAGATGTGAAATTGATCATTGGAAATGTTAGATTGA
GAAAAGTCACAGCTGGATTAATAGCTTCAGAAGTGTGTTTGCAGACAGTTGCAACTAAAGTAAT
AAGAATAGATGGCCTTGGCCGGGCGCGGTGGCTCACGCCTGTAATCCCAGTACTTTGGGAGGCT
GAGGCGAGCAAATCACGAGGTCAGGAGTTCAAGACCAGCCTGGCCCACATGGTGAAACCCCGTC
TTTATTAAAAATACAAAAATTAGCTGTGCACAGTGGTGCACGCCTGTAATCCCAGCTACTCGGG
AGGCTGAGACAGGAGAATCGCTTGAACCTGGGAGGTGGAGGTTGCAGTGAGCTGAGATCAGTGT
GACTGCACTCCAGCCCGGTGACAGAGTGAGACTCTGTGTAAAAAAATAAAATAAATAAAATAAT
GGCCGTAAGCAAGTAAAGAAGGATGGCCAGCTCTTATTGGGAATGCCTAAATCTAAGGCTTGAT
CAGAAGTAATGAAACCGTTGGGGCCCTACATTGCTATGACATCCAAAGGGCCATGAATATCAGG
AAGAAAGATAATTAACAGGGTCTAATGTTACAGAGAGGTTGAGAGCAAGGAGATTTGATTAAAA
GGGTCTTTAGAGCTGATGTCAGGTGTATGATGCCTTTAAGAGCAGTTTTTATAGTGCAGGGGGT
GGTCAAAAGAGAAAATAGGTGCTTTCTGAGGTGACGGAGCCTTGAGACTAGCTTATAGTAGTAA
CTGGGTTATGTCGTGACTTTTATTCTGTGCACCACCCTGTAACATGTACATTTTTATTCCTATT
TTCGTAGCATGCTCTAAAGAATGGTGACATTTGTGAAACTTCGGGTAAACCAAAAACCACACCT
AAAAGAAAACCTGCTAAGAAGAATAAAAGCCAAAAGAAGAATACTGCAGCTTCCTTACAACAGG
TTATTTTAAAATGTTGAGATTTAACTTCAAAGGATGTCTCATTAGTCCTTATTTAATAGTGTAA
AATGTCTTTAACTTAAGTGATTAGTACAGTGTTTCTATTGACATATACTTATACAACTTCAAAA
ACAACTATTAAATTTTCTGTTATTTAGGAACATGCATATTAGTCATGAAAGTATAAAGAATTAG
ATGGGAATGATAAATGCTAAAATCAGGACATGTGTTCCATTTGTGAATGGAAGGCAGGGAGAAG
GTGCCGTTTGGAAGGAGTACCCAAGAGCCGTAAGCTGAATTGGCAGTGTTTTACATCTTAAGCT
GAGAGATAGATTTTTTTTTCCCCTTTTTCTTTAAAAACTCTAAAACTGTTAATTCCAAGGAACC
CAGAAGTCTAGGTAGATTATTTCTGCTAGTTAAAAGCAGTAGTCCTGAAAGCTGAATATTTTGG
TGTCTTTTGAGCCAACTTTAGTTTCATCATTACCAAGGGGGAAGAGAGCTAACAGTTGATGAGC
ACTTGCTCTAGGCCAGTCCAGAGTGCTGGGCACCATACGCATTTTATCTCCCTCCCGCTATTCA
CAACAAATATGGGAGGTAGTTTATATTATAGCCATCTAATAAGATGGGGAAACTAAGACTCAAA
GAGATTCAGAAACTTGTCCATGATTATAAATGTAAGAGAGTTGGAATTCAGATTTATGTATTTA
GACCCCAAGCCTTTCTCATTACATCATTTTGCCTTCCAAATCTCTACCCTCTATCCTTCACCTC
CCCACTGATCAAAACGAGATGATAGTTTGCCCTCTTCAAAAGAAATGTGTGCATGTATATATCT
TTGATTTCTTTTGTAGTGGAAAGTTGGGGACAAATGTTCTGCCATTTGGTCAGAAGACGGTTGC
ATTTACCCAGCTACCATTGCTTCAATTGATTTTAAGAGAGAAACCTGTGTTGTGGTTTACACTG
GATATGGAAATAGAGAGGAGCAAAATCTGTCCGATCTACTTTCCCCAATCTGTGAAGTAGCTAA
TAATATAGAACAAAATGCTCAAGAGGTAAGGATACAAAAAAAAAAAAATTCAATTTCTGGAAGC
AGAGACTAGATGAGAAACTGTTAAACAGTATACACAGTTGTCAGTTTGATCCACCGAGGCATTA
ATTTTTTCTTAATCACACCCTTATAACAAAAACCTGCATATTTTTTCTTTTTAAAGAATGAAAA
TGAAAGCCAAGTTTCAACAGATGAAAGTGAGAACTCCAGGTCTCCTGGAAATAAATCAGATAAC
ATCAAGCCCAAATCTGCTCCATGGAACTCTTTTCTCCCTCCACCACCCCCCATGCCAGGGCCAA
GACTGGGACCAGGAAAGGTAAACCTTCTATGAAAGTTTTCCAGAAAATAGTTAATGTCGGGACA
TTTAACCTCTCTGTTAACTAATTTGTAGCTCTCCCATGAAACTTTTGTAGCTTAAATACACAAG
AATTTTTTGAAAAGGAAATAAGATAATGATGCAAAATAGTTAATTTTTTAAAAAAATGTTAGAC
ACTGCAGTGGATGCAACAAAATACTTTATATGAAAGATTTATCCAGTTAACTTTTGTGGAGTAT
TAGGTATTAGACTAATAATTAGCACACTTACTTAAGTTAGAAAGTATAATAATGCGCCGGACGC
GGTAGCTCACGCCTGTAATCCCAGCACTTTGGGAGGCCAAGGTGGGCGGATCACAAGGTCAGGA
GATCGAGACCATCCTGGCTAACACGGTGAAACCCCATCTCTACTGAAAATACAAAAAAATTTGC
CGGGCGTGATGGCGGGCACCTGTAGTCCCAGCTACTCGGGAGGCTGAGGCAGGAGGATGGTGTG
AACCCCGGAGGCAGAGCTTGCAGTGAGTCAAGATCGTGCCACTGCACTCCAACCTGGGCGACAG
AATGAGACTCCATCTCAAACAAAAAAACAAAACAAAACAAAAAAAAGTGTAATAATAATTTATC
ATTAGCTGGATGATATGCTGTTGTTTCCCATGTCACCTGTATAAGATATGTAAAATAAGAACAC
ATTATTTACATCTAATATAGATAAAATCCTGAGGCGCTCTCAGATTGTTTTGTAGAGTTCAAAT
GTAAATATTGTTTTCATTTATGGTCCTTTTGGTTATAAGTAACAGAAATCAACTCTAAAAAGAT
TTTTATTATAGGTTAGATTATGTCATGGAACCTTAAGGCTTGTCCCTTTCTAGTTCTTTTGTGT
AAAGCGGTGATTTCTTCCATGGAGGGAATGGTATTTAGGCAATTTTTTTTTTTTTTTCGAGATG
GAGTCTTGCTCTGTCGCTCAGGCTGGAGTGCAGTGGCACCATTTCAGCTCACTGCAACTTCCAC
CTCCTGGGTTCAAGTGATTCTCCTGCTTCAGCCTCCCAAGTAGCTGAGATTACAGGCACCCGCC
ACCACACCCGGCTTATTTTGTATTTTTAGTAGAGATGGGGTTTCACCATGTTGGCCAGGCTGGT
CTTGAACTCCTGACCTCAAGTGATCTCCCCACCTTGGCCTTCCAAAGTGCTAGGATTACAGGCG
CCTAGCCTAGGCAGTCATTTTCAAAAAACAAGCATGACTCACCAAAAGTTTTAAGATTTTCTGT
GATAATGTTCTTATTGAGGCTTACATTATATTACAGTTTCTTGAATCTAAAATGATGTACCCTC
TTAGAATATATACATCATGCTTCATTGGTCTCAGGGGGCTGATTTTTATAAGGAGAGATTTGCT
AGTTTTCACAATATGTCCTCTAAGTTGGCATGTATAGCTAAACAGGCTTTCATAAAAATATACA
ATTTAGTTAATGAAATTTGGGATATAGTCTTTTATGATTGAAATAATTTTGCTAAATAGACTGT
CTCTGATTTATTAGGTAATCACCACTCTTATTTTGTTTTACTTCCTTAATGTCTACATAGAAAG
GAAATGAGAAAAATCCAGAGGTTGTCATTTGACTTATGAGTCTGTTTGACTTCAGGATTTGGTA
CATGAAATTTCACTTAATCTTTTTGATATGTATAAAACAAATATTCTGGGTAATTATTTTTATC
CTTTTGGTTTTGAGTCCTTTTTATTCCTATCATATTGAAATTGGTAAGTTAATTTTCCTTTGAA
ATATTCCTTATAGCCAGGTCTAAAATTCAATGGCCCACCACCGCCACCGCCACCACCACCACCC
CACTTACTATCATGCTGGCTGCCTCCATTTCCTTCTGGACCACCAGTAAGTAAAAAAGAGTATA
GGTTAGATTTTGCTTTCACATACAATTTGATAATTAGCAGAATAGAGGATTGTAAAATGTCATT
GTAGAACATCCCTTGGGCCAGATTCTAATGGGTAGAAATTTGAACTAAACCTCTGGGTTTTGTT
TGTTTTTAATGCCTTTCTGTTACCCAGATGCAGTGCTCTTGTAGTCCCAAGTCTAAGCTCTAGG
TTGCCTTCTTTCCTGGCAGAAGTTGGTGTCTATGCCATAAGGAGGTAGTTCCTGTTAGAAGGGA
TTTAATTATACCTTATATAAGGAATTAGTGTTTGCCCTTCTAGGTATAGTTGGATGTTAGCTTC
TGATGTAAACTGGATTTCTTTTTCTTTCTCTCTCTTTTTTTTTTTTTGTTTTGGAGGCAGAGTT
TTGCCCTTGTACCCCAGGCTGGAGTGCAGTGGTGTGATCTCAGCTCACAGCAACCTCCGCCTCC
TGGGTTCAAGCAATTCTGCCTCGGCCTCCCAAGTAGCTGGGATTACAGGCGACTGCCACCACAC
CCGGCTAATTTTTGTTTTATTAGTAGAGATGGGGTTTCACCATGTTGGCCAGACTGATCTTGAA
CTCCTGACCTCAGGTGATCCACCCGCCTTGGCCTCCCAAAGCGCTGGGATTACAGGCGTGAGCT
GCCGCACCCAGCTGTAAACTGGATTTCTAATGGTAGATTTTTAGGTATTAACAATAGATAAAAA
GATACTTTTTGGCATACTGTGTATTGGGATGGGGTTAGAACAGGTGTTCTACCCAAGACATTTA
CTTAAAATCGCCCTCGAAATGCTATGTGAGCTGTGTGTGTGTGTGTGTGTGTGTGTGTATTAAG
GAAAAGCATGAAAGTATTTATGCTTGATTTTTTTTTTTTACTCATAGCTTCATAGTGGAACAGA
TACATAGTCTAAATCAAAATGTTTAAACTTTTTATGTCACTTGCTGTCTTTTCGTCCTCGTTAA
ATTTAATTTTGTTGGTCTTTTGTTGTTATTGGTTGGTTTTCTCCAAATGCTAGCTATGTTAAGA
AATTTAAGGCCAGGTACAGTGGCTCATGCCTGTAATCCCGGCATTTTAGAAGGCTGAGGCAGGA
GGATCACTTGAGCTCAGGAGTTTGAGACCAGTCTGGGCAACATAGCAAGACCTCGTCTTTGTTT
AGGGGAAAAAAAAGAAATTTAAGTAGGAGATTATATAAGCAAAAATACAATTAATTTCCAGCAT
TCACTATATAATATAAATCTCCAGACTTTACTTTTTTGTTTACTGGATATAAACAATATCTTTT
TCTGTCTCCAGATAATTCCCCCACCACCTCCCATATGTCCAGATTCTCTTGATGATGCTGATGC
TTTGGGAAGTATGTTAATTTCATGGTACATGAGTGGCTATCATACTGGCTATTATATGGTAAGT
AATCACTCAGCATCTTTTCCTGACAATTTTTTTGTAGTTATGTGACTTTGTTTTGTAAATTTAT
AAAATACTACTTGCTTCTCTCTTTATATTACTAAAAAATAAAAATAAAAAAATACAACTGTCTG
AGGCTTAAATTACTCTTGCATTGTCCCTAAGTATAATTTTAGTTAATTTTAAAAAGCTTTCATG
CTATTGTTAGATTATTTTGATTATACACTTTTGAATTGAAATTATACTTTTTCTAAATAATGTT
TTAATCTCTGATTTGAAATTGATTGTAGGGAATGGAAAAGATGGGATAATTTTTCATAAATGAA
AAATGAAATTCTTTTTTTTTTTTTTTTTTTTTTGAGACGGAGTCTTGCTCTGTTGCCCAGGCTG
GAGTGCAATGGCGTGATCTTGGCTCACAGCAAGCTCTGCCTCCTGGATTCACGCCATTCTCCTG
CCTCAGCCTCAGAGGTAGCTGGGACTACAGGTGCCTGCCACCACGCCTGTCTAATTTTTTGTAT
TTTTTTGTAAAGACAGGGTTTCACTGTGTTAGCCAGGATGGTCTCAATCTCCTGACCCCGTGAT
CCACCCGCCTCGGCCTTCCAAGAGAAATGAAATTTTTTTAATGCACAAAGATCTGGGGTAATGT
GTACCACATTGAACCTTGGGGAGTATGGCTTCAAACTTGTCACTTTATACGTTAGTCTCCTACG
GACATGTTCTATTGTATTTTAGTCAGAACATTTAAAATTATTTTATTTTATTTTATTTTTTTTT
TTTTTTTGAGACGGAGTCTCGCTCTGTCACCCAGGCTGGAGTACAGTGGCGCAGTCTCGGCTCA
CTGCAAGCTCCGCCTCCCGGGTTCACGCCATTCTCCTGCCTCAGCCTCTCCGAGTAGCTGGGAC
TACAGGCGCCCGCCACCACGCCCGGCTAATTTTTTTTTATTTTTAGTAGAGACGGGGTTTCACC
GTGGTCTCGATCTCCTGACCTCGTGATCCACCCGCCTCGGCCTCCCAAAGTGCTGGGATTACAA
GCGTGAGCCACCGCGCCCGGCCTAAAATTATTTTTAAAAGTAAGCTCTTGTGCCCTGCTAAAAT
TATGATGTGATATTGTAGGCACTTGTATTTTTAGTAAATTAATATAGAAGAAACAACTGACTTA
AAGGTGTATGTTTTTAAATGTATCATCTGTGTGTGCCCCCATTAATATTCTTATTTAAAAGTTA
AGGCCAGACATGGTGGCTTACAACTGTAATCCCAACAGTTTGTGAGGCCGAGGCAGGCAGATCA
CTTGAGGTCAGGAGTTTGAGACCAGCCTGGCCAACATGATGAAACCTTGTCTCTACTAAAAATA
CCAAAAAAAATTTAGCCAGGCATGGTGGCACATGCCTGTAATCCGAGCTACTTGGGAGGCTGTG
GCAGGAAAATTGCTTTAATCTGGGAGGCAGAGGTTGCAGTGAGTTGAGATTGTGCCACTGCACT
CCACCCTTGGTGACAGAGTGAGATTCCATCTCAAAAAAAGAAAAAGGCCTGGCACGGTGGCTCA
CACCTATAATCCCAGTACTTTGGGAGGTAGAGGCAGGTGGATCACTTGAGGTTAGGAGTTCAGG
ACCAGCCTGGCCAACATGGTGACTACTCCATTTCTACTAAATACACAAAACTTAGCCCAGTGGC
GGGCAGTTGTAATCCCAGCTACTTGAGAGGTTGAGGCAGGAGAATCACTTGAACCTGGGAGGCA
GAGGTTGCAGTGAGCCGAGATCACACCGCTGCACTCTAGCCTGGCCAACAGAGTGAGAATTTGC
GGAGGGAAAAAAAAGTCACGCTTCAGTTGTTGTAGTATAACCTTGGTATATTGTATGTATCATG
AATTCCTCATTTTAATGACCAAAAAGTAATAAATCAACAGCTTGTAATTTGTTTTGAGATCAGT
TATCTGACTGTAACACTGTAGGCTTTTGTGTTTTTTAAATTATGAAATATTTGAAAAAAATACA
TAATGTATATATAAAGTATTGGTATAATTTATGTTCTAAATAACTTTCTTGAGAAATAATTCAC
ATGGTGTGCAGTTTACCTTTGAAAGTATACAAGTTGGCTGGGCACAATGGCTCACGCCTGTAAT
CCCAGCACTTTGGGAGGCCAGGGCAGGTGGATCACGAGGTCAGGAGATCGAGACCATCCTGGCT
AACATGGTGAAACCCCGTCTCTACTAAAAGTACAAAAACAAATTAGCCGGGCATGTTGGCGGGC
ACCTTTTGTCCCAGCTGCTCGGGAGGCTGAGGCAGGAGAGTGGCGTGAACCCAGGAGGTGGAGC
TTGCAGTGAGCCGAGATTGTGCCAGTGCACTCCAGCCTGGGCGACAGAGCGAGACTCTGTCTCA
AAAAATAAAATAAAAAAGAAAGTATACAAGTCAGTGGTTTTGGTTTTCAGTTATGCAACCATCA
CTACAATTTAAGAACATTTTCATCACCCCAAAAAGAAACCCTGTTACCTTCATTTTCCCCAGCC
CTAGGCAGTCAGTACACTTTCTGTCTCTATGAATTTGTCTATTTTAGATATTATATATAAACGG
AATTATACGATATGTGGTCTTTTGTGTCTGGCTTCTTTCACTTAGCATGCTATTTTCAAGATTC
ATCCATGCTGTAGAATGCACCAGTACTGCATTCCTTCTTATTGCTGAATATTCTGTTGTTTGGT
TATATCACATTTTATCCATTCATCAGTTCATGGACATTTAGGTTGTTTTTATTTTTGGGCTATA
ATGAATAATGTTGCTATGAACATTCGTTTGTGTTCTTTTTGTTTTTTTGGTTTTTTGGGTTTTT
TTTGTTTTGTTTTTGTTTTTGAGACAGTCTTGCTCTGTCTCCTAAGCTGGAGTGCAGTGGCATG
ATCTTGGCTTACTGCAAGCTCTGCCTCCCGGGTTCACACCATTCTCCTGCCTCAGCCCGACAAG
TAGCTGGGACTACAGGCGTGTGCCACCATGCACGGCTAATTTTTTGTATTTTTAGTAGAGATGG
GGTTTCACCGTGTTAGCCAGGATGGTCTCGATCTCCTGACCTCGTGATCTGCCTGCCTAGGCCT
CCCAAAGTGCTGGGATTACAGGCGTGAGCCACTGCACCTGGCCTTAAGTGTTTTTAATACGTCA
TTGCCTTAAGCTAACAATTCTTAACCTTTGTTCTACTGAAGCCACGTGGTTGAGATAGGCTCTG
AGTCTAGCTTTTAACCTCTATCTTTTTGTCTTAGAAATCTAAGCAGAATGCAAATGACTAAGAA
TAATGTTGTTGAAATAACATAAAATAGGTTATAACTTTGATACTCATTAGTAACAAATCTTTCA
ATACATCTTACGGTCTGTTAGGTGTAGATTAGTAATGAAGTGGGAAGCCACTGCAAGCTAGTAT
ACATGTAGGGAAAGATAGAAAGCATTGAAGCCAGAAGAGAGACAGAGGACATTTGGGCTAGATC
TGACAAGAAAAACAAATGTTTTAGTATTAATTTTTGACTTTAAATTTTTTTTTTATTTAGTGAA
TACTGGTGTTTAATGGTCTCATTTTAATAAGTATGACACAGGTAGTTTAAGGTCATATATTTTA
TTTGATGAAAATAAGGTATAGGCCGGGCACGGTGGCTCACACCTGTAATCCCAGCACTTTGGGA
GGCCGAGGCAGGCGGATCACCTGAGGTCGGGAGTTAGAGACTAGCCTCAACATGGAGAAACCCC
GTCTCTACTAAAAAAAATACAAAATTAGGCGGGCGTGGTGGTGCATGCCTGTAATCCCAGCTAC
TCAGGAGGCTGAGGCAGGAGAATTGCTTGAACCTGGGAGGTGGAGGTTGCGGTGAGCCGAGATC
ACCTCATTGCACTCCAGCCTGGGCAACAAGAGCAAAACTCCATCTCAAAAAAAAAAAAATAAGG
TATAAGCGGGCTCAGGAACATCATTGGACATACTGAAAGAAGAAAAATCAGCTGGGCGCAGTGG
CTCACGCCGGTAATCCCAACACTTTGGGAGGCCAAGGCAGGCGAATCACCTGAAGTCGGGAGTT
CCAGATCAGCCTGACCAACATGGAGAAACCCTGTCTCTACTAAAAATACAAAACTAGCCGGGCA
TGGTGGCGCATGCCTGTAATCCCAGCTACTTGGGAGGCTGAGGCAGGAGAATTGCTTGAACCGA
GAAGGCGGAGGTTGCGGTGAGCCAAGATTGCACCATTGCACTCCAGCCTGGGCAACAAGAGCGA
AACTCCGTCTCAAAAAAAAAAGGAAGAAAAATATTTTTTTAAATTAATTAGTTTATTTATTTTT
TAAGATGGAGTTTTGCCCTGTCACCCAGGCTGGGGTGCAATGGTGCAATCTCGGCTCACTGCAA
CCTCCGCCTCCTGGGTTCAAGTGATTCTCCTGCCTCAGCTTCCCGAGTAGCTGTGATTACAGCC
ATATGCCACCACGCCCAGCCAGTTTTGTGTTTTGTTTTGTTTTTTGTTTTTTTTTTTTGAGAGG
GTGTCTTGCTCTGTCCCCCAAGCTGGAGTGCAGCGGCGCGATCTTGGCTCACTGCAAGCTCTGC
CTCCCAGGTTCACACCATTCTCTTGCCTCAGCCTCCCGAGTAGCTGGGACTACAGGTGCCCGCC
ACCACACCCGGCTAATTTTTTTGTGTTTTTAGTAGAGATGGGGTTTCACTGTGTTAGCCAGGAT
GGTCTCGATCTCCTGACCTTTTGATCCACCCGCCTCAGCCTCCCCAAGTGCTGGGATTATAGGC
GTGAGCCACTGTGCCCGGCCTAGTCTTGTATTTTTAGTAGAGTCGGGATTTCTCCATGTTGGTC
AGGCTGTTCTCCAAATCCGACCTCAGGTGATCCGCCCGCCTTGGCCTCCAAAAGTGCAAGGCAA
GGCATTACAGGCATGAGCCACTGTGACCGGCAATGTTTTTAAATTTTTTACATTTAAATTTTAT
TTTTTAGAGACCAGGTCTCACTCTATTGCTCAGGCTGGAGTGCAAGGGCACATTCACAGCTCAC
TGCAGCCTTGACCTCCAGGGCTCAAGCAGTCCTCTCACCTCAGTTTCCCGAGTAGCTGGGACTA
CAGTGATAATGCCACTGCACCTGGCTAATTTTTATTTTTATTTATTTATTTTTTTTTGAGACAG
AGTCTTGCTCTGTCACCCAGGCTGGAGTGCAGTGGTGTAAATCTCAGCTCACTGCAGCCTCCGC
CTCCTGGGTTCAAGTGATTCTCCTGCCTCAACCTCCCAAGTAGCTGGGATTAGAGGTCCCCACC
ACCATGCCTGGCTAATTTTTTGTACTTTCAGTAGAAACGGGGTTTTGCCATGTTGGCCAGGCTG
TTCTCGAACTCCTGAGCTCAGGTGATCCAACTGTCTCGGCCTCCCAAAGTGCTGGGATTACAGG
CGTGAGCCACTGTGCCTAGCCTGAGCCACCACGCCGGCCTAATTTTTAAATTTTTTGTAGAGAC
AGGGTCTCATTATGTTGCCCAGGGTGGTGTCAAGCTCCAGGTCTCAAGTGATCCCCCTACCTCC
GCCTCCCAAAGTTGTGGGATTGTAGGCATGAGCCACTGCAAGAAAACCTTAACTGCAGCCTAAT
AATTGTTTTCTTTGGGATAACTTTTAAAGTACATTAAAAGACTATCAACTTAATTTCTGATCAT
ATTTTGTTGAATAAAATAAGTAAAATGTCTTGTGAAACAAAATGCTTTTTAACATCCATATAAA
GCTATCTATATATAGCTATCTATATCTATATAGCTATTTTTTTTAACTTCCTTTATTTTCCTTA
CAGGGTTTTAGACAAAATCAAAAAGAAGGAAGGTGCTCACATTCCTTAAATTAAGGAGTAAGTC
TGCCAGCATTATGAAAGTGAATCTTACTTTTGTAAAACTTTATGGTTTGTGGAAAACAAATGTT
TTTGAACATTTAAAAAGTTCAGATGTTAGAAAGTTGAAAGGTTAATGTAAAACAATCAATATTA
AAGAATTTTGATGCCAAAACTATTAGATAAAAGGTTAATCTACATCCCTACTAGAATTCTCATA
CTTAACTGGTTGGTTGTGTGGAAGAAACATACTTTCACAATAAAGAGCTTTAGGATATGATGCC
ATTTTATATCACTAGTAGGCAGACCAGCAGACTTTTTTTTATTGTGATATGGGATAACCTAGGC
ATACTGCACTGTACACTCTGACATATGAAGTGCTCTAGTCAAGTTTAACTGGTGTCCACAGAGG
ACATGGTTTAACTGGAATTCGTCAAGCCTCTGGTTCTAATTTCTCATTTGCAGGAAATGCTGGC
ATAGAGCAGCACTAAATGACACCACTAAAGAAACGATCAGACAGATCTGGAATGTGAAGCGTTA
TAGAAGATAACTGGCCTCATTTCTTCAAAATATCAAGTGTTGGGAAAGAAAAAAGGAAGTGGAA
TGGGTAACTCTTCTTGATTAAAAGTTATGTAATAACCAAATGCAATGTGAAATATTTTACTGGA
CTCTATTTTGAAAAACCATCTGTAAAAGACTGAGGTGGGGGTGGGAGGCCAGCACGGTGGTGAG
GCAGTTGAGAAAATTTGAATGTGGATTAGATTTTGAATGATATTGGATAATTATTGGTAATTTT
ATGAGCTGTGAGAAGGGTGTTGTAGTTTATAAAAGACTGTCTTAATTTGCATACTTAAGCATTT
AGGAATGAAGTGTTAGAGTGTCTTAAAATGTTTCAAATGGTTTAACAAAATGTATGTGAGGCGT
ATGTGGCAAAATGTTACAGAATCTAACTGGTGGACATGGCTGTTCATTGTACTGTTTTTTTCTA
TCTTCTATATGTTTAAAAGTATATAATAAAAATATTTAATTTTTTTTTAAATTAGCTGTATCTG
TGATTGTATTTCTTTTTTGCATATTATTTTGCCCTTTGGCCCATATTTTGATATGGATGCCACC
ATAGCATTTTGTGTATGTGCATGTGTATTCCCACTTAATGTCACATTTTTCATGTCTTTACATA
TTCTTATTTTTGTTTGTTTTTGAGACAGAGTCTCGCTCTGCTGCCCACGCTGGAGTGCAGTGGT
GCAATCTCAGCTCACTGCAACCTCTGCTATCCGGGTTCAAGCAGTTCTCGTGCCTCACCCACGT
GAGTAGTTGGGATTACAGGCATGTGGCACCATGCCCCACTAAGTTTTGTATTTTTAGTAGAGAT
GGAGTTTCACCATGTTGGCCAGGCTGGTCTCAAACTCCTGCCCTCAAGTGATTCGACCACCCTG
GCCTCCCAAAGTGCTGGGATTACAGCCGTGAGCCACCGCACACGGCCTCTCTATTTATTTCTAT
ACATAGCTTTTCACATTATATTATGTTTATATATTGTTTATATCTGTATTTCCTCTTTCATTAG
AGAAAAGGTAGTACATCTTATTCTTCATGGTGTCTACAATATCTGGCAGTTTTTGGAAGTCAAG
CGTGAGCTTAGAGCATAGACTGGTGGGATTGTCAAAGAAGAGGGCAACTGGAAGAGAACTGTCA
GTTATTTTTGGATCAGTCTTTAATTCATCATGACGGGTTAGGCATTAGTTGTATTTCTTGCTAA
TTTTGAAGAAGACTTATTAACAAATCCTACATTAGGTAAATGGTTTTGAAAGTTGAGTTAATCA
TAATGGTGTTTGACCTAGGACTATTTTTAGGCCCTATTTATCTTAATATCGAATAATGAAGCAG
CTTCCCCCTTAGATATAGACAGAAAACATCAAAGCCACCACACTACCTGGCTGGATTTATCCTA
GTAATAAAATCAAAACTGAGC.

In embodiments, the Exons of each of the SMN1 and SMN2 polynucleotides differ only by an alteration(s) in Exon 7 (see Gladman, et al., “A humanized Smn gene containing the SMN2 nucleotide alteration in Exon 7 mimics SMN2 splicing and the SMA disease phenotype,” Human Molecular Genetics, 19:4239-4252 (2010), the disclosure of which is incorporated herein in its entirety for all purposes). In embodiments, the alteration is a C>T nucleotide transition(s) in Exon 7 of an SMN2 polynucleotide relative to Exon 7 of an SMN1 polynucleotide, optionally where the alteration is associated with spinal muscular atrophy (SMA).

The term “about” or “approximately” means within an acceptable error range for the particular value as determined by one of ordinary skill in the art, which will depend in part on how the value is measured or determined, i.e., the limitations of the measurement system.

By “adenine” or “9H-Purin-6-amine” is meant a purine nucleobase with the molecular formula C₅H₅N₅, having the structure

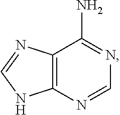

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

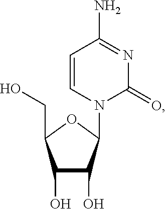

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, and PCT/US2017/045381.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 15, one of the combinations of alterations listed in Table 15, or an alteration at one or more of the amino acid positions listed in Table 15, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10% 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease. In an embodiment, the disease is SMA.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences with about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n(D10A) polypeptide. Non-limiting examples of cytidine deaminase domains include rAPOBEC, ppAPOBEC, RrA3F, AmAPOBECI, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:

| | |
|---|---|
| Glutamine | CAG → TAG Stop codon |
| | CAA → TAA |
| Arginine | CGA → TGA |
| Tryptophan | TGG → TGA |
| | TGG → TAG |
| | TGG → TAA |

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

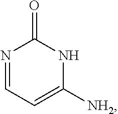

and corresponding to CAS No. 71-30-7.

By “cytidine” is meant a cytosine molecule attached to a ribose sugar via a glycosidic bond, having the structure

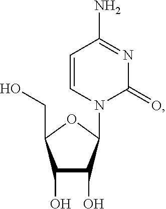

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence, or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include spinal muscular atrophy. In embodiments, the spinal muscular atrophy is type 0, type I, type II, type III, or type IV.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease. In an embodiment, an effective amount of a base editor system described herein is sufficient to increase expression of SMN2. In another embodiment, an effective amount of a base editor system described herein is sufficient to increase muscle function in a subject having SMA.

The term “endonuclease” refers to a protein or polypeptide capable of catalyzing the cleavage of internal regions in a polynucleotide.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “gene” is meant a polynucleotide sequence that is transcribed as a single unit.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In one embodiment, SMN1 or SMN2 polypeptide or polynucleotides are markers associated with SMA.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). In one embodiment, the term “mutation” refers to a genomic alteration that results in the loss of or a decrease in SMN1 polypeptide expression.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′-e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
or
(SEQ ID NO: 196)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference.

Exemplary nucleic acid programmable DNA binding proteins and nucleic acid sequences encoding nucleic acid programmable DNA binding proteins are provided in the Sequence Listing as SEQ ID NOs: 197-230, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, human, non-human primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder. In one embodiment, a subject in need suffers from SMA.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest.

In one embodiment, a reference is a cell or subject expressing wild-type SMN1.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the invention, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at about 60%, 80%, 85%, 90%, 95%, 96%, 97%, 98%, 99%, 99.9%, or even 99.99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

Nucleic acid molecules useful in the methods of the invention include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

The term “target site” refers to a sequence within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>sp|P14739|UNGI_BPPB2 Uracil-DNA glycosylase
inhibitor
(SEQ ID NO: 231)
MTNLSDIIEKETGKQLVIQESILMLPEEVEEVIGNKPESDILVHTAYDE
STDENVMLLTSDAPEYKPWALVIQDSNGENKIKML

In some embodiments, the agent inhibiting the uracil-excision repair system is a uracil stabilizing protein (USP). See, e.g., WO 2022015969 A1, incorporated herein by reference . . . .

As used herein, the term “vector” refers to a means of introducing a nucleic acid sequence into a cell, resulting in a transformed cell. Vectors include plasmids, transposons, phages, viruses, liposomes, lipid nanoparticles, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a ribbon diagram of an SMN2 polypeptide and a schematic representation of alterations that may be introduced to an SMN2 polynucleotide sequence using the methods and base editors provided herein to treat SMA. In embodiments, Intron 7 of SMN2 is modified by introducing a C10>T alteration, which disrupts local secondary structure. The precise conversion of Exon 7 C6U or C6T (i.e., altering T6 of SMN2 to be a C, as in SMN1) introduces an alteration that mimics an SMN1 sequence. Introducing an Exon 7 A54G alteration in SMN2 alters splicing.

FIG. 2 provides a bar graph showing percent C-to-T conversion (%) at C10, C7, and C11 of Intron 7 of an SMN2 polynucleotide. The base editors used to effect the edits were a BE4 and a pmCDA-BE4.

FIG. 3 provides a bar graph showing percent C-to-T conversion (%) at C10 of Intron 7 of an SMN2 polynucleotide. The base editors used to effect the edits were a BE4 and a pmCDA-BE4.

FIG. 4 provides a bar graph showing percent C-to-T conversion (%) at C7 of Intron 7 of an SMN2 polynucleotide. The base editors used to effect the edits were a BE4 and a pmCDA-BE4.

FIG. 5 provides a bar graph showing percent C-to-T conversion (%) at C11 of Intron 7 of an SMN2 polynucleotide. The base editors used to effect the edits were a BE4 and a pmCDA-BE4.

FIG. 6 provides a schematic diagram illustrating how the severity of spinal muscular atrophy (SMA) can depend upon the number of copies of SMN2 present in the genome of a subject deficient for expression of functional SMN1 polypeptides from SMN1. Each copy of SMN2 in the genome provides for a small amount of expression of functional survival motor neuron (SMN) protein. The most severe form of SMA is Type 1, which generally corresponds to a subject with a genome containing about 2 copies of SMN2. Type 2 SMA corresponds to a subject with a genome containing about 2 copies of SMN2. Type 3 SMA, which is the least severe form of the disease, generally corresponds to a subject with a genome containing about 3 to 4 copies of SMN2. As shown in FIG. 6, the transcription from the SMN2 gene results in the production of about 90% transcripts spliced to omit Exon 7 and that encode a protein containing a degron, and about 10% transcripts spliced to include Exon 7 and encoding a full-length SMN protein (i.e., SMN2). In FIG. 6, the left portion of the figure depicts the SMN1 and SMN2 genes and the right portion of the figure depicts spliced mRNA transcripts expressed from the gene. In FIG. 6, the C above the SMN1 gene indicates the C6 nucleotide of Exon 7 that corresponds to T6 in Exon 7 of the SMN2 gene. Altering T6 of SMN2 to be a C can increase levels of incorporation of Exon 7 into spliced transcripts expressed from SMN2.

FIG. 7 provides a schematic diagram showing targets for base editing in the SMN2 gene The adenosine on the DNA strand complementary to T6 in Exon 7 of SMN2 is deaminated to alter the nucleotide to a G and, thereby, alter T6 to C6. SMN1 contains a cytidine at position 6 of Exon 7 and this cytidine plays a role in proper splicing of transcripts expressed from SMN1 to include Exon 7. Alteration of A54 in Exon 7 or of C10 in the intron downstream of Exon 7 can also improve levels of incorporation of Exon 7 into spliced transcripts expressed from SMN2. These alterations are associated with an increase in levels of SMN expressed in a cell.

FIGS. 8A-8C provide bar graphs and schematic diagrams showing levels of expression of the SMN2 transcripts indicated both above and beneath each plot in SMA patient fibroblast cells (GM03813 cells) edited using the indicated base editor systems (x-axis) (see Tables 2B and 2C). FIG. 8A provides a bar graph showing total levels of spliced SMN transcripts measured in the cells. The transcript levels were measured using qRT-PCR in combination with a probe targeting the region of the spliced SMIN2 transcript depicted in the schematic below the plot straddling Exon 1 and Exon 2 in combination with a forward (F) and reverse (R) primer complementary to the indicated regions of the transcript. FIG. 8B provides a bar graph showing total levels of spliced SMN2 transcripts measured in the cells that contained Exon 7. The transcript levels were measured using qRT-PCR in combination with a probe targeting the region of the spliced SMN2 transcript depicted in the schematic below the plot within Exon 7 in combination with a forward (2F) and reverse (1R) primer complementary to the indicated regions of the transcript. FIG. 8C provides a bar graph showing total levels of spliced SMN transcripts measured in the cells that were lacking Exon 7. The transcript levels were measured using qRT-PCR in combination with a probe targeting the region of the spliced SMN2 transcript depicted in the schematic below the plot straddling Exon 6 and Exon 8 in combination with a forward (1F) and reverse (1R) primer complementary to the indicated regions of the transcript. In FIGS. 8A-8C untreated cells (untreated) and mock transfected cells (Mock transf.) were used as negative controls, transfection with 100 nM nusinersen was used as a positive control, and the base editing system containing a cytidine base editor (CBE) and guide number 88, which did not target the SMN2 gene, was used as a positive control for base editing. In FIGS. 8A-8C, “ASO” indicates transfection with the antisense oligonucleotide (ASO) nusinersen. In FIGS. 8A-8C, transcript levels were normalized to levels measured for the TATA-box binding protein (TBP) housekeeping gene.

FIGS. 9A-9C provide bar graphs and a schematic showing base editing efficiencies achieved in SMA patient fibroblast cells (GM03813 cells) using the guide polynucleotide gRNA1962 (see Tables 2B and 2C for sequence) in combination with a cytidine base editor (CBE) (FIG. 9A) or an adenosine base editor (ABE) (FIG. 9B). In FIGS. 9A and 9B, untreated cells (untreated) were used as a negative control, and the base editing system containing a cytidine base editor (CBE) and guide number 88, which did not target the SMN2 gene, was used as a positive control for base editing. The base editor system CBE+gRNA1962 facilitated base editing near the 5′ splice site. FIG. 9C provides a schematic depicting the two adenosines (boxed) targeted by a base editing system comprising an adenosine base editor and gRNA9162 and the two adenosines (boxed) targeted by a base editing system comprising a cytidine base editor (CBE) and gRNA9162. Editing of the first A (lower strand, first from left in box) to a G results in a silent mutation (i.e., AAT→AAC; Asn→Asn). Editing of the second A (lower strand, second from left in box) to G removes a stop codon from Exon 7. Both C's boxed within the lower strand depicted in FIG. 9C fall outside the coding sequence (CDS) of SMN2. Not intending to be bound by theory, altering either of the boxed cytidines may affect RNA stem loop structure and a nearby 5′ splice site, improving Exon 7 inclusion (see Singh, et al. “Splicing of a Critical Exon of Human Survival Motor Neuron Is Regulated by a Unique Silencer Element Located in the Last Intron,” Molecular and Cellular Biology, 26:1333-1346 (2006), the disclosure of which is incorporated herein by reference in its entirety for all purposes). In FIG. 9C, the nucleotide sequences depicted are complementary to one another and the top strand has the following sequence:

(SEQ ID NO: 617)
AGGGTTTTAGACAAAATCAAAAAGAAGGAAGGTGCTCACATTCCTTAAA
TTAAGGAGTAAGTC.

FIGS. 10A-10C provide bar graphs and schematics showing levels of expression of the SMN2 transcripts indicated both above and below each plot in SMA patient fibroblast cells (GM03813 cells) edited using the indicated base editor systems (x-axis) (see Tables 2B and 2C). FIG. 10A provides a bar graph showing total levels of spliced SMN transcripts measured in the cells. The transcript levels were measured using qRT-PCR in combination with a probe targeting the region of the spliced SMN2 transcript depicted in the schematic below the plot straddling Exon 1 and Exon 2 in combination with a forward (F) and reverse (R) primer complementary to the indicated regions of the transcript. FIG. 10B provides a bar graph showing total levels of spliced SMN transcripts measured in the cells that contained Exon 7. The transcript levels were measured using qRT-PCR in combination with a probe targeting the region of the spliced SMN2 transcript depicted in the schematic below the plot within Exon 7 in combination with a forward (2F) and reverse (1R) primer complementary to the indicated regions of the transcript. FIG. 10C provides a bar graph showing total levels of spliced SMN transcripts measured in the cells that were lacking Exon 7. The transcript levels were measured using qRT-PCR in combination with a probe targeting the region of the spliced SMN2 transcript depicted in the schematic below the plot straddling Exon 6 and Exon 8 in combination with a forward (1F) and reverse (1R) primer complementary to the indicated regions of the transcript. In FIGS. 10A-10C untreated cells (untreated) and mock transfected cells (Mock transf.) were used as negative controls, transfection with 30 nM nusinersen was used as a positive control, and the base editing system containing a cytidine base editor (CBE) and guide number 88, which did not target the SMN2 gene, was used as a positive control for base editing. In FIGS. 10A-10C, “ASO” indicates transfection with the antisense oligonucleotide (ASO) nusinersen. In FIGS. 10A-10C, transcript levels were normalized to levels measured for the TATA-box binding protein (TBP) housekeeping gene.

FIGS. 11A and 11B provide a bar graph and a schematic diagram showing base editing efficiencies achieved in SMA patient fibroblast cells (GM03813 cells) using the guide polynucleotide gRNA1973 (see sequence provided in Tables 2B and 2C) in combination with an adenosine base editor (ABE). In FIG. 11A, untreated cells (untreated) were used as a negative control, and the base editing system containing a cytidine base editor (CBE) and guide number 88, which did not target the SMN2 gene, was used as a positive control for base editing. FIG. 11B provides a schematic showing three adenines (boxed) within Exon 7 of SMN2 that are targeted by the base editing system containing gRNA1973 and the ABE). In FIG. 11B, the nucleotide sequences depicted are complementary to one another and the top strand has the following sequence:

(SEQ ID NO: 617)
AGGGTTTTAGACAAAATCAAAAAGAAGGAAGGTGCTCACATTCCTTAAA
TTAAGGAGTAAGTC.

FIGS. 12A-12C provide bar graphs and schematic diagrams showing levels of expression of the SMN2 transcripts indicated both above and below each plot in SMA patient fibroblast cells (GM03813 cells) edited using the indicated base editor systems (x-axis) (see Tables 2B and 2C). FIG. 12A provides a bar graph showing total levels of spliced SMN transcripts measured in the cells. The transcript levels were measured using qRT-PCR in combination with a probe targeting the region of the spliced SMN2 transcript depicted in the schematic below the plot straddling Exon 1 and Exon 2 in combination with a forward (F) and reverse (R) primer complementary to the indicated regions of the transcript. FIG. 12B provides a bar graph showing total levels of spliced SMN2 transcripts measured in the cells that contained Exon 7. The transcript levels were measured using qRT-PCR in combination with a probe targeting the region of the spliced SMN2 transcript depicted in the schematic below the plot within Exon 7 in combination with a forward (2F) and reverse (1R) primer complementary to the indicated regions of the transcript. FIG. 12C provides a bar graph showing total levels of spliced SMN2 transcripts measured in the cells that were lacking Exon 7. The transcript levels were measured using qRT-PCR in combination with a probe targeting the region of the spliced SMN2 transcript depicted in the schematic below the plot straddling Exon 6 and Exon 8 in combination with a forward (1F) and reverse (1R) primer complementary to the indicated regions of the transcript. In FIGS. 12A-12C untreated cells (untreated) and mock transfected cells (Mock transf.) were used as negative controls, transfection with 30 nM nusinersen was used as a positive control, and the base editing system containing a cytidine base editor (CBE) and guide number 88, which did not target the SMN2 gene, was used as a positive control for base editing. In FIGS. 12A-12C, “ASO” indicates transfection with the antisense oligonucleotide (ASO) nusinersen. In FIGS. 12A-12C, transcript levels were normalized to levels measured for the TATA-box binding protein (TBP) housekeeping gene.

FIGS. 13A-13C provide bar graphs and schematics showing levels of expression of the transcripts indicated both above and beneath each plot in SMA patient fibroblast cells (GM03813 cells) edited using the indicated base editor systems (x-axis) (see Tables 2B and 2C). FIG. 13A provides a bar graph showing total levels of spliced SMN transcripts measured in the cells. The transcript levels were measured using qRT-PCR in combination with a probe targeting the region of the spliced SMN2 transcript depicted in the schematic below the plot straddling Exon 1 and Exon 2 in combination with a forward (F) and reverse (R) primer complementary to the indicated regions of the transcript. FIG. 13B provides a bar graph showing total levels of spliced SMN transcripts measured in the cells that contained Exon 7. The transcript levels were measured using qRT-PCR in combination with a probe targeting the region of the spliced SMN2 transcript depicted in the schematic below the plot within Exon 7 in combination with a forward (2F) and reverse (1R) primer complementary to the indicated regions of the transcript. FIG. 13C provides a bar graph showing total levels of spliced SMN transcripts measured in the cells that were lacking Exon 7. The transcript levels were measured using qRT-PCR in combination with a probe targeting the region of the spliced SMN2 transcript depicted in the schematic below the plot straddling Exon 6 and Exon 8 in combination with a forward (1F) and reverse (1R) primer complementary to the indicated regions of the transcript. In FIGS. 13A-13C untreated cells (untreated) and mock transfected cells (Mock transf.) were used as negative controls, transfection with 30 nM nusinersen was used as a positive control, and the base editing system containing a cytidine base editor (CBE) and guide number 88, which did not target the SMN2 gene, was used as a positive control for base editing. In FIGS. 13A-13C, “ASO” indicates transfection with the antisense oligonucleotide (ASO) nusinersen. In FIGS. 13A-13C, transcript levels were normalized to levels measured for the TATA-box binding protein (TBP) housekeeping gene.

FIGS. 14A and 14B provide bar graphs from an experiment undertaken to evaluate the effect of transfection with the antisense oligonucleotide (ASO) nusinersen at levels ranging from 0 to 100 nM on splicing of SMN2 transcripts in SMA patient fibroblast cells (GM03813 cells). The cells were transfected using Lipofectamine MessengerMAX and nusinersen at a concentration of 0 nM, 1 nM, 10 nM, or 100 nM. Cells were lysed after 5 days of being transfected with nusinersen and transcripts were analyzed using qRT-PCR. FIG. 14A provides a bar graph showing levels of spliced SMN2 transcripts containing Exon 7 measured in the cells. Transcript levels were normalized to transcript levels measured for the TATA-box binding protein (TBP) housekeeping gene. FIG. 14B provides a bar graph showing levels of spliced SMN2 transcripts lacking Exon 7 measured in the cells. Transcript levels were normalized to transcript levels measured for the housekeeping gene RPLP0.

FIG. 15 provides a bar graph from an experiment undertaken to compare the effect of base editing and transfection with the antisense oligonucleotide (ASO) nusinersen at levels ranging from 0 to 100 nM on SMN2 polypeptide levels measured in SMA patient fibroblast cells (GM03813 cells). The base edited cells (i.e., CBE/g1962) were transfected with the guide polynucleotide g1962 (see sequence provided in Tables 2B and 2C) and mRNA encoding the CBE using lipofectamine MessengerMAX. At five (5) days post-transfection, levels of SMN2 polypeptide were measured using an enzyme-linked immunosorbent assay (ELISA). Polypeptide levels were normalized to total protein. Total protein was measured using a Bicinchoninic acid (BCA) assay. In FIG. 15, guide 1962 was used in combination with a cytidine deaminase base editor containing a ppAPOBEC1 deaminase domain and an SpCas9 nucleic acid programmable DNA binding protein domain that recognized an NGA PAM sequence.

FIG. 16 provides a schematic diagram depicting Exon 7 of SMN2 (“SMN2 mRNA”) and portions of the two surrounding introns and providing annotations for various regions targeted by available antisense oligonucleotide (ASO) therapeutics, sites or regions targeted for base editing to influence splicing, and sites targeted by guides described herein. The amino acid sequence depicted in FIG. 16 is GFRQNQKEGRCSHSLN (SEQ ID NO: 618). The two nucleic acid sequences depicted in FIG. 16 are complementary to one another and the top strand has the following sequence:

(SEQ ID NO: 619)
AATAAAATAAGTAAAATGTCTTGTGAAACAAAATGCTTTTTAACATCCA
TATAAAGCTATCTATATATAGCTATCTATATCTATATAGCTATTTTTTT
TAACTTCCTTTATTTTCCTTACAGGGTTTTAGACAAAATCAAAAAGAAG
GAAGGTGCTCACATTCCTTAAATTAAGGAGTAAGTCTGCCAGCATTATG
AAAGTGAATCTTACTTTTGTAAAACTTTATGGTTTGTGGAAAACAAATG
TTTTTGAACATTTAAAAAGTTCAGATGTTAGAAAGTTGAAAGGTTAATG
TAA .

FIG. 17 provides a bar graph showing base editing efficiencies (A→G or C→T) measured for editing of HepG2 cells using the indicated guide polynucleotides (see sequences provided in Tables 2B and 2C) in combination with an adenosine base editor (ABE8.20) or a cytidine base editor (ppAPOBEC1). Base editing efficiencies were measured using biological triplicates. The error intervals depicted at the top of each bar show one standard deviation (STD) from the mean. The cells were transfected with the indicated guide polynucleotide and mRNA encoding the base editor using lipofectamine MessengerMAX.

FIG. 18 provides a bar graph showing base editing efficiencies measured for editing of HepG2 cells using base editing systems containing the indicated inlaid base editors (IBEs) or non-inlaid base editors (i.e., ABE8.20 or ppAPOBEC1) and the indicated guide polynucleotides. Base editing systems containing ABE8.20 or ppAPOBEC1 and guide number 088, which did not target the SMN2 gene, were used as positive controls for base editing. Base editing efficiencies were measured using biological triplicates. The error intervals depicted at the top of each bar show one standard deviation (STD) from the mean. The cells were transfected with the indicated guide polynucleotide and mRNA encoding the base editor using lipofectamine MessengerMAX.

FIG. 19 provides a series of bar graphs showing levels of expression of the transcripts indicated above each plot in SMA patient fibroblast cells (GM03813 cells) edited using the indicated base editor systems (x-axis) (see Tables 2B and 2C). In FIG. 19 “Total SMN” indicates total spliced SMN transcripts measured, “SMN+E7” indicates the total number of spliced SMN transcripts containing Exon 7 that were measured, and “SMNΔEx7 indicates the total number of spliced SMN transcripts lacking Exon 7 that were measured. Transcript measurements were carried out using qRT-PCR as described in FIGS. 8A-8C, 10A-10C, and 12A-13C. In FIG. 19, untreated cells (untreated) and mock transfected cells (Mock transfection) were used as negative controls, transfection with 100 nM nusinersen was used as a positive control, and the base editing system containing a cytidine base editor (CBE) and guide number 88, which did not target the SMN2 gene, was used as a positive control for base editing. In FIG. 19, “ASO” indicates transfection with the antisense oligonucleotide (ASO) nusinersen. In FIG. 19, transcript levels were normalized to levels measured for the TATA-box binding protein (TBP) housekeeping gene. In FIG. 19, the upper bar graphs correspond to cells transfected with 300 ng of the guide polynucleotide and 100 ng of mRNA encoding the base editor, and the lower bar graphs correspond to cells transfected with 60 ng of the guide polynucleotide and 30 ng of mRNA encoding the base editor. In FIG. 19, the guide gRNA1992 was used in combination with a cytidine deaminase base editor containing a ppAPOBEC1 deaminase domain and an SaCas9 nucleic acid programmable DNA binding protein that had specificity for an NNNRRT PAM sequence.

FIGS. 20A and 20B provide bar graphs showing base editing efficiencies measured for editing of SMA patient fibroblast cells (GM03813 cells) using base editing systems containing a cytidine base editor (CBE) or adenosine base editor (ABE) and a guide polynucleotide (see sequences provided in Tables 2B and 2C) indicated on the x-axis of the bar graphs. The left y-axis of each plot shows base editing rates for ABEs and the right y-axis of each plot shows base editing for CBEs. FIG. 20A shows base editing efficiencies measured for cells transfected using 300 ng of mRNA encoding the base editor and 100 ng of the guide polynucleotide. FIG. 20B shows base editing efficiencies measured for cells transfected using 60 ng of mRNA encoding the base editor and 30 ng or the guide polynucleotide. In FIGS. 20A and 20B, untreated cells (untreated) and mock transfected cells (Mock transfection) were used as negative controls, transfection with 100 nM nusinersen was used as a positive control, and the base editing system containing a cytidine base editor (CBE) and guide number 88, which did not target the SMN2 gene, was used as a positive control for base editing.

FIG. 21 provides a bar graph showing maximum base editing efficiencies (A→G or C→T) measured for HepG2 cells contacted with base editor systems containing ABE8.20 or ppAPOBEC1 and a guide polynucleotide (see sequences provided in Tables 2B and 2C) indicated on the x-axis.

FIG. 22 provides a bar graph showing levels of SMN2 polypeptide in SMA patient fibroblast cells (GM0318 cells) edited using base editing systems containing a cytidine base editor (CBE) or adenosine base editor (ABE) and a guide polynucleotide (see sequences provided in Tables 2B and 2C), as indicated on the x-axis. In FIG. 22, untreated cells (Untreated) were used as a negative control, and SMN2 polypeptide levels were normalized to total protein levels.

FIG. 23 provides a bar graph showing maximum A to G or C to T percent (%) base editing measured in SMA patient fibroblast cells (GM0318 cells) edited using base editing systems containing a cytidine base editor (CBE) or adenosine base editor (ABE) and a guide polynucleotide (see sequences provided in Tables 2B and 2C), as indicated on the x-axis. In FIG. 23, untreated cells (Untreated) were used as a negative control.

FIG. 24 provides a bar graph showing maximum A to G or C to T percent (%) base editing measured in SMA patient fibroblast cells (GM0318 cells) edited using base editing systems containing a cytidine base editor (CBE), adenosine base editor (ABE), or inlaid base editor (IBE) and a guide polynucleotide (see sequences provided in Tables 2B and 2C), as indicated on the x-axis. In FIG. 24, untreated cells (Untreated) and mock transfected cells (Mock transf.) were used as negative controls, transfection with 30 nM nusinersen (ASO) was used as a positive control, and the base editing system containing a cytidine base editor (CBE) or an adenosine base editor (ABE) and guide number 88, which did not target the SMN2 gene, was used as a positive control for base editing.

FIG. 25 provides a bar graph showing maximum A to G percent (%) base editing measured in SMA patient fibroblast cells (GM0318 cells) edited using base editing systems containing a cytidine base editor (CBE), adenosine base editor (ABE), or inlaid base editor (IBE) and a guide polynucleotide (see sequences provided in Tables 2B and 2C), as indicated on the x-axis. In FIG. 25, a base editing system containing a cytidine base editor (CBE) or an adenosine base editor (ABE) and guide number 88, which did not target the SMN2 gene, was used as a positive control for base editing.

FIG. 26 provides a bar graph showing levels of SMN1 transcripts measured in SMA patient fibroblast cells (GM0318 cells) edited using base editing systems containing a cytidine base editor (CBE), adenosine base editor (ABE), or inlaid base editor (IBE) and a guide polynucleotide (see sequences provided in Tables 2B and 2C), as indicated on the x-axis. In FIG. 26, untreated cells (Untreated) and mock transfected cells (Mock transf) were used as negative controls, transfection with 30 nM nusinersen (ASO) was used as a positive control, and the base editing system containing a cytidine base editor (CBE) or an adenosine base editor (ABE) and guide number 88, which did not target the SMN2 gene, was used as a positive control for base editing. In FIG. 26, transcript levels were normalized to levels measured for the TATA-box binding protein (TBP) housekeeping gene.

FIG. 27 provides a bar graph showing maximum A to G or C to T percent (%) base editing measured in SMA patient fibroblast cells (GM00232 cells) edited using base editing systems containing a cytidine base editor (CBE) or adenosine base editor (ABE), and a guide polynucleotide (see sequences provided in Tables 2B and 2C), as indicated on the x-axis. In FIG. 27, untreated cells (Untreated) and mock transfected cells (Mock transf.) were used as negative controls, and transfection with 30 nM nusinersen (ASO) was used as a positive control.

FIG. 28 provides a bar graph showing levels of SMN2 polypeptide in the SMA patient fibroblast cells (GM00232 cells) described in FIG. 27. In FIG. 28, SMN2 polypeptide levels were normalized to total protein levels.

FIG. 29 provides a bar graph showing maximum A to G or C to T percent (%) base editing measured in SMA patient fibroblast cells (GM03813 cells) edited using base editing systems containing a cytidine base editor (CBE) or adenosine base editor (ABE), and a guide polynucleotide (see sequences provided in Tables 2B and 2C), as indicated on the x-axis. In FIG. 29, untreated cells (Untreated) and mock transfected cells (Mock transf.) were used as negative controls, and transfection with 30 nM nusinersen (ASO) was used as a positive control.

FIG. 30 provides a bar graph showing levels of SMN2 polypeptide in the SMA patient fibroblast cells (GM03813 cells) described in FIG. 29. In FIG. 30, SMN2 polypeptide levels were normalized to total protein levels.

FIG. 31 provides a bar graph showing maximum A to G or C to T percent (%) base editing measured in SMA patient fibroblast cells (GM00232 cells) edited using base editing systems containing a cytidine base editor (CBE) or adenosine base editor (ABE), and a guide polynucleotide (see sequences provided in Tables 2B and 2C), as indicated on the x-axis. In FIG. 31, untreated cells (Untreated) and mock transfected cells (Mock transf.) were used as negative controls, and transfection with 30 nM nusinersen (ASO) was used as a positive control.

FIG. 32 provides a bar graph showing maximum A to G or C to T percent (%) base editing measured in SMA patient fibroblast cells (GM09677cells) edited using base editing systems containing a cytidine base editor (CBE) or adenosine base editor (ABE), and a guide polynucleotide (see sequences provided in Tables 2B and 2C), as indicated on the x-axis. In FIG. 31, untreated cells (Untreated), mock transfected cells (Mock transf.), or cells transfected with mRNA encoding a base editor variant with low-to-no base editing activity (e.g., deadABE or deadCBE) were used as negative controls, and transfection with 30 nM nusinersen (ASO) was used as a positive control.

FIGS. 33A-33C provide bar graphs showing levels of expression of the SMN2 transcripts indicated above each plot in the base edited SMA patient fibroblast cells (GM009677 cells) of FIG. 32. FIG. 33A provides a bar graph showing total levels of spliced SMN transcripts measured in the cells that were lacking Exon 7. FIG. 33B provides a bar graph showing total levels of spliced SMN transcripts measured in the cells. FIG. 33C provides a bar graph showing total levels of spliced SMN2 transcripts measured in the cells that contained Exon 7. In FIGS. 33A-33C, transcript levels were normalized to levels measured for the TATA-box binding protein (TBP) housekeeping gene.

FIGS. 34A-34C provide bar graphs showing levels of expression of the SMN2 transcripts indicated above each plot in the base edited SMA patient fibroblast cells (GM00232 cells) of FIG. 31. FIG. 34A provides a bar graph showing total levels of spliced SMN transcripts measured in the cells that were lacking Exon 7. FIG. 34B provides a bar graph showing total levels of spliced SMN transcripts measured in the cells. FIG. 34C provides a bar graph showing total levels of spliced SMN2 transcripts measured in the cells that contained Exon 7. In FIGS. 34A-34C, transcript levels were normalized to levels measured for the TATA-box binding protein (TBP) housekeeping gene.

Throughout the figures, the letter “g” is used as an abbreviation for “gRNA” (e.g., “g1976,” rather than “gRNA1976”), or a gRNA sequence is referenced by a number (e.g., “1966,” rather than “gRNA1966”)

### DETAILED DESCRIPTION OF THE INVENTION

Provided herein are compositions (e.g., base editors, endonucleases, and guide RNAs (gRNAs)) and methods for editing, modifying, or altering target polynucleotide, survival of motor neuron 2 (SMN2) polynucleotide, and the use of such compositions and methods for treating Spinal Muscular Atrophy (SMA).

The invention is based, at least in part, upon the discovery that targeted base editing of an SMN2 polynucleotide increases the expression of an SMN2 polypeptide in a cell, and that such editing can be used for the treatment of spinal muscular atrophy (SMA). In particular, increasing activity and/or expression of the SMN2 polypeptide in a subject diagnosed with SMA can be an effective treatment strategy. This increase in activity and/or expression can be effected using any of the base editing systems provided herein. Accordingly, the invention features compositions and methods for editing an SMN2 polynucleotide. The edit to the SMN2 polynucleotide is associated with an increase in expression of a functional SMN2 polypeptide in a cell of a subject, as well as with a reduction in symptoms associated with SMA.

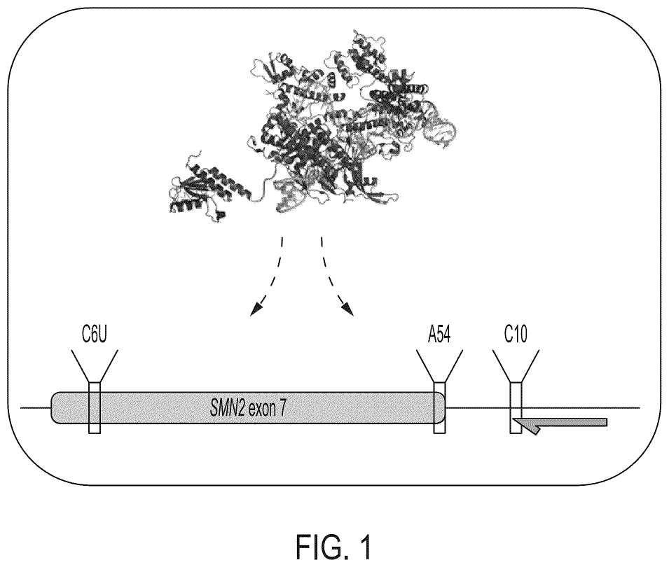

In embodiments, the methods of the present disclosure include altering splicing of an SMN2 polynucleotide transcript. For example, the base editors or base editor systems provided herein can be used for editing a nucleobase associated with splicing efficiency. For example, the base editors or base editor systems provided herein can be used to modify Intron 7 of an SMN2 polynucleotide to disrupt a local secondary structure, to introduce a C10>T alteration, to modify Exon 7 of an SMN2 polynucleotide to introduce a U6C or T6C alteration, to modify Exon 7 of an SMN2 polynucleotide to have an A54G alteration, and/or to introduce a C10T, C7T, and/or C11T alteration in Intron 7 of an SMN2 polynucleotide (see FIG. 1). In embodiments, the methods of the invention include introducing an alteration to an SMN2 polynucleotide sequence. In some instances, the base editor is a BE4 or pmCDA-BE4 base editor.

In some embodiments, the target sequence is a regulatory element associated with an exon (e.g., Exon 7) or intron of the SMN2 polynucleotide and is associated with a change (e.g., an increase) in expression levels (e.g., an increase in SMN2 transcript and/or polypeptide levels) relative to expression levels associated with an unaltered sequence (e.g., a wild-type SMN2 polynucleotide sequence). In some embodiments, the deamination of an A or C nucleobase in the regulatory element results in improved transcription, splicing, and/or translation of the SMN2 mRNA transcript. In some embodiments, the subject has or has the potential to develop spinal muscular atrophy (SMA).

In some instances, the methods of the present disclosure include modifying an SMN2 polynucleotide to introduce a synonymous mutation, missense mutation, or other alteration associated with an increase in levels or activity of the SMN2 polynucleotide and/or polypeptide. The alterations can be effected by a base editor system, such as those described herein.

In some embodiments, the present disclosure provides base editors or nucleases that efficiently generate an intended mutation, such as a point mutation or indel, in a nucleic acid molecule (e.g., a nucleic acid within a genome of a subject) without generating a significant number of unintended mutations, such as unintended point mutations. In some embodiments, an intended mutation is a mutation that is generated by a specific base editor (e.g., an adenosine base editor or a cytidine base editor) bound to a guide polynucleotide (e.g., gRNA), specifically designed to generate the intended mutation. In some embodiments, the intended mutation is an adenine (A) to guanine (G) point mutation within the non-coding or coding region of a gene. In some embodiments, the intended mutation is a cytosine (C) to thymine (T) point mutation within the non-coding or coding region of a gene. In some embodiments, the intended mutation is a mutation of regulatory element associated with an exon (e.g., Exon 7 of an SMN2 polynucleotide) and/or intron (e.g., Intron 7 of an SMN2 polynucleotide) of a gene associated with a disease or disorder (e.g., spinal muscular atrophy). In some embodiments, the intended mutation is a missense mutation. In some embodiments, the intended mutation is a mutation improves transcription, splicing, and/or translation of a complete transcript of a gene, for example, an A to G or C to T change in a regulatory element of an SMN2 polynucleotide (e.g., a regulatory element associated with Exon 7). In some embodiments, the intended mutation is a mutation in a regulatory element of the SMN2 polynucleotide that improves transcription, splicing, and/or translation of a gene transcript and is associated with an increase in SMN2 polypeptide levels in a cell, optionally where the cell is in a subject.

In some embodiments, any of the base editors or endonucleases provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

In some embodiments, editing of a plurality of nucleobase pairs in one or more genes using the methods provided herein results in formation of at least one intended mutation. In some embodiments, the formation of the at least one intended mutation is in a regulatory element (e.g., a regulatory element associated with an intron or exon of an SMN2 polynucleotide) of a disease-associated gene and is associated with increased in SMN2 polypeptide levels in a cell, optionally where the cell is in a subject. It should be appreciated that multiplex editing can be accomplished using any method or combination of methods provided herein.

The present disclosure provides methods for the treatment of a subject diagnosed with spinal muscular atrophy. For example, in some embodiments, a method is provided that comprises administering to a subject having or having a propensity to develop spinal muscular atrophy an effective amount of a nucleobase editor (e.g., an adenosine deaminase base editor or a cytidine deaminase base editor) to effect an alteration in an SMN2 polynucleotide sequence.

### Spinal Muscular Atrophy

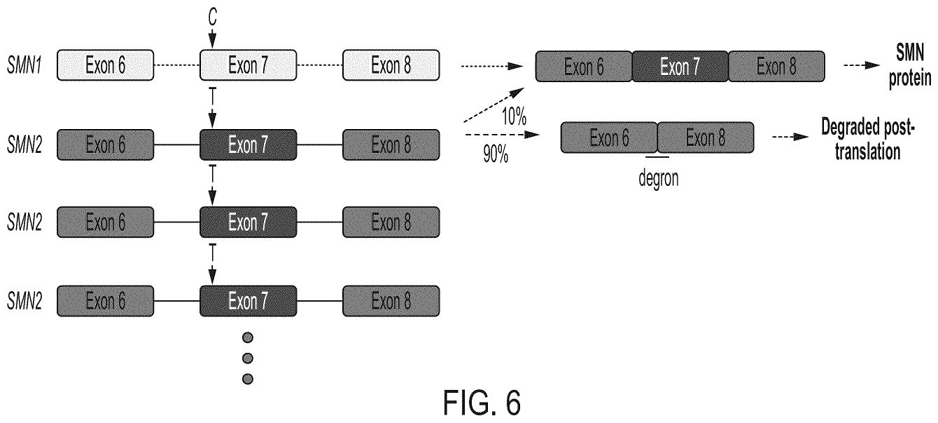

Proximal spinal muscular atrophy (SMA) is a disease characterized by the loss of alpha-motor neurons resulting in progressive muscle atrophy, which often leads to paralysis and death. SMA occurs in approximately 1 in 10,000 live births. Typically, a healthy individual produces most functional SMN protein (e.g., an SMN1 polypeptide) from the SMN1 gene, with a small amount produced from the SMN2 gene. The number of copies of the SMN2 gene in an individual can vary from 1 to up to 8 or more copies (see, e.g., FIG. 6). SMA occurs when there is a homozygous deletion and/or mutation of the Survival Motor Neuron 1 (SMN1) gene located on chromosome 5q13.

SMN2, a nearly identical gene also located on chromosomal segment 5q13, can produce the same protein (SMN) generated by SMN1. This is due to the small number of nucleotide differences between SMN1 and SMN2, most of which have no effect on SMN1 and/or SMN2 polypeptide levels or protein function due to their location in introns. However, SMN2's translationally silent T at nucleotide+6 of Exon 7 instead of SMN1's C causes the final RNA product to be improperly regulated. In SMN2, the majority of the pre-mRNA transcripts generated results in transcripts lacking Exon 7 (FIG. 6). SMN2 does, however, produce a small amount of full-length transcript and thus full-length protein. Improper regulation of the SMN2 gene on account of various alterations relative to the SMN1 gene results in reduced levels of polypeptides expressed from the SMN2 gene. Not intending to be bound by theory, the silent T at nucleotide+6 of Exon 7 disrupts binding of the exonic splicing enhancer SF2/ASF and creates an exonic splicing silencer hnRNP A1 binding site. Also, alterations in the SMN2 gene relative to the SMN1 gene are associated with reductions in splicing efficiency (e.g., an A54G alteration to Exon 7).

Severe disease symptoms occur with lower levels of survival of motor neuron polypeptide (e.g., SMN1 or SMN2 polypeptide), and complete absence of the Smn gene (e.g., an SMN1 and/or SMN2 polynucleotide) is embryonic lethal in mice. Thus, the methods of the present invention provide methods for introducing alterations to an SMN2 polynucleotide sequence to increase production of functional full-length SMN2 transcripts in a subject.

There are different types (0-IV) of muscular atrophy. Spinal muscular atrophy type 0 is evident before birth and is the rarest and most severe form of the condition. Affected infants move less in the womb, and as a result they are often born with joint deformities (contractures). They have extremely weak muscle tone (hypotonia) at birth. Their respiratory muscles are very weak and they often do not survive past infancy due to respiratory failure. Some infants with spinal muscular atrophy type 0 also have heart defects that are present from birth (congenital).

Spinal muscular atrophy type I (also called Werdnig-Hoffmann disease) is the most common form of the condition. It is a severe form of the disorder with muscle weakness evident at birth or within the first few months of life. Most affected children cannot control their head movements or sit unassisted. Children with this type may have swallowing problems that can lead to difficulty feeding and poor growth. They can also have breathing problems due to weakness of respiratory muscles and an abnormally bell-shaped chest that prevents the lungs from fully expanding. Most children with spinal muscular atrophy type I often do not survive past early childhood due to respiratory failure.

Spinal muscular atrophy type II (also called Dubowitz disease) is characterized by muscle weakness that develops in children between ages 6 and 8 months. Children with this type can sit without support, although they may need help getting to a seated position. However, as the muscle weakness worsens later in childhood, affected individuals may need support to sit. Individuals with spinal muscular atrophy type II cannot stand or walk unaided since the disease affects their lower limbs. They often have involuntary trembling (tremors) in their fingers, a spine that curves side-to-side (scoliosis), and respiratory muscle weakness that can be life-threatening. The life span of individuals with spinal muscular atrophy type II varies, but many people with this condition often live into their twenties or thirties.

Spinal muscular atrophy type III (also called Kugelberg-Welander disease) typically causes muscle weakness after early childhood. Individuals with this condition can stand and walk unaided, but over time, walking and climbing stairs may become increasingly difficult. Many affected individuals require wheelchair assistance later in life. People with spinal muscular atrophy type III typically remain mobile and have a normal life expectancy.

Spinal muscular atrophy type IV is rare and often begins in early adulthood. Affected individuals usually experience mild to moderate muscle weakness, tremors, and mild breathing problems. People with spinal muscular atrophy type IV typically have a normal life expectancy.

Non-limiting examples of symptoms of spinal muscular atrophy include areflexia, particularly in extremities; overall muscle weakness; poor muscle tone; limpness or a tendency to flop; difficulty achieving developmental milestones; difficulty sitting/standing/walking; adopting of a frog-leg position when sitting (hips abducted and knees flexed), particularly in children; loss of strength of the respiratory muscles: weak cough, weak cry (infants); accumulation of secretions in the lungs or throat; respiratory distress; bell-shaped torso; fasciculations (twitching) of the tongue; difficulty sucking or swallowing; and/or poor feeding.

Non-limiting examples of agents suitable for use in treatments for spinal muscular atrophy (SMA) include the following: splicing modulators, onasemnogene abeparvovec-xioi, nusinersen, risdiplam, branaplam, RG3039, PTK-SMA1, RG7800, sodium orthovanadate, aclarubicin, SMN2 gene activators, albuterol, butyrates (sodium bytyrate and sodium phenylbuterate), valproic acid, hydroxycarbamide, growth hormone, histone deacetylase inhibitors, benzamide M344, hydroxamic acids (e.g., CBHA, SBHA, entinostat, Panobinostat, trichostatin A, vorinostat, prolactin, reservatrol, curcumin, celecoxib, p38 pathway activators, SMN polynucleotide stabilizers, aminoglycosides, ibuprofen, neuroprotectors, olesoxime, thyrotropin-releasing hormone (TRH), riluzole, beta-lactam antibiotics (e.g., ceftriaxone), follistatin, muscle restoration drugs, reldesemtiv, and apitegromab. Such agents may be used in combination with the methods described herein for the treatment of SMA.

Editing of Target Genes To produce the gene edits described above, cells are contacted with one or more guide RNAs (e.g., a guide RNA containing a sequence listed in any one of Tables 2A to 2C) and a nucleobase editor polypeptide comprising a nucleic acid programmable DNA binding protein (napDNAbp) (e.g., spCas9 or saCas9) and a cytidine deaminase (e.g., ppABOBEC1) or adenosine deaminase (e.g., TadA*8.20), or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid molecule, wherein the at least one nucleic acid molecule encodes one or more guide RNAs and a nucleobase editor polypeptide comprising a nucleic acid programmable DNA binding protein (napDNAbp) and a cytidine deaminase. In some instances, the one or more gRNAs are added directly to a cell.

Exemplary gRNAs that can be used to produce the polynucleotide edits described (e.g., alterations to a regulatory element near or associated with Exon 7 of SMN2 and/or SMN1, etc.) herein include those comprising the spacer sequences listed in Tables 2A or 2C below, extensions thereof, or fragments (e.g., functional fragments) thereof. To produce the polynucleotide edits, cells (e.g., cells in or from a subject, such as a neuron) of a subject are contacted with one or more guide RNAs containing one or more of the spacer sequences listed in Tables 2A or 2C below, extensions thereof, or fragments thereof (e.g., functional fragments thereof) and a nucleobase editor polypeptide or complex containing a nucleic acid programmable DNA binding protein (napDNAbp, such as a Cas9) and a cytidine deaminase or adenosine deaminase. In embodiments, the Cas9 is an spyCas9 or an saCas9. In embodiments, the base editor is introduced to the cell using a polynucleotide sequence (e.g., mRNA) encoding the base editor. The gRNAs containing the spacer sequences listed in Table 2A or 2C, fragments thereof (e.g., functional fragments), or extensions thereof can be used to effect the edits described above and/or described in Table 2A. In some embodiments, the gRNA comprises nucleotide analogs. These nucleotide analogs can inhibit degradation of the gRNA by cellular processes. Tables 2A and 2C provide spacer sequences to be used for gRNAs.

In some embodiments, a guide polynucleotide contains one of the following spacer sequences:

SpCas9:
(SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU
or
SaCas9:
(SEQ ID NO: 425)
GUUUUAGUACUCUGUAAUGAAAAUUACAGAAUCUACUAAAACAAGGCAA
AAUGCCGUGUUUAUCUCGUCAACUUGUUGGCGAGAUUUU

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

TABLE 2A
Exemplary spacer sequences.

| | | SEQ ID | | | |
|---|---|---|---|---|---|
| Name | Spacer Sequence | NO | PAM | Editor(s) | Comments |
| NGG guide 1 | UAUAUAUAGAUAGCUUUAUA | 446 | NGG | ABE | Intron 6, transitions to rat/cyno SMN1 |
| NGG guide 2 | AAACCCUGUAAGGAAAAUAA | 447 | NGG | ABE | intron/exon junction |
| NGG guide 4 | ACUUCCUUUAUUUUCCUUAC | 448 | NGG | ABE/CBE | Intron 6, several transitions to rat SMN1 |
| NGG guide 5 | CUUCCUUUAUUUUCCUUACA | 449 | NGG | ABE/CBE | Intron 6, several transitions to rat SMN1 |
| NGG guide 6 | CAGACUUACUCCUUAAUUUA | 450 | NGG | ABE/CBE | intron/exon junction |
| NGG guide 7 | AAGAUUCACUUUCAUAAUGC | 451 | NGG | ABE/CBE | Intron 7; overlap with nusinersen site |
| NGG guide 8 | CUUACUUUUGUAAAACUUUA | 452 | NGG | ABE/CBE | Intron 7; at TIA1 site |
| NGG guide 9 | UUGUAAAACUUUAUGGUUUG | 453 | NGG | ABE | Intron 7; at TIAl site, one rat transition |
| NGG guide 10 | UCAGAUGUUAGAAAGUUGAA | 454 | NGG | ABE | Intron 7; hnRNP site |
| NGG guide 11 | AUUUUGUCUAAAACCCUGUA | 455 | NGG | ABE | Exon 7, C6U site |
| NGA guide 1 | AAAAAGCAUUUUGUUUCACA | 456 | NGA | ABE/CBE | Intron 6, several transitions to rat SMN1 |
| NGA guide 2 | AAAAUAAGUAAAAUGUCUUG | 457 | NGA | ABE | Intron 6 ahead of element 1 |
| NGA guide 3 | AUAUAUAGAUAGCUUUAUAU | 458 | NGA | ABE | Intron 6, transitions to rat/cyno SMN1 |
| NGA guide 4 | AGAUAUAGAUAGCUAUAUAU | 459 | NGA | ABE | Intron 6, several transitions to rat SMN1 |
| NGA guide 5 | AAAAAUAGCUAUAUAGAUAU | 460 | NGA | ABE/CBE | Intron 6, several transitions to rat SMN1 |
| NGA guide 6 | GUUAAAAAAAAUAGCUAUAU | 461 | NGA | ABE | Intron 6, several transitions to rat SMN1 |
| NGA guide 8 | UUUUGUCUAAAACCCUGUAA | 462 | NGA | ABE | Another Exon 7 C6U guide |
| NGA guide 9 | UUCACUUUCAUAAUGCUGGC | 463 | NGA | ABE/CBE | Intron 7; overlap with nusinersen site |
| NGA guide 10 | GAGUAAGUCUGCCAGCAUUA | 464 | NGA | ABE/CBE | Intron 7; just upstream of nusinersen |
| NGA guide 11 | CAUAAAGUUUUACAAAAGUA | 465 | NGA | ABE | Intron 7; at TIA1 site |
| NGA guide 12 | UGUAAAACUUUAUGGUUUGU | 466 | NGA | ABE | Intron 7; at TIAl site, one rat transition |
| NGA guide 13 | UUGUGGAAAACAAAUGUUUU | 467 | NGA | ABE | Intron 7; at Element 2 |
| NGA guide 14 | CCUUUCAACUUUCUAACAUC | 468 | NGA | ABE/CBE | Intron 7; rat transition |
| NGA guide 15 | AUUUAAAAAGUUCAGAUGUU | 469 | NGA | ABE | Intron 7 |
| NGA guide 16 | UUUGAACAUUUAAAAAGUUC | 470 | NGA | ABE/CBE | Intron 7; URC3 site |
| NGA guide 17 | AAGUUCAGAUGUUAGAAAGU | 471 | NGA | ABE/CBE | Intron 7; rat transition |
| NGA guide 18 | GUCUGCCAGCAUUAUGAAAG | 472 | NGA | CBE | Intron 7; C10 site |
| NNGRRT guide | GCUAUAUAUAGAUAGCUUUAU | 473 | NNGRRT | ABE | Intron 6 |
| 1 | | | (Sa) | | |
| NNGRRT guide | UAACUUCCUUUAUUUUCCUUA | 474 | NNGRRT | CBE | Intron 6; rat transition |
| 2 | | | (Sa) | | |
| NNGRRT guide | GCAGACUUACUCCUUAAUUUA | 475 | NNGRRT | CBE | Intron 7; rat transition |
| 3 | | | (Sa) | | |
| NNGRRT guide | AAGUCUGCCAGCAUUAUGAAA | 476 | NNGRRT | ABE/CBE | Intron 7; overlap with nusinersen site |
| 4 | | | (Sa) | | |
| NNNRRT guide | UAAGUAAAAUGUCUUGUGAAA | 477 | NNNRRT | ABE | Intron 6 |
| 1 | | | (SaKKH) | | |
| NNNRRT guide | UAUAGAUAUAGAUAGCUAUAU | 478 | NNNRRT | ABE | Intron 6, transition to rat SMN1 |
| 2 | | | (SaKKH) | | |
| NNNRRT guide | AAAAAAAAUAGCUAUAUAGAU | 479 | NNNRRT | ABE | Intron 6, transition to rat SMN1 |
| 3 | | | (SaKKH) | | |
| NNNRRT guide | AAACCCUGUAAGGAAAAUAAA | 480 | NNNRRT | ABE/CBE | intron/exon junction; hnRNP binding site |
| 4 | | | (SaKKH) | | |
| NNNRRT guide | UUUUGUCUAAAACCCUGUAAG | 481 | NNNRRT | ABE | Another Exon 7 C6U guide |
| 5 | | | (SaKKH) | | |
| NNNRRT guide | AUAAUGCUGGCAGACUUACUC | 482 | NNNRRT | ABE/CBE | Intron 7; overlap with nusinersen site |
| 6 | | | (SaKKH) | | |
| NNNRRT guide | UACAAAAGUAAGAUUCACUUU | 483 | NNNRRT | ABE | Intron 7; rat transition |
| 7 | | | (SaKKH) | | |
| NNNRRT guide | AACCAUAAAGUUUUACAAAAG | 484 | NNNRRT | ABE | Intron 7; rat transition |
| 8 | | | (SaKKH) | | |
| NNNRRT guide | AAUCUUACUUUUGUAAAACUU | 485 | NNNRRT | ABE/CBE | Intron 7; at TIA1 site |
| 9 | | | (SaKKH) | | |
| NNNRRT guide | CCACAAACCAUAAAGUUUUAC | 486 | NNNRRT | ABE/CBE | Intron 7; at TIA1 site, rat transitions |
| 10 | | | (SaKKH) | | |
| NNNRRT guide | ACAUUUGUUUUCCACAAACCA | 487 | NNNRRT | CBE | Intron 7; at Element 2 |
| 11 | | | (SaKKH) | | |
| NNNRRT guide | GUUUUUGAACAUUUAAAAAGU | 488 | NNNRRT | ABE/CBE | Intron 7; URC3 site |
| 12 | | | (SaKKH) | | |
| NNNRRT guide | AUUUAAAAAGUUCAGAUGUUA | 489 | NNNRRT | ABE | Intron 7 |
| 13 | | | (SaKKH) | | |
| NNNRRT guide | AGUUCAGAUGUUAGAAAGUUG | 490 | NNNRRT | ABE | Intron 7; rat transition |
| 14 | | | (SaKKH) | | |
| NNNRRT guide | CAGAUGUUAGAAAGUUGAAAG | 491 | NNNRRT | ABE | Intron 7; hnRNP site |
| 15 | | | (SaKKH) | | |
| NNNRRT guide | AAAGUUGAAAGGUUAAUGUAA | 492 | NNNRRT | ABE | Intron 7; rat transition |
| 16 | | | (SaKKH) | | |
| NNNRRT guide | GAGUAAGUCUGCCAGCAUUAU | 493 | NNNRRT | CBE | Intron 7; overlap with nusinersen site |
| 17 | | | (SaKKH) | | |
| NNNRRT guide | GAAGUUAAAAAAAAUAGCUAU | 494 | NNNRRT | ABE | Intron 6 |
| 18 | | | (SaKKH) | | |
| NNNRRT guide | AGGAAAAUAAAGGAAGUUAAA | 495 | NNNRRT | ABE | Intron 6; hnRNP site |
| 19 | | | (SaKKH) | | |
| NNNRRT guide | AAACUUUAUGGUUUGUGGAAA | 496 | NNNRRT | ABE | Intron 7; at TIA1 site |
| 20 | | | (SaKKH) | | |
| NGC guide 1 | AUUUUGUCUAAAACCCUGUA | 497 | NGC | ABE | Exon 7 A54 site |
| NGC guide 2 | AAUUAAGGAGUAAGUCUGCC | 498 | NGC | ABE | Exon 7 A54 site |

TABLE 2B
Exemplary guide polynucleotide sequences and measured maximum editing efficiencies (C > T% and
A > G%) measured for base editor systems containing guide polynucleotides containing the listed
guide polynucleotide sequences and a cytidine base editor (CBE; C > T) or an adenosine base
editor (ABE; A > G). In Table 2B, mN = 2′-OMe; Ns = phosphorothioate (PS), where “N” represents
the any nucleotide, as would be understood by one having skill in the art. In some cases, a
nucleotide (N) may contain two modifications, for example, both a 2'-OMe and a PS modification.
For example, a nucleotide with a phosphorothioate and 2' OMe is denoted as “mNs;” when there
are two modifications next to each other, the notation is “mNsmNs.

| | Alternative | | | | |
|---|---|---|---|---|---|
| Guide | Guide | | SEQ | Max | Max |
| Name | Name | sgRNA Nucleotide Sequence | ID NO | A > G% | C > T% |
| gRNA1958 | TSBTx4905 | mUsmUsmAsUUCAACAAAAUAUGAUCGUUUUAGAGCUAG | 499 | 2.7 | 10.1 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1959 | TSBTx4906 | mAsmAsmCsCCUGUAAGGAAAAUAAAGUUUUAGAGCUAG | 500 | 17.3 | 10.9 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1960 | TSBTx4907 | mUsmUsmUsUGUCUAAAACCCUGUAAGUUUUAGAGCUAG | 501 | 5.6 | 38.9 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1961 | TSBTx4908 | mGsmUsmGsAGCACCUUCCUUCUUUUGUUUUAGAGCUAG | 502 | 20.8 | 4.4 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1962 | TSBTx4909 | mAsmCsmUsCCUUAAUUUAAGGAAUGGUUUUAGAGCUAG | 503 | 56.1 | 71.6 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1963 | TSBTx4910 | mAsmGsmAsCUUACUCCUUAAUUUAAGUUUUAGAGCUAG | 504 | 14.8 | 2.2 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1964 | TSBTx4911 | mUsmAsmUsUUUCCUUACAGGGUUUUGUUUUAGAGCUAG | 505 | 0.5 | 9.5 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1965 | TSBTx4912 | mUsmUsmAsGACAAAAUCAAAAAGAAGUUUUAGAGCUAG | 506 | 16.3 | 4.1 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1966 | TSBTx4913 | mGsmCsmUsCACAUUCCUUAAAUUAAGUUUUAGAGCUAG | 507 | 66.9 | 72.9 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1967 | TSBTx4914 | mGsmAsmGsUAAGUCUGCCAGCAUUAGUUUUAGAGCUAG | 508 | 41.4 | 5.2 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1968 | TSBTx4915 | mUsmCsmCsUUAAUUUAAGGAAUGUGGUUUUAGAGCUAG | 509 | | 1.1 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1969 | TSBTx4916 | mCsmUsmUsAAAUUAAGGAGUAAGUCGUUUUAGAGCUAG | 510 | | 0.2 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1970 | TSBTx4917 | mAsmAsmAsCCCUGUAAGGAAAAUAAGUUUUAGAGCUAG | 511 | 41.4 | 2.1 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1971 | TSBTx4918 | mCsmAsmGsACUUACUCCUUAAUUUAGUUUUAGAGCUAG | 512 | 66.6 | 6.3 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1972 | TSBTx4919 | mUsmUsmUsAGACAAAAUCAAAAAGAGUUUUAGAGCUAG | 513 | 62.5 | 2.7 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1973 | TSBTx4920 | mGsmAsmCsAAAAUCAAAAAGAAGGAGUUUUAGAGCUAG | 514 | 52.1 | 2.7 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1974 | TSBTx4921 | mUsmGsmCsUCACAUUCCUUAAAUUAGUUUUAGAGCUAG | 515 | 67 | 28.5 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA1975 | TSBTx4922 | mGsmCsmUsAUAUAUAGAUAGCUUUAUGUUUUAGUACUC | 516 | 53 | 0.6 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1976 | TSBTx4923 | mGsmCsmAsGACUUACUCCUUAAUUUAGUUUUAGUACUC | 517 | 35.7 | 61.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1977 | TSBTx4924 | mGsmUsmGsCUCACAUUCCUUAAAUUAGUUUUAGUACUC | 518 | 27.7 | 59.7 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1978 | TSBTx4925 | mUsmAsmCsUUAUUUUAUUCAACAAAAGUUUUAGUACUC | 519 | 0 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1979 | TSBTx4926 | mCsmAsmUsUUUACUUAUUUUAUUCAAGUUUUAGUACUC | 520 | 0 | 0.2 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1980 | TSBTx4927 | mAsmAsmAsAAAAAUAGCUAUAUAGAUGUUUUAGUACUC | 521 | 0.5 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1981 | TSBTx4928 | mAsmAsmAsCCCUGUAAGGAAAAUAAAGUUUUAGUACUC | 522 | 45.5 | 12.7 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1982 | TSBTx4929 | mUsmUsmUsUGUCUAAAACCCUGUAAGGUUUUAGUACUC | 523 | 17.1 | 48.6 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1983 | TSBTx4930 | mAsmAsmUsGUGAGCACCUUCCUUCUUGUUUUAGUACUC | 524 | 0 | 0.3 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1984 | TSBTx4931 | mAsmUsmAsAUGCUGGCAGACUUACUCGUUUUAGUACUC | 525 | 3.4 | 1.3 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1985 | TSBTx4932 | mUsmUsmCsUGAUCAUAUUUUGUUGAAGUUUUAGUACUC | 526 | 0 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1986 | TSBTx4933 | mGsmAsmUsCAUAUUUUGUUGAAUAAAGUUUUAGUACUC | 527 | 0.2 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1987 | TSBTx4934 | mUsmAsmUsUUUGUUGAAUAAAAUAAGGUUUUAGUACUC | 528 | 1.6 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1988 | TSBTx4935 | mUsmAsmAsGUAAAAUGUCUUGUGAAAGUUUUAGUACUC | 529 | 0 | 0.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1989 | TSBTx4936 | mUsmUsmUsUCCUUACAGGGUUUUAGAGUUUUAGUACUC | 530 | 28.5 | 36.9 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1990 | TSBTx4937 | mUsmUsmAsGACAAAAUCAAAAAGAAGGUUUUAGUACUC | 531 | 2.9 | 24.5 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1991 | TSBTx4938 | mGsmAsmAsGGAAGGUGCUCACAUUCCGUUUUAGUACUC | 532 | 0.2 | 5.8 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA1992 | TSBTx4939 | mUsmCsmAsCAUUCCUUAAAUUAAGGAGUUUUAGUACUC | 533 | 22.6 | 57.4 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2013 | TSBTx4966 | mUsmUsmCsACUUUCAUAAUGCUGGCGUUUUAGAGCUAG | 534 | 0 | 0.1 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2014 | TSBTx4967 | mCsmCsmUsUUCAACUUUCUAACAUCGUUUUAGAGCUAG | 535 | | |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2015 | TSBTx4968 | mGsmUsmCsUGCCAGCAUUAUGAAAGGUUUUAGAGCUAG | 536 | 2.3 | 8.5 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2016 | TSBTx4969 | mUsmGsmUsAAAACUUUAUGGUUUGUGUUUUAGAGCUAG | 537 | 0.7 | 0 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2017 | TSBTx4970 | mAsmGsmAsUUCACUUUCAUAAUGCUGUUUUAGAGCUAG | 538 | | 38.5 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2018 | TSBTx4971 | mAsmAsmUsUAAGGAGUAAGUCUGCCGUUUUAGAGCUAG | 539 | | 0.2 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2019 | TSBTx4972 | mAsmAsmGsAUUCACUUUCAUAAUGCGUUUUAGAGCUAG | 540 | 56.1 | 15.3 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2020 | TSBTx4973 | mUsmUsmGsUAAAACUUUAUGGUUUGGUUUUAGAGCUAG | 541 | 15.3 | 0 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2021 | TSBTx4974 | mUsmCsmAsGAUGUUAGAAAGUUGAAGUUUUAGAGCUAG | 542 | 58.4 | 0.3 |
| | | AAAUAGCAAGUUAAAAUAAGGCUAGUCCGUUAUCAACUU | | | |
| | | GAAAAAGUGGCACCGAGUCGGUGCmUsmUsmUsU | | | |
| gRNA2022 | TSBTx4975 | mAsmAsmGsUCUGCCAGCAUUAUGAAAGUUUUAGUACUC | 543 | 12.2 | 23.8 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2023 | TSBTx4976 | mAsmAsmUsGUAAAACAAUCAAUAUUAGUUUUAGUACUC | 544 | 5.6 | 11.2 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2024 | TSBTx4977 | mUsmAsmCsAAAAGUAAGAUUCACUUUGUUUUAGUACUC | 545 | 0.1 | 0.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2025 | TSBTx4978 | mCsmCsmAsCAAACCAUAAAGUUUUACGUUUUAGUACUC | 546 | 45.1 | 44.4 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2026 | TSBTx4979 | mAsmCsmAsUUUGUUUUCCACAAACCAGUUUUAGUACUC | 547 | 9.8 | 54.4 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2027 | TSBTx4980 | mGsmAsmGsUAAGUCUGCCAGCAUUAUGUUUUAGUACUC | 548 | 16.1 | 64.7 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2028 | TSBTx4981 | mAsmAsmUsCUUACUUUUGUAAAACUUGUUUUAGUACUC | 549 | 0 | 0.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2029 | TSBTx4982 | mAsmAsmAsCUUUAUGGUUUGUGGAAAGUUUUAGUACUC | 550 | 0.7 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2030 | TSBTx4983 | mAsmCsmAsAAUGUUUUUGAACAUUUAGUUUUAGUACUC | 551 | 1.1 | 0.6 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2031 | TSBTx4984 | mGsmUsmUsUUUGAACAUUUAAAAAGUGUUUUAGUACUC | 552 | 0 | 0.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2032 | TSBTx4985 | mAsmUsmUsUAAAAAGUUCAGAUGUUAGUUUUAGUACUC | 553 | 1.2 | 13 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2033 | TSBTx4986 | mAsmGsmUsUCAGAUGUUAGAAAGUUGGUUUUAGUACUC | 554 | 4.6 | 11.5 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2034 | TSBTx4987 | mCsmAsmGsAUGUUAGAAAGUUGAAAGGUUUUAGUACUC | 555 | 0.2 | 0.1 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2035 | TSBTx4988 | mAsmAsmAsGUUGAAAGGUUAAUGUAAGUUUUAGUACUC | 556 | 0.6 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2036 | TSBTx4989 | mUsmUsmGsAAAGGUUAAUGUAAAACAGUUUUAGUACUC | 557 | 1.9 | 0 |
| | | UGUAAUGAAAAUUACAGAAUCUACUAAAACAAGGCAAAA | | | |
| | | UGCCGUGUUUAUCUCGUCAACUUGUUGGCGAGAUsmUsm | | | |
| | | UsmU | | | |
| gRNA2345 | TSBTx5227 | mGsmUsmCsUAAAACCCUGUAAGGAAGUUUUAGAGCUAGAAAU | 629 | | |
| | | AGCAAGUUAAAAUAAGGCUAGUCCGUUAUCAACUUGAAAAAGU | | | |
| | | GGCACCGAGUCGGUGCmUsmUsmUsU | | | |
| gRNA2349 | TSBTx5234 | mUsmUsmAsAGGAGUAAGUCUGCCAGGUUUUAGAGCUAGAAAU | 630 | | |
| | | AGCAAGUUAAAAUAAGGCUAGUCCGUUAUCAACUUGAAAAAGU | | | |
| | | GGCACCGAGUCGGUGCmUsmUsmUsU | | | |
| gRNA2352 | TSBTx5238 | mAsmAsmGsUCUGCCAGCAUUAUGAAGUUUUAGAGCUAGAAAU | 631 | | |
| | | AGCAAGUUAAAAUAAGGCUAGUCCGUUAUCAACUUGAAAAAGU | | | |
| | | GGCACCGAGUCGGUGCmUsmUsmUsU | | | |
| gRNA2354 | TSBTx5242 | mCsmUsmGsCCAGCAUUAUGAAAGUGGUUUUAGAGCUAGAAAU | 632 | | |
| | | AGCAAGUUAAAAUAAGGCUAGUCCGUUAUCAACUUGAAAAAGU | | | |
| | | GGCACCGAGUCGGUGCmUsmUsmUsU | | | |

TABLE 2C
Exemplary spacer sequences.

| | Alternative | | SEQ ID | |
|---|---|---|---|---|
| Guide Name | Guide Name | Spacer Sequence | NO | PAM Sequence |
| gRNA1958 | TSBTx4905 | UUAUUCAACAAAAUAUGAUC | 558 | AGA |
| gRNA1959 | TSBTx4906 | AACCCUGUAAGGAAAAUAAA | 559 | GGA |
| gRNA1960 | TSBTx4907 | UUUUGUCUAAAACCCUGUAA | 560 | GGA |
| gRNA1961 | TSBTx4908 | GUGAGCACCUUCCUUCUUUU | 561 | TGA |
| gRNA1962 | TSBTx4909 | ACUCCUUAAUUUAAGGAAUG | 562 | TGA |
| gRNA1963 | TSBTx4910 | AGACUUACUCCUUAAUUUAA | 563 | GGA |
| gRNA1964 | TSBTx4911 | UAUUUUCCUUACAGGGUUUU | 564 | AGA |
| gRNA1965 | TSBTx4912 | UUAGACAAAAUCAAAAAGAA | 565 | GGA |
| gRNA1966 | TSBTx4913 | GCUCACAUUCCUUAAAUUAA | 566 | GGA |
| gRNA1967 | TSBTx4914 | GAGUAAGUCUGCCAGCAUUA | 567 | TGA |
| gRNA1968 | TSBTx4915 | UCCUUAAUUUAAGGAAUGUG | 568 | AGC |
| gRNA1969 | TSBTx4916 | CUUAAAUUAAGGAGUAAGUC | 569 | TGC |
| gRNA1970 | TSBTx4917 | AAACCCUGUAAGGAAAAUAA | 570 | AGG |
| gRNA1971 | TSBTx4918 | CAGACUUACUCCUUAAUUUA | 571 | AGG |
| gRNA1972 | TSBTx4919 | UUUAGACAAAAUCAAAAAGA | 572 | AGG |
| gRNA1973 | TSBTx4920 | GACAAAAUCAAAAAGAAGGA | 573 | AGG |
| gRNA1974 | TSBTx4921 | UGCUCACAUUCCUUAAAUUA | 574 | AGG |
| gRNA1975 | TSBTx4922 | GCUAUAUAUAGAUAGCUUUAU | 575 | ATGGAT |
| gRNA1976 | TSBTx4923 | GCAGACUUACUCCUUAAUUUA | 576 | AGGAAT |
| gRNA1977 | TSBTx4924 | GUGCUCACAUUCCUUAAAUUA | 577 | AGGAGT |
| gRNA1978 | TSBTx4925 | UACUUAUUUUAUUCAACAAAA | 578 | TATGAT |
| gRNA1979 | TSBTx4926 | CAUUUUACUUAUUUUAUUCAA | 579 | CAAAAT |
| gRNA1980 | TSBTx4927 | AAAAAAAAUAGCUAUAUAGAU | 580 | ATAGAT |
| gRNA1981 | TSBTx4928 | AAACCCUGUAAGGAAAAUAAA | 581 | GGAAGT |
| gRNA1982 | TSBTx4929 | UUUUGUCUAAAACCCUGUAAG | 582 | GAAAAT |
| gRNA1983 | TSBTx4930 | AAUGUGAGCACCUUCCUUCUU | 583 | TTTGAT |
| gRNA1984 | TSBTx4931 | AUAAUGCUGGCAGACUUACUC | 584 | CTTAAT |
| gRNA1985 | TSBTx4932 | UUCUGAUCAUAUUUUGUUGAA | 585 | TAAAAT |
| gRNA1986 | TSBTx4933 | GAUCAUAUUUUGUUGAAUAAA | 586 | ATAAGT |
| gRNA1987 | TSBTx4934 | UAUUUUGUUGAAUAAAAUAAG | 587 | TAAAAT |
| gRNA1988 | TSBTx4935 | UAAGUAAAAUGUCUUGUGAAA | 588 | CAAAAT |
| gRNA1989 | TSBTx4936 | UUUUCCUUACAGGGUUUUAGA | 589 | CAAAAT |
| gRNA1990 | TSBTx4937 | UUAGACAAAAUCAAAAAGAAG | 590 | GAAGGT |
| gRNA1991 | TSBTx4938 | GAAGGAAGGUGCUCACAUUCC | 591 | TTAAAT |
| gRNA1992 | TSBTx4939 | UCACAUUCCUUAAAUUAAGGA | 592 | GTAAGT |
| gRNA2013 | TSBTx4966 | UUCACUUUCAUAAUGCUGGC | 593 | AGA |
| gRNA2014 | TSBTx4967 | CCUUUCAACUUUCUAACAUC | 594 | TGA |
| gRNA2015 | TSBTx4968 | GUCUGCCAGCAUUAUGAAAG | 595 | TGA |
| gRNA2016 | TSBTx4969 | UGUAAAACUUUAUGGUUUGU | 596 | GGA |
| gRNA2017 | TSBTx4970 | AGAUUCACUUUCAUAAUGCU | 597 | GGC |
| gRNA2018 | TSBTx4971 | AAUUAAGGAGUAAGUCUGCC | 598 | AGC |
| gRNA2019 | TSBTx4972 | AAGAUUCACUUUCAUAAUGC | 599 | TGG |
| gRNA2020 | TSBTx4973 | UUGUAAAACUUUAUGGUUUG | 600 | TGG |
| gRNA2021 | TSBTx4974 | UCAGAUGUUAGAAAGUUGAA | 601 | AGG |
| gRNA2022 | TSBTx4975 | AAGUCUGCCAGCAUUAUGAAA | 602 | GTGAAT |
| gRNA2023 | TSBTx4976 | AAUGUAAAACAAUCAAUAUUA | 603 | AAGAAT |
| gRNA2024 | TSBTx4977 | UACAAAAGUAAGAUUCACUUU | 604 | CATAAT |
| gRNA2025 | TSBTx4978 | CCACAAACCAUAAAGUUUUAC | 605 | AAAAGT |
| gRNA2026 | TSBTx4979 | ACAUUUGUUUUCCACAAACCA | 606 | TAAAGT |
| gRNA2027 | TSBTx4980 | GAGUAAGUCUGCCAGCAUUAU | 607 | GAAAGT |
| gRNA2028 | TSBTx4981 | AAUCUUACUUUUGUAAAACUU | 608 | TATGGT |
| gRNA2029 | TSBTx4982 | AAACUUUAUGGUUUGUGGAAA | 609 | ACAAAT |
| gRNA2030 | TSBTx4983 | ACAAAUGUUUUUGAACAUUUA | 610 | AAAAGT |
| gRNA2031 | TSBTx4984 | GUUUUUGAACAUUUAAAAAGU | 611 | TCAGAT |
| gRNA2032 | TSBTx4985 | AUUUAAAAAGUUCAGAUGUUA | 612 | GAAAGT |
| gRNA2033 | TSBTx4986 | AGUUCAGAUGUUAGAAAGUUG | 613 | AAAGGT |
| gRNA2034 | TSBTx4987 | CAGAUGUUAGAAAGUUGAAAG | 614 | GTTAAT |
| gRNA2035 | TSBTx4988 | AAAGUUGAAAGGUUAAUGUAA | 615 | AACAAT |
| gRNA2036 | TSBTx4989 | UUGAAAGGUUAAUGUAAAACA | 616 | ATCAAT |
| gRNA2345 | TSBTx5227 | GUCUAAAACCCUGUAAGGAA | 625 | AATA |
| gRNA2349 | TSBTx5234 | UUAAGGAGUAAGUCUGCCAG | 626 | CATT |
| gRNA2352 | TSBTx5238 | AAGUCUGCCAGCAUUAUGAA | 627 | AG |
| gRNA2354 | TSBTx5242 | CUGCCAGCAUUAUGAAAGUG | 628 | AATC |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors provided herein comprise one or more features that improve base editing activity. For example, any of the nucleobase editors provided herein may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an M1 strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### High Fidelity Cas9 Domains

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 197 and 200) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid. In some embodiments, the high fidelity Cas9 enzyme is SpCas9(K855A), eSpCas9(1.1), SpCas9-HF1, or hyper accurate Cas9 variant (HypaCas9). In some embodiments, the modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

Cas9 Domains with Reduced Exclusivity

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins or complexes provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequence Listing as SEQ ID NOs: 197, 201, and 234-237. Accordingly, in some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Nickases

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain comprises an H840A mutation, while the amino acid residue at position 10 remains a D. In some embodiments, a nickase can be derived from a fully catalytically active (e.g., natural) form of a polynucleotide programmable nucleotide binding domain by removing all or a portion (e.g., a functional portion) of a nuclease domain that is not required for the nickase activity. For example, where a polynucleotide programmable nucleotide binding domain comprises a nickase domain derived from Cas9, the Cas9-derived nickase domain can comprise a deletion of all or a portion (e.g., a functional portion) of the RuvC domain or the HNH domain.

In some embodiments, wild-type Cas9 corresponds to, or comprises the following amino acid sequence:

(SEQ ID NO: 197)
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD

(single underline: HNH domain; double underline: RuvC domain).

In some embodiments, the strand of a nucleic acid duplex target polynucleotide sequence that is cleaved by a base editor comprising a nickase domain (e.g., Cas9-derived nickase domain, Cas12-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain, Cas12-derived nickase domain) can cleave the strand of a DNA molecule which is being targeted for editing. In such embodiments, the non-targeted strand is not cleaved.

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

The amino acid sequence of an exemplary catalytically Cas9 nickase (nCas9) is as follows:

(SEQ ID NO: 201)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some embodiments, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications because a population of cells expressing Cas9 and a gRNA or a guide polynucleotide can result in a diverse array of mutations. In most embodiments, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalytically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain comprises one or more deletions of all or a portion (e.g., a functional portion) of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion (e.g., a functional portion) of a nuclease domain, dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, WI 126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors, H840A, P475A, W476A, N477A, D1125A, WI 126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and WI 126A mutations or when the variant Cas9 protein harbors P475A, W476A, N477A, D1125A, WI 126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided in the Sequence Listing submitted herewith.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRV PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

In some embodiments, one of the Cas9 domains present in the fusion protein or complexes may be replaced with a guide nucleotide sequence-programmable DNA-binding protein domain that has no requirements for a PAM sequence. In some embodiments, the Cas9 is an SaCas9. Residue A579 of SaCas9 can be mutated from N579 to yield a SaCas9 nickase. Residues K781, K967, and H1014 can be mutated from E781, N967, and R1014 to yield a SaKKH Cas9.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9.

Furthermore, Cpf1, unlike Cas9, does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins that are more similar to types I and III than type II systems. Functional Cpf1 does not require the trans-activating CRISPR RNA (tracrRNA), therefore, only CRISPR (crRNA) is required. This benefits genome editing because Cpf1 is not only smaller than Cas9, but also it has a smaller sgRNA molecule (approximately half as many nucleotides as Cas9). The Cpf1-crRNA complex cleaves target DNA or RNA by identification of a protospacer adjacent motif 5′-YTN-3′ or 5′-TTN-3′ in contrast to the G-rich PAM targeted by Cas9. After identification of PAM, Cpf1 introduces a sticky-end-like DNA double-stranded break having an overhang of 4 or 5 nucleotides.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g., a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 or a corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 3A-3D).

TABLE 3A
SpCas9 Variants and PAM specificity

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| PAM | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 3B
SpCas9 Variants and PAM specificity

| | SpCas9 amino acid position | | | | | | | | | | | | | | | | | | |
|---|---|---|---|---|---|---|---|---|---|---|---|---|---|---|---|---|---|---|---|
| | 1114 | 1134 | 1135 | 1137 | 1139 | 1151 | 1180 | 1188 | 1211 | 1219 | 1221 | 1256 | 1264 | 1290 | 1318 | 1317 | 1320 | 1323 | 1333 |
| PAM | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 3C
SpCas9 Variants and PAM specificity

| | SpCas9 amino acid position | | | | | | | | | | |
|---|---|---|---|---|---|---|---|---|---|---|---|
| | 1114 | 1131 | 1135 | 1150 | 1156 | 1180 | 1191 | 1218 | 1219 | 1221 | 1227 |
| PAM | R | Y | D | E | K | D | K | G | E | Q | A |
| SacB.TAT | | | N | | | | N | | V | H | |
| SacB.TAT | | | N | | | | | S | V | H | |
| AAT | | | N | | | | | S | V | H | V |
| TAT | G | | N | | | G | | S | V | H | |
| TAT | G | | N | | | G | | S | V | H | |
| TAT | G | C | N | | | G | | S | V | H | |
| TAT | G | C | N | | | G | | S | V | H | |
| TAT | G | C | N | | | G | | S | V | H | |
| TAT | G | C | N | | E | G | | S | V | H | |
| TAT | G | C | N | V | | G | | S | V | H | |
| TAT | | C | N | | | G | | S | V | H | |
| TAT | G | C | N | | | G | | S | V | H | |

| | SpCas9 amino acid position | | | | | | | | |
|---|---|---|---|---|---|---|---|---|---|
| | 1249 | 1253 | 1286 | 1293 | 1320 | 1321 | 1332 | 1335 | 1339 |
| PAM | P | E | N | A | A | P | D | R | T |
| SacB.TAT | | | | | V | S | | L | |
| SacB.TAT | S | | | | | S | G | L | |
| AAT | S | | K | T | | S | G | L | I |
| TAT | S | K | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |

TABLE 3D
SpCas9 Variants and PAM specificity

| | SpCas9 amino acid position | | | | | | | | | | | | | |
|---|---|---|---|---|---|---|---|---|---|---|---|---|---|---|
| | 1114 | 1127 | 1135 | 1180 | 1207 | 1219 | 1234 | 1286 | 1301 | 1332 | 1335 | 1337 | 1338 | 1349 |
| PAM | R | D | D | D | E | E | N | N | P | D | R | T | S | H |

| | | | | | | | | | | | | | |
|---|---|---|---|---|---|---|---|---|---|---|---|---|---|
| SacB.CAC | | | N | | | V | | | N | Q | N | | |
| AAC | G | | N | | | V | | | N | Q | N | | |
| AAC | G | | N | | | V | | | N | Q | N | | |
| TAC | G | | N | | | V | | | N | Q | N | | |
| TAC | G | | N | | | V | | H | N | Q | N | | |
| TAC | G | | N | | G | V | D | H | N | Q | N | | |
| TAC | G | | N | | | V | | | N | Q | N | | |
| TAC | G | G | N | E | | V | | H | N | Q | N | | |
| TAC | G | | N | | | V | | H | N | Q | N | | |
| TAC | G | | N | | | V | | | N | Q | N | T | R |

Further exemplary Cas9 (e.g., SaCas9) polypeptides with modified PAM recognition are described in Kleinstiver, et al. “Broadening the targeting range of Staphylococcus aureus CRISPR-Cas9 by modifying PAM recognition,” Nature Biotechnology, 33:1293-1298 (2015) DOI: 10.1038/nbt.3404, the disclosure of which is incorporated herein by reference in its entirety for all purposes. In some embodiments, a Cas9 variant (e.g., a SaCas9 variant) comprising one or more of the alterations E782K, N929R, N968K, and/or R1015H has specificity for, or is associated with increased editing activities relative to a reference polypeptide (e.g., SaCas9) at an NNNRRT or NNHRRT PAM sequence, where N represents any nucleotide, H represents any nucleotide other than G (i.e., “not G”), and R represents a purine. In embodiments, the Cas9 variant (e.g., a SaCas9 variant) comprises the alterations E782K, N968K, and R1015H or the alterations E782K, K929R, and R1015H.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

In some embodiments, the napDNAbp is a circular permutant (e.g., SEQ ID NO: 238).

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins or complexes provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, anapDNAbp refers to Cas12c. In some embodiments, the Cas12c protein is a Cas12c1 (SEQ ID NO: 239) or a variant of Cas12c1. In some embodiments, the Cas12 protein is a Cas12c2 (SEQ ID NO: 240) or a variant of Cas12c2. In some embodiments, the Cas12 protein is a Cas12c protein from Oleiphilus sp. H10009 (i.e., OspCas12c; SEQ ID NO: 241) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12c1, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 242-245. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins or complexes provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

Fusion Proteins or Complexes with Internal Insertions

Provided herein are fusion proteins or complexes comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide. In some embodiments, the cytidine deaminase is an APOBEC deaminase (e.g., APOBEC1). In some embodiments, the adenosine deaminase is a TadA (e.g., TadA*7.10 or TadA*8). In some embodiments, the TadA is a TadA*8 or a TadA*9. TadA sequences (e.g., TadA7.10 or TadA*8) as described herein are suitable deaminases for the above-described fusion proteins or complexes.

In some embodiments, the fusion protein comprises the structure:
NH₂-[N-terminal fragment of a napDNAbp]-[deaminase]-[C-terminal fragment of a napDNAbp]-COOH;
NH₂-[N-terminal fragment of a Cas9]-[adenosine deaminase]-[C-terminal fragment of a Cas9]-COOH;
NH₂-[N-terminal fragment of a Cas12]-[adenosine deaminase]-[C-terminal fragment of a Cas12]-COOH;
NH₂-[N-terminal fragment of a Cas9]-[cytidine deaminase]-[C-terminal fragment of a Cas9]-COOH;
NH₂-[N-terminal fragment of a Cas12]-[cytidine deaminase]-[C-terminal fragment of a Cas12]-COOH;
wherein each instance of “]−[” indicates the optional presence of a linker (i.e., the linker is optionally present).

The deaminase can be a circular permutant deaminase. For example, the deaminase can be a circular permutant adenosine deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. In some embodiments, the fusion protein or complex comprises one or two deaminase. The two or more deaminases in a fusion protein or complex can be an adenosine deaminase, a cytidine deaminase, or a combination thereof. The two or more deaminases can be homodimers or heterodimers. The two or more deaminases can be inserted in tandem in the napDNAbp. In some embodiments, the two or more deaminases may not be in tandem in the napDNAbp.

In some embodiments, the napDNAbp in the fusion protein or complex is a Cas9 polypeptide or a fragment (e.g., a functional fragment) thereof. The Cas9 polypeptide can be a variant Cas9 polypeptide. In some embodiments, the Cas9 polypeptide is a Cas9 nickase (nCas9) polypeptide or a fragment (e.g., a functional fragment) thereof. In some embodiments, the Cas9 polypeptide is a nuclease dead Cas9 (dCas9) polypeptide or a fragment (e.g., a functional fragment) thereof. The Cas9 polypeptide in a fusion protein or complex can be a full-length Cas9 polypeptide. In some cases, the Cas9 polypeptide in a fusion protein or complex may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment (e.g., a functional fragment), a portion (e.g., a functional portion), or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments (e.g., functional fragments) or variants of any of the Cas9 polypeptides described herein.

In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH₂-[Cas9(adenosine deaminase)]-[cytidine deaminase]-COOH;
NH₂-[cytidine deaminase]-[Cas9(adenosine deaminase)]-COOH;
NH₂-[Cas9(cytidine deaminase)]-[adenosine deaminase]-COOH; or
NH₂-[adenosine deaminase]-[Cas9(cytidine deaminase)]-COOH.

In some embodiments, the “−” used in the general architecture above indicates the optional presence of linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:
NH₂-[Cas9(TadA*8)]-[cytidine deaminase]-COOH;
NH₂-[cytidine deaminase]-[Cas9(TadA*8)]-COOH;
NH₂-[Cas9(cytidine deaminase)]-[TadA*8]-COOH; or
NH₂-[TadA*8]-[Cas9(cytidine deaminase)]-COOH.

In some embodiments, the “−” used in the general architecture above indicates the optional presence of a linker.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g. adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase)can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2c1 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Cu atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Cu atom with a B-factor that is 50%, 60%, 70%, 80%, 90%, 100%, 110%, 120%, 130%, 140%, 150%, 160%, 170%, 180%, 190%, 200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1027, 1029, 1030, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1027, 1029, 1030, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1027, 1029, 1030, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 1027, 1030, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 1027, 1030, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 1027, 1030, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 1027 1030, 768, 1067, 1248, 1052, and 1246 as numbered in the above Cas9 reference sequence, or a corresponding amino acid residue in another Cas9 polypeptide.

In some embodiments, a cytidine deaminase (e.g., APOBEC) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors (alternatively “inlaid base editors”) are provided in Table 4 below:

TABLE 4
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

Exemplary internal fusion base editor amino acid and nucleotide sequences are provided below, where a SpCas9 domain is indicated by plain text, a linker is indicated by bold text, a TadA*8.20 deaminase domain is indicated by italic-underlined text, and a bipartite nuclear localization signal is indicated by underlined text. The terms “NGA” and “NGC” indicate the PAM sequence recognized by the SpCas9 domain.

NGA_IBE11 amino acid sequence:
(SEQ ID NO: 633)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEGSSGSETPGTSESATPESSGSEVEFSHEYWMRHALTLAKRARDEREVPVGAVLVLNNRVIGE
GWNRAIGLHDPTAHAEIMALRQGGLVMQNYRLYDATLYSTFEPCVMCAGAMIHSRIGRVVFGVR
NAKTGAAGSLMDVLHHPGMNHRVEITEGILADECAALLCRFFRMPRRVFNAQKKAQSSTDQEIG
KATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASARELQK
GNELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKQYRSTKEVLDATLIHQ
SITGLYETRIDLSQLGGDEGADKRTADGSEFESPKKKRKV
NGA_IBE11 nucleotide sequence:
(SEQ ID NO: 634)
atggacaagaagtactctatcggactggccatcggcaccaactctgttggatgggccgtgatca
ccgacgagtacaaggtgcccagcaagaaattcaaggtgctgggcaacaccgaccggcacagcat
caagaagaatctgatcggcgccctgctgttcgactctggcgaaacagccgaagccaccagactg
aagagaaccgccaggcggagatacacccggcggaagaaccggatctgctacctgcaagagatct
tcagcaacgagatggccaaggtggacgacagcttcttccacagactggaagagtccttcctggt
ggaagaggacaagaagcacgagcggcaccccatcttcggcaacatcgtggatgaggtggcctac
cacgagaagtaccccaccatctaccacctgagaaagaaactggtggacagcaccgacaaggccg
acctgagactgatctacctggctctggcccacatgatcaagttccggggccactttctgatcga
gggcgatctgaaccccgacaacagcgacgtggacaagctgttcatccagctggtgcagacctac
aaccagctgttcgaggaaaaccccatcaacgcctctggcgtggacgccaaggctatcctgtctg
ccagactgagcaagagcagaaggctggaaaacctgatcgcccagctgcctggcgagaagaagaa
tggcctgttcggcaacctgattgccctgagcctgggactgacccctaacttcaagagcaacttc
gacctggccgaggatgccaaactgcagctgagcaaggacacctacgacgacgacctggacaatc
tgctggcccagatcggcgatcagtacgccgacttgtttctggccgccaagaacctgtccgacgc
catcctgctgagcgatatcctgagagtgaacaccgagatcacaaaggcccctctgagcgcctct
atgatcaagagatacgacgagcaccaccaggatctgaccctgctgaaggccctcgttagacagc
agctgccagagaagtacaaagagattttcttcgatcagtccaagaacggctacgccggctacat
tgatggcggagccagccaagaggaattctacaagttcatcaagcccatcctggaaaagatggac
ggcaccgaggaactgctggtcaagctgaacagagaggacctgctgcggaagcagcggaccttcg
acaatggctctatccctcaccagatccacctgggagagctgcacgccattctgcggagacaaga
ggacttttacccattcctgaaggacaaccgggaaaagatcgagaagatcctgaccttcaggatc
ccctactacgtgggaccactggccagaggcaatagcagattcgcctggatgaccagaaagagcg
aggaaaccatcacaccctggaacttcgaggaagtggtggacaagggcgccagcgctcagtcctt
catcgagcggatgaccaacttcgataagaacctgcctaacgagaaggtgctgcccaagcactcc
ctgctgtatgagtacttcaccgtgtacaacgagctgaccaaagtgaaatacgtgaccgagggaa
tgagaaagcccgcctttctgagcggcgagcagaaaaaggccattgtggatctgctgttcaagac
caaccggaaagtgaccgtgaagcagctgaaagaggactacttcaagaaaatcgagtgcttcgac
agcgtggaaatcagcggcgtggaagatcggttcaatgccagcctgggcacataccacgacctgc
tgaaaattatcaaggacaaggacttcctggacaacgaagagaacgaggacattctcgaggacat
cgtgctgaccctgacactgtttgaggacagagagatgatcgaggaacggctgaaaacatacgcc
cacctgttcgacgacaaagtgatgaagcaactgaagcggaggcggtacacaggctggggcagac
tgtctcggaagctgatcaacggcatccgggataagcagtccggcaagacaatcctggatttcct
gaagtccgacggcttcgccaacagaaacttcatgcagctgatccacgacgacagcctgaccttt
aaagaggacatccagaaagcccaggtgtccggccaaggcgattctctgcacgagcacattgcca
acctggccggatctcccgccattaagaagggcatcctgcagacagtgaaggtggtggacgagct
tgtgaaagtgatgggcagacacaagcccgagaacatcgtgatcgaaatggccagagagaaccag
accacacagaagggccagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaaccgg
ctgagcgactacgatgtggaccatatcgtgccccagagctttctgaaggacgactccatcgata
acaaggtcctgaccagaagcgacaagaaccggggcaagagcgataacgtgccctccgaagaggt
ggtcaagaagatgaagaactactggcgacagctgctgaacgccaagctgattacccagcggaag
ttcgataacctgaccaaggccgagagaggcggcctgagcgaacttgataaggccggcttcatta
agcggcagctggtggaaacccggcagatcaccaaacacgtggcacagattctggactcccggat
gaacactaagtacgacgagaatgacaagctgatccgggaagtgaaagtcatcaccctgaagtct
aagctggtgtccgatttccggaaggatttccagttctacaaagtgcgggaaatcaacaactacc
atcacgcccacgacgcctacctgaatgccgttgttggaacagccctgatcaagaagtatcccaa
gctggaaagcgagttcgtgtacggcgactacaaggtgtacgacgtgcggaagatgatcgccaag
agcgaaggatcttctggaagcgaaaccccaggcaccagcgagtctgccacaccagaatcatctg
gcagcgaggtcgagttctctcacgaatattggatgagacacgctctcaccctggctaagagagc
cagggacgaaagagaggtgccagttggcgctgtcctggtgttgaacaatcgcgtcatcggagaa
ggatggaatcgcgccattggcctgcacgatccaaccgcacatgccgaaattatggctctgcggc
aaggcggcctcgtgatgcaaaattacagactgtacgatgctaccctctactccaccttcgagcc
ctgtgtcatgtgtgctggggcaatgattcactcccggattggccgcgtggtgtttggagtgcgg
aatgccaagactggcgccgctggatctctgatggacgtcctgcaccatcctgggatgaaccacc
gggtcgagatcacagagggaattctggctgacgagtgcgctgccctgctgtgcaggttctttag
aatgcctagaagggtgttcaacgcccagaaaaaagctcagagcagcaccgatcaagagatcggc
aaggctaccgccaagtactttttctacagcaacatcatgaactttttcaagacagagatcaccc
tggccaacggcgagatccggaaaagacccctgatcgagacaaacggcgaaaccggggagatcgt
gtgggataagggcagagattttgccacagtgcggaaagtgctgagcatgccccaagtgaatatc
gtgaagaaaaccgaggtgcagacaggcggcttcagcaaagagtctatcctgcctaagcggaaca
gcgataagctgatcgccagaaagaaggactgggaccccaagaaatacggcggcttcgtgtctcc
taccgtggcctattctgtgctggtggtggccaaagtggaaaagggcaagtccaaaaagctcaag
agcgtgaaagagctgctggggatcaccatcatggaaagaagcagctttgagaagaacccgatcg
actttctggaagccaagggctacaaagaagtcaagaaggacctcatcatcaagctccccaagta
cagcctgttcgagctggaaaatggccggaagcggatgctggcctctgctagagaactgcagaag
ggaaacgagctggccctgcctagcaaatatgtgaacttcctgtacctggccagccactatgaga
agctgaagggcagccccgaggacaatgagcaaaagcagctgtttgtggaacagcacaagcacta
cctggacgagatcatcgagcagatcagcgagttctccaagagagtgatcctggccgacgctaac
ctggataaggtgctgtctgcctataacaagcaccgggacaagcctatcagagagcaggccgaga
atatcatccacctgtttaccctgaccaacctgggagcccctgccgccttcaagtacttcgacac
caccatcgaccggaagcagtaccggtccaccaaagaggtgctggacgccacactgatccaccag
tctatcaccggcctgtacgaaacccggatcgacctgtctcagctcggaggcgacgaaggcgccg
ataagagaaccgccgatggctctgagttcgagagccccaagaaaaagcgcaaagtgtaa
NGA_IBE12 amino acid sequence:
(SEQ ID NO: 635)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGSSGSETPGTSESATPESSGSEVEFSHEYWMRHALTLAKRARDEREVPVGAVLVLNNRV
IGEGWNRAIGLHDPTAHAEIMALRQGGLVMQNYRLYDATLYSTFEPCVMCAGAMIHSRIGRVVF
GVRNAKTGAAGSLMDVLHHPGMNHRVEITEGILADECAALLCRFFRMPRRVFNAQKKAQSSTDG
KATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASARELQK
GNELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKQYRSTKEVLDATLIHQ
SITGLYETRIDLSQLGGDEGADKRTADGSEFESPKKKRKV
NGA_IBE12 nucleotide sequence:
(SEQ ID NO: 636)
atggacaagaagtactctatcggactggccatcggcaccaactctgttggatgggccgtgatca
ccgacgagtacaaggtgcccagcaagaaattcaaggtgctgggcaacaccgaccggcacagcat
caagaagaatctgatcggcgccctgctgttcgactctggcgaaacagccgaagccaccagactg
aagagaaccgccaggcggagatacacccggcggaagaaccggatctgctacctgcaagagatct
tcagcaacgagatggccaaggtggacgacagcttcttccacagactggaagagtccttcctggt
ggaagaggacaagaagcacgagcggcaccccatcttcggcaacatcgtggatgaggtggcctac
cacgagaagtaccccaccatctaccacctgagaaagaaactggtggacagcaccgacaaggccg
acctgagactgatctacctggctctggcccacatgatcaagttccggggccactttctgatcga
gggcgatctgaaccccgacaacagcgacgtggacaagctgttcatccagctggtgcagacctac
aaccagctgttcgaggaaaaccccatcaacgcctctggcgtggacgccaaggctatcctgtctg
ccagactgagcaagagcagaaggctggaaaacctgatcgcccagctgcctggcgagaagaagaa
tggcctgttcggcaacctgattgccctgagcctgggactgacccctaacttcaagagcaacttc
gacctggccgaggatgccaaactgcagctgagcaaggacacctacgacgacgacctggacaatc
tgctggcccagatcggcgatcagtacgccgacttgtttctggccgccaagaacctgtccgacgc
catcctgctgagcgatatcctgagagtgaacaccgagatcacaaaggcccctctgagcgcctct
atgatcaagagatacgacgagcaccaccaggatctgaccctgctgaaggccctcgttagacagc
agctgccagagaagtacaaagagattttcttcgatcagtccaagaacggctacgccggctacat
tgatggcggagccagccaagaggaattctacaagttcatcaagcccatcctggaaaagatggac
ggcaccgaggaactgctggtcaagctgaacagagaggacctgctgcggaagcagcggaccttcg
acaatggctctatccctcaccagatccacctgggagagctgcacgccattctgcggagacaaga
ggacttttacccattcctgaaggacaaccgggaaaagatcgagaagatcctgaccttcaggatc
ccctactacgtgggaccactggccagaggcaatagcagattcgcctggatgaccagaaagagcg
aggaaaccatcacaccctggaacttcgaggaagtggtggacaagggcgccagcgctcagtcctt
catcgagcggatgaccaacttcgataagaacctgcctaacgagaaggtgctgcccaagcactcc
ctgctgtatgagtacttcaccgtgtacaacgagctgaccaaagtgaaatacgtgaccgagggaa
tgagaaagcccgcctttctgagcggcgagcagaaaaaggccattgtggatctgctgttcaagac
caaccggaaagtgaccgtgaagcagctgaaagaggactacttcaagaaaatcgagtgcttcgac
agcgtggaaatcagcggcgtggaagatcggttcaatgccagcctgggcacataccacgacctgc
tgaaaattatcaaggacaaggacttcctggacaacgaagagaacgaggacattctcgaggacat
cgtgctgaccctgacactgtttgaggacagagagatgatcgaggaacggctgaaaacatacgcc
cacctgttcgacgacaaagtgatgaagcaactgaagcggaggcggtacacaggctggggcagac
tgtctcggaagctgatcaacggcatccgggataagcagtccggcaagacaatcctggatttcct
gaagtccgacggcttcgccaacagaaacttcatgcagctgatccacgacgacagcctgaccttt
aaagaggacatccagaaagcccaggtgtccggccaaggcgattctctgcacgagcacattgcca
acctggccggatctcccgccattaagaagggcatcctgcagacagtgaaggtggtggacgagct
tgtgaaagtgatgggcagacacaagcccgagaacatcgtgatcgaaatggccagagagaaccag
accacacagaagggccagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaaccgg
ctgagcgactacgatgtggaccatatcgtgccccagagctttctgaaggacgactccatcgata
acaaggtcctgaccagaagcgacaagaaccggggcaagagcgataacgtgccctccgaagaggt
ggtcaagaagatgaagaactactggcgacagctgctgaacgccaagctgattacccagcggaag
ttcgataacctgaccaaggccgagagaggcggcctgagcgaacttgataaggccggcttcatta
agcggcagctggtggaaacccggcagatcaccaaacacgtggcacagattctggactcccggat
gaacactaagtacgacgagaatgacaagctgatccgggaagtgaaagtcatcaccctgaagtct
aagctggtgtccgatttccggaaggatttccagttctacaaagtgcgggaaatcaacaactacc
atcacgcccacgacgcctacctgaatgccgttgttggaacagccctgatcaagaagtatcccaa
gctggaaagcgagttcgtgtacggcgactacaaggtgtacgacgtgcggaagatgatcgccaag
agcgaacaagagatcggatcttctggaagcgaaaccccaggcaccagcgagtctgccacaccag
aatcatctggcagcgaggtcgagttctctcacgaatattggatgagacacgctctcaccctggc
taagagagccagggacgaaagagaggtgccagttggcgctgtcctggtgttgaacaatcgcgtc
atcggagaaggatggaatcgcgccattggcctgcacgatccaaccgcacatgccgaaattatgg
ctctgcggcaaggcggcctcgtgatgcaaaattacagactgtacgatgctaccctctactccac
cttcgagccctgtgtcatgtgtgctggggcaatgattcactcccggattggccgcgtggtgttt
ggagtgcggaatgccaagactggcgccgctggatctctgatggacgtcctgcaccatcctggga
tgaaccaccgggtcgagatcacagagggaattctggctgacgagtgcgctgccctgctgtgcag
gttctttagaatgcctagaagggtgttcaacgcccagaaaaaagctcagagcagcaccgatggc
aaggctaccgccaagtactttttctacagcaacatcatgaactttttcaagacagagatcaccc
tggccaacggcgagatccggaaaagacccctgatcgagacaaacggcgaaaccggggagatcgt
gtgggataagggcagagattttgccacagtgcggaaagtgctgagcatgccccaagtgaatatc
gtgaagaaaaccgaggtgcagacaggcggcttcagcaaagagtctatcctgcctaagcggaaca
gcgataagctgatcgccagaaagaaggactgggaccccaagaaatacggcggcttcgtgtctcc
taccgtggcctattctgtgctggtggtggccaaagtggaaaagggcaagtccaaaaagctcaag
agcgtgaaagagctgctggggatcaccatcatggaaagaagcagctttgagaagaacccgatcg
actttctggaagccaagggctacaaagaagtcaagaaggacctcatcatcaagctccccaagta
cagcctgttcgagctggaaaatggccggaagcggatgctggcctctgctagagaactgcagaag
ggaaacgagctggccctgcctagcaaatatgtgaacttcctgtacctggccagccactatgaga
agctgaagggcagccccgaggacaatgagcaaaagcagctgtttgtggaacagcacaagcacta
cctggacgagatcatcgagcagatcagcgagttctccaagagagtgatcctggccgacgctaac
ctggataaggtgctgtctgcctataacaagcaccgggacaagcctatcagagagcaggccgaga
atatcatccacctgtttaccctgaccaacctgggagcccctgccgccttcaagtacttcgacac
caccatcgaccggaagcagtaccggtccaccaaagaggtgctggacgccacactgatccaccag
tctatcaccggcctgtacgaaacccggatcgacctgtctcagctcggaggcgacgaaggcgccg
ataagagaaccgccgatggctctgagttcgagagccccaagaaaaagcgcaaagtgtaa
NGA_IBE16 amino acid sequence:
(SEQ ID NO: 637)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFVSPTVAYSVLVVAKVEKG
KSKKLKSVKELLGITIMERSSFEKNPIDFLEAKGYKEVKKDLIIKLPKYSLFELENGRKRMLAS
ARELQKGNELALPSKYVNFLYLASHYEKLKGGSSGSETPGTSESATPESSGSEVEFSHEYWMRH
ALTLAKRARDEREVPVGAVLVLNNRVIGEGWNRAIGLHDPTAHAEIMALRQGGLVMQNYRLYDA
TLYSTFEPCVMCAGAMIHSRIGRVVFGVRNAKTGAAGSLMDVLHHPGMNHRVEITEGILADECA
ALLCRFFRMPRRVFNAQKKAQSSTDSPEDNEQKQLFVEQHKHYLDEIIEQISEFSKRVILADAN
LDKVLSAYNKHRDKPIREQAENIIHLFTLTNLGAPAAFKYFDTTIDRKQYRSTKEVLDATLIHQ
SITGLYETRIDLSQLGGDEGADKRTADGSEFESPKKKRKV
NGA_IBE16 nucleotide sequence:
(SEQ ID NO: 638)
atggacaagaagtactctatcggactggccatcggcaccaactctgttggatgggccgtgatca
ccgacgagtacaaggtgcccagcaagaaattcaaggtgctgggcaacaccgaccggcacagcat
caagaagaatctgatcggcgccctgctgttcgactctggcgaaacagccgaagccaccagactg
aagagaaccgccaggcggagatacacccggcggaagaaccggatctgctacctgcaagagatct
tcagcaacgagatggccaaggtggacgacagcttcttccacagactggaagagtccttcctggt
ggaagaggacaagaagcacgagcggcaccccatcttcggcaacatcgtggatgaggtggcctac
cacgagaagtaccccaccatctaccacctgagaaagaaactggtggacagcaccgacaaggccg
acctgagactgatctacctggctctggcccacatgatcaagttccggggccactttctgatcga
gggcgatctgaaccccgacaacagcgacgtggacaagctgttcatccagctggtgcagacctac
aaccagctgttcgaggaaaaccccatcaacgcctctggcgtggacgccaaggctatcctgtctg
ccagactgagcaagagcagaaggctggaaaacctgatcgcccagctgcctggcgagaagaagaa
tggcctgttcggcaacctgattgccctgagcctgggactgacccctaacttcaagagcaacttc
gacctggccgaggatgccaaactgcagctgagcaaggacacctacgacgacgacctggacaatc
tgctggcccagatcggcgatcagtacgccgacttgtttctggccgccaagaacctgtccgacgc
catcctgctgagcgatatcctgagagtgaacaccgagatcacaaaggcccctctgagcgcctct
atgatcaagagatacgacgagcaccaccaggatctgaccctgctgaaggccctcgttagacagc
agctgccagagaagtacaaagagattttcttcgatcagtccaagaacggctacgccggctacat
tgatggcggagccagccaagaggaattctacaagttcatcaagcccatcctggaaaagatggac
ggcaccgaggaactgctggtcaagctgaacagagaggacctgctgcggaagcagcggaccttcg
acaatggctctatccctcaccagatccacctgggagagctgcacgccattctgcggagacaaga
ggacttttacccattcctgaaggacaaccgggaaaagatcgagaagatcctgaccttcaggatc
ccctactacgtgggaccactggccagaggcaatagcagattcgcctggatgaccagaaagagcg
aggaaaccatcacaccctggaacttcgaggaagtggtggacaagggcgccagcgctcagtcctt
catcgagcggatgaccaacttcgataagaacctgcctaacgagaaggtgctgcccaagcactcc
ctgctgtatgagtacttcaccgtgtacaacgagctgaccaaagtgaaatacgtgaccgagggaa
tgagaaagcccgcctttctgagcggcgagcagaaaaaggccattgtggatctgctgttcaagac
caaccggaaagtgaccgtgaagcagctgaaagaggactacttcaagaaaatcgagtgcttcgac
agcgtggaaatcagcggcgtggaagatcggttcaatgccagcctgggcacataccacgacctgc
tgaaaattatcaaggacaaggacttcctggacaacgaagagaacgaggacattctcgaggacat
cgtgctgaccctgacactgtttgaggacagagagatgatcgaggaacggctgaaaacatacgcc
cacctgttcgacgacaaagtgatgaagcaactgaagcggaggcggtacacaggctggggcagac
tgtctcggaagctgatcaacggcatccgggataagcagtccggcaagacaatcctggatttcct
gaagtccgacggcttcgccaacagaaacttcatgcagctgatccacgacgacagcctgaccttt
aaagaggacatccagaaagcccaggtgtccggccaaggcgattctctgcacgagcacattgcca
acctggccggatctcccgccattaagaagggcatcctgcagacagtgaaggtggtggacgagct
tgtgaaagtgatgggcagacacaagcccgagaacatcgtgatcgaaatggccagagagaaccag
accacacagaagggccagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaaccgg
ctgagcgactacgatgtggaccatatcgtgccccagagctttctgaaggacgactccatcgata
acaaggtcctgaccagaagcgacaagaaccggggcaagagcgataacgtgccctccgaagaggt
ggtcaagaagatgaagaactactggcgacagctgctgaacgccaagctgattacccagcggaag
ttcgataacctgaccaaggccgagagaggcggcctgagcgaacttgataaggccggcttcatta
agcggcagctggtggaaacccggcagatcaccaaacacgtggcacagattctggactcccggat
gaacactaagtacgacgagaatgacaagctgatccgggaagtgaaagtcatcaccctgaagtct
aagctggtgtccgatttccggaaggatttccagttctacaaagtgcgggaaatcaacaactacc
atcacgcccacgacgcctacctgaatgccgttgttggaacagccctgatcaagaagtatcccaa
gctggaaagcgagttcgtgtacggcgactacaaggtgtacgacgtgcggaagatgatcgccaag
agcgaacaagagatcggcaaggctaccgccaagtactttttctacagcaacatcatgaactttt
tcaagacagagatcaccctggccaacggcgagatccggaaaagacccctgatcgagacaaacgg
cgaaaccggggagatcgtgtgggataagggcagagattttgccacagtgcggaaagtgctgagc
atgccccaagtgaatatcgtgaagaaaaccgaggtgcagacaggcggcttcagcaaagagtcta
tcctgcctaagcggaacagcgataagctgatcgccagaaagaaggactgggaccccaagaaata
cggcggcttcgtgtctcctaccgtggcctattctgtgctggtggtggccaaagtggaaaagggc
aagtccaaaaagctcaagagcgtgaaagagctgctggggatcaccatcatggaaagaagcagct
ttgagaagaacccgatcgactttctggaagccaagggctacaaagaagtcaagaaggacctcat
catcaagctccccaagtacagcctgttcgagctggaaaatggccggaagcggatgctggcctct
gctagagaactgcagaagggaaacgagctggccctgcctagcaaatatgtgaacttcctgtacc
tggccagccactatgagaagctgaagggcggatcttctggaagcgaaaccccaggcaccagcga
gtctgccacaccagaatcatctggcagcgaggtcgagttctctcacgaatattggatgagacac
gctctcaccctggctaagagagccagggacgaaagagaggtgccagttggcgctgtcctggtgt
tgaacaatcgcgtcatcggagaaggatggaatcgcgccattggcctgcacgatccaaccgcaca
tgccgaaattatggctctgcggcaaggcggcctcgtgatgcaaaattacagactgtacgatgct
accctctactccaccttcgagccctgtgtcatgtgtgctggggcaatgattcactcccggattg
gccgcgtggtgtttggagtgcggaatgccaagactggcgccgctggatctctgatggacgtcct
gcaccatcctgggatgaaccaccgggtcgagatcacagagggaattctggctgacgagtgcgct
gccctgctgtgcaggttctttagaatgcctagaagggtgttcaacgcccagaaaaaagctcaga
gcagcaccgatagccccgaggacaatgagcaaaagcagctgtttgtggaacagcacaagcacta
cctggacgagatcatcgagcagatcagcgagttctccaagagagtgatcctggccgacgctaac
ctggataaggtgctgtctgcctataacaagcaccgggacaagcctatcagagagcaggccgaga
atatcatccacctgtttaccctgaccaacctgggagcccctgccgccttcaagtacttcgacac
caccatcgaccggaagcagtaccggtccaccaaagaggtgctggacgccacactgatccaccag
tctatcaccggcctgtacgaaacccggatcgacctgtctcagctcggaggcgacgaaggcgccg
ataagagaaccgccgatggctctgagttcgagagccccaagaaaaagcgcaaagtgtaa
NGC_IBE11 amino acid sequence:
(SEQ ID NO: 639)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MVKRYDEHHQDLTLLKALVRQQLPEKYKEIFFDQSKNGYAGYIDGGASQEEFYKFIKPILEKMD
GTEELLVKLNREDLLRKQRTFDNGIIPHQIHLGELHAILRRQGDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRLRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGGHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHIVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEGSSGSETPGTSESATPESSGSEVEFSHEYWMRHALTLAKRARDEREVPVGAVLVLNNRVIGE
GWNRAIGLHDPTAHAEIMALRQGGLVMQNYRLYDATLYSTFEPCVMCAGAMIHSRIGRVVFGVR
NAKTGAAGSLMDVLHHPGMNHRVEITEGILADECAALLCRFFRMPRRVFNAQKKAQSSTDQEIG
KATAKYFFYSNIMNFFKTEITLANGEIRKRPLIETNGETGEIVWDKGRDFATVRKVLSMPQVNI
VKKTEVQTGGFSKESILPKGNSDKLIARKKDWDPKKYGGFMQPTVAYSVLVVAKVEKGKSKKLK
SVKELLGITIMERSSFEKNPIDFLEAKGYKEVKKDLIIKLPKYSLFELENGRKRMLASAKFLQK
GNELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPRAFKYFDTTIARKEYRSTKEVLDATLIHQ
SITGLYETRIDLSQLGGDEGADKRTADGSEFESPKKKRKV
NGC_IBE11 nucleotide sequence:
(SEQ ID NO: 640)
atggacaagaagtattctatcggactggccatcggcaccaactctgttggatgggccgtgatca
ccgacgagtacaaggtgcccagcaagaaattcaaggtgctgggcaacaccgacaggcacagcat
caagaagaacctgatcggcgcactgctgttcgactctggcgaaacagccgaggccaccagactg
aagagaacagcccgcagacggtacaccagaagaaagaaccggatctgctacctccaagagatct
tcagcaacgagatggccaaggtggacgacagcttcttccacagactggaagagtccttcctggt
ggaagaggacaagaagcacgagagacaccccatcttcggcaacatcgtggacgaggtggcctac
cacgagaagtaccccaccatctaccacctgagaaagaaactggtggacagcaccgacaaggccg
acctgagactgatctatctggccctggctcacatgatcaagttccggggccacttcctgatcga
gggcgacctgaatcctgacaacagcgacgtggacaagctgttcatccagctggtgcagacctac
aaccagctgttcgaggaaaaccccatcaacgccagcggagtggatgccaaggccatcctgtctg
ccagactgagcaagagcagacggctggaaaatctgatcgcccagctgcctggcgagaagaagaa
tggcctgttcggcaacctgattgccctgagcctgggcctgacacctaacttcaagagcaacttc
gacctggccgaggacgccaaactgcagctgagcaaggacacctacgacgacgacctggacaatc
tgctggcccagatcggcgatcagtacgccgacttgtttctggccgccaagaatctgagcgacgc
catcctgctgtccgacatcctgagagtgaacaccgagatcaccaaggcacctctgagcgcctct
atggtcaagagatacgacgagcaccaccaggatctgaccctgctgaaggccctcgttagacagc
agctgccagagaagtacaaagagattttcttcgaccagagcaagaacggctacgccggctacat
tgatggcggagccagccaagaggaattctacaagttcatcaagcccatcctcgagaagatggac
ggcaccgaggaactgctggtcaagctgaacagagaggacctgctgagaaagcagagaaccttcg
acaacggcatcatccctcaccagatccacctgggagaactgcacgccattctgcggagacaagg
ggacttttacccattcctgaaggacaaccgggaaaagatcgagaaaatcctgaccttcaggatc
ccctactacgtgggaccactggccagaggcaatagcagattcgcctggatgaccagaaagagcg
aggaaaccatcactccctggaacttcgaggaagtggtggacaagggcgccagcgctcagtcctt
catcgagcggatgaccaacttcgataagaacctgcctaacgagaaggtgctgcccaagcacagc
ctgctgtacgagtacttcaccgtgtacaacgagctgaccaaagtgaaatacgtgaccgagggaa
tgagaaagcccgcctttctgagcggcgagcagaaaaaggccatcgtggatctgctgttcaagac
caaccggaaagtgaccgtgaagcagctgaaagaggactacttcaagaaaatcgagtgcttcgac
agcgtcgagatctccggcgtggaagatcggttcaatgccagcctgggcacataccacgatctgc
tgaaaattatcaaggacaaggacttcctggacaacgaagagaacgaggacatccttgaggacat
cgtgctgacactgaccctgtttgaggacagagagatgatcgaggaacggctgaaaacatacgcc
cacctgttcgacgacaaagtgatgaagcaactgaagcggctgagatacaccggctggggcagac
tgtctcggaagctgatcaacggcatccgggataagcagtccggcaagaccatcctggactttct
gaagtccgacggcttcgccaacagaaacttcatgcagctgattcacgacgacagcctcaccttc
aaagaggatatccagaaagcccaggtgtccggccagggcgattctctgcatgagcacattgcca
acctggccggctctcccgccattaagaaaggcatcctgcagacagtgaaggtggtggacgagct
tgtgaaagtgatgggcggacacaagcccgagaacatcgtgatcgaaatggccagagagaaccag
accacacagaagggacagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaacaga
ctgtccgactacgatgtggaccatatcgtgccccagtcttttctgaaggacgactccatcgaca
acaaggtcctgaccagatccgacaagaatcggggcaagagcgacaacgtgccctccgaagaggt
ggtcaagaagatgaagaactactggcgacagctgctgaacgccaagctgattacccagcggaag
ttcgacaatctgaccaaggccgaaagaggcggcctgagcgaactggataaggccggcttcatca
agagacagctggtggaaacccggcagatcacaaagcacgtggcacagattctggactctcggat
gaacactaagtacgacgagaacgacaaactgatccgcgaagtgaaagtcatcaccctgaagtcc
aagctggtgtccgatttccggaaggatttccagttctacaaagtgcgcgagatcaacaactacc
atcacgcccacgacgcctacctgaatgccgttgttggaacagccctgatcaaaaagtaccctaa
gctggaaagcgagttcgtgtacggcgactacaaggtgtacgacgtgcggaagatgatcgccaag
agcgagggatcttctggaagcgaaaccccaggcaccagcgagtctgccacaccagaatcatctg
gcagcgaggtcgagttctctcacgaatattggatgagacacgctctcaccctggctaagagagc
cagggacgaaagagaggtgccagttggcgctgtcctggtgttgaacaatcgcgtcatcggagaa
ggatggaatcgcgccattggcctgcacgatccaaccgcacatgccgaaattatggctctgcggc
aaggcggcctcgtgatgcaaaattacagactgtacgatgctaccctctactccaccttcgagcc
ctgtgtcatgtgtgctggggcaatgattcactcccggattggccgcgtggtgtttggagtgcgg
aatgccaagactggcgccgctggatctctgatggacgtcctgcaccatcctgggatgaaccacc
gggtcgagatcacagagggaattctggctgacgagtgcgctgccctgctgtgcaggttctttag
aatgcctagaagggtgttcaacgcccagaaaaaagctcagagcagcaccgatcaagagattggc
aaggcaaccgccaagtacttcttctacagcaacatcatgaactttttcaagacagagatcaccc
tcgccaacggcgagatcagaaagcggcctctgatcgagacaaacggcgaaaccggcgagattgt
gtgggataagggcagagactttgccacagtgcggaaagtgctgagcatgccccaagtgaatatc
gtgaagaaaaccgaggtgcagacaggcggcttcagcaaagagtctatcctgcctaaggggaact
ccgacaagctgatcgccagaaagaaggactgggaccccaagaaatacggcggctttatgcagcc
caccgtggcctattctgttctggtggtggccaaagtggaaaagggcaagtccaagaaactcaag
agcgtgaaagagctgctggggatcaccatcatggaaagaagcagcttcgagaagaatccgatcg
atttcctcgaggccaagggttacaaagaagtgaaaaaggacctgatcatcaagctccccaagta
ctccctgttcgagctggaaaacggccggaagagaatgctggcctctgccaagttcctgcagaag
ggaaacgaactggccctgcctagcaaatatgtgaacttcctgtacctggccagccactatgaga
agctgaagggcagccccgaggacaatgagcaaaagcagctgtttgtggaacagcacaagcacta
cctggacgagatcatcgagcagatcagcgagtttagcaagagagtgattctggccgacgccaat
ctggacaaagtgctgtccgcctacaacaagcaccgggacaagcctatcagagagcaggccgaga
atatcatccacctgtttaccctgaccaacctgggagcccctagagccttcaagtactttgacac
caccatcgcccggaaggagtaccggtccaccaaagaggtgctggacgccactctgatccaccag
tctatcaccggcctgtacgagacacggatcgacctgtctcaactcggaggcgacgaaggcgccg
ataagagaaccgccgatggctctgagttcgagagccccaagaaaaagcgcaaagtgtaa
NGC_IBE12 amino acid sequence:
(SEQ ID NO: 641)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MVKRYDEHHQDLTLLKALVRQQLPEKYKEIFFDQSKNGYAGYIDGGASQEEFYKFIKPILEKMD
GTEELLVKLNREDLLRKQRTFDNGIIPHQIHLGELHAILRRQGDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRLRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGGHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHIVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGSSGSETPGTSESATPESSGSEVEFSHEYWMRHALTLAKRARDEREVPVGAVLVLNNRV
IGEGWNRAIGLHDPTAHAEIMALRQGGLVMQNYRLYDATLYSTFEPCVMCAGAMIHSRIGRVVF
GVRNAKTGAAGSLMDVLHHPGMNHRVEITEGILADECAALLCRFFRMPRRVFNAQKKAQSSTDG
KATAKYFFYSNIMNFFKTEITLANGEIRKRPLIETNGETGEIVWDKGRDFATVRKVLSMPQVNI
VKKTEVQTGGFSKESILPKGNSDKLIARKKDWDPKKYGGFMQPTVAYSVLVVAKVEKGKSKKLK
SVKELLGITIMERSSFEKNPIDFLEAKGYKEVKKDLIIKLPKYSLFELENGRKRMLASAKFLQK
GNELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPRAFKYFDTTIARKEYRSTKEVLDATLIHQ
SITGLYETRIDLSQLGGDEGADKRTADGSEFESPKKKRKV
NGC_IBE12 nucleotide sequence:
(SEQ ID NO: 642)
atggacaagaagtattctatcggactggccatcggcaccaactctgttggatgggccgtgatca
ccgacgagtacaaggtgcccagcaagaaattcaaggtgctgggcaacaccgacaggcacagcat
caagaagaacctgatcggcgcactgctgttcgactctggcgaaacagccgaggccaccagactg
aagagaacagcccgcagacggtacaccagaagaaagaaccggatctgctacctccaagagatct
tcagcaacgagatggccaaggtggacgacagcttcttccacagactggaagagtccttcctggt
ggaagaggacaagaagcacgagagacaccccatcttcggcaacatcgtggacgaggtggcctac
cacgagaagtaccccaccatctaccacctgagaaagaaactggtggacagcaccgacaaggccg
acctgagactgatctatctggccctggctcacatgatcaagttccggggccacttcctgatcga
gggcgacctgaatcctgacaacagcgacgtggacaagctgttcatccagctggtgcagacctac
aaccagctgttcgaggaaaaccccatcaacgccagcggagtggatgccaaggccatcctgtctg
ccagactgagcaagagcagacggctggaaaatctgatcgcccagctgcctggcgagaagaagaa
tggcctgttcggcaacctgattgccctgagcctgggcctgacacctaacttcaagagcaacttc
gacctggccgaggacgccaaactgcagctgagcaaggacacctacgacgacgacctggacaatc
tgctggcccagatcggcgatcagtacgccgacttgtttctggccgccaagaatctgagcgacgc
catcctgctgtccgacatcctgagagtgaacaccgagatcaccaaggcacctctgagcgcctct
atggtcaagagatacgacgagcaccaccaggatctgaccctgctgaaggccctcgttagacagc
agctgccagagaagtacaaagagattttcttcgaccagagcaagaacggctacgccggctacat
tgatggcggagccagccaagaggaattctacaagttcatcaagcccatcctcgagaagatggac
ggcaccgaggaactgctggtcaagctgaacagagaggacctgctgagaaagcagagaaccttcg
acaacggcatcatccctcaccagatccacctgggagaactgcacgccattctgcggagacaagg
ggacttttacccattcctgaaggacaaccgggaaaagatcgagaaaatcctgaccttcaggatc
ccctactacgtgggaccactggccagaggcaatagcagattcgcctggatgaccagaaagagcg
aggaaaccatcactccctggaacttcgaggaagtggtggacaagggcgccagcgctcagtcctt
catcgagcggatgaccaacttcgataagaacctgcctaacgagaaggtgctgcccaagcacagc
ctgctgtacgagtacttcaccgtgtacaacgagctgaccaaagtgaaatacgtgaccgagggaa
tgagaaagcccgcctttctgagcggcgagcagaaaaaggccatcgtggatctgctgttcaagac
caaccggaaagtgaccgtgaagcagctgaaagaggactacttcaagaaaatcgagtgcttcgac
agcgtcgagatctccggcgtggaagatcggttcaatgccagcctgggcacataccacgatctgc
tgaaaattatcaaggacaaggacttcctggacaacgaagagaacgaggacatccttgaggacat
cgtgctgacactgaccctgtttgaggacagagagatgatcgaggaacggctgaaaacatacgcc
cacctgttcgacgacaaagtgatgaagcaactgaagcggctgagatacaccggctggggcagac
tgtctcggaagctgatcaacggcatccgggataagcagtccggcaagaccatcctggactttct
gaagtccgacggcttcgccaacagaaacttcatgcagctgattcacgacgacagcctcaccttc
aaagaggatatccagaaagcccaggtgtccggccagggcgattctctgcatgagcacattgcca
acctggccggctctcccgccattaagaaaggcatcctgcagacagtgaaggtggtggacgagct
tgtgaaagtgatgggcggacacaagcccgagaacatcgtgatcgaaatggccagagagaaccag
accacacagaagggacagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaacaga
ctgtccgactacgatgtggaccatatcgtgccccagtcttttctgaaggacgactccatcgaca
acaaggtcctgaccagatccgacaagaatcggggcaagagcgacaacgtgccctccgaagaggt
ggtcaagaagatgaagaactactggcgacagctgctgaacgccaagctgattacccagcggaag
ttcgacaatctgaccaaggccgaaagaggcggcctgagcgaactggataaggccggcttcatca
agagacagctggtggaaacccggcagatcacaaagcacgtggcacagattctggactctcggat
gaacactaagtacgacgagaacgacaaactgatccgcgaagtgaaagtcatcaccctgaagtcc
aagctggtgtccgatttccggaaggatttccagttctacaaagtgcgcgagatcaacaactacc
atcacgcccacgacgcctacctgaatgccgttgttggaacagccctgatcaaaaagtaccctaa
gctggaaagcgagttcgtgtacggcgactacaaggtgtacgacgtgcggaagatgatcgccaag
agcgagcaagagattggatcttctggaagcgaaaccccaggcaccagcgagtctgccacaccag
aatcatctggcagcgaggtcgagttctctcacgaatattggatgagacacgctctcaccctggc
taagagagccagggacgaaagagaggtgccagttggcgctgtcctggtgttgaacaatcgcgtc
atcggagaaggatggaatcgcgccattggcctgcacgatccaaccgcacatgccgaaattatgg
ctctgcggcaaggcggcctcgtgatgcaaaattacagactgtacgatgctaccctctactccac
cttcgagccctgtgtcatgtgtgctggggcaatgattcactcccggattggccgcgtggtgttt
ggagtgcggaatgccaagactggcgccgctggatctctgatggacgtcctgcaccatcctggga
tgaaccaccgggtcgagatcacagagggaattctggctgacgagtgcgctgccctgctgtgcag
gttctttagaatgcctagaagggtgttcaacgcccagaaaaaagctcagagcagcaccgatggc
aaggcaaccgccaagtacttcttctacagcaacatcatgaactttttcaagacagagatcaccc
tcgccaacggcgagatcagaaagcggcctctgatcgagacaaacggcgaaaccggcgagattgt
gtgggataagggcagagactttgccacagtgcggaaagtgctgagcatgccccaagtgaatatc
gtgaagaaaaccgaggtgcagacaggcggcttcagcaaagagtctatcctgcctaaggggaact
ccgacaagctgatcgccagaaagaaggactgggaccccaagaaatacggcggctttatgcagcc
caccgtggcctattctgttctggtggtggccaaagtggaaaagggcaagtccaagaaactcaag
agcgtgaaagagctgctggggatcaccatcatggaaagaagcagcttcgagaagaatccgatcg
atttcctcgaggccaagggttacaaagaagtgaaaaaggacctgatcatcaagctccccaagta
ctccctgttcgagctggaaaacggccggaagagaatgctggcctctgccaagttcctgcagaag
ggaaacgaactggccctgcctagcaaatatgtgaacttcctgtacctggccagccactatgaga
agctgaagggcagccccgaggacaatgagcaaaagcagctgtttgtggaacagcacaagcacta
cctggacgagatcatcgagcagatcagcgagtttagcaagagagtgattctggccgacgccaat
ctggacaaagtgctgtccgcctacaacaagcaccgggacaagcctatcagagagcaggccgaga
atatcatccacctgtttaccctgaccaacctgggagcccctagagccttcaagtactttgacac
caccatcgcccggaaggagtaccggtccaccaaagaggtgctggacgccactctgatccaccag
tctatcaccggcctgtacgagacacggatcgacctgtctcaactcggaggcgacgaaggcgccg
ataagagaaccgccgatggctctgagttcgagagccccaagaaaaagcgcaaagtgtaa
NGC_IBE16 amino acid sequence:
(SEQ ID NO: 643)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MVKRYDEHHQDLTLLKALVRQQLPEKYKEIFFDQSKNGYAGYIDGGASQEEFYKFIKPILEKMD
GTEELLVKLNREDLLRKQRTFDNGIIPHQIHLGELHAILRRQGDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRLRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGGHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKGNSDKLIARKKDWDPKKYGGFMQPTVAYSVLVVAKVEKG
KSKKLKSVKELLGITIMERSSFEKNPIDFLEAKGYKEVKKDLIIKLPKYSLFELENGRKRMLAS
AKFLQKGNELALPSKYVNFLYLASHYEKLKGGSSGSETPGTSESATPESSGSEVEFSHEYWMRH
ALTLAKRARDEREVPVGAVLVLNNRVIGEGWNRAIGLHDPTAHAEIMALRQGGLVMQNYRLYDA
TLYSTFEPCVMCAGAMIHSRIGRVVFGVRNAKTGAAGSLMDVLHHPGMNHRVEITEGILADECA
ALLCRFFRMPRRVFNAQKKAQSSTDSPEDNEQKQLFVEQHKHYLDEIIEQISEFSKRVILADAN
LDKVLSAYNKHRDKPIREQAENIIHLFTLTNLGAPRAFKYFDTTIARKEYRSTKEVLDATLIHQ
SITGLYETRIDLSQLGGDEGADKRTADGSEFESPKKKRKV
NGC_IBE16 nucleotide sequence:
(SEQ ID NO: 644)
atggacaagaagtattctatcggactggccatcggcaccaactctgttggatgggccgtgatca
ccgacgagtacaaggtgcccagcaagaaattcaaggtgctgggcaacaccgacaggcacagcat
caagaagaacctgatcggcgcactgctgttcgactctggcgaaacagccgaggccaccagactg
aagagaacagcccgcagacggtacaccagaagaaagaaccggatctgctacctccaagagatct
tcagcaacgagatggccaaggtggacgacagcttcttccacagactggaagagtccttcctggt
ggaagaggacaagaagcacgagagacaccccatcttcggcaacatcgtggacgaggtggcctac
cacgagaagtaccccaccatctaccacctgagaaagaaactggtggacagcaccgacaaggccg
acctgagactgatctatctggccctggctcacatgatcaagttccggggccacttcctgatcga
gggcgacctgaatcctgacaacagcgacgtggacaagctgttcatccagctggtgcagacctac
aaccagctgttcgaggaaaaccccatcaacgccagcggagtggatgccaaggccatcctgtctg
ccagactgagcaagagcagacggctggaaaatctgatcgcccagctgcctggcgagaagaagaa
tggcctgttcggcaacctgattgccctgagcctgggcctgacacctaacttcaagagcaacttc
gacctggccgaggacgccaaactgcagctgagcaaggacacctacgacgacgacctggacaatc
tgctggcccagatcggcgatcagtacgccgacttgtttctggccgccaagaatctgagcgacgc
catcctgctgtccgacatcctgagagtgaacaccgagatcaccaaggcacctctgagcgcctct
atggtcaagagatacgacgagcaccaccaggatctgaccctgctgaaggccctcgttagacagc
agctgccagagaagtacaaagagattttcttcgaccagagcaagaacggctacgccggctacat
tgatggcggagccagccaagaggaattctacaagttcatcaagcccatcctcgagaagatggac
ggcaccgaggaactgctggtcaagctgaacagagaggacctgctgagaaagcagagaaccttcg
acaacggcatcatccctcaccagatccacctgggagaactgcacgccattctgcggagacaagg
ggacttttacccattcctgaaggacaaccgggaaaagatcgagaaaatcctgaccttcaggatc
ccctactacgtgggaccactggccagaggcaatagcagattcgcctggatgaccagaaagagcg
aggaaaccatcactccctggaacttcgaggaagtggtggacaagggcgccagcgctcagtcctt
catcgagcggatgaccaacttcgataagaacctgcctaacgagaaggtgctgcccaagcacagc
ctgctgtacgagtacttcaccgtgtacaacgagctgaccaaagtgaaatacgtgaccgagggaa
tgagaaagcccgcctttctgagcggcgagcagaaaaaggccatcgtggatctgctgttcaagac
caaccggaaagtgaccgtgaagcagctgaaagaggactacttcaagaaaatcgagtgcttcgac
agcgtcgagatctccggcgtggaagatcggttcaatgccagcctgggcacataccacgatctgc
tgaaaattatcaaggacaaggacttcctggacaacgaagagaacgaggacatccttgaggacat
cgtgctgacactgaccctgtttgaggacagagagatgatcgaggaacggctgaaaacatacgcc
cacctgttcgacgacaaagtgatgaagcaactgaagcggctgagatacaccggctggggcagac
tgtctcggaagctgatcaacggcatccgggataagcagtccggcaagaccatcctggactttct
gaagtccgacggcttcgccaacagaaacttcatgcagctgattcacgacgacagcctcaccttc
aaagaggatatccagaaagcccaggtgtccggccagggcgattctctgcatgagcacattgcca
acctggccggctctcccgccattaagaaaggcatcctgcagacagtgaaggtggtggacgagct
tgtgaaagtgatgggcggacacaagcccgagaacatcgtgatcgaaatggccagagagaaccag
accacacagaagggacagaagaacagccgcgagagaatgaagcggatcgaagagggcatcaaag
agctgggcagccagatcctgaaagaacaccccgtggaaaacacccagctgcagaacgagaagct
gtacctgtactacctgcagaatggacgggatatgtacgtggaccaagagctggacatcaacaga
ctgtccgactacgatgtggaccatatcgtgccccagtcttttctgaaggacgactccatcgaca
acaaggtcctgaccagatccgacaagaatcggggcaagagcgacaacgtgccctccgaagaggt
ggtcaagaagatgaagaactactggcgacagctgctgaacgccaagctgattacccagcggaag
ttcgacaatctgaccaaggccgaaagaggcggcctgagcgaactggataaggccggcttcatca
agagacagctggtggaaacccggcagatcacaaagcacgtggcacagattctggactctcggat
gaacactaagtacgacgagaacgacaaactgatccgcgaagtgaaagtcatcaccctgaagtcc
aagctggtgtccgatttccggaaggatttccagttctacaaagtgcgcgagatcaacaactacc
atcacgcccacgacgcctacctgaatgccgttgttggaacagccctgatcaaaaagtaccctaa
gctggaaagcgagttcgtgtacggcgactacaaggtgtacgacgtgcggaagatgatcgccaag
agcgagcaagagattggcaaggcaaccgccaagtacttcttctacagcaacatcatgaactttt
tcaagacagagatcaccctcgccaacggcgagatcagaaagcggcctctgatcgagacaaacgg
cgaaaccggcgagattgtgtgggataagggcagagactttgccacagtgcggaaagtgctgagc
atgccccaagtgaatatcgtgaagaaaaccgaggtgcagacaggcggcttcagcaaagagtcta
tcctgcctaaggggaactccgacaagctgatcgccagaaagaaggactgggaccccaagaaata
cggcggctttatgcagcccaccgtggcctattctgttctggtggtggccaaagtggaaaagggc
aagtccaagaaactcaagagcgtgaaagagctgctggggatcaccatcatggaaagaagcagct
tcgagaagaatccgatcgatttcctcgaggccaagggttacaaagaagtgaaaaaggacctgat
catcaagctccccaagtactccctgttcgagctggaaaacggccggaagagaatgctggcctct
gccaagttcctgcagaagggaaacgaactggccctgcctagcaaatatgtgaacttcctgtacc
tggccagccactatgagaagctgaagggcggatcttctggaagcgaaaccccaggcaccagcga
gtctgccacaccagaatcatctggcagcgaggtcgagttctctcacgaatattggatgagacac
gctctcaccctggctaagagagccagggacgaaagagaggtgccagttggcgctgtcctggtgt
tgaacaatcgcgtcatcggagaaggatggaatcgcgccattggcctgcacgatccaaccgcaca
tgccgaaattatggctctgcggcaaggcggcctcgtgatgcaaaattacagactgtacgatgct
accctctactccaccttcgagccctgtgtcatgtgtgctggggcaatgattcactcccggattg
gccgcgtggtgtttggagtgcggaatgccaagactggcgccgctggatctctgatggacgtcct
gcaccatcctgggatgaaccaccgggtcgagatcacagagggaattctggctgacgagtgcgct
gccctgctgtgcaggttctttagaatgcctagaagggtgttcaacgcccagaaaaaagctcaga
gcagcaccgatagccccgaggacaatgagcaaaagcagctgtttgtggaacagcacaagcacta
cctggacgagatcatcgagcagatcagcgagtttagcaagagagtgattctggccgacgccaat
ctggacaaagtgctgtccgcctacaacaagcaccgggacaagcctatcagagagcaggccgaga
atatcatccacctgtttaccctgaccaacctgggagcccctagagccttcaagtactttgacac
caccatcgcccggaaggagtaccggtccaccaaagaggtgctggacgccactctgatccaccag
tctatcaccggcctgtacgagacacggatcgacctgtctcaactcggaggcgacgaaggcgccg
ataagagaaccgccgatggctctgagttcgagagccccaagaaaaagcgcaaagtgtaa

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an deaminase can be at an amino acid residue selected from the group consisting of 1015, 1022, 1029, 1040, 1068, 1247, 1054, 1026, 1027, 1030, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e., the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e., the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein or complex described herein can effect targeted deamination with reduced deamination at non-target sites (e.g., off-target sites), such as reduced genome wide spurious deamination. The fusion protein or complex described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein or complex deaminates no more than two nucleobases within the range of an R-loop. In some embodiments, the deaminase of the fusion protein or complex deaminates no more than three nucleobases within the range of the R-loop. In some embodiments, the deaminase of the fusion protein or complex deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g., a guide RNA, hybridizes with and displaces with a portion of a target polynucleotide, e.g., a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein or complex described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein or complex can comprise more than one heterologous polypeptide. For example, the fusion protein or complex can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 246), (GGGGS)n (SEQ ID NO: 247), (G)n, (EAAAK)n (SEQ ID NO: 248), (GGS)n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or

(SEQ ID NO: 253)
GGCTCTTCTGGATCTGAAACACCTGGCACAAGCGAGAGCGCCACCCCTG
AGAGCTCTGGC.

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:
NH₂-[Cas12(adenosine deaminase)]-[cytidine deaminase]-COOH;
NH₂-[cytidine deaminase]-[Cas12(adenosine deaminase)]-COOH;
NH₂-[Cas12(cytidine deaminase)]-[adenosine deaminase]-COOH; or
NH₂-[adenosine deaminase]-[Cas12(cytidine deaminase)]-COOH;

In some embodiments, the “−” used in the general architecture above indicates the optional presence of a linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:
N-[Cas12(TadA*8)]-[cytidine deaminase]-C;
N-[cytidine deaminase]-[Cas12(TadA*8)]-C;
N-[Cas12(cytidine deaminase)]-[TadA*8]-C; or
N-[TadA*8]-[Cas12(cytidine deaminase)]-C.

In some embodiments, the “−” used in the general architecture above indicates the optional presence of a linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 254). In other embodiments, the Cas12 polypeptide has at least about 90% amino acid sequence identity to Bacillus hisashii Cas12b (SEQ ID NO: 255), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 256), Bacillus sp. V3-13 Cas12b (SEQ ID NO: 257), or Alicyclobacillus acidiphilus Cas12b. In other embodiments, the Cas12 polypeptide contains or consists essentially of a fragment (e.g., a functional fragment) of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap—5′ UTR—bhCas12b—STOP sequence—3′ UTR—120polyA tail (SEQ ID NOs: 258-260).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/Cas4. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/Cas4. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:
ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein or complex further contains a tag (e.g., an influenza hemagglutinin tag).

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 5 below.

TABLE 5
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

In some embodiments, adenosine base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion (e.g., a functional portion) of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises a D108X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in a TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in a TadA reference sequence, or a corresponding mutation in another adenosine deaminase (e.g. ecTadA).

In some embodiments, the adenosine deaminase comprises a E155X mutation in a TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in a TadA reference sequence, or a corresponding mutation in another adenosine deaminase (e.g., ecTadA).

In some embodiments, the adenosine deaminase comprises a D147X mutation in a TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in a TadA reference sequence, or a corresponding mutation in another adenosine deaminase (e.g., ecTadA).

In some embodiments, the adenosine deaminase comprises an A106X, E155X, or D147X, mutation in a TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should also be appreciated that any of the mutations provided herein may be made individually or in any combination in ecTadA or another adenosine deaminase. For example, an adenosine deaminase may contain a D108N, a A106V, a E155V, and/or a D147Y mutation in a TadA reference sequence, or a corresponding mutation in another adenosine deaminase (e.g., ecTadA). In some embodiments, an adenosine deaminase comprises the following group of mutations (groups of mutations are separated by a “;”) in a TadA reference sequence (e.g., TadA*7.10 (SEQ ID NO: 1), or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises a combination of mutations in a TadA reference sequence (e.g., TadA*7.10 (SEQ ID NO: 1)), or corresponding mutations in another adenosine deaminase: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N.

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, 1156D, and/or K157R mutation in a TadA reference sequence, or one or more corresponding mutations in another adenosine deaminase.

In some embodiments, the adenosine deaminase comprises one or more of a H8X, D108X, and/or N127X mutation in a TadA reference sequence, or one or more corresponding mutations in another adenosine deaminase, where X indicates the presence of any amino acid. In some embodiments, the adenosine deaminase comprises one or more of a H8Y, D108N, and/or N127S mutation in a TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in a TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in a TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in a TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X a TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in a TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in a TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in a TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in a TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in a TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in a TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in a TadA reference sequence, or a corresponding mutation or mutations in another adenosine deaminase (e.g. ecTadA). In some embodiments, the adenosine deaminase comprises one, two, three, four, or five, mutations selected from the group consisting of H8Y, D108N, A109T, N127S, and E155G in a TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in a TadA reference sequence, or corresponding mutations in another adenosine deaminase. In some embodiments, the adenosine deaminase comprises a A106V and D108N mutation in a TadA reference sequence, or corresponding mutations in another adenosine deaminase. In some embodiments, the adenosine deaminase comprises R107C and D108N mutations in a TadA reference sequence, or corresponding mutations in another adenosine deaminase. In some embodiments, the adenosine deaminase comprises a H8Y, D108N, N127S, D147Y, and Q154H mutation in a TadA reference sequence, or corresponding mutations in another adenosine deaminase. In some embodiments, the adenosine deaminase comprises a H8Y, D108N, N127S, D147Y, and E155V mutation in a TadA reference sequence, or corresponding mutations in another adenosine deaminase. In some embodiments, the adenosine deaminase comprises a D108N, D147Y, and E155V mutation in a TadA reference sequence, or corresponding mutations in another adenosine deaminase. In some embodiments, the adenosine deaminase comprises a H8Y, D108N, and N127S mutation in a TadA reference sequence, or corresponding mutations in another adenosine deaminase. In some embodiments, the adenosine deaminase comprises a A106V, D108N, D147Y, and E155V mutation in a TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in a TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in a TadA reference sequence, or a corresponding mutation in another adenosine deaminase (e.g., ecTadA).

In some embodiments, the adenosine deaminase comprises an H123X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in a TadA reference sequence, or a corresponding mutation in another adenosine deaminase.

In some embodiments, the adenosine deaminase comprises an I156X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in a TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in a TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in a TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in a TadA reference sequence.

In some embodiments, the adenosine deaminase comprises one, two, three, four, or five mutations selected from the group consisting of H8Y, A106T, D108N, N127S, and K160S in a TadA reference sequence, or a corresponding mutation or mutations in another adenosine deaminase.

In some embodiments, the adenosine deaminase comprises one or more of a E25X, R26X, R107X, A142X, and/or A143X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in a TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in a TadA reference sequence, or a corresponding mutation in another adenosine deaminase (e.g., ecTadA).

In some embodiments, the adenosine deaminase comprises an R26X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in a TadA reference sequence, or a corresponding mutation in another adenosine deaminase (e.g., ecTadA).

In some embodiments, the adenosine deaminase comprises an R107X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in a TadA reference sequence, or a corresponding mutation in another adenosine deaminase (e.g., ecTadA).

In some embodiments, the adenosine deaminase comprises an A142X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in a TadA reference sequence, or a corresponding mutation in another adenosine deaminase (e.g., ecTadA).

In some embodiments, the adenosine deaminase comprises an A143X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in a TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in a TadA reference sequence, or one or more corresponding mutations in another adenosine deaminase (e.g., ecTadA).

In some embodiments, the adenosine deaminase comprises an H36X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in a TadA reference sequence, or a corresponding mutation in another adenosine deaminase.

In some embodiments, the adenosine deaminase comprises an N37X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in a TadA reference sequence, or a corresponding mutation in another adenosine deaminase.

In some embodiments, the adenosine deaminase comprises an P48X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in a TadA reference sequence, or a corresponding mutation in another adenosine deaminase.

In some embodiments, the adenosine deaminase comprises an R51X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in a TadA reference sequence, or a corresponding mutation in another adenosine deaminase.

In some embodiments, the adenosine deaminase comprises an S146X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in a TadA reference sequence, or a corresponding mutation in another adenosine deaminase.

In some embodiments, the adenosine deaminase comprises an K157X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in a TadA reference sequence, or a corresponding mutation in another adenosine deaminase.

In some embodiments, the adenosine deaminase comprises an P48X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in a TadA reference sequence, or a corresponding mutation in another adenosine deaminase.

In some embodiments, the adenosine deaminase comprises an A142X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in a TadA reference sequence, or a corresponding mutation in another adenosine deaminase.

In some embodiments, the adenosine deaminase comprises an W23X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in a TadA reference sequence, or a corresponding mutation in another adenosine deaminase.

In some embodiments, the adenosine deaminase comprises an R152X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in a TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “−” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I156F),
(E25D_R26G_L84F_A106V_R107K_D108N_H123Y_A142N_A143G_D147Y_E155V_1156F), (R26Q_L84F_A106V_D108N_H123Y_A142N_D147Y_E155V_I156F),
(E25M_R26G_L84F_A106V_R107P_D108N_H123Y_A142N_A143D_D147Y_E155V_1156F), (R26C_L84F_A106V_R107H_D108N_H123Y_A142N_D147Y_E155V_1156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_I49V_E155V_I156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F104I_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157N),
(N37S_L84F_A106V_D108N_H123Y_A142N_D147Y_E155V_I156F_K161T),
(L84F_A106V_D108N_D147Y_E155V_1156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_1156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F
(H36L_P48T_I49V_R51L_L84F_A106V_D108N_H123Y_S146C_D147Y_E155V_I156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I 156F_K157N),
(W23L_H36L_P48A_R51L_L84F_A106V_D108N_H123Y_A142A_S146C_D147Y_R152P E155V_I156F_K157N),
(W23L_H36L_P48A_R51L_L84F_A106V_D108N_H123Y_S146R_D147Y_E155V_I156F_K161T),
(W23R H36L_P48A_R51L_L84F_A106V_D108N_H123Y_S146C_D147Y_R152P_E155V_I156F_K157N),
(H36L_P48A_R51L_L84F_A106V_D108N_H123Y_A142N_S146C_D147Y_R152P_E155V_I156F_K157N).

In some embodiments, the TadA deaminase is a TadA variant. In some embodiments, the TadA variant is TadA*7.10. In particular embodiments, the fusion proteins or complexes comprise a single TadA*7.10 domain (e.g., provided as a monomer). In other embodiments, the fusion protein comprises TadA*7.10 and TadA(wt), which are capable of forming heterodimers. In one embodiment, a fusion protein of the invention comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA*8) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to a TadA reference sequence (e.g., TadA*7.10 (SEQ ID NO: 1)), or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to a TadA reference sequence (e.g., TadA*7.10 (SEQ ID NO: 1)), or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1)), or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to a TadA reference sequence (e.g., TadA*7.10 (SEQ ID NO: 1)), or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1)), or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1)), or a corresponding mutation in another TadA.

In some embodiments, an adenosine deaminase variant (e.g., MSP828) is a monomer comprising one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1)), or a corresponding mutation in another TadA. In some embodiments, an adenosine deaminase variant (e.g., MSP828) is a monomer comprising V82G, Y147T/D, Q154S, and one or more of L36H, 176Y, F149Y, N157K, and D167N, relative to a TadA reference sequence (e.g., TadA*7.10 (SEQ ID NO: 1)), or a corresponding mutation in another TadA. In other embodiments, the adenosine deaminase variant (TadA variant) is a monomer comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+176Y+V82G+Y147D+F149Y+Q154S+N157K+D167N, relative to a TadA reference sequence (e.g., TadA*7.10 (SEQ ID NO: 1)), or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1)), or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; 176Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and 176Y+V82S+Y123H+Y147R+Q154R, relative to a TadA reference sequence (e.g., TadA*7.10 (SEQ ID NO: 1)), or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y47D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T11R+D119N+H122N+Y147D+F149Y+T166I+D167N, relative to a TadA reference sequence (e.g., TadA*7.10 (SEQ ID NO: 1)), or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1)), or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, I76Y, F149Y, N157K, and D167N, relative to a TadA reference sequence (e.g., TadA*7.10 (SEQ ID NO: 1)), or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to a TadA reference sequence (e.g., TadA*7.10 (SEQ ID NO: 1)), or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1)), or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1)), or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1)), or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, 176Y, V82G, Y147T, Y147D, F149Y, Q154S, N157K, and/or D167N, relative to a TadA reference sequence (e.g., TadA*7.10 (SEQ ID NO: 1)), or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, 176Y, F149Y, N157K, and D167N, relative to a TadA reference sequence (e.g., TadA*7.10 (SEQ ID NO: 1)), or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+176Y+V82G+Y147D+F149Y+Q154S+N157K+D167N, relative to a TadA reference sequence (e.g., TadA*7.10 (SEQ ID NO: 1)), or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1)), or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1)), or a corresponding mutation in another TadA.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 316)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCT
FFRMPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1)), or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1)), or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1)), or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1)), or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, 176Y, V82G, Y147T, Y147D, F149Y, Q154S, N157K, and/or D167N, relative to a TadA reference sequence (e.g., TadA*7.10 (SEQ ID NO: 1)), or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, 176Y, F149Y, N157K, and D167N, relative to a TadA reference sequence (e.g., TadA*7.10 (SEQ ID NO: 1)), or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+176Y+V82G+Y147D+F149Y+Q154S+N157K+D167N, relative to a TadA reference sequence (e.g., TadA*7.10 (SEQ ID NO: 1)), or a corresponding mutation in another TadA.

In some embodiments, the TadA*8 is a variant as shown in Table 6. Table 6 shows certain amino acid position numbers in the TadA amino acid sequence and the amino acids present in those positions in the TadA-7.10 adenosine deaminase. Table 6 also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

TABLE 6
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 6.1. Table 6.1 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 6.1
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein or complex of the invention comprises a wild-type TadA is linked to an adenosine deaminase variant described herein (e.g., TadA*8), which is linked to Cas9 nickase. In particular embodiments, the fusion proteins or complexes comprise a single TadA*8 domain (e.g., provided as a monomer). In other embodiments, the fusion protein or complex comprises TadA*8 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| (SEQ ID NO: 1) | |
|---|---|
| MSEVEFSHEY WMRHALTLAK RARDEREVPV GAVLVLNNRV IGEGWNRAIG | 50 |
| LHDPTAHAEI MALRQGGLVM QNYRLIDATL YVTFEPCVMC AGAMIHSRIG | 100 |
| RVVFGVRNAK TGAAGSLMDV LHYPGMNHRV EITEGILADE CAALLCYFFR | 150 |
| MPRQVFNAQK KAQSSTD | |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to a TadA reference sequence (e.g., TadA*7.10 (SEQ ID NO: 1)), or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1)), or a corresponding mutation in another TadA.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein or complex of the invention comprises a wild-type TadA is linked to an adenosine deaminase variant described herein (e.g., TadA*8), which is linked to Cas9 nickase. In particular embodiments, the fusion proteins or complexes comprise a single TadA*8 domain (e.g., provided as a monomer). In other embodiments, the base editor comprises TadA*8 and TadA(wt), which are capable of forming heterodimers.

In particular embodiments, the fusion proteins or complexes comprise a single (e.g., provided as a monomer) TadA*8. In some embodiments, the TadA*8 is linked to a Cas9 nickase. In some embodiments, the fusion proteins or complexes of the invention comprise as a heterodimer of a wild-type TadA (TadA(wt)) linked to a TadA*8. In other embodiments, the fusion proteins or complexes of the invention comprise as a heterodimer of a TadA*7.10 linked to a TadA*8. In some embodiments, the base editor is ABE8 comprising a TadA*8 variant monomer. In some embodiments, the base editor is ABE8 comprising a heterodimer of a TadA*8 and a TadA(wt). In some embodiments, the base editor is ABE8 comprising a heterodimer of a TadA*8 and TadA*7.10. In some embodiments, the base editor is ABE8 comprising a heterodimer of a TadA*8. In some embodiments, the TadA*8 is selected from Table 6, 12, or 13. In some embodiments, the ABE8 is selected from Table 12, 13, or 15.

In some embodiments, the adenosine deaminase is a TadA*9 variant. In some embodiments, the adenosine deaminase is a TadA*9 variant selected from the variants described below and with reference to the following sequence (termed TadA*7.10):

(SEQ ID NO: 1)
MSEVEFSHEY WMRHALTLAK RARDEREVPV GAVLVLNNRV
IGEGWNRAIG LHDPTAHAEI MALROGGLVM QNYRLIDATL
YVTFEPCVMC AGAMIHSRIG RVVFGVRNAK TGAAGSLMDV
LHYPGMNHRV EITEGILADE CAALLCYFFR MPRQVFNAQK
KAQSSTD

In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, an adenosine deaminase comprises one or more of the following combinations of alterations: Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+176Y+V82S+Y123H+Y147R+Q154R; P54C+I76Y+V82S+Y123H+Y147R+Q154R; R21N+I76Y+V82S+Y123H+Y147R+Q154R; 176Y+V82S+Y123H+D139M+Y147R+Q154R; Y73S+176Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant comprises the alterations described in Table 16 as described herein. In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/US2020/049975, which is incorporated herein by reference for its entirety.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion (e.g., a functional portion) of an APOBEC1 deaminase. In some embodiments, a deaminase incorporated into a base editor comprises all or a portion (e.g., a functional portion) of APOBEC2 deaminase. In some embodiments, a deaminase incorporated into a base editor comprises all or a portion (e.g., a functional portion) of is an APOBEC3 deaminase. In some embodiments, a deaminase incorporated into a base editor comprises all or a portion (e.g., a functional portion) of an APOBEC3A deaminase. In some embodiments, a deaminase incorporated into a base editor comprises all or a portion (e.g., a functional portion) of APOBEC3B deaminase. In some embodiments, a deaminase incorporated into a base editor comprises all or a portion (e.g., a functional portion) of APOBEC3C deaminase. In some embodiments, a deaminase incorporated into a base editor comprises all or a portion (e.g., a functional portion) of APOBEC3D deaminase. In some embodiments, a deaminase incorporated into a base editor comprises all or a portion (e.g., a functional portion) of APOBEC3E deaminase. In some embodiments, a deaminase incorporated into a base editor comprises all or a portion (e.g., a functional portion) of APOBEC3F deaminase. In some embodiments, a deaminase incorporated into a base editor comprises all or a portion (e.g., a functional portion) of APOBEC3G deaminase. In some embodiments, a deaminase incorporated into a base editor comprises all or a portion (e.g., a functional portion) of APOBEC3H deaminase. In some embodiments, a deaminase incorporated into a base editor comprises all or a portion (e.g., a functional portion) of APOBEC4 deaminase. In some embodiments, a deaminase incorporated into a base editor comprises all or a portion (e.g., a functional portion) of activation-induced deaminase (AID). In some embodiments a deaminase incorporated into a base editor comprises all or a portion (e.g., a functional portion) of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, orangutan, alligator, pig, cow, dog, rat, or mouse. In some embodiments, the deaminase domain of the base editor is derived from rat (e.g., rat APOBEC1).

In some embodiments, the deaminase domain of the base editor is derived from an orangutan polypeptide (e.g., a Pongo pygmaeus (Orangutan) APOBEC). In some embodiments, the deaminase domain of the base editor is derived from a golden snub-nosed monkey polypeptide (e.g., a Rhinopithecus roxellana (golden snub-nosed monkey) APOBEC3F (A3F)). In some embodiments, the deaminase domain of the base editor is derived from an American Alligator polypeptide (e.g., an Alligator mississippiensis (American alligator) APOBEC1). In some embodiments, the deaminase domain of the base editor is derived from a pig polypeptide (e.g., a Sus scrofa (pig) APOBEC3B). In some embodiments, the deaminase domain of the base editor is human APOBEC1. In some embodiments, the deaminase domain of the base editor is pmCDA1.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor comprises one or more mutations selected from the group consisting of H121X, H122X, R126X, R126X, RI 18X, W90X, W90X, and R132X of rAPOBEC1, or one or more corresponding mutations in another APOBEC deaminase, wherein X is any amino acid. In some embodiments, an APOBEC deaminase incorporated into a base editor can comprise one or more mutations selected from the group consisting of H121R, H122R, R126A, R126E, RI 18A, W90A, W90Y, and R132E of rAPOBEC1, or one or more corresponding mutations in another APOBEC deaminase.

In some embodiments, an APOBEC deaminase incorporated into a base editor comprises one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins or complexes provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor comprises a H121R and a H122R mutation of rAPOBEC1, or one or more corresponding mutations in another APOBEC deaminase. In some embodiments an APOBEC deaminase incorporated into a base editor comprises an APOBEC deaminase comprising a R126A mutation of rAPOBEC1, or one or more corresponding mutations in another APOBEC deaminase. In some embodiments, an APOBEC deaminase incorporated into a base editor comprises an APOBEC deaminase comprising a R126E mutation of rAPOBEC1, or one or more corresponding mutations in another APOBEC deaminase. In some embodiments, an APOBEC deaminase incorporated into a base editor comprises an APOBEC deaminase comprising a R118A mutation of rAPOBEC1, or one or more corresponding mutations in another APOBEC deaminase. In some embodiments, an APOBEC deaminase incorporated into a base editor comprises an APOBEC deaminase comprising a W90A mutation of rAPOBEC1, or one or more corresponding mutations in another APOBEC deaminase. In some embodiments, an APOBEC deaminase incorporated into a base editor comprises an APOBEC deaminase comprising a W90Y mutation of rAPOBEC1, or one or more corresponding mutations in another APOBEC deaminase. In some embodiments, an APOBEC deaminase incorporated into a base editor comprises an APOBEC deaminase comprising a R132E mutation of rAPOBEC1, or one or more corresponding mutations in another APOBEC deaminase. In some embodiments an APOBEC deaminase incorporated into a base editor comprises an APOBEC deaminase comprising a W90Y and a R126E mutation of rAPOBEC1, or one or more corresponding mutations in another APOBEC deaminase. In some embodiments, an APOBEC deaminase incorporated into a base editor comprises an APOBEC deaminase comprising a R126E and a R132E mutation of rAPOBEC1, or one or more corresponding mutations in another APOBEC deaminase. In some embodiments, an APOBEC deaminase incorporated into a base editor comprises an APOBEC deaminase comprising a W90Y and a R132E mutation of rAPOBEC1, or one or more corresponding mutations in another APOBEC deaminase. In some embodiments, an APOBEC deaminase incorporated into a base editor comprises an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor comprises an APOBEC deaminase comprising a D316R and a D317R mutation of hAPOBEC3G, or one or more corresponding mutations in another APOBEC deaminase. In some embodiments, any of the fusion proteins or complexes provided herein comprise an APOBEC deaminase comprising a R320A mutation of hAPOBEC3G, or one or more corresponding mutations in another APOBEC deaminase. In some embodiments, an APOBEC deaminase incorporated into a base editor comprises an APOBEC deaminase comprising a R320E mutation of hAPOBEC3G, or one or more corresponding mutations in another APOBEC deaminase. In some embodiments, an APOBEC deaminase incorporated into a base editor comprises an APOBEC deaminase comprising a R313A mutation of hAPOBEC3G, or one or more corresponding mutations in another APOBEC deaminase. In some embodiments, an APOBEC deaminase incorporated into a base editor comprises an APOBEC deaminase comprising a W285A mutation of hAPOBEC3G, or one or more corresponding mutations in another APOBEC deaminase. In some embodiments, an APOBEC deaminase incorporated into a base editor comprises an APOBEC deaminase comprising a W285Y mutation of hAPOBEC3G, or one or more corresponding mutations in another APOBEC deaminase. In some embodiments, an APOBEC deaminase incorporated into a base editor comprises an APOBEC deaminase comprising a R326E mutation of hAPOBEC3G, or one or more corresponding mutations in another APOBEC deaminase. In some embodiments, an APOBEC deaminase incorporated into a base editor comprises an APOBEC deaminase comprising a W285Y and a R320E mutation of hAPOBEC3G, or one or more corresponding mutations in another APOBEC deaminase. In some embodiments, an APOBEC deaminase incorporated into a base editor comprises an APOBEC deaminase comprising a R320E and a R326E mutation of hAPOBEC3G, or one or more corresponding mutations in another APOBEC deaminase. In some embodiments, an APOBEC deaminase incorporated into a base editor comprises an APOBEC deaminase comprising a W285Y and a R326E mutation of hAPOBEC3G, or one or more corresponding mutations in another APOBEC deaminase. In some embodiments, an APOBEC deaminase incorporated into a base editor comprises an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the invention comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In embodiments, a fusion protein of the invention comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (T_{AD}C)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, the adenosine deaminase variants of the invention comprise one or more alterations. In some embodiments, an adenosine deaminase variant of the invention is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some embodiments, the adenosine deaminase variant is a bacterial TadA deaminase variant (e.g., ecTadA). In some embodiments, the adenosine deaminase variant is a truncated TadA deaminase variant. In some embodiments, the adenosine deaminase variant is a fragment of a TadA deaminase variant. In some embodiments, an adenosine deaminase variant is a TadA*8 variant comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least about 30%, 40%, 50% or more of the adenosine deaminase activity of a reference adenosine deaminase (e.g., TadA*8.20 or TadA*8.19). In some embodiments, an adenosine deaminase variant is a TadA*8.20 adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least 30%, 40%, 50% of the activity of a reference adenosine deaminase (e.g., TadA*8.20 or TadA*8.19). In some embodiments, an adenosine deaminase variant as provided herein has an increased cytosine deaminase activity of at least about 10-fold, 20-fold, 30-fold, 40-fold, 50-fold, 60-fold, 70-fold, 80-fold, 90-fold, 100-fold or more relative to a reference adenosine deaminase (e.g., TadA*8.20 or TadA*8.19). In some embodiments, an adenosine deaminase variant as provided herein maintains a level of adenosine deaminase activity that is at least about 30%, 40%, 50%, 60%, 70% of the activity of a reference adenosine deaminase (e.g., TadA*8.20 or TadA*8.19). In some embodiments, the reference adenosine deaminase is TadA*8.20 or TadA*8.19.

In some embodiments, the adenosine deaminase variant is an adenosine deaminase comprising one or more alterations that increase cytosine deaminase activity and has an amino acid sequence that is at least about 70%, 75%, 80%, 85%, 90%, 95%, 98%, 99% or greater identity to SEQ ID NO: 1 below:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD.

In some embodiments, the adenosine deaminase variant is an adenosine deaminase comprising the amino acid sequence of SEQ ID NO: 1 and one or more alterations that increase cytosine deaminase activity. In various embodiments, the one or more alterations of the invention do not include a R amino acid at position 48 of SEQ ID NO: 1, or a corresponding mutation in another adenosine deaminase.

In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase. In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. In some embodiments, the two or more alterations are at an amino acid position selected from the group consisting of S2X, V4X, F6X, H8X, R13X, T17X, R23X, E27X, P29X, V30X, R47X, A48X, I49X, G67X, Y76X, D77X, S82X, F84X, H96X, G100X, R107X, G112X, A114X, G115X, M118X, D119X, H122X, N127X, A142X, A143X, R147X, Y147X, F149X, A158X, Q159X, A162X, S165X, T166X, and D167X of an amino acid sequence having at least about 70%, 75%, 80%, 85%, 90%, 95%, 98%, 99% or greater identity to SEQ ID NO: 1, or a corresponding alteration in another deaminase. In various embodiments, the alterations of the invention do not include a 48R mutation. In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising a combination of amino acid alterations selected from: E27H, Y76I, and F84M; E27H, I49K, and Y76I; E27S, I49K, Y76I, and A162N; E27K and D119N; E27H and Y76I; E27S, I49K, and G67W; E27S, 149K, and Y76I; I49T, G67W, and H96N; E27C, Y76I, and D119N; R13G, E27Q, and N127K; T17A, E27H, 149M, Y76I, and M118L; I49Q, Y76I, and G115M; S2H, I49K, Y76I, and G112H; R47S and R107C; H8Q, I49Q, and Y76I; T17A, A48G, S82T, and A142E; E27G and I49N; E27G, D77G, and S165P; E27S, I49K, and S82T; E27S, I49K, S82T, and G115M; E27S, V30I, I49K, and S82T; E27S, V30F, I49K, S82T, F84A, R107C, and A142E; E27S, V30F, I49K, S82T, F84A, G112H, and A142E; E27S, V30F, I49K, S82T, F84A, G115M, and A142E; E27S, I49K, S82T, F84L, and R107C; E27S, I49K, S82T, F84L, and G112H; E27S, I49K, S82T, F84L, and G115M; E27S, I49K, S82T, F84L, R107C, and G112H; E27S, I49K, S82T, F84L, R107C, and G115M; E27S, I49K, S82T, F84L, R107C, and A142E; E27S, I49K, S82T, F84L, G112H, and A142E; E27S, I49K, S82T, F84L, G115M, and A142E; E27S, I49K, S82T, F84L, R107C, G112H, G115M, and A142E; E27S, V30I, I49K, S82T, and F84L; E27S, P29G, I49K, and S82T; E27S, P29G, I49K, S82T, and G115M; E27S, P29G, I49K, S82T, and A142E; P29G, I49K, and S82T; E27G, I49K, and S82T; E27G, I49K, S82T, R107C, and A142E; V4K, E27H, 149K, Y76I, and A114C; V4K, E27H, I49K, Y76I, and D77G; F6Y, E27H, I49K, Y76I, G100A, and H122R; V4T, E27H, I49K, Y76R, and H122G; F6Y, E27H, I49K, and Y76W; F6Y, E27H, I49K, Y76I, and D119N; F6Y, E27H, I49K, Y76I, and A114C; F6Y, E27H, I49K, and Y76I; V4K, E27H, 149K, Y76W, and H122T; F6G, E27H, I49K, Y76R, and G100K; F6H, E27H, I49K, Y76I, and H122N; E27H, I49K, Y76I, and A114C; F6Y, E27H, I49K, Y76H, H122R, and T166I; E27H, 149K, Y76I, and N127P; R23Q, E27H, I49K, and Y76R; E27H, I49K, Y76H, H122R, and A158V; F6Y, E27H, I49K, Y76I, and T111H; E27H, I49K, Y76I, and R147H; E27H, I49K, Y76I, and A143E; F6Y, E27H, I49K, and Y76R; T17W, E27H, I49K, Y76H, H122G, and A158V; V4S, E27H, I49K, A143E, and Q159S; E27H, I49K, Y76I, N127I, and A162Q; T17A, E27H, and A48G; T17A, E27K, and A48G; T17A, E27S, and A48G; T17A, E27S, A48G, and I49K; T17A, E27G, and A48G; T17A, A48G, and I49N; T17A, E27G, A48G, and I49N; T17A, E27Q, and A48G; E27S, I49K, S82T, and R107C; E27S, I49K, S82T, and G112H; E27S, I49K, S82T, and A142E; E27S, I49K, S82T, R107C, and G112H; E27S, I49K, S82T, R107C, and G115M; E27S, I49K, S82T, R107C, and A142E; E27S, I49K, S82T, G112H, and A142E; E27S, 149K, S82T, G115M, and A142E; E27S, I49K, S82T, R107C, G112H, G115M, and A142E; E27S, V30I, I49K, S82T, and R107C; E27S, V30I, I49K, S82T, and G112H; E27S, V30I, I49K, S82T, and G15M; E27S, V30I, I49K, S82T, and A142E; E27S, V30I, I49K, S82T, R107C, and G112H; E27S, V30I, I49K, S82T, R107C, and G115M; E27S, V30I, I49K, S82T, R107C, and A142E; E27S, V30I, I49K, S82T, G112H, and A142E; E27S, V30I, I49K, S82T, G115M, and A142E; E27S, V30I, I49K, S82T, R107C, G112H, G115M, and A142E; E27S, V30L, I49K, and S82T; E27S, V30L, I49K, S82T, and R107C; E27S, V30L, I49K, S82T, and G112H; E27S, V30L, 149K, S82T, and G115M; E27S, V30L, I49K, S82T, and A142E; E27S, V30L, I49K, S82T, R107C, and G112H; E27S, V30L, I49K, S82T, R107C, and G115M; E27S, V30L, I49K, S82T, R107C, and A142E; E27S, V30L, I49K, S82T, G112H, and A142E; E27S, V30L, I49K, S82T, G115M, and A142E; E27S, V30L, I49K, S82T, R107C, G112H, G115M, and A142E; E27S, V30F, 149K, S82T, and F84A; E27S, V30F, I49K, S82T, F84A, and R107C; E27S, V30F, I49K, S82T, F84A, and G112H; E27S, V30F, I49K, S82T, F84A, and G115M; E27S, V30F, I49K, S82T, F84A, and A142E; E27S, V30F, I49K, S82T, F84A, R107C, and G112H; E27S, V30F, I49K, S82T, F84A, R107C, and G115M; E27S, V30F, I49K, S82T, F84A, R107C, G112H, G115M, and A142E; E27S, I49K, S82T, and F84L; E27S, I49K, S82T, F84L, and A142E; E27S, V30I, I49K, S82T, F84L, and R107C; E27S, V30I, I49K, S82T, F84L, and G112H; E27S, V30I, I49K, S82T, F84L, and G115M; E27S, V30I, I49K, S82T, F84L, and A142E; E27S, V30I, I49K, S82T, F84L, R107C, and G112H; E27S, V30I, I49K, S82T, F84L, R107C, and G115M; E27S, V30I, I49K, S82T, F84L, R107C, and A142E; E27S, V30I, I49K, S82T, F84L, G112H, and A142E; E27S, V30I, I49K, S82T, F84L, G115M, and A142E; E27S, V30I, I49K, S82T, F84L, R107C, G112H, G115M, and A142E; E27S, P29G, I49K, S82T, and R107C; E27S, P29G, 149K, S82T, and G112H; E27S, P29G, I49K, S82T, R107C, and G112H; E27S, P29G, I49K, S82T, R107C, and G115M; E27S, P29G, I49K, S82T, R107C, and A142E; E27S, P29G, 149K, S82T, G112H, and A142E; E27S, P29G, I49K, S82T, G115M, and A142E; E27S, P29G, I49K, S82T, R107C, G112H, G115M, and A142E; P29G, I49K, S82T, and R107C; P29G, 149K, S82T, and G112H; P29G, I49K, S82T, and G115M; P29G, I49K, S82T, and A142E; P29G, I49K, S82T, R107C, and G112H; P29G, I49K, S82T, R107C, and G115M; P29G, I49K, S82T, R107C, and A142E; P29G, I49K, S82T, G112H, and A142E; P29G, I49K, S82T, G115M, and A142E; P29G, I49K, S82T, R107C, G112H, G115M, and A142E; P29K, I49K, and S82T; P29K, 149K, S82T, and R107C; P29K, I49K, S82T, and G112H; P29K, I49K, S82T, and G115M; P29K, I49K, S82T, and A142E; P29K, I49K, S82T, R107C, and G112H; P29K, I49K, S82T, R107C, and G115M; P29K, I49K, S82T, R107C, and A142E; P29K, I49K, S82T, G112H, and A142E; P29K, I49K, S82T, G115M, and A142E; P29K, I49K, S82T, R107C, G112H, G115M, and A142E; P29K, V30I, I49K, and S82T; P29K, V30I, I49K, S82T, and R107C; P29K, V30I, I49K, S82T, and G112H; P29K, V30I, I49K, S82T, and G115M; P29K, V30I, I49K, S82T, and A142E; P29K, V30I, I49K, S82T, R107C, and G112H; P29K, V30I, I49K, S82T, R107C, and G15M; P29K, V30I, I49K, S82T, R107C, and A142E; P29K, V30I, I49K, S82T, G112H, and A142E; P29K, V30I, I49K, S82T, G115M, and A142E; P29K, V30I, I49K, S82T, R107C, G112H, G115M, and A142E; P29K, I49K, S82T, and F84L; P29K, I49K, S82T, F84L, and R107C; P29K, I49K, S82T, F84L, and G112H; P29K, I49K, S82T, F84L, and G115M; P29K, I49K, S82T, F84L, and A142E; P29K, I49K, S82T, F84L, R107C, and G112H; P29K, I49K, S82T, F84L, R107C, and G115M; P29K, I49K, S82T, F84L, R107C, and A142E; P29K, I49K, S82T, F84L, G112H, and A142E; P29K, I49K, S82T, F84L, G115M, and A142E; P29K, I49K, S82T, F84L, R107C, G112H, G115M, and A142E; P29K, V30I, I49K, S82T, and F84L; P29K, V30I, I49K, S82T, F84L, and R107C; P29K, V30I, I49K, S82T, F84L, and G112H; P29K, V30I, I49K, S82T, F84L, and G115M; P29K, V30I, I49K, S82T, F84L, and A142E; P29K, V30I, I49K, S82T, F84L, R107C, and G112H; P29K, V30I, I49K, S82T, F84L, R107C, and G115M; P29K, V30I, I49K, S82T, F84L, R107C, and A142E; P29K, V30I, I49K, S82T, F84L, G112H, and A142E; P29K, V30I, I49K, S82T, F84L, G115M, and A142E; P29K, V30I, I49K, S82T, F84L, R107C, G112H, G115M, and A142E; E27G, I49K, S82T, and R107C; E27G, 149K, S82T, and G112H; E27G, I49K, S82T, and G115M; E27G, I49K, S82T, and A142E; E27G, I49K, S82T, R107C, and G112H; E27G, I49K, S82T, R107C, and G115M; E27G, I49K, S82T, G112H, and A142E; E27G, I49K, S82T, G115M, and A142E; E27G, I49K, S82T, R107C, G112H, G115M, and A142E; E27H, I49K, and S82T; E27H, I49K, S82T, and R107C; E27H, 149K, S82T, and G112H; E27H, I49K, S82T, and G115M; E27H, I49K, S82T, and A142E; E27H, I49K, S82T, R107C, and G112H; E27H, I49K, S82T, R107C, and G115M; E27H, I49K, S82T, R107C, and A142E; E27H, I49K, S82T, G112H, and A142E; E27H, I49K, S82T, G115M, and A142E; E27H, I49K, S82T, R107C, G112H, G115M, and A142E; E27S, and S82T; E27S, S82T, and R107C; E27S, S82T, and G112H; E27S, S82T, and G115M; E27S, S82T, and A142E; E27S, S82T, R107C, and G112H; E27S, S82T, R107C, and G115M; E27S, S82T, R107C, and A142E; E27S, S82T, G112H, and A142E; E27S, S82T, G115M, and A142E; E27S, S82T, R107C, G112H, G115M, and A142E; P29A, and S82T; P29A, S82T, and R107C; P29A, S82T, and G112H; P29A, S82T, and G115M; P29A, S82T, and A142E; P29A, S82T, R107C, and G112H; P29A, S82T, R107C, and G115M; P29A, S82T, R107C, and A142E; P29A, S82T, G112H, and A142E; P29A, S82T, G115M, and A142E; P29A, S82T, R107C, G112H, G115M, and A142E; E27S, V30I, and S82T; E27S, V30I, S82T, and R107C; E27S, V30I, S82T, and G112H; E27S, V30I, S82T, and G115M; E27S, V30I, S82T, and A142E; E27S, V30I, S82T, R107C, and G112H; E27S, V30I, S82T, R107C, and G115M; E27S, V30I, S82T, R107C, and A142E; E27S, V30I, S82T, G112H, and A142E; E27S, V30I, S82T, G115M, and A142E; E27S, V30I, S82T, R107C, G112H, G115M, and A142E; P29A, V30I, S82T, and F84L; P29A, V30I, S82T, F84L, and R107C; P29A, V30I, S82T, F84L, and G112H; P29A, V30I, S82T, F84L, and G115M; P29A, V30I, S82T, F84L, and A142E; P29A, V30I, S82T, F84L, R107C, and G112H; P29A, V30I, S82T, F84L, R107C, and G115M; P29A, V30I, S82T, F84L, R107C, and A142E; P29A, V30I, S82T, F84L, G112H, and A142E; P29A, V30I, S82T, F84L, G115M, and A142E; P29A, V30I, S82T, F84L, R107C, G112H, G115M, and A142E; E27S, P29A, V30L, I49K, S82T, F84L, R107C, G112H, G115M, and A142E; V4K, and A114C; V4K, and D77G; F6Y, G100A, and H122R; V4T, I76R, and H122G; F6Y, and I76W; F6Y, and D119N; F6Y, and A114C; V4K, I76W, and H122T; F6G, I76R, and G100K; F6H, and H122N; F6Y, 176H, H122R, and T166I; R23Q, and I76R; I76H, H122R, and A158V; F6Y, and T111H; T111H, H122G, and A162C; F6Y, and I76R; T17W, I76H, H122G, and A158V; V4S, I76Y, A143E, and Q159S; N127I, A162Q; E27H, Y76I, F84M, and F149Y; E27H, I49K, Y76I, and F149Y; T17A, E27H, 149M, Y76I, M118L, and F149Y; T17A, A48G, S82T, A142E, and F149Y; E27G, and F149Y; E27G, I49N, and F149Y; E27H, Y76I, F84M, Y147D, F149Y, T166I, and D167N; E27H, I49K, Y76I, Y147D, F149Y, T166I, D167N; T17A, E27H, I49M, Y76I, M118L, Y147D, F149Y, T166I, and D167N; T17A, A48G, S82T, A142E, Y147D, F149Y, T166I, and D167N; E27G, Y147D, F149Y, T166I, and D167N; E27G, I49N, Y147D, F149Y, T166I, and D167N; F6Y, E27H, I49K, Y76W, S82T, R107C, G112H, G115M, and A142E; F6Y, E27H, I49K, D77G, S82T, R107C, G112H, G115M, and A142E; F6Y, E27H, I49K, S82T, R107C, G112H, A114C, G115M, and A142E; F6Y, E27H, I49K, S82T, R107C, G112H, G115M, D119N, and A142E; F6Y, E27H, I49K, S82T, R107C, G112H, G115M, H122G, and A142E; F6Y, E27H, I49K, S82T, R107C, G112H, G115M, N127P, and A142E; F6Y, E27H, I49K, S82T, R107C, G112H, G115M, A142E, and A143E; F6Y, E27H, I49K, S82T, R107C, G112H, G115M, and A143E; F6Y, E27H, I49K, Y76W, D77G, S82T, R107C, G112H, G115M, and A142E; F6Y, E27H, I49K, Y76W, S82T, R107C, G112H, A114C, G115M, and A142E; F6Y, E27H, I49K, Y76W, S82T, R107C, G112H, G115M, D119N, and A142E; F6Y, E27H, I49K, Y76W, S82T, R107C, G112H, G115M, H122G, and A142E; F6Y, E27H, I49K, Y76W, S82T, R107C, G112H, G115M, N127P, and A142E; F6Y, E27H, I49K, Y76W, S82T, R107C, G112H, G115M, A142E, and A143E; F6Y, E27H, I49K, Y76W, S82T, R107C, G112H, G115M, and A143E; F6Y, E27H, I49K, D77G, S82T, R107C, G112H, A114C, G115M, and A142E; F6Y, E27H, I49K, D77G, S82T, R107C, G112H, G115M, D119N, and A142E; F6Y, E27H, I49K, D77G, S82T, R107C, G112H, G115M, H122G, and A142E; F6Y, E27H, I49K, D77G, S82T, R107C, G112H, G115M, N127P, and A142E; F6Y, E27H, I49K, D77G, S82T, R107C, G112H, G115M, A142E, and A143E; F6Y, E27H, I49K, D77G, S82T, R107C, G112H, G115M, and A143E; F6Y, E27H, I49K, S82T, R107C, G112H, A114C, G115M, D119N, and A142E; F6Y, E27H, I49K, S82T, R107C, G112H, A114C, G115M, H122G, and A142E; F6Y, E27H, I49K, S82T, R107C, G112H, A114C, G115M, N127P, and A142E; F6Y, E27H, I49K, S82T, R107C, G112H, G115M, D119N, H122G, and A142E; F6Y, E27H, 149K, S82T, R107C, G112H, G115M, D119N, N127P, and A142E; F6Y, E27H, I49K, S82T, R107C, G112H, G115M, H122G, N127P, and A142E; F6Y, E27H, I49K, Y76W, D77G, S82T, R107C, G112H, A114C, G115M, and A142E; F6Y, E27H, I49K, Y76W, D77G, S82T, R107C, G112H, G115M, D119N, and A142E; F6Y, E27H, I49K, Y76W, D77G, S82T, R107C, G112H, G115M, H122G, and A142E; F6Y, E27H, I49K, Y76W, D77G, S82T, R107C, G112H, G115M, N127P, and A142E; F6Y, E27H, I49K, Y76W, D77G, S82T, R107C, G112H, G115M, A142E, and A143E; F6Y, E27H, I49K, Y76W, D77G, S82T, R107C, G112H, G115M, and A143E; F6Y, E27H, I49K, Y76W, S82T, R107C, G112H, A114C, G115M, D119N, and A142E; F6Y, E27H, I49K, Y76W, S82T, R107C, G112H, A114C, G115M, H122G, and A142E; F6Y, E27H, I49K, Y76W, S82T, R107C, G112H, A114C, G115M, N127P, and A142E; F6Y, E27H, I49K, Y76W, S82T, R107C, G112H, A114C, G115M, A142E, and A143E; F6Y, E27H, I49K, Y76W, S82T, R107C, G112H, A114C, G115M, and A143E; F6Y, E27H, I49K, Y76W, S82T, R107C, G112H, G115M, D119N, H122G, and A142E; F6Y, E27H, I49K, Y76W, S82T, R107C, G112H, G115M, D119N, N127P, and A142E; F6Y, E27H, I49K, Y76W, S82T, R107C, G112H, G115M, H122G, N127P, and A142E; F6Y, E27H, I49K, Y76W, S82T, R107C, G112H, A114C, G115M, D119N, H122G, and A142E; F6Y, E27H, I49K, Y76W, S82T, R107C, G112H, A114C, G115M, D119N, N127P, and A142E; F6Y, E27H, I49K, Y76W, S82T, R107C, G112H, A114C, G115M, H122G, N127P, and A142E; F6Y, E27H, I49K, Y76W, D77G, S82T, R107C, G112H, A114C, G115M, D119N, and A142E; F6Y, E27H, I49K, Y76W, D77G, S82T, R107C, G112H, A114C, G115M, H122G, and A142E; F6Y, E27H, I49K, Y76W, D77G, S82T, R107C, G112H, A114C, G115M, N127P, and A142E; F6Y, E27H, I49K, Y76W, D77G, S82T, R107C, G112H, A114C, G115M, A142E, and A143E; F6Y, E27H, I49K, Y76W, D77G, S82T, R107C, G112H, A114C, G115M, and A143E; F6Y, E27H, I49K, Y76W, D77G, S82T, R107C, G112H, A114C, G115M, D119N, H122G, and A142E; F6Y, E27H, I49K, Y76W, D77G, S82T, R107C, G112H, A114C, G115M, D119N, N127P, and A142E; F6Y, E27H, I49K, Y76W, D77G, S82T, R107C, G112H, A114C, G115M, H122G, N127P, and A142E; F6Y, E27H, I49K, Y76W, S82T, R107C, G112H, A114C, G115M, D119N, H122G, N127P, A142E, and A143E; F6Y, E27H, I49K, Y76W, S82T, R107C, G112H, A114C, G115M, D119N, H122G, N127P, and A143E; F6Y, E27H, I49K, Y76W, D77G, S82T, R107C, G112H, A114C, G115M, D119N, H122G, N127P, A142E, and A143E; and F6Y, E27H, I49K, Y76W, D77G, S82T, R107C, G112H, A114C, G115M, D119N, H122G, N127P, and A143E; of an amino acid sequence having at least about 70%, 75%, 80%, 85%, 90%, 95%, 98%, 99% or greater identity to SEQ ID NO: 1, or a corresponding combination of alterations in another deaminase.

In some embodiments, the adenosine deaminase variant is an adenosine deaminase comprising an amino acid alteration or combination of amino acid alterations selected from those listed in any of Tables 6.2A-6.2F.

The residue identity of exemplary adenosine deaminase variants that are capable of deaminating adenine and/or cytidine in a target polynucleotide (e.g., DNA) is provided in Tables 6.2A-6.2F below. Further examples of adenosine deaminse variants include the following variants of 1.17 (see Table 6.2A): 1.17+E27H; 1.17+E27K; 1.17+E27S; 1.17+E27S+149K; 1.17+E27G; 1.17+149N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, base editing is carried out to induce therapeutic changes in the genome of a cell of a subject (e.g., human). Cells are collected from a subject and contacted with one or more guide RNAs and a nucleobase editor polypeptide comprising a nucleic acid programmable DNA binding protein (napDNAbp) (e.g., Cas9) and an adenosine deaminase variant capable of deaminating both adenine and cytosine in a target polynucleotide (e.g., DNA). In some embodiments, cells are contacted with one or more guide RNAs and a fusion protein comprising a nucleic acid programmable DNA binding protein (napDNAbp) (e.g., Cas9) and an adenosine deaminase variant capable of deaminating both adenine and cytosine in a target polynucleotide (e.g., DNA). In some embodiments, the napDNAbp is a Cas9.

In some embodiments, cells are contacted with a multi-molecular complex. In some embodiments, cells are contacted with a base editor system as provided herein. In some embodiments, the base editor systems as provided herein comprise an adenosine base editor (ABE) variant (e.g., a CABE). In some embodiments, the CABE variant is an ABE8 variant. In some embodiments, the ABE8 variant is an ABE8.20 variant. In some embodiments, CABEs as provided herein have both A to G and C to T base editing activity. Therefore, multiple edits may be introduced into the genome of a subject (e.g., human). The ability to target both A to G and C to T base editing activity allows for diverse targeting of polynucleotides in the genome in a subject to treat a genetic disease or disorder.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6.2A
Adenosine Deaminase Variants (CABE-1; T_{AD}AC-1). Mutations are indicated with reference to TadA*8.20.

| | location in structure | | | | | | | | | | | | | |
|---|---|---|---|---|---|---|---|---|---|---|---|---|---|---|
| | | | | | near | near | near | near | | | | | | |
| | | surface | surface | surface | active | active | active | active | | | | | | |
| | N/A | h1 | h1 | h1 | site | site | site | site | | surface | | | | |
| | Amino Acid No. (*START Met is AA#1) | | | | | | | | | | | | | |
| | 2 | 8 | 13 | 17 | 27 | 47 | 48 | 49 | 67 | 76 | 77 | | | |
| | TadA*8.20 | | | | | | | | | | | | | |
| | S | H | R | T | E | R | A | I | G | Y | D | | | |
| | TadA*8.19 | | | | | | | | | | | Plasmid #s | | |
| | | | | | | | | | | I | | without ugi | with ugi | bacterial |
| 1.1 | | | | | H | | | | | I | | pDKL-11 | pDKL-23 | pDKL-35 |
| 1.2 | | | | | H | | | K | | I | | pDKL-12 | pDKL-24 | pDKL-36 |
| 1.3 | | | | | S | | | K | | I | | pDKL-13 | pDKL-25 | pDKL-37 |
| 1.4 | | | | | S | | | K | | I | | pDKL-14 | pDKL-26 | pDKL-38 |
| 1.5 | | | | | K | | | | | | | PDKL-15 | pDKL-27 | pDKL-39 |
| 1.6 | | | | | K | | | | | | | pDKL-16 | pDKL-28 | pDKL-40 |
| 1.7 | | | | | H | | | | | I | | pDKL-17 | pDKL-29 | pDKL-41 |
| 1.8 | | | | | S | | | K | W | | | pDKL-18 | pDKL-30 | pDKL-42 |
| 1.9 | | | | | | | | T | W | | | pDKL-19 | pDKL-31 | pDKL-43 |
| 1.10 | | | | | C | | | | | I | | pDKL-20 | pDKL-32 | pDKL-44 |
| 1.11 | | | G | | Q | | | | | | | pDKL-21 | pDKL-33 | pDKL-45 |
| 1.12 | | | | A | H | | | M | | I | | pDKL-22 | pDKL-34 | pDKL-46 |
| 1.13 | | | | | | | | Q | | I | | pDKL-47 | pDKL-55 | pDKL-63 |
| 1.14 | H | | | | | | | K | | I | | pDKL-48 | pDKL-56 | pDKL-64 |
| 1.15 | | | | | | S | | | | | | pDKL-49 | pDKL-57 | pDKL-65 |
| 1.16 | | Q | | | | | | Q | | I | | pDKL-50 | pDKL-58 | pDKL-66 |
| 1.17 | | | | A | | | G | | | | | pDKL-51 | pDKL-59 | pDKL-67 |
| 1.18 | | | | | G | | | | | | | pDKL-52 | pDKL-60 | pDKL-68 |
| 1.19 | | | | | G | | | N | | | | pDKL-53 | pDKL-61 | pDKL-69 |
| 1.20 | | | | | G | | | | | | G | pDKL-54 | pDKL-62 | pDKL-70 |

| | location in structure | | | | | | | | | | | | | | |
|---|---|---|---|---|---|---|---|---|---|---|---|---|---|---|---|
| | | near | | near | | | | | | | | | | | |
| | | active | | active | | | | | | | | | | | |
| | internal | site | | site | surface | | surface | surface | surface | | surface | | | | |
| | Amino Acid No. (*START Met is AA#1) | | | | | | | | | | | | | | |
| | 82 | 84 | 96 | 107 | 112 | 115 | 118 | 119 | 127 | 142 | 162 | 165 | | | |
| | TadA*8.20 | | | | | | | | | | | | | | |
| | S | F | H | R | G | G | M | D | N | A | A | S | Plasmid #s | | |
| | TadA*8.19 | | | | | | | | | | | | without ugi | with ugi | bacterial |
| 1.1 | | M | | | | | | | | | | | pDKL-11 | pDKL-23 | pDKL-35 |
| 1.2 | | | | | | | | | | | | | pDKL-12 | pDKL-24 | pDKL-36 |
| 1.3 | | | | | | | | | | | | | pDKL-13 | pDKL-25 | pDKL-37 |
| 1.4 | | | | | | | | | | | N | | pDKL-14 | pDKL-26 | pDKL-38 |
| 1.5 | | | | | | | | | | | | | pDKL-15 | pDKL-27 | pDKL-39 |
| 1.6 | | | | | | | | N | | | | | pDKL-16 | pDKL-28 | pDKL-40 |
| 1.7 | | | | | | | | | | | | | pDKL-17 | pDKL-29 | pDKL-41 |
| 1.8 | | | | | | | | | | | | | pDKL-18 | pDKL-30 | pDKL-42 |
| 1.9 | | | N | | | | | | | | | | pDKL-19 | pDKL-31 | pDKL-43 |
| 1.10 | | | | | | | | N | | | | | pDKL-20 | pDKL-32 | pDKL-44 |
| 1.11 | | | | | | | | | K | | | | pDKL-21 | pDKL-33 | pDKL-45 |
| 1.12 | | | | | | | L | | | | | | pDKL-22 | pDKL-34 | pDKL-46 |
| 1.13 | | | | | | M | | | | | | | pDKL-47 | pDKL-55 | pDKL-63 |
| 1.14 | | | | | H | | | | | | | | pDKL-48 | pDKL-56 | pDKL-64 |
| 1.15 | | | | C | | | | | | | | | pDKL-49 | pDKL-57 | pDKL-65 |
| 1.16 | | | | | | | | | | | | | pDKL-50 | pDKL-58 | pDKL-66 |
| 1.17 | T | | | | | | | | | E | | | pDKL-51 | pDKL-59 | pDKL-67 |
| 1.18 | | | | | | | | | | | | | pDKL-52 | pDKL-60 | pDKL-68 |
| 1.19 | | | | | | | | | | | | | pDKL-53 | pDKL-61 | pDKL-69 |
| 1.20 | | | | | | | | | | | | P | pDKL-54 | pDKL-62 | pDKL-70 |

TABLE 6.2B
Rationally Designed Candidate Editors (CABE-2s; TADAC-
2s). Mutations are indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6.2C
Candidate base editors (CABE-2e; TADAC-2e). Mutations are indicated with reference to variant 1.2 (Table 6.2A).

| Variant | Alternative Variant | Residue identity (START Met is amino acid #1) | | | | | | | | | | | | | | | | | |
|---|---|---|---|---|---|---|---|---|---|---|---|---|---|---|---|---|---|---|---|
| Name | Names | 4 | 6 | 17 | 23 | 76 | 77 | 100 | 111 | 114 | 119 | 122 | 127 | 143 | 147 | 158 | 159 | 162 | 166 |
| Reference | 1.2 (see Table 6.2A) | V | F | T | R | I | D | G | T | A | D | H | N | A | R | A | Q | A | T |
| TadAC2.1 | pDKL-135; 2.1 | K | | | | | | | | C | | | | | | | | | |
| TadAC2.2 | pDKL-136; 2.2 | K | | | | | G | | | | | | | | | | | | |
| TadAC2.3 | pDKL-137; 2.3 | | Y | | | | | A | | | | R | | | | | | | |
| TadAC2.4 | pDKL-138; 2.4 | T | | | | R | | | | | | G | | | | | | | |
| TadAC2.5 | pDKL-139; 2.5 | | Y | | | W | | | | | | | | | | | | | |
| TadAC2.6 | pDKL-140; 2.6 | | Y | | | | | | | | N | | | | | | | | |
| TadAC2.7 | pDKL-141; 2.7 | | Y | | | | | | | C | | | | | | | | | |
| TadAC2.8 | pDKL-142; 2.8 | | Y | | | | | | | | | | | | | | | | |
| TadAC2.9 | pDKL-143; 2.9 | K | | | | W | | | | | | T | | | | | | | |
| TadAC2.10 | pDKL-144; 2.10 | | G | | | R | | K | | | | | | | | | | | |
| TadAC2.11 | pDKL-145; 2.11 | | H | | | | | | | | | N | | | | | | | |
| TadAC2.12 | pDKL-146; 2.12 | | | | | | | | | C | | | | | | | | | |
| TadAC2.13 | pDKL-147; 2.13 | | Y | | | H | | | | | | R | | | | | | | I |
| TadAC2.14 | pDKL-148; 2.14 | | | | | | | | | | | | P | | | | | | |
| TadAC2.15 | pDKL-149; 2.15 | | | | Q | R | | | | | | | | | | | | | |
| TadAC2.16 | pDKL-150; 2.16 | | | | | H | | | | | | R | | | | V | | | |
| TadAC2.17 | pDKL-151; 2.17 | | Y | | | | | | H | | | | | | | | | | |
| TadAC2.18 | pDKL-152; 2.18 | | | | | W | | | | | | | | | | | | | |
| TadAC2.19 | pDKL-153; 2.19 | | | | | | | | H | | | G | | | | | | C | |
| TadAC2.20 | pDKL-154; 2.20 | | | | | | | | | | | | | E | | | | | |
| TadAC2.21 | pDKL-155; 2.21 | | Y | | | R | | | | | | | | | | | | | |
| TadAC2.22 | pDKL-156; 2.22 | | | W | | H | | | | | | G | | | | V | | | |
| TadAC2.23 | pDKL-157; 2.23 | S | | | | Y | | | | | | | | E | | | S | | |
| TadAC2.24 | pDKL-158; 2.24 | | | | | | | | | | | | I | | | | | Q | |

TABLE 6.2D
Rationally Designed Candidate Editors (CBE-Ts; T_{AD}Cs).
Mutations are indicated with reference to ABE8.20m.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| | TadA8.20 | | | | | | | | | | | | | | |
| | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table 6.2C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6.2E
Hybrid constructs. Mutations are indicated with reference to ABE7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| ABE7.10 | I | V | A | T | D | H | Y | Y | F | Q | T | D |
| ABE8e | | | S | R | N | N | | D | Y | | I | N |
| ABE8.20 | Y | S | | | | | H | R | | R | | |
| ABE8.17 | | S | | | | | | | | R | | |
| pNMG- | Y | S | | | | | H | D | | R | | |
| B878 | | | | | | | | | | | | |
| pNMG- | Y | S | | | | | H | R | Y | R | | |
| B879 | | | | | | | | | | | | |
| pNMG- | Y | S | | | | | H | R | | R | I | |
| B880 | | | | | | | | | | | | |
| pNMG- | Y | S | | | | | H | R | | R | | N |
| B881 | | | | | | | | | | | | |
| pNMG- | Y | S | | | | | H | D | Y | R | I | N |
| B882 | | | | | | | | | | | | |
| pNMG- | Y | S | | R | N | | H | R | | R | | |
| B883 | | | | | | | | | | | | |
| pNMG- | Y | S | S | R | N | N | H | R | | R | | |
| B884 | | | | | | | | | | | | |
| pNMG- | Y | S | S | | | | H | R | | R | | |
| B885 | | | | | | | | | | | | |
| pNMG- | Y | S | | R | | | H | R | | R | | |
| B886 | | | | | | | | | | | | |
| pNMG- | Y | S | | | N | | H | R | | R | | |
| B887 | | | | | | | | | | | | |
| pNMG- | Y | S | | | | N | H | R | | R | | |
| B888 | | | | | | | | | | | | |
| pNMG- | Y | S | S | R | | | H | R | | R | | |
| B889 | | | | | | | | | | | | |
| pNMG- | Y | S | | | N | N | H | R | | R | | |
| B890 | | | | | | | | | | | | |
| pNMG- | Y | S | S | R | N | N | H | D | Y | R | I | N |
| B891 | | | | | | | | | | | | |

TABLE 6.2F
Base editor variants. Mutations are indicated with reference to ABE8.19m/8.20m.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., et al. Science 337:816-821(2012), the entire contents of which is hereby incorporated by reference. Cas9 recognizes a short motif in the CRISPR repeat sequences (the PAM or protospacer adjacent motif) to help distinguish self versus non-self. See e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference).

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In an embodiment, a guide polynucleotide described herein can be RNA or DNA. In one embodiment, the guide polynucleotide is a gRNA. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

A guide polynucleotide may include natural or non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome.

In other embodiments, a guide polynucleotide comprises both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion (e.g., a functional portion) of multiple separate molecules that are for instance hybridized along a region of complementarity. In some embodiments, a protein-binding segment of a DNA-targeting RNA that comprises two separate molecules comprises (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex and can include regions of RNA molecules that are of any total length and can include regions with complementarity to other molecules.

The guide polynucleotides can be synthesized chemically, synthesized enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include T7, T3, SP6 promoter sequences, or variations thereof. In embodiments in which the gRNA comprises two separate molecules (e.g., crRNA and tracrRNA), the crRNA can be chemically synthesized and the tracrRNA can be enzymatically synthesized.

A guide polynucleotide may be expressed, for example, by a DNA that encodes the gRNA, e.g., a DNA vector comprising a sequence encoding the gRNA. The gRNA may be encoded alone or together with an encoded base editor. Such DNA sequences may be introduced into an expression system, e.g., a cell, together or separately. For example, DNA sequences encoding a polynucleotide programmable nucleotide binding domain and a gRNA may be introduced into a cell, each DNA sequence can be part of a separate molecule (e.g., one vector containing the polynucleotide programmable nucleotide binding domain coding sequence and a second vector containing the gRNA coding sequence) or both can be part of a same molecule (e.g., one vector containing coding (and regulatory) sequence for both the polynucleotide programmable nucleotide binding domain and the gRNA). An RNA can be transcribed from a synthetic DNA molecule, e.g., a gBlocks® gene fragment. A gRNA molecule can be transcribed in vitro.

A gRNA or a guide polynucleotide can comprise three regions: a first region at the 5′ end that can be complementary to a target site in a chromosomal sequence, a second internal region that can form a stem loop structure, and a third 3′ region that does not form a secondary structure or bind a target site. A first region of each gRNA can also be different such that each gRNA guides a fusion protein or complex to a specific target site. Further, second and third regions of each gRNA can be identical in all gRNAs.

A first region of a gRNA or a guide polynucleotide can be complementary to sequence at a target site in a chromosomal sequence such that the first region of the gRNA can base pair with the target site. In some cases, a first region of a gRNA comprises from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a gRNA and a target site in a chromosomal sequence can be or can be about 10, 11, 12, 13, 14, 15, 16, 17, 18, 19, 20, 22, 23, 24, 25, or more nucleotides in length. Sometimes, a first region of a gRNA can be or can be about 19, 20, or 21 nucleotides in length.

A gRNA or a guide polynucleotide can also comprise a second region that forms a secondary structure. For example, a secondary structure formed by a gRNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A gRNA or a guide polynucleotide can also comprise a third region at the 3′ end that can be essentially single-stranded. For example, a third region is sometimes not complementarity to any chromosomal sequence in a cell of interest and is sometimes not complementarity to the rest of a gRNA. Further, the length of a third region can vary. A third region can be more than or more than about 4 nucleotides in length. For example, the length of a third region can range from or from about 5 to 60 nucleotides in length.

A gRNA or a guide polynucleotide can target any exon or intron of a gene target. In some cases, a guide can target Exon 1 or 2 of a gene, in other cases; a guide can target Exon 3 or 4 of a gene. In some embodiments, a composition comprises multiple gRNAs that all target the same exon or multiple gRNAs that target different exons. An exon and/or an intron of a gene can be targeted.

A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

Methods for selecting, designing, and validating guide polynucleotides, e.g., gRNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on single strand DNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a gRNA for use with Cas9s may be identified using a DNA sequence searching algorithm. gRNA design is carried out using custom gRNA design software based on the public tool cas-OFFinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of gRNAs, e.g., crRNAs, are ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

A gRNA can then be introduced into a cell or embryo as an RNA molecule or a non-RNA nucleic acid molecule, e.g., DNA molecule. In one embodiment, a DNA encoding a gRNA is operably linked to promoter control sequence for expression of the gRNA in a cell or embryo of interest. A RNA coding sequence can be operably linked to a promoter sequence that is recognized by RNA polymerase III (Pol III). Plasmid vectors that can be used to express gRNA include, but are not limited to, px330 vectors and px333 vectors. In some cases, a plasmid vector (e.g., px333 vector) comprises at least two gRNA-encoding DNA sequences. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a gRNA can also be linear. A DNA molecule encoding a gRNA or a guide polynucleotide can also be circular.

In some embodiments, a reporter system is used for detecting base-editing activity and testing candidate guide polynucleotides. In some embodiments, a reporter system comprises a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein or complex. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide comprises at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In a particular embodiment, the chemical modifications are 2′-O-methyl (2′-OMe) modifications. The modified guide RNAs may improve saCas9 efficacy and also specificity. The effect of an individual modification varies based on the position and combination of chemical modifications used as well as the inter- and intramolecular interactions with other modified nucleotides. By way of example, S-cEt has been used to improve oligonucleotide intramolecular folding.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises four modified nucleosides at the 5′ end and four modified nucleosides at the 3′ end of the guide. In some embodiments, the modified nucleoside comprises a 2′ O-methyl or a phosphorothioate.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. For such modifications, mN=2′-OMe; Ns=phosphorothioate (PS), where “N” represents the any nucleotide, as would be understood by one having skill in the art. In some cases, a nucleotide (N) may contain two modifications, for example, both a 2′-OMe and a PS modification. For example, a nucleotide with a phosphorothioate and 2′ OMe is denoted as “mNs;” when there are two modifications next to each other, the notation is “mNsmNs.

In some embodiments of the modified gRNA, the gRNA comprises one or more chemical modifications selected from the group consisting of 2′-O-methyl (2′-OMe), phosphorothioate (PS), 2′-O-methyl thioPACE (MSP), 2′-O-methyl-PACE (MP), 2′-O-methyl thioPACE (MSP), 2′-fluoro RNA (2′-F-RNA), and constrained ethyl (S-cEt). In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A guide polynucleotide can be transferred into a cell by transfecting the cell with an isolated gRNA or a plasmid DNA comprising a sequence coding for the guide RNA and a promoter. A gRNA or a guide polynucleotide can also be transferred into a cell in other way, such as using virus-mediated gene delivery. A gRNA or a guide polynucleotide can be isolated. For example, a gRNA can be transfected in the form of an isolated RNA into a cell or organism. A gRNA can be prepared by in vitro transcription using any in vitro transcription system known in the art. A gRNA can be transferred to a cell in the form of isolated RNA rather than in the form of plasmid comprising encoding sequence for a gRNA.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

In some embodiments, the guide RNA is designed such that base editing results in disruption of a splice site (i.e., a splice acceptor (SA) or a splice donor (SD)). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion (e.g., a functional portion) of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 7 below.

TABLE 7
Cas9 proteins and corresponding PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, the PAM is NGT. In some embodiments, the NGT PAM is recognized by a Cas9 variant. In some embodiments, the Cas9 variant is generated through targeted mutations at one or more residues 1335, 1337, 1135, 1136, 1218, and/or 1219 of spCas9 (SEQ ID No: 197), or a corresponding mutation in another Cas9. In some embodiments, the NGT PAM Cas9 variant is created through targeted mutations at one or more residues 1219, 1335, 1337, 1218 of spCas9 (SEQ ID No: 197), or a corresponding mutation in another Cas9. In some embodiments, the NGT PAM Cas9 variant is created through targeted mutations at one or more residues 1135, 1136, 1218, 1219, and 1335 of spCas9 (SEQ ID No: 197, or a corresponding mutation in another Cas9. In some embodiments, the NGT PAM Cas9 variant is selected from the set of targeted mutations provided in Tables 8A and 8B below.

TABLE 8A
NGT PAM Variant Mutations at residues 1219,
1335, 1337, 1218 of spCas9 (SEQ ID No: 197),
or a corresponding mutation in another Cas9

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 8B
NGT PAM Variant Mutations at residues 1135, 1136,
1218, 1219, and 1335 of spCas9 (SEQ ID No: 197),
or a corresponding mutation in another Cas9

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM Cas9 variant is selected from variant 5, 7, 28, 31, or 36 in Table 8A and Table 8B. In some embodiments, the variants have improved NGT PAM recognition.

In some embodiments, the NGT PAM Cas9 variants have mutations at residues 1219, 1335, 1337, and/or 1218. In some embodiments, the NGT PAM Cas9 variant is selected with mutations for improved recognition from the variants provided in Table 9 below.

TABLE 9
NGT PAM Variant Mutations at residues 1219, 1335,
1337, and 1218 of spCas9 (SEQ ID No: 197), or
a corresponding mutation in another Cas9

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM Cas9 variant is selected from the variants provided in Table 10 below.

TABLE 10
NGT PAM variants, where the amino acid residue locations are referenced to
of spCas9 (SEQ ID No: 197), or a corresponding mutation in another Cas9

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN Cas9 variant is variant 1. In some embodiments, the NGTN Cas9 variant is variant 2. In some embodiments, the NGTN Cas9 variant is variant 3. In some embodiments, the NGTN Cas9 variant is variant 4. In some embodiments, the NGTN variant is variant 5. In some embodiments, the NGTN Cas9 variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPRI and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow: In some embodiments, engineered SpCas9 variants are capable of recognizing protospacer adjacent motif (PAM) sequences flanked by a 3′ H (non-G PAM) (see Tables 3A-3D). In some embodiments, the SpCas9 variants recognize NRNH PAMs (where R is A or G and H is A, C or T). In some embodiments, the non-G PAM is NRRH, NRTH, or NRCH (see e.g., Miller, S. M., et al. Continuous evolution of SpCas9 variants compatible with non-G PAMs, Nat. Biotechnol. (2020), the contents of which is incorporated herein by reference in its entirety).

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Chatterjee, et al., “A Cas9 with PAM recognition for adenine dinucleotides”, Nature Communications, vol. 11, article no. 2474 (2020), and is in the Sequence Listing as SEQ ID NO: 237.

In some embodiments, a variant Cas9 protein harbors, H840A, P475A, W476A, N477A, D1125A, W1126A, and D1218A mutations relative to a reference Cas9 sequence (e.g., spCas9 (SEQ ID No: 197)), or to a corresponding mutation in another Cas9, such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1218A mutations relative to a reference Cas9 sequence (e.g., spCas9 (SEQ ID No: 197)), or to a corresponding mutation in another Cas9, such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1218A mutations relative to a reference Cas9 sequence (e.g., spCas9 (SEQ ID No: 197)), or to a corresponding mutation in another Cas9, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted) relative to a reference Cas9 sequence (e.g., spCas9 (SEQ ID No: 197)), or to a corresponding mutation in another Cas9. Also, mutations other than alanine substitutions are suitable.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:
NH₂-[A-B-C]-COOH;
NH₂-[A-B-C-D]-COOH; or
NH₂-[A-B-C-D-E]-COOH;
   wherein A and C or A, C, and E, each comprises one or more of the following:
an adenosine deaminase domain or an active fragment thereof,
a cytidine deaminase domain or an active fragment thereof, and
wherein B or B and D, each comprises one or more domains having nucleic acid sequence specific binding activity.

In some embodiments, the fusion protein comprises the following structure:
NH₂-[Aₙ-Bₒ-Cₙ]-COOH;
NH₂-[Aₙ-Bₒ-Cₙ-Dₒ]-COOH; or
NH₂-[Aₙ-Bₒ-Cₚ-Dₒ-E_{q}]-COOH;
   wherein A and C or A, C, and E, each comprises one or more of the following:
an adenosine deaminase domain or an active fragment thereof,
a cytidine deaminase domain or an active fragment thereof, and
   wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:
NH₂-[adenosine deaminase]-[Cas9 domain]-COOH;
NH₂-[Cas9 domain]-[adenosine deaminase]-COOH;
NH₂-[cytidine deaminase]-[Cas9 domain]-COOH;
NH₂-[Cas9 domain]-[cytidine deaminase]-COOH;
NH₂-[cytidine deaminase]-[Cas9 domain]-[adenosine deaminase]-COOH;
NH₂-[adenosine deaminase]-[Cas9 domain]-[cytidine deaminase]-COOH;
NH₂-[adenosine deaminase]-[cytidine deaminase]-[Cas9 domain]-COOH;
NH₂-[cytidine deaminase]-[adenosine deaminase]-[Cas9 domain]-COOH;
NH₂-[Cas9 domain]-[adenosine deaminase]-[cytidine deaminase]-COOH; or
NH₂-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:
NH₂-[adenosine deaminase]-[Cas12 domain]-COOH;
NH₂-[Cas12 domain]-[adenosine deaminase]-COOH;
NH₂-[cytidine deaminase]-[Cas12 domain]-COOH;
NH₂-[Cas12 domain]-[cytidine deaminase]-COOH;
NH₂-[cytidine deaminase]-[Cas12 domain]-[adenosine deaminase]-COOH;
NH₂-[adenosine deaminase]-[Cas12 domain]-[cytidine deaminase]-COOH;
NH₂-[adenosine deaminase]-[cytidine deaminase]-[Cas12 domain]-COOH;
NH₂-[cytidine deaminase]-[adenosine deaminase]-[Cas12 domain]-COOH;
NH₂-[Cas12 domain]-[adenosine deaminase]-[cytidine deaminase]-COOH; or
NH₂-[Cas12 domain]-[cytidine deaminase]-[adenosine deaminase]-COOH.

In some embodiments, the adenosine deaminase is a TadA*8. Exemplary fusion protein structures include the following:
NH₂-[TadA*8]-[Cas9 domain]-COOH;
NH₂-[Cas9 domain]-[TadA*8]-COOH;
NH₂-[TadA*8]-[Cas12 domain]-COOH; or
NH₂-[Cas12 domain]-[TadA*8]-COOH.

In some embodiments, the adenosine deaminase of the fusion protein or complex comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:
NH₂-[TadA*8]-[Cas9/Cas12]-[adenosine deaminase]-COOH;
NH₂-[adenosine deaminase]-[Cas9/Cas12]-[TadA*8]-COOH;
NH₂-[TadA*8]-[Cas9/Cas12]-[cytidine deaminase]-COOH; or
NH₂-[cytidine deaminase]-[Cas9/Cas12]-[TadA*8]-COOH.

In some embodiments, the adenosine deaminase of the fusion protein comprises a TadA*9 and a cytidine deaminase and/or an adenosine deaminase. Exemplary fusion protein structures include the following:
NH₂-[TadA*9]-[Cas9/Cas12]-[adenosine deaminase]-COOH;
NH₂-[adenosine deaminase]-[Cas9/Cas12]-[TadA*9]-COOH;
NH₂-[TadA*9]-[Cas9/Cas12]-[cytidine deaminase]-COOH; or
NH₂-[cytidine deaminase]-[Cas9/Cas12]-[TadA*9]-COOH.

In some embodiments, the fusion protein comprises a deaminase flanked by an N-terminal fragment and a C-terminal fragment of a Cas9 or Cas12 polypeptide. In some embodiments, the fusion protein comprises a cytidine deaminase flanked by an N-terminal fragment and a C-terminal fragment of a Cas9 or Cas12 polypeptide. In some embodiments, the fusion protein comprises an adenosine deaminase flanked by an N-terminal fragment and a C-terminal fragment of a Cas9 or Cas 12 polypeptide.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “−” used in the general architecture above indicates the optional presence of a linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence PKKKRKVEGADKRTADGSEFESPKKKRKV (SEQ ID NO: 328), KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRKPKKKRKV (SEQ ID NO: 329), or MDSLLMNRRKFLYQFKNVRWAKGRRETYLC (SEQ ID NO: 196).

In some embodiments, the fusion proteins or complexes comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present. In some embodiments, a linker is present between the cytidine deaminase and adenosine deaminase domains and the napDNAbp. In some embodiments, the “−” used in the general architecture below indicates the optional presence of a linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided herein.

In some embodiments, the general architecture of exemplary napDNAbp (e.g., Cas9 or Cas12) fusion proteins with a cytidine or adenosine deaminase and a napDNAbp (e.g., Cas9 or Cas12) domain comprises any one of the following structures, where NLS is a nuclear localization sequence (e.g., any NLS provided herein), NH₂ is the N-terminus of the fusion protein, and COOH is the C-terminus of the fusion protein:
NH₂-NLS-[cytidine deaminase]-[napDNAbp domain]-COOH;
NH₂-NLS [napDNAbp domain]-[cytidine deaminase]-COOH;
NH₂-[cytidine deaminase]-[napDNAbp domain]-NLS—COOH;
NH₂-[napDNAbp domain]-[cytidine deaminase]-NLS—COOH;
NH₂-NLS-[adenosine deaminase]-[napDNAbp domain]-COOH;
NH₂-NLS [napDNAbp domain]-[adenosine deaminase]-COOH;
NH₂-[adenosine deaminase]-[napDNAbp domain]-NLS—COOH;
NH₂-[napDNAbp domain]-[adenosine deaminase]-NLS—COOH;
NH₂-NLS-[cytidine deaminase]-[napDNAbp domain]-[adenosine deaminase]-COOH;
NH₂-NLS-[adenosine deaminase]-[napDNAbp domain]-[cytidine deaminase]-COOH;
NH₂-NLS-[adenosine deaminase] [cytidine deaminase]-[napDNAbp domain]-COOH;
NH₂-NLS-[cytidine deaminase]-[adenosine deaminase]-[napDNAbp domain]-COOH;
NH₂-NLS-[napDNAbp domain]-[adenosine deaminase]-[cytidine deaminase]-COOH;
NH₂-NLS-[napDNAbp domain]-[cytidine deaminase]-[adenosine deaminase]-COOH;
NH₂-[cytidine deaminase]-[napDNAbp domain]-[adenosine deaminase]-NLS—COOH;
NH₂-[adenosine deaminase]-[napDNAbp domain]-[cytidine deaminase]-NLS—COOH;
NH₂-[adenosine deaminase] [cytidine deaminase]-[napDNAbp domain]-NLS—COOH;
NH₂-[cytidine deaminase]-[adenosine deaminase]-[napDNAbp domain]-NLS—COOH;
NH₂-[napDNAbp domain]-[adenosine deaminase]-[cytidine deaminase]-NLS—COOH; or
NH₂-[napDNAbp domain]-[cytidine deaminase]-[adenosine deaminase]-NLS—COOH. In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

In some embodiments, a base editor comprises as a domain all or a portion (e.g., a functional portion) of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitutions in any domain does not change the length of the base editor.

Non-limiting examples of such base editors, where the length of all the domains is the same as the wild type domains, can include:
NH₂-[nucleobase editing domain]-Linker1-[APOBEC1]-Linker2-[nucleobase editing domain]-COOH;
NH₂-[nucleobase editing domain]-Linker1-[APOBEC1]-[nucleobase editing domain]-COOH;
NH₂-[nucleobase editing domain]-[APOBEC1]-Linker2-[nucleobase editing domain]-COOH;
NH₂-[nucleobase editing domain]-[APOBEC1]-[nucleobase editing domain]-COOH;
NH₂-[nucleobase editing domain]-Linker1-[APOBEC1]-Linker2-[nucleobase editing domain]-[UGI]-COOH;
NH₂-[nucleobase editing domain]-Linker1-[APOBEC1]-[nucleobase editing domain]-[UGI]-COOH;
NH₂-[nucleobase editing domain]-[APOBEC1]-Linker2-[nucleobase editing domain]-[UGI]-COOH;
NH₂-[nucleobase editing domain]-[APOBEC1]-[nucleobase editing domain]-[UGI]-COOH;
NH₂-[UGI]-[nucleobase editing domain]-Linker1-[APOBEC1]-Linker2-[nucleobase editing domain]-COOH;
NH₂-[UGI]-[nucleobase editing domain]-Linker1-[APOBEC1]-[nucleobase editing domain]-COOH;
NH₂-[UGI]-[nucleobase editing domain]-[APOBEC1]-Linker2-[nucleobase editing domain]-COOH; or
NH₂-[UGI]-[nucleobase editing domain]-[APOBEC1]-[nucleobase editing domain]-COOH.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

In some embodiments, a base editing system as provided herein provides a new approach to genome editing that uses a fusion protein or complex containing a catalytically defective Streptococcus pyogenes Cas9, a deaminase (e.g., cytidine or adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/US2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system (e.g., the deaminase component) comprises an additional heterologous portion or domain (e.g., polynucleotide binding domain such as an RNA or DNA binding protein) that is capable of interacting with, associating with, or capable of forming a complex with a heterologous portion or segment (e.g., a polynucleotide motif), or antigen of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the agent inhibiting the uracil-excision repair system is a uracil stabilizing protein (USP). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component comprises an additional heterologous portion or domain that is capable of interacting with, associating with, or capable of forming a complex with a corresponding additional heterologous portion, antigen, or domain that is part of a polynucleotide programmable nucleotide binding domain. In some embodiments, the polynucleotide programming nucleotide binding domain component, and/or a guide polynucleotide (e.g., a guide RNA) complexed therewith, comprises an additional heterologous portion or domain that is capable of interacting with, associating with, or capable of forming a corresponding heterologous portion, antigen, or domain that is part of an inhibitor of base excision repair component. In some embodiments, the inhibitor of base excision repair can be targeted to the target nucleotide sequence by the guide polynucleotide. For example, in some embodiments, the inhibitor of base excision repair comprises an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MH1D2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voβ, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. Non-limiting examples of polypeptides that can dimerize and their corresponding dimerizing agents are provided in Table 10.1 below.

TABLE 10.1
Chemically induced dimerization systems.

| | |
|---|---|
| Dimerizing Polypeptides | Dimerizing agent |

| | | |
|---|---|---|
| FKBP | FKBP | FK1012 |
| FKBP | Calcineurin A (CNA) | FK506 |
| FKBP | CyP-Fas | FKCsA |
| FKBP | FRB (FKBP-rapamycin- | Rapamycin |
| | binding) domain of mTOR | |
| GyrB | GyrB | Coumermycin |
| GAI | GID1 (gibberellin insensitive | Gibberellin |
| | dwarf 1) | |
| ABI | PYL | Abscisic acid |
| ABI | PYRMandi | Mandipropamid |
| SNAP-tag | HaloTag | HaXS |
| eDHFR | HaloTag | TMP-HTag |
| Bcl-xL | Fab (AZ1) | ABT-737 |

In embodiments, the additional heterologous portion is part of a guide RNA molecule. In some instances, the additional heterologous portion contains or is an RNA motif. The RNA motif may be positioned at the 5′ or 3′ end of the guide RNA molecule or various positions of a guide RNA molecule. In embodiments, the RNA motif is positioned within the guide RNA to reduce steric hindrance, optionally where such hindrance is associated with other bulky loops of an RNA scaffold. In some instances, it is advantageous to link the RNA motif is linked to other portions of the guide RNA by way of a linker, where the linker can be about, at least about, or no more than about 1, 2, 3, 4, 5, 6, 7, 8, 9, 10, or more nucleotides in length. Optionally, the linker contains a GC-rich nucleotide sequence. The guide RNA can contain 1, 2, 3, 4, 5, or more copies of the RNA motif, optionally where they are positioned consecutively, and/or optionally where they are each separated from one another by a linker(s). The RNA motif may include any one or more of the polynucleotide modifications described herein. Non-limiting examples of suitable modifications to the RNA motif include 2′ deoxy-2-aminopurine, 2′ribose-2-aminopurine, phosphorothioate mods, 2′-Omethyl mods, 2′-Fluro mods and LNA mods. Advantageously, the modifications help to increase stability and promote stronger bonds/folding structure of a hairpin(s) formed by the RNA motif.

In some embodiments, the RNA motif is modified to include an extension. In embodiments, the extension contains about, at least about, or no more than about 2, 3, 4, 5, 10, 15, 20, or 25 nucleotides. In some instances, the extension results in an alteration in the length of a stem formed by the RNA motif (e.g., a lengthening or a shortening). It can be advantageous for a stem formed by the RNA motif to be about, at least about, or no more than about 5, 10, 15, 20, 25, 30, 35, 40, 45, 50, 55, 60, 65, 70, 75, 80, 85, 90, 95, or 100 nucleotides in length. In various embodiments, the extension increases flexibility of the RNA motif and/or increases binding with a corresponding RNA motif.

In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins or complexes provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. Protein domains can be a heterologous functional domain, for example, having one or more of the following activities: transcriptional activation activity, transcriptional repression activity, transcription release factor activity, gene silencing activity, chromatin modifying activity, epigenetic modifying activity, histone modification activity, RNA cleavage activity, and nucleic acid binding activity. Such heterologous functional domains can confer a function activity, such as modification of a target polypeptide associated with target DNA (e.g., a histone, a DNA binding protein, etc.), leading to, for example, histone methylation, histone acetylation, histone ubiquitination, and the like. Other functions and/or activities conferred can include transposase activity, integrase activity, recombinase activity, ligase activity, ubiquitin ligase activity, deubiquitinating activity, adenylation activity, deadenylation activity, methyltransferase activity, demethylase activity, acetyltransferase activity, deacetylase activity, kinase activity, phosphatase activity, ribosylation activity, deribosylation activity, myristoylation activity, demyristoylation activity, polymerase activity, helicase activity, or nuclease activity, SUMOylation activity, deSUMOylation activity, or any combination of the above. In some embodiments, the Cas9 protein is fused to a histone demethylase, a transcriptional activator or a deaminase.

Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n(D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂ (SEQ ID NO: 330)-XTEN-(SGGS)₂ (SEQ ID NO: 330)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and I156F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 11 below. In some embodiments, the ABE is a sixth generation ABE. In some embodiments, the ABE is ABE6.1, ABE6.2, ABE6.3, ABE6.4, ABE6.5, or ABE6.6, as shown in Table 11 below. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE7.9, or ABE7.10, as shown in Table 11 below.

TABLE 11
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | Y | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | L | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | L | N | A | | L | N | F | S | V | N | Y | G | A | C | Y | P | V | F | N | K |
| ABE7.8 | L | R | L | N | A | | L | N | F | S | V | N | Y | G | N | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24).

In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 12 below.

TABLE 12
Adenosine Base Editor 8 (ABE8) Variants

| ABE8 | Adenosine Deaminase | Adenosine Deaminase Description |
|---|---|---|
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA*7.10 + Y147R_Q154R_Y123H |
| ABE8.9-m | TadA*8.9 | Monomer_TadA*7.10 + Y147R_Q154R_I76Y |
| ABE8.10-m | TadA*8.10 | Monomer_TadA*7.10 + Y147R_Q154R_T166R |
| ABE8.11-m | TadA*8.11 | Monomer_TadA*7.10 + Y147T_Q154R |
| ABE8.12-m | TadA*8.12 | Monomer_TadA*7.10 + Y147T_Q154S |
| ABE8.13-m | TadA*8.13 | Monomer_TadA*7.10 + |
| | | Y123H_Y147R_Q154R_I76Y |
| ABE8.14-m | TadA*8.14 | Monomer_TadA*7.10 + I76Y_V82S |
| ABE8.15-m | TadA*8.15 | Monomer_TadA*7.10 + V82S_Y147R |
| ABE8.16-m | TadA*8.16 | Monomer_TadA*7.10 + V82S_Y123H_Y147R |
| ABE8.17-m | TadA*8.17 | Monomer_TadA*7.10 + V82S_Q154R |
| ABE8.18-m | TadA*8.18 | Monomer_TadA*7.10 + V82S_Y123H_Q154R |
| ABE8.19-m | TadA*8.19 | Monomer_TadA*7.10 + |
| | | V82S_Y123H_Y147R_Q154R |
| ABE8.20-m | TadA*8.20 | Monomer_TadA*7.10 + |
| | | I76Y_V82S_Y123H_Y147R_Q154R |
| ABE8.21-m | TadA*8.21 | Monomer_TadA*7.10 + Y147R_Q154S |
| ABE8.22-m | TadA*8.22 | Monomer_TadA*7.10 + V82S_Q154S |
| ABE8.23-m | TadA*8.23 | Monomer_TadA*7.10 + V82S_Y123H |
| ABE8.24-m | TadA*8.24 | Monomer_TadA*7.10 + V82S_Y123H_Y147T |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + |
| | | Y147R_Q154R_Y123H) |
| ABE8.9-d | TadA*8.9 | Heterodimer_(WT) + (TadA*7.10 + |
| | | Y147R_Q154R_I76Y) |
| ABE8.10-d | TadA*8.10 | Heterodimer_(WT) + (TadA*7.10 + |
| | | Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_I76Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_Y147R) |
| ABE8.16-d | TadA*8.16 | Heterodimer_(WT) + (TadA*7.10 + |
| | | V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + |
| | | V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer_(WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + |
| | | V82S_Y123H_Y147T) |

In some embodiments, the ABE8 is ABE8a-m, which has a monomeric construct containing TadA*7.10 with R26C, A109S, T111R, D119N, H122N, Y1471D, F149Y, T166I, and D167N mutations (TadA*8a). In some embodiments, the ABE8 is ABE8b-m, which has a monomeric construct containing TadA*7.10 with V88A, A109S, T111R, D119N, H122N, F149Y, T166I, and D167N mutations (TadA*8b). In some embodiments, the ABE8 is ABE8c-m, which has a monomeric construct containing TadA*7.10 with R26C, A109S, T111R, D119N, H122N, F149Y, T166I, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T166I, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T166I, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T166I, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T166I, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, D119N, H122N, Y147D, F149Y, T166I, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T166I, and D167N mutations (TadA*8a). In some embodiments, the ABE8 is ABE8b-7, which has a heterodimeric construct containing TadA*7.10 fused to TadA*7.10 with V88A, A109S, T111R, D119N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, D119N, H122N, F149Y, T166I, and D167N mutations (TadA*8c). In some embodiments, the ABE8 is ABE8d-7, which has a heterodimeric construct containing TadA*7.10 fused to TadA*7.10 with V88A, T111R, D119N, and F149Y mutations (TadA*8d). In some embodiments, the ABE8 is ABE8e-7, which has a heterodimeric construct containing TadA*7.10 fused to TadA*7.10 with A109S, T111R, D119N, H122N, Y147D, F149Y, T166I, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 13 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 13, off-target RNA and DNA editing were reduced by introducing a V106W substitution into the TadA domain (as described in M. Richter et al., 2020, Nature Biotechnology, doi.org/10.1038/s41587-020-0453-z, the entire contents of which are incorporated by reference herein).

TABLE 13
Additional Adenosine Base Editor 8 Variants. In the table, “monomer”
indicates an ABE comprising a single TadA*7.10 comprising the indicated alterations
and “heterodimer” indicates an ABE comprising a TadA*7.10 comprising
the indicated alterations fused to an E. coli TadA adenosine deaminase.

| ABE8 Base | Adenosine | |
|---|---|---|
| Editor | Deaminase | Adenosine Deaminase Description |
| ABE8a-m | TadA*8a | Monomer_TadA*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D + F149Y + T166I + D167N |
| ABE8a-d | TadA*8a | Heterodimer_(WT) + (TadA*7.10 + R26C + A109S + T111R + |
| | | D119N + H122N + Y147D + F149Y + T166I + D167N) |
| ABE8b-d | TadA*8b | Heterodimer_(WT) + (TadA*7.10 + V88A + A109S + T111R + |
| | | D119N + H122N + F149Y + T166I + D167N) |
| ABE8c-d | TadA*8c | Heterodimer_(WT) + (TadA*7.10 + R26C + A109S + T111R + |
| | | D119N + H122N + F149Y + T166I + D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + |
| | | D119N + H122N + Y147D + F149Y + T166I + D167N) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABET.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g., ABE7.9, ABE7.10, or ABE8) is an NGC PAM CP6 variant (S. pyogenes Cas9 or spVRQR Cas9). In some embodiments, the base editor (e.g. ABE7.9, ABE7.10, or ABE8) is an AGA PAM CP6 variant (S. pyogenes Cas9 or spVRQR Cas9).

In some embodiments, the ABE has a genotype as shown in Table 14 below.

TABLE 14
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 15 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 15 below.

TABLE 15
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10-m | | | | | | | | | | | | R | | R | | | | R |
| ABE8.11-m | | | | | | | | | | | | T | | R | | | | |
| ABE8.12-m | | | | | | | | | | | | T | | S | | | | |
| ABE8.13-m | | | | | Y | | | | | H | | R | | R | | | | |
| ABE8.14-m | | | | | Y | S | | | | | | | | | | | | |
| ABE8.15-m | | | | | | S | | | | | | R | | | | | | |
| ABE8.16-m | | | | | | S | | | | H | | R | | | | | | |
| ABE8.17-m | | | | | | S | | | | | | | | R | | | | |
| ABE8.18-m | | | | | | S | | | | H | | | | R | | | | |
| ABE8.19-m | | | | | | S | | | | H | | R | | R | | | | |
| ABE8.20-m | | | | | Y | S | | | | H | | R | | R | | | | |
| ABE8.21-m | | | | | | | | | | | | R | | S | | | | |
| ABE8.22-m | | | | | | S | | | | | | | | S | | | | |
| ABE8.23-m | | | | | | S | | | | H | | | | | | | | |
| ABE8.24-m | | | | | | S | | | | H | | T | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21-d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:

>ABE8.1_Y147T_CP5_NGC PAM_monomer
(SEQ ID NO: 331)
MSEVEFSHEYWMRHALTLAKRARDEREVPVGAVLVLNNRVIGEGWNRAIGLHDPTAHAEIMALR
QGGLVMQNYRLIDATLYVTFEPCVMCAGAMIHSRIGRVVFGVRNAKTGAAGSLMDVLHYPGMNH
RVEITEGILADECAALLCTFFRMPRQVFNAQKKAQSSTDSGGSSGGSSGSETPGTSESATPESS
GGSSGGSEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFMQPTVAYSVLVVAK
VEKGKSKKLKSVKELLGITIMERSSFEKNPIDFLEAKGYKEVKKDLIIKLPKYSLFELENGRKR
MLASAKFLQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPRAFKYFDTTIARKEYRSTK
EVLDATLIHQSITGLYETRIDLSQLGGDGGSGGSGGSGGSGGSGGSGGMDKKYSIGLAIGTNSV
GWAVITDEYKVPSKKFKVLGNTDRHSIKKNLIGALLFDSGETAEATRLKRTARRRYTRRKNRIC
YLQEIFSNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEGADKRTADGSEF
ESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ ID NOs: 332-354).

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE7*10 reference sequence, as described herein. Exemplary ABE9 variants are listed in Table 16. Details of ABE9 base editors are described in International PCT Application No. PCT/US2020/049975, which is incorporated herein by reference for its entirety.

TABLE 16
Adenosine Base Editor 9 (ABE9) Variants. In the table, “monomer”
indicates an ABE comprising a single TadA*7.10 comprising
the indicated alterations and “heterodimer” indicates
an ABE comprising a TadA*7.10 comprising the indicated alterations
fused to an E. coli TadA adenosine deaminase.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2_heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R21N, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27_heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y_V82S_Y123H_Y147R_Q154R |
| ABE9.34_monomer | P54C_I76Y_V82S_Y123H_Y147R_Q154R |
| ABE9.35_monomer | R21N_I76Y_V82S_Y123H_Y147R_Q154R |
| ABE9.36_monomer | I76Y_V82S_Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T_Y123H_Y147R_Q154R |
| ABE9.40_heterodimer | N38G_I76Y_V82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_I76Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K_V82S, Y123H, Y147R, Q154R |
| ABE9.48_monomer | Q71M_V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, |
| | A158K |
| ABE9.52_monomer | M70V, Q71M, N72K, V82S, Y123H, Y147R, |
| | Q154R |
| ABE9.53_heterodimer | N72K_V82S, Y123H, Y147R, Q154R |
| ABE9.54_heterodimer | Q71M_V82S, Y123H, Y147R, Q154R |
| ABE9.55_heterodimer | M70V, V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, |
| | A158K |
| ABE9.58_heterodimer | M70V, Q71M, N72K, V82S, Y123H, Y147R, |
| | Q154R |

In some embodiments, the base editor includes an adenosine deaminase variant comprising an amino acid sequence, which contains alterations relative to an ABE 7*10 reference sequence, as described herein. The term “monomer” as used in Table 16.1 refers to a monomeric form of TadA*7.10 comprising the alterations described. The term “heterodimer” as used in Table 16.1 refers to the specified wild-type E. coli TadA adenosine deaminase fused to a TadA*7.10 comprising the alterations as described.

TABLE 16.1
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain. In some embodiments, the base editor comprises a domain comprising all or a portion (e.g., a functional portion) of a nucleic acid polymerase. In some embodiments, a base editor comprises as a domain all or a portion (e.g., a functional portion) of a nucleic acid polymerase (NAP). For example, a base editor can comprise all or a portion (e.g., a functional portion) of a eukaryotic NAP. In some embodiments, a NAP or portion thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor comprises multiple domains. For example, the base editor comprising a polynucleotide programmable nucleotide binding domain derived from Cas9 can comprise a REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)n (SEQ ID NO: 247), and (G)n to more rigid linkers of the form (EAAAK)n (SEQ ID NO: 248), (SGGS)n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS. In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

In another embodiment, the base editor system comprises a component (protein) that interacts non-covalently with a deaminase (DNA deaminase), e.g., an adenosine or a cytidine deaminase, and transiently attracts the adenosine or cytidine deaminase to the target nucleobase in a target polynucleotide sequence for specific editing, with minimal or reduced bystander or target-adjacent effects. Such a non-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase or cytidine deaminase) without blocking or interfering with the active (catalytic) site of the deaminase from engaging the target nucleobase (e.g., adenosine or cytidine, respectively). Such as system, termed “MagnEdit,” involves interacting proteins tethered to a Cas9 and gRNA complex and can attract a co-expressed adenosine or cytidine deaminase (either exogenous or endogenous) to edit a specific genomic target site, and is described in McCann, J. et al., 2020, “MagnEdit-interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase or cytidine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 October 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Provided herein are compositions and methods for base editing in cells. Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 7 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR, or YTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins or complexes disclosed herein, to a target site, e.g., a site comprising a mutation to be edited, it is typically necessary to co-express the fusion protein or complex together with a guide RNA. As explained in more detail elsewhere herein, a guide RNA typically comprises a tracrRNA framework allowing for napDNAbp (e.g., Cas9 or Cas12) binding, and a guide sequence, which confers sequence specificity to the napDNAbp:nucleic acid editing enzyme/domain fusion protein or complex. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins or complexes to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins or complexes to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety.

The domains of the base editor disclosed herein can be arranged in any order. Non-limiting examples of a base editor comprising a fusion protein comprising e.g., a polynucleotide-programmable nucleotide-binding domain (e.g., Cas9 or Cas12) and a deaminase domain (e.g., cytidine or adenosine deaminase) can be arranged as follows:
NH₂-[nucleobase editing domain]-Linker1-[nucleobase editing domain]-COOH;
NH₂-[deaminase]-Linker1-[nucleobase editing domain]-COOH;
NH₂-[deaminase]-Linker1-[nucleobase editing domain]-Linker2-[UGI]-COOH;
NH₂-[deaminase]-Linker1-[nucleobase editing domain]-COOH;
NH₂-[adenosine deaminase]-Linker1-[nucleobase editing domain]-COOH;
NH₂-[nucleobase editing domain]-[deaminase]-COOH;
NH₂-[deaminase]-[nucleobase editing domain]-[inosine BER inhibitor]-COOH;
NH₂-[deaminase]-[inosine BER inhibitor]-[nucleobase editing domain]-COOH;
NH₂-[inosine BER inhibitor]-[deaminase]-[nucleobase editing domain]-COOH;
NH₂-[nucleobase editing domain]-[deaminase]-[inosine BER inhibitor]-COOH;
NH₂-[nucleobase editing domain]-[inosine BER inhibitor]-[deaminase]-COOH;
NH₂-[inosine BER inhibitor]-[nucleobase editing domain]-[deaminase]-COOH;
NH₂-[nucleobase editing domain]-Linker1-[deaminase]-Linker2-[nucleobase editing domain]-COOH;
NH₂-[nucleobase editing domain]-Linker1-[deaminase]-[nucleobase editing domain]-COOH;
NH₂-[nucleobase editing domain]-[deaminase]-Linker2-[nucleobase editing domain]-COOH;
NH₂-[nucleobase editing domain]-[deaminase]-[nucleobase editing domain]-COOH;
NH₂-[nucleobase editing domain]-Linker1-[deaminase]-Linker2-[nucleobase editing domain]-[inosine BER inhibitor]-COOH;
NH₂-[nucleobase editing domain]-Linker1-[deaminase]-[nucleobase editing domain]-[inosine BER inhibitor]-COOH;
NH₂-[nucleobase editing domain]-[deaminase]-Linker2-[nucleobase editing domain]-[inosine BER inhibitor]-COOH;
NH₂-[nucleobase editing domain]-[deaminase]-[nucleobase editing domain]-[inosine BER inhibitor]-COOH;
NH₂-[inosine BER inhibitor]-[nucleobase editing domain]-Linker1-[deaminase]-Linker2-[nucleobase editing domain]-COOH;
NH₂-[inosine BER inhibitor]-[nucleobase editing domain]-Linker1-[deaminase]-[nucleobase editing domain]-COOH;
NH₂-[inosine BER inhibitor]-[nucleobase editing domain]-[deaminase]-Linker2-[nucleobase editing domain]-COOH; or
NH₂-[inosine BER inhibitor]NH₂-[nucleobase editing domain]-[deaminase]-[nucleobase editing domain]-COOH.

In some embodiments, the base editing fusion proteins or complexes provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein or complex include a deaminase domain (e.g., adenosine deaminase or cytidine deaminase), a uracil glycosylase inhibitor (UGI) domain, epitope tags, reporter gene sequences, and/or protein domains having one or more of the activities described herein.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the invention is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins or complexes comprising a Cas9 domain and a cytidine or adenosine deaminase, as disclosed herein, to a target site, e.g., a site comprising a mutation to be edited, it is typically necessary to co-express the fusion protein or complex together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein or complex. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins or complexes to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins or complexes to specific target sequences are provided herein.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations. In some embodiments, an intended mutation is a mutation that is generated by a specific base editor (e.g., adenosine base editor or cytidine base editor) bound to a guide polynucleotide (e.g., gRNA), specifically designed to generate the intended mutation. In some embodiments, the intended mutation is in a gene associated with a target antigen associated with a disease or disorder, e.g., spinal muscular atrophy. In some embodiments, the intended mutation is an adenine (A) to guanine (G) point mutation (e.g., SNP) in a gene associated with a target antigen associated with a disease or disorder, e.g. spinal muscular atrophy. In some embodiments, the intended mutation is an adenine (A) to guanine (G) point mutation within the coding region or non-coding region of a gene (e.g., regulatory region or element). In some embodiments, the intended mutation is a cytosine (C) to thymine (T) point mutation (e.g., SNP) in a gene associated with a target antigen associated with a disease or disorder, e.g., spinal muscular atrophy. In some embodiments, the intended mutation is a cytosine (C) to thymine (T) point mutation within the coding region or non-coding region of a gene (e.g., regulatory region or element). In some embodiments, the intended mutation is a point mutation that generates a STOP codon, for example, a premature STOP codon within the coding region of a gene. In some embodiments, the intended mutation is a mutation that eliminates a stop codon.

The base editors of the invention advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein may be applied to any of the fusion proteins or complexes, or methods of using the fusion proteins or complexes provided herein.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% increase of the gene targeted expression. In some embodiments, the base editing efficiency may result in at least 10% increase of the gene targeted expression. In some embodiments, the base editing efficiency may result in at least 20% increase of the gene targeted expression. In some embodiments, the base editing efficiency may result in at least 30% increase of the gene targeted expression. In some embodiments, the base editing efficiency may result in at least 40% increase of the gene targeted expression. In some embodiments, the base editing efficiency may result in at least 50% increase of the gene targeted expression. In some embodiments, the base editing efficiency may result in at least 60% increase of the targeted gene expression. In some embodiments, the base editing efficiency may result in at least 70% increase of the targeted gene expression. In some embodiments, the base editing efficiency may result in at least 80% increase of the targeted gene expression. In some embodiments, the base editing efficiency may result in at least 90% increase of the targeted gene expression. In some embodiments, the base editing efficiency may result in at least 91% increase of the targeted gene expression. In some embodiments, the base editing efficiency may result in at least 92% increase of the targeted gene expression. In some embodiments, the base editing efficiency may result in at least 93% increase of the targeted gene expression. In some embodiments, the base editing efficiency may result in at least 94% increase of the targeted gene expression. In some embodiments, the base editing efficiency may result in at least 95% increase of the targeted gene expression. In some embodiments, the base editing efficiency may result in at least 96% increase of the targeted gene expression. In some embodiments, the base editing efficiency may result in at least 97% increase of the targeted gene expression. In some embodiments, the base editing efficiency may result in at least 98% increase of the targeted gene expression. In some embodiments, the base editing efficiency may result in at least 99% increase of the targeted gene expression. In some embodiments, the base editing efficiency may result in a 1.1, 1.2, 1.3, 1.4, 1.5, 2, 3, 4, 5, 6, 7, 8, 9, 10, 20, 30, 40, 50, 60, 70, 80, 90, or 100-fold increase of the targeted gene expression.

In some embodiments, any of the base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of the base editor systems comprising one of the ABE8 base editor variants described herein result in less than 0.8% indel formation in the target polynucleotide sequence. In some embodiments, any of the base editor systems comprising one of the ABE8 base editor variants described herein result in at most 0.8% indel formation in the target polynucleotide sequence. In some embodiments, any of the base editor systems comprising one of the ABE8 base editor variants described herein result in less than 0.3% indel formation in the target polynucleotide sequence. In some embodiments, any of the base editor systems comprising one of the ABE8 base editor variants described results in lower indel formation in the target polynucleotide sequence compared to a base editor system comprising one of ABE7 base editors. In some embodiments, any of the base editor systems comprising one of the ABE8 base editor variants described herein results in lower indel formation in the target polynucleotide sequence compared to a base editor system comprising an ABE7.10.

In some embodiments, any of the base editor systems comprising one of the ABE8 base editor variants described herein has reduction in indel frequency compared to a base editor system comprising one of the ABE7 base editors. In some embodiments, any of the base editor systems comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The invention provides adenosine deaminase variants (e.g., ABE8 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE8 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 base editor variants described herein has reduced bystander editing or mutations compared to a base editor system comprising an ABE7 base editor, e.g., ABE7.10. In some embodiments, any of the base editing system comprising one of the ABE8 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editor systems provided herein result in less than 70%, less than 65%, less than 60%, less than 55%, 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% bystander editing of one or more nucleotides (e.g., an off-target nucleotide).

In some embodiments, any of the base editing system comprising one of the ABE8 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 base editor variants described herein has reduced spurious editing compared to a base editor system comprising an ABE7 base editor, e.g., ABE7.10. In some embodiments, any of the base editing system comprising one of the ABE8 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA. In some embodiments, an ABE8 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 base editor delivered by an mRNA system has higher base editing efficiency compared to an ABE8 base editor delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 base editor variants described herein has lower guided off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 base editor variants described herein has at least about 2.2 fold decrease in guided off-target editing efficiency when delivered by an mRNA system compared to when delivered by a plasmid or vector system.

In some embodiments, any of the ABE8 base editor variants described herein has lower guide-independent off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event. In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold . . . .

In embodiments, the cell population is a population of cells contacted with a base editor, complex, or base editor system of the present disclosure. In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins or complexes, or methods of using the fusion proteins or complexes provided herein.

Details of base editor efficiency are described in International PCT Application Nos. PCT/2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. In some embodiments, the multiplex editing comprises at least one guide polynucleotide that does or does not require a PAM sequence to target binding to a target polynucleotide sequence. In some embodiments, the multiplex editing comprises a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the plurality of nucleobase pairs are in one more genes. In some embodiments, the plurality of nucleobase pairs is in the same gene. In some embodiments, at least one gene in the one more genes is located in a different locus.

In some embodiments, the editing is editing of the plurality of nucleobase pairs in at least one protein coding region, in at least one protein non-coding region, or in at least one protein coding region and at least one protein non-coding region.

In some embodiments, the editing is in conjunction with one or more guide polynucleotides. In some embodiments, the base editor system comprises one or more base editor systems. In some embodiments, the base editor system comprises one or more base editor systems in conjunction with a single guide polynucleotide or a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence or with at least one guide polynucleotide that requires a PAM sequence to target binding to a target polynucleotide sequence, or with a mix of at least one guide polynucleotide that does not require a PAM sequence to target binding to a target polynucleotide sequence and at least one guide polynucleotide that does require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors. In some embodiments, the base editor system capable of multiplex editing comprising one of the ABE8 base editor variants described herein has higher multiplex editing efficiency compared to the base editor system capable of multiplex editing comprising one of ABE7 base editors. In some embodiments, the base editor system capable of multiplex editing comprising one of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% higher multiplex editing efficiency compared the base editor system capable of multiplex editing comprising one of ABE7 base editors. In some embodiments, the base editor system capable of multiplex editing comprising one of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 4.0 fold, at least 4.5 fold, at least 5.0 fold, at least 5.5 fold, or at least 6.0 fold higher multiplex editing efficiency compared the base editor system capable of multiplex editing comprising one of ABE7 base editors. In some embodiments, use of a base editor system capable of multiplex editing of a plurality of nucleobase pairs in one or more genes described herein does not comprise a risk or occurance of chromosomal translocations.

### Expression of Fusion Proteins or Complexes in a Host Cell

Fusion proteins or complexes of the invention comprising an adenosine deaminase variant may be expressed in virtually any host cell of interest, including but not limited to animal cells using routine methods known to the skilled artisan. For example, a DNA encoding an adenosine deaminase of the invention can be cloned by designing suitable primers for the upstream and downstream of CDS based on the cDNA sequence The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector.

As the expression vector, animal cell expression plasmids (e.g., pA1-11, pXT1, pRc/CMV, pRc/RSV, pcDNAI/Neo) are known in the art; animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present invention, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like are preferable.

Expression vectors for use in the present invention, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the invention can be intracellularly expressed by introducing into the cell an expression vector comprising a nucleic acid sequence encoding the fusion protein or complex.

Mammalian cells contemplated in the present invention include, but are not limited to, cell lines such as Human Embryonic Kidney (HEK) cells, monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present invention, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium is preferably about 6 to about 8. The culture is performed at generally about 30° C. to about 40° C. Where necessary, aeration and stirring may be performed.

When a higher eukaryotic cell, such as animal cell, is used as a host cell, a DNA encoding a base editing system of the present invention (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such as animal cell are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Reducing Expression of Target Genes in Cells

In some embodiments, provided herein is a cell, such as a neuron (e.g., a motor neuron, such as an alpha-motor neuron), with at least one modification in an endogenous gene or regulatory elements thereof. In some embodiments, the cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the cell is a neuron.

In some embodiments, the gene is an SMN1 and/or SMN2 polynucleotide.

In some embodiments, the editing of the endogenous gene reduces expression of the gene. In some embodiments, the editing of the endogenous gene reduces expression of the gene by at least 50% as compared to a control cell without the alteration. In some embodiments, the editing of the endogenous gene reduces expression of the gene by at least 60% as compared to a control cell without the alteration. In some embodiments, the editing of the endogenous gene reduces expression of the gene by at least 70% as compared to a control cell without the alteration. In some embodiments, the editing of the endogenous gene reduces expression of the gene by at least 80% as compared to a control cell without the alteration. In some embodiments, the editing of the endogenous gene reduces expression of the gene by at least 90% as compared to a control cell without the alteration. In some embodiments, the editing of the endogenous gene reduces expression of the gene by at least 100% as compared to a control cell without the alteration. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced or increased expression of a gene. In some embodiments, base editors (e.g., ABE, CBE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE) are used to edit exons by creating STOP codons.

In some embodiments, provided herein is a cell (e.g., a motor neuron, such as an alpha-motor neuron) with at least one modification in one or more endogenous genes. In some embodiments, the cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Delivery System

The suitability of nucleobase editors to target one or more nucleotides in a polynucleotide sequence (e.g., an SMN2 polynucleotide) is evaluated as described herein. In one embodiment, a single cell of interest is transfected, transduced, or otherwise modified with a nucleic acid molecule or molecules encoding a base editing system described herein together with a small amount of a vector encoding a reporter (e.g., GFP). These cells can be any cell line known in the art, including neuron cell lines. Alternatively, primary cells (e.g., human) may be used. Cells may also be obtained from a subject or individual, such as from tissue biopsy, surgery, blood, plasma, serum, or other biological fluid. Such cells may be relevant to the eventual cell target.

Delivery may be performed using a viral vector (e.g., an adeno-associated virus (AAV) vector). In one embodiment, transfection may be performed using lipid transfection (such as Lipofectamine or Fugene) or by electroporation. Transfection may be performed using lipid nanoparticles (LNPs). Following transfection, expression of a reporter (e.g., GFP) can be determined either by fluorescence microscopy or by flow cytometry to confirm consistent and high levels of transfection. These preliminary transfections can comprise different nucleobase editors to determine which combinations of editors give the greatest activity. The system can comprise one or more different vectors. In one embodiment, the base editor is codon optimized for expression of the desired cell type, preferentially a eukaryotic cell, preferably a mammalian cell or a human cell. The activity of the nucleobase editor is assessed as described herein, i.e., by sequencing the genome of the cells to detect alterations in a target sequence. For Sanger sequencing, purified PCR amplicons are cloned into a plasmid backbone, transformed, miniprepped and sequenced with a single primer. Sequencing may also be performed using next generation sequencing (NGS) techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). The fusion proteins or complexes that induce the greatest levels of target specific alterations in initial tests can be selected for further evaluation.

In particular embodiments, the nucleobase editors are used to target polynucleotides of interest. In one embodiment, a nucleobase editor of the invention is delivered to cells (e.g., motor neurons, such as alpha-motor neurons) in conjunction with one or more guide RNAs that are used to target one or more nucleic acid sequences of interest within the genome of a cell, thereby altering the target gene(s) (e.g., an SMN2 polynucleotide). In some embodiments, a base editor is targeted by one or more guide RNAs to introduce one or more edits to the sequence of one or more genes of interest (e.g., an SMN2 polynucleotide). In some embodiments, the one or more edits to the sequence of one or more genes of interest decrease or eliminate expression of the protein encoded by the gene in the host cell (e.g., a motor neuron, such as an alpha-motor neuron). In some embodiments, expression of one or more proteins encoded by one or more genes of interest (e.g., an SMN2 polynucleotide) is increased in the host cell (e.g., a motor neuron, such as an alpha-motor neuron).

In some embodiments, the host cell is selected from a bacterial cell, plant cell, insect cell, human cell, or mammalian cell. In some embodiments, the host cell is a mammalian cell. In some embodiments, the host cell is a human cell. In some embodiments, the cell is in vitro. In some embodiments, the cell is in vivo.

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions.

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g. lipid and/or polymer) nanoparticles are suitable for use as delivery vehicles in certain embodiments of this disclosure. Exemplary lipids for use in nanoparticle formulations, and/or gene transfer are shown in Table 17 (below). Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

TABLE 17
Lipids used for gene transfer.
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3B-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 18 lists exemplary polymers for use in gene transfer and/or nanoparticle formulations.

TABLE 18
Polymers used for gene transfer.
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 19 summarizes delivery methods for a polynucleotide encoding a fusion protein or complex described herein.

TABLE 19
Delivery methods.

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of base editor system components or nucleic acids encoding such components, for example, a polynucleotide programmable nucleotide binding domain (e.g., Cas9) such as, for example, Cas9 or variants thereof, and a gRNA targeting a nucleic acid sequence of interest, may be accomplished by delivering the ribonucleoprotein (RNP) to cells. The RNP comprises a polynucleotide programmable nucleotide binding domain (e.g., Cas9), in complex with the targeting gRNA. RNPs or polynucleotides described herein may be delivered to cells using known methods, such as electroporation, nucleofection, or cationic lipid-mediated methods, for example, as reported by Zuris, J. A. et al., 2015, Nat. Biotechnology, 33(1):73-80, which is incorporated by reference in its entirety. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

Nucleic acid molecules encoding a base editor system can be delivered directly to cells (e.g., motor neuron cells, such as alpha-motor neurons) as naked DNA or RNA by means of transfection or electroporation, for example, or can be conjugated to molecules (e.g., N-acetylgalactosamine) promoting uptake by the target cells. Vectors encoding base editor systems and/or their components can also be used. In particular embodiments, a polynucleotide, e.g. a mRNA encoding a base editor system or a functional component thereof, may be co-electroporated with one or more guide RNAs as described herein.

Nucleic acid vectors can comprise one or more sequences encoding a domain of a fusion protein or complex described herein. A vector can also encode a protein component of a base editor system operably linked to a nuclear localization signal, nucleolar localization signal, or mitochondrial localization signal. As one example, a vector can include a Cas9 coding sequence that includes one or more nuclear localization sequences (e.g., a nuclear localization sequence from SV40), and one or more deaminases.

The vector can also include any suitable number of regulatory/control elements, e.g., promoters, enhancers, introns, polyadenylation signals, Kozak consensus sequences, or internal ribosome entry sites (IRES). These elements are well known in the art.

Vectors according to this disclosure include recombinant viral vectors. Exemplary viral vectors are set forth herein above. Other viral vectors known in the art can also be used. In addition, viral particles can be used to deliver base editor system components in nucleic acid and/or protein form. For example, “empty” viral particles can be assembled to contain a base editor system or component as cargo. Viral vectors and viral particles can also be engineered to incorporate targeting ligands to alter target tissue specificity.

Vectors described herein may comprise regulatory elements to drive expression of a base editor system or component thereof. Such vectors include adeno-associated viruses with inverted long terminal repeats (AAV ITR). The use of AAV-ITR can be advantageous for eliminating the need for an additional promoter element, which can take up space in the vector. The additional space freed up can be used to drive the expression of additional elements, such as a guide nucleic acid or a selectable marker. ITR activity can be used to reduce potential toxicity due to over expression.

Any suitable promoter can be used to drive expression of a base editor system or component thereof and, where appropriate, the guide nucleic acid. For ubiquitous expression, promoters include CMV, CBA, CBH, CAG, CBh, PGK, SV40, Ferritin heavy or light chains. For brain or other CNS cell expression, suitable promoters include: SynapsinI for all neurons, CaMKIIalpha for excitatory neurons, GAD67 or GAD65 or VGAT for GABAergic neurons. For neuron (e.g., a motor neuron, such as an alpha-motor neuron) cell expression, suitable promoters include the Albumin promoter. For lung cell expression, suitable promoters include SP-B. For endothelial cells, suitable promoters include ICAM. For hematopoietic cell expression suitable promoters include IFNbeta or CD45. For osteoblast expression suitable promoters can include OG-2.

In some embodiments, a base editor system of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters, such as U6 or H1 Use of Pol II promoter and intronic cassettes to express gRNA Adeno Associated Virus (AAV).

In particular embodiments, a fusion protein or complex of the invention is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, AAV-PHP.B and variants thereof), or a suitable capsid protein of any viral vector. Thus, in some aspects, the disclosure relates to the viral delivery of a fusion protein or complex. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In some aspects, the methods described herein for editing specific genes in a cell can be used to genetically modify the cell (e.g., a motor neuron, such as an alpha-motor neuron).

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors. For example, a base editor and guide nucleic acid can be encoded on a single viral vector. In other embodiments, the base editor and guide nucleic acid are encoded on different viral vectors. In either case, the base editor and guide nucleic acid can each be operably linked to a promoter and terminator. The combination of components encoded on a viral vector can be determined by the cargo size constraints of the chosen viral vector.

The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor of the present disclosure is of sufficient size so as to enable efficient packaging and delivery into a target cell via a retroviral vector. In some embodiments, a base editor is of a size so as to allow efficient packing and delivery even when expressed together with a guide nucleic acid and/or other components of a targetable nuclease system.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, Adeno-associated virus (“AAV”) vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. AAV vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In some embodiments, AAV vectors are used to transduce a cell of interest with a polynucleotide encoding a base editor or base editor system as provided herein. AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein or complex of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-U or 5-Methyl-C.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment (e.g., a functional fragment) of a fusion protein or complex of the invention can vary in length. In some embodiments, a protein fragment (e.g., a functional fragment) ranges from 2 amino acids to about 1000 amino acids in length. In some embodiments, a protein fragment (e.g., a functional fragment) ranges from about 5 amino acids to about 500 amino acids in length. In some embodiments, a protein fragment (e.g., a functional fragment) ranges from about 20 amino acids to about 200 amino acids in length. In some embodiments, a protein fragment (e.g., a functional fragment) ranges from about 10 amino acids to about 100 amino acids in length. Suitable protein fragments (e.g., functional fragments) of other lengths will be apparent to a person of skill in the art.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

### Inteins

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of a new peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion. In some embodiments, the split intein is selected from Gp41.1, IMPDH.1, NrdJ.1 and Gp41.8 (Carvajal-Vallejos, Patricia et al. “Unprecedented rates and efficiencies revealed for new natural split inteins from metagenomic sources.” J. Biol. Chem., vol. 287, 34 (2012)).

Non-limiting examples of inteins include any intein or intein-pair known in the art, which include a synthetic intein based on the dnaE intein, the Cfa-N(e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limitine examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ TD NOs: 370-377.

Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Further non-limiting examples of amino acid and nucleotide sequences for N-inteins and C-inteins suitable for use as intein pairs include those with at least 8500 sequence identity to an amino acid or nucleotide sequence listed in the following Tables 20A-20C, or a fragments thereof that function as part of a split intein pair.

TABLE 20A
Exemplary amino acid and nucleotide sequences for N-Inteins.

| | | SEQ ID |
|---|---|---|
| N-Intein | Amino Acid or Nucleotide Sequence | NO |
| Cfa(GEP) | TGCCTGAGCTACGATACCGAGATCCTGACCGTGGAATACGGCTT | 389 |
| (nucleotide | CCTGCCTATCGGCAAGATCGTCGAGGAACGGATCGAGTGCACAG | |
| sequence) | TGTACACCGTGGATAAGAATGGCTTCGTGTACACCCAGCCTATC | |
| | GCTCAGTGGCACAACAGAGGCGAGCAAGAGGTGTTCGAGTACTG | |
| | CCTGGAAGATGGCAGCATCATCCGGGCCACCAAGGACCACAAGT | |
| | TTATGACCACCGACGGCCAGATGCTGCCCATCGACGAGATCTTT | |
| | GAGAGAGGCCTGGACCTGAAACAGGTGGACGGACTGCCT | |
| Cfa(GEP) | CLSYDTEILTVEYGFLPIGKIVEERIECTVYTVDKNGFVYTQPI | 390 |
| (amino acid | AQWHNRGEQEVFEYCLEDGSIIRATKDHKFMTTDGQMLPIDEIF | |
| sequence) | ERGLDLKQVDGLP | |
| Gp41.1 | TGTCTGGACCTCAAGACCCAAGTGCAGACACCTCAGGGCATGAA | 391 |
| (nucleotide | AGAGATTAGCAATATCCAGGTGGGCGACCTGGTCCTGAGCAACA | |
| sequence) | CCGGCTACAACGAGGTGCTGAACGTGTTCCCTAAGTCCAAGAAG | |
| | AAATCTTATAAGATCACCCTGGAAGATGGCAAGGAAATCATCTG | |
| | CAGCGAGGAACACCTGTTCCCCACCCAGACCGGCGAGATGAACA | |
| | TCAGCGGCGGACTGAAGGAGGGCATGTGCCTGTACGTGAAGGAG | |
| Gp41.1 (amino | CLDLKTQVQTPQGMKEISNIQVGDLVLSNTGYNEVLNVFPKSKK | 392 |
| acid sequence) | KSYKITLEDGKEIICSEEHLFPTQTGEMNISGGLKEGMCLYVKE | |
| Gp41.8 | TGCCTGAGCCTGGACACCATGGTGGTGACAAACGGCAAGGCCAT | 393 |
| (nucleotide | CGAGATCAGAGATGTGAAGGTGGGAGATTGGCTGGAAAGCGAAT | |
| sequence) | GTGGCCCAGTGCAGGTTACAGAGGTGCTGCCTATCATCAAGCAG | |
| | CCTGTCTTTGAGATTGTGCTGAAAAGCGGAAAAAAGATCCGGGT | |
| | GTCCGCTAATCACAAGTTCCCCACCAAGGACGGCCTCAAGACCA | |
| | TCAACAGCGGCCTGAAGGTGGGCGACTTCCTGAGAAGCAGAGCC | |
| | AAG | |
| Gp41.8 (amino | CLSLDTMVVTNGKAIEIRDVKVGDWLESECGPVQVTEVLPIIKQ | 394 |
| acid sequence) | PVFEIVLKSGKKIRVSANHKFPTKDGLKTINSGLKVGDFLRSRA | |
| | K | |
| IMPDH.1 | TGTTTTGTGCCTGGCACCCTGGTGAACACAGAGAATGGCCTGAA | 395 |
| (nucleotide | GAAAATCGAGGAAATCAAGGTGGGCGACAAGGTGTTCAGCCATA | |
| sequence) | CAGGCAAGCTGCAGGAGGTGGTGGACACCCTGATCTTCGACCGG | |
| | GACGAGGAAATCATCTCTATCAACGGCATTGATTGCACCAAGAA | |
| | CCACGAGTTCTACGTGATCGATAAGGAAAACGCTAATAGAGTGA | |
| | ACGAGGACAACATCCACCTCTTCGCCAGATGGGTCCACGCCGAG | |
| | GAACTGGATATGAAAAAGCACCTGCTGATCGAGCTGGAA | |
| IMPDH. 1 | CFVPGTLVNTENGLKKIEEIKVGDKVFSHTGKLQEVVDTLIFDR | 396 |
| (amino acid | DEEIISINGIDCTKNHEFYVIDKENANRVNEDNIHLFARWVHAE | |
| sequence) | ELDMKKHLLIELE | |
| NrdJ.1 | TGCCTGGTGGGCTCTAGCGAGATTATCACAAGAAACTACGGCAA | 397 |
| (nucleotide | GACCACCATCAAGGAAGTGGTCGAGATCTTCGACAACGACAAGA | |
| sequence) | ATATCCAGGTGCTGGCCTTCAACACCCACACCGATAATATCGAG | |
| | TGGGCCCCTATCAAGGCCGCTCAGCTGACCAGACCTAACGCCGA | |
| | GCTGGTTGAACTGGAAATCGACACCCTGCACGGCGTGAAAACAA | |
| | TCCGGTGCACCCCTGACCACCCCGTGTACACCAAGAACAGAGGC | |
| | TACGTGCGGGCCGACGAGCTGACAGATGATGACGAGCTCGTGGT | |
| | GGCTATC | |
| NrdJ.1 (amino | CLVGSSEIITRNYGKTTIKEVVEIFDNDKNIQVLAFNTHTDNIE | 398 |
| acid sequence) | WAPIKAAQLTRPNAELVELEIDTLHGVKTIRCTPDHPVYTKNRG | |
| | YVRADELTDDDELVVAI | |
| Npu | TGCCTGAGCTACGAGACAGAGATCCTGACCGTGGAATATGGCCT | 399 |
| (nucleotide | GCTGCCAATCGGAAAGATCGTGGAAAAGCGGATCGAGTGCACCG | |
| sequence) | TCTACAGCGTGGACAACAACGGAAATATCTATACACAGCCTGTG | |
| | GCCCAATGGCACGACCGGGGCGAACAGGAGGTGTTTGAGTACTG | |
| | CCTGGAAGATGGTTCTCTGATTAGAGCCACCAAGGACCACAAGT | |
| | TCATGACCGTCGACGGCCAGATGCTGCCCATCGACGAAATCTTC | |
| | GAGCGGGAACTCGACCTGATGAGAGTGGATAACCTGCCCAAT | |
| Npu (amino | CLSYETEILTVEYGLLPIGKIVEKRIECTVYSVDNNGNIYTQPV | 400 |
| acid sequence) | AQWHDRGEQEVFEYCLEDGSLIRATKDHKFMTVDGQMLPIDEIF | |
| | ERELDLMRVDNLPN | |
| Cfa N-intein | CLSYDTEILTVEYGFLPIGKIVEERIECTVYTVDKNGFVYTQPI | 401 |
| | AQWHNRGEQEVFEYCLEDGSIIRATKDHKFMTTDGQMLPIDEIF | |
| | ERGLDLKQVDGLP | |
| Npu N-intein | CLSYETEILTVEYGLLPIGKIVEKRIECTVYSVDNNGNIYTQPV | 402 |
| | AQWHDRGEQEVFEYCLEDGSLIRATKDHKFMTVDGQMLPIDEIF | |
| | ERELDLMRVDNLPN | |

TABLE 20B
Further exemplary amino acid and nucleotide sequences for N-Inteins.

| | | SEQ |
|---|---|---|
| N-Intein-SC | Amino Acid or Nucleotide Sequence | ID NO |
| Gp41.1 | ACAAGAAGCGGATACTGTCTGGACCTCAAGACCCAAGTGCAGACA | 403 |
| (nucleotide | CCTCAGGGCATGAAAGAGATTAGCAATATCCAGGTGGGCGACCTG | |
| sequence) | GTCCTGAGCAACACCGGCTACAACGAGGTGCTGAACGTGTTCCCT | |
| | AAGTCCAAGAAGAAATCTTATAAGATCACCCTGGAAGATGGCAAG | |
| | GAAATCATCTGCAGCGAGGAACACCTGTTCCCCACCCAGACCGGC | |
| | GAGATGAACATCAGCGGCGGACTGAAGGAGGGCATGTGCCTGTAC | |
| | GTGAAGGAG | |
| Gp41.1 | TRSGYCLDLKTQVQTPQGMKEISNIQVGDLVLSNTGYNEVLNVFP | 404 |
| (amino acid | KSKKKSYKITLEDGKEIICSEEHLFPTQTGEMNISGGLKEGMCLY | |
| sequence) | VKE | |
| Gp41.8 | TCTCAGCTGAACCGGTGCCTGAGCCTGGACACCATGGTGGTGACA | 405 |
| (nucleotide | AACGGCAAGGCCATCGAGATCAGAGATGTGAAGGTGGGAGATTGG | |
| sequence) | CTGGAAAGCGAATGTGGCCCAGTGCAGGTTACAGAGGTGCTGCCT | |
| | ATCATCAAGCAGCCTGTCTTTGAGATTGTGCTGAAAAGCGGAAAA | |
| | AAGATCCGGGTGTCCGCTAATCACAAGTTCCCCACCAAGGACGGC | |
| | CTCAAGACCATCAACAGCGGCCTGAAGGTGGGCGACTTCCTGAGA | |
| | AGCAGAGCCAAG | |
| Gp41.8 | SQLNRCLSLDTMVVTNGKAIEIRDVKVGDWLESECGPVQVTEVLP | 406 |
| (amino acid | IIKQPVFEIVLKSGKKIRVSANHKFPTKDGLKTINSGLKVGDFLR | |
| sequence) | SRAK | |
| IMPDH.1 | GGCATCGGCGGAGGATGTTTTGTGCCTGGCACCCTGGTGAACACA | 407 |
| (nucleotide | GAGAATGGCCTGAAGAAAATCGAGGAAATCAAGGTGGGCGACAAG | |
| sequence) | GTGTTCAGCCATACAGGCAAGCTGCAGGAGGTGGTGGACACCCTG | |
| | ATCTTCGACCGGGACGAGGAAATCATCTCTATCAACGGCATTGAT | |
| | TGCACCAAGAACCACGAGTTCTACGTGATCGATAAGGAAAACGCT | |
| | AATAGAGTGAACGAGGACAACATCCACCTCTTCGCCAGATGGGTC | |
| | CACGCCGAGGAACTGGATATGAAAAAGCACCTGCTGATCGAGCTG | |
| | GAA | |
| IMPDH.1 | GIGGGCFVPGTLVNTENGLKKIEEIKVGDKVFSHTGKLQEVVDTL | 408 |
| (amino acid | IFDRDEEIISINGIDCTKNHEFYVIDKENANRVNEDNIHLFARWV | |
| sequence) | HAEELDMKKHLLIELE | |
| NrdJ.1 | GGAACAAACCCATGTTGCCTGGTGGGCTCTAGCGAGATTATCACA | 409 |
| (nucleotide | AGAAACTACGGCAAGACCACCATCAAGGAAGTGGTCGAGATCTTC | |
| sequence) | GACAACGACAAGAATATCCAGGTGCTGGCCTTCAACACCCACACC | |
| | GATAATATCGAGTGGGCCCCTATCAAGGCCGCTCAGCTGACCAGA | |
| | CCTAACGCCGAGCTGGTTGAACTGGAAATCGACACCCTGCACGGC | |
| | GTGAAAACAATCCGGTGCACCCCTGACCACCCCGTGTACACCAAG | |
| | AACAGAGGCTACGTGCGGGCCGACGAGCTGACAGATGATGACGAG | |
| | CTCGTGGTGGCTATC | |
| NrdJ.1 (amino | GTNPCCLVGSSEIITRNYGKTTIKEVVEIFDNDKNIQVLAFNTHT | 410 |
| acid | DNIEWAPIKAAQLTRPNAELVELEIDTLHGVKTIRCTPDHPVYTK | |
| sequence) | NRGYVRADELTDDDELVVAI | |

TABLE 20C
Exemplary amino acid and nucleotide sequences for C-Inteins.

| | | SEQ ID |
|---|---|---|
| C-Intein | Amino Acid or Nucleotide Sequence | NO |
| Cfa(GEP) | GTCAAGATCATCAGCAGAAAGAGCCTGGGCACCCAGAACGTGTA | 411 |
| (nucleotide | CGATATCGGAGTGGGCGAGCCCCACAACTTTCTGCTCAAGAATG | |
| sequence) | GCCTGGTGGCCAGCAAC | |
| Cfa(GEP) | VKIISRKSLGTQNVYDIGVGEPHNFLLKNGLVASN | 412 |
| (amino acid | | |
| sequence) | | |
| Gp41.1 | ATGATGCTGAAAAAGATCCTGAAGATCGAGGAACTGGATGAGAG | 413 |
| (nucleotide | AGAGCTGATCGACATCGAAGTGTCTGGCAATCACCTGTTCTACG | |
| sequence) | CCAACGACATCCTGACCCACAACAGC | |
| Gp41.1 (amino | MMLKKILKIEELDERELIDIEVSGNHLFYANDILTHNS | 414 |
| acid sequence) | | |
| Gp41.8 | ATGTGCGAAATCTTCGAGAACGAGATTGATTGGGACGAAATCGC | 415 |
| (nucleotide | CTCTATCGAGTACGTGGGCGTGGAAGAGACAATCGACATCAACG | |
| sequence) | TGACCAACGACAGACTGTTTTTCGCCAATGGCATCCTGACCCAC | |
| | AACAGC | |
| Gp41.8 (amino | MCEIFENEIDWDEIASIEYVGVEETIDINVTNDRLFFANGILTH | 416 |
| acid sequence) | NS | |
| IMPDH.1 | ATGAAATTCAAGCTGAAGGAAATCACCAGCATCGAGACAAAGCA | 417 |
| (nucleotide | CTACAAGGGCAAGGTGCACGATCTGACCGTGAACCAGGACCACA | |
| sequence) | GCTACAACGTCAGAGGCACCGTGGTGCATAATTCT | |
| IMPDH.1 | MKFKLKEITSIETKHYKGKVHDLTVNQDHSYNVRGTVVHNS | 418 |
| (amino acid | | |
| sequence) | | |
| NrdJ.1 | ATGGAAGCCAAGACCTACATCGGCAAGCTGAAATCTAGAAAGAT | 419 |
| (nucleotide | CGTGTCCAACGAGGATACATACGACATCCAGACCAGCACCCACA | |
| sequence) | ATTTCTTCGCCAACGACATCCTGGTGCACAACAGC | |
| NrdJ.1 (amino | MEAKTYIGKLKSRKIVSNEDTYDIQTSTHNFFANDILVHNS | 420 |
| acid sequence) | | |
| Npu | ATGATCAAGATCGCCACAAGAAAGTACCTGGGCAAGCAGAACGT | 421 |
| (nucleotide | GTACGACATCGGCGTGGAGAGAGACCACAACTTCGCCCTGAAGA | |
| sequence) | ACGGCTTTATCGCCTCTAAT | |
| Npu (amino | MIKIATRKYLGKQNVYDIGVERDHNFALKNGFIASN | 422 |
| acid sequence) | | |
| Cfa C-intein | MVKIISRKSLGTQNVYDIGVGEPHNFLLKNGLVASN | 423 |
| (amino acid | | |
| sequence) | | |
| Npu C-intein | IKIATRKYLGKQNVYDIGVERDHNFALKNGFIASN | 424 |
| (amino acid | | |
| sequence) | | |

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]--C. In some embodiments, an intein-C is fused to the N-terminus of the C-terminal portion of the split Cas9, i.e., to form a structure of N-[intein-C]--[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. The mechanism of intein-mediated protein splicing for joining the proteins the inteins are fused to (e.g., split Cas9) is known in the art, e.g., as described in Shah et al., Chem Sci. 2014; 5(1):446-461, incorporated herein by reference. Methods for designing and using inteins are known in the art and described, for example by WO2014004336, WO2017132580, WO2013045632A1, US20150344549, and US20180127780, each of which is incorporated herein by reference in their entirety.

In some embodiments, a portion or fragment of a nuclease (e.g., Cas9) is fused to an intein. The nuclease can be fused to the N-terminus or the C-terminus of the intein. In some embodiments, a portion or fragment of a fusion protein or complex is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. In some embodiments, an N-terminal fragment of a base editor (e.g., ABE, CBE) is fused to a split intein-N and a C-terminal fragment is fused to a split intein-C. In some embodiments, an N-terminal fragment of a nucleic acid programmable DNA binding protein (napDNAbp) domain (e.g., Cas9) is fused to a split intein-N and a C-terminal fragment is fused to a split intein-C. In some embodiments, an N-terminal fragment of a deaminase domain (e.g., adenosine or cytidine deaminase) fused to a split intein-N and a C-terminal fragment is fused to a split intein-C. In some embodiments, a portion (e.g., a functional portion) or fragment of a nuclease (e.g., Cas9) is fused to an intein. The nuclease can be fused to the N-terminus or the C-terminus of the intein. In some embodiments, a portion (e.g., a functional portion) or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. These fragments are then packaged into two or more AAV vectors. In some embodiments, the N-terminus of an intein is fused to the C-terminus of a fusion protein and the C-terminus of the intein is fused to the N-terminus of an AAV capsid protein.

In one embodiment, inteins are utilized to join fragments or portions of a cytidine or adenosine base editor protein that is grafted onto an AAV capsid protein. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in capital letters in the sequence below (called the “Cas9 reference sequence”).

| (SEQ ID NO: 197) | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvq tynqlfeenp inasgvdaka ilsarlsksr rlenliaqlp gekknglfgn |
| 241 | lialslgltp nfksnfdlae daklqlskdt yddddnlla qigdqyadlf laaknlsdai |
| 301 | llSdilrvnT eiTkaplsas mikrydehhq dltllkalvr qqlpekykei ffdqSkngya |
| 361 | gyidggasqe efykfikpil ekmdgteell vklnredllr kqrtfdngsi phqihlgelh |
| 421 | ailrrqedfy pflkdnreki ekiltfripy yvgplArgnS rfAwmTrkSe eTiTpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kieCfdSvei sgvedrfnAS lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdqeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhahdayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi lpkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskrv iladanldkv lsaynkhrdk pireqaenii hlftltnlga |
| 1321 | paafkyfdtt idrkrytstk evldatlihq sitglyetri dlsqlggd |

### Pharmaceutical Compositions

In some aspects, the present invention provides a pharmaceutical composition comprising any of the cells, polynucleotides, vectors, base editors, base editor systems, guide polynucleotides, fusion proteins, complexes, or the fusion protein-guide polynucleotide complexes described herein

The pharmaceutical compositions of the present invention can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In addition to a modified cell, or population thereof, and a carrier, the pharmaceutical compositions of the present invention can include at least one additional therapeutic agent useful in the treatment of disease. For example, some embodiments of the pharmaceutical composition described herein further comprises a chemotherapeutic agent. In some embodiments, the pharmaceutical composition further comprises a cytokine peptide or a nucleic acid sequence encoding a cytokine peptide. In some embodiments, the pharmaceutical compositions comprising the cell or population thereof can be administered separately from an additional therapeutic agent.

Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., skeletal muscle, the central nervous system, the brain, and/or a neuron, such as an alpha-motor neuron). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et al., 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present invention provide methods of treating a subject in need, the method comprising administering to a subject in need an effective therapeutic amount of a pharmaceutical composition as described herein. More specifically, the methods of treatment include administering to a subject in need thereof one or more pharmaceutical compositions described herein, optionally comprising one or more cells having at least one edited gene. In other embodiments, the methods of the invention comprise expressing or introducing into a cell a base editor polypeptide and one or more guide RNAs capable of targeting a nucleic acid molecule encoding at least one polypeptide.

In embodiments the subject is an animal and the pharmaceutical composition is administered by intracerebroventricular injection, intra-thecal injection (e.g., a lumbar intra-thecal injection), and/or intracisternal magna injection (intracisternal injection). In embodiments, the pharmaceutical composition contains an AAV vector, or a lipid nanoparticle (LNP).

In embodiments, the methods provided herein are associated with the reduction of a symptom of spinal muscular atrophy in a subject. In embodiments, the methods are associated with improved health in a subject and/or improved blood pressure levels in a subject.

### Kits

The invention provides kits for the treatment of spinal muscular atrophy in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. A Cytidine Base Editor (CBE) Introduced a C-to-T Alteration to an SMN2 Polynucleotide

In this example, a cytidine base editor (CBE) was used to introduce a precise C-to-T alteration in an SMN2 polynucleotide, where the alterations are likely associated with increased levels of an SMN polypeptide in a cell. Such an increase in SMN polypeptide levels can compensate for low levels of SMN1 polynucleotide and/or SMN1 activity in the cell and can be used as an effective treatment for spinal muscular atrophy. The alterations are likely associated with increased production of full-length transcripts from the SMN2 polynucleotide. The C-to-T base change results in a C10T, C7T, or C11T alteration in Intron 7 of the SMN1 polynucleotide. The guide RNAs used to target the CBE to effect these alterations contained the spacers provided in Table 2A.

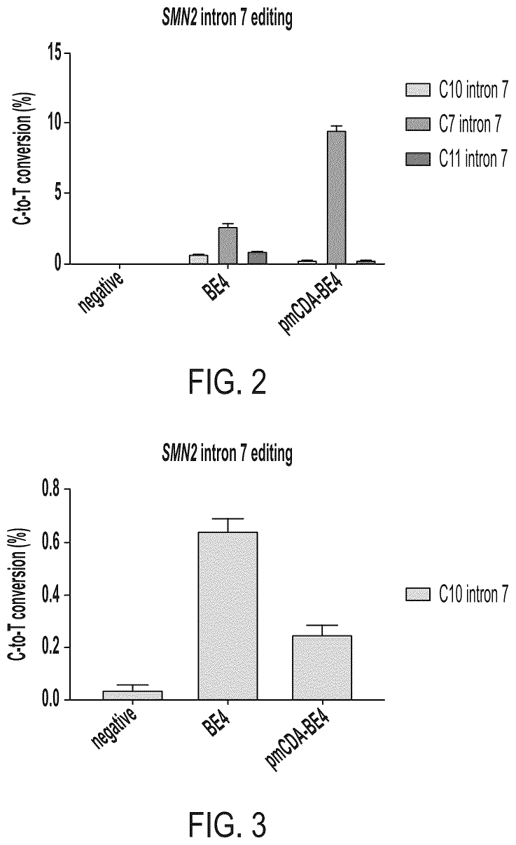

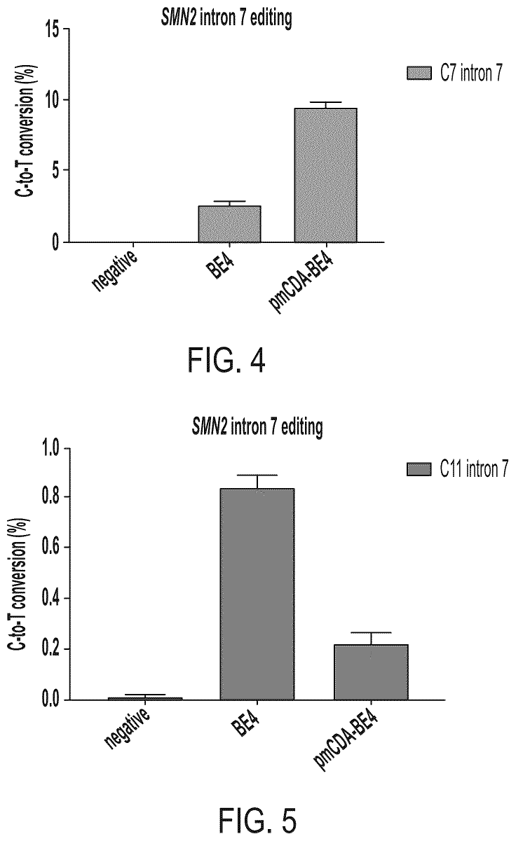

Base editing was tested in a lenti-HEK cell line. Guide RNAs (gRNA) containing the spacers listed in Table 2A were used in conjunction with BE4 or pmCDA-BE4 (see FIGS. 2-5). The base editor systems containing the guide RNAs and the CBEs effected the C10T, C7T, and C11T alterations to Intron 7 of the SMN2 polynucleotide (see FIGS. 2-5).

### Example 2. Using Base Editors to Introduce Alterations to Regulatory Elements of an SMN2 Polynucleotide Sequence

In this example, adenosine base editors (ABEs) and cytidine base editors (CBEs) are used to introduce precise A-to-G or C-to-T alterations, respectively, in an SMN2 polynucleotide. The alterations are likely associated with increased levels of an SMN polypeptide in a cell. Such an increase in SMN polypeptide levels can compensate for low levels of SMN1 polynucleotide and/or SMN1 activity in the cell and can be used as an effective treatment for spinal muscular atrophy. The alterations are likely associated with increased production of full-length transcripts from the SMN2 polynucleotide. Non-limiting examples of target alterations include a T6C and/or A54G alteration in Exon 7 of an SMN2 polynucleotide, and a C10T alteration in Intron 7 of an SMN2 polynucleotide.

As a first step in effecting alterations to the SMN2 polynucleotide to increase production of full-length transcripts, in silico screens are completed to identify candidate guide RNAs for splice site modulation. Guides with canonical PAMs are prioritized, with other base editor variants considered as well. All guides compatible with various regulatory regions surrounding Exon 7 (about 125 bp upstream and downstream) are identified, with priority given to ABE targets proximal to canonical PAMs. Guides demonstrating undesired guide-dependent off-target profiles are eliminated.

Next, guide RNAs are screened in modified HEK293T cells. HEK cells are modified with a GFP reporter insertion to easily distinguish SMN2 from SMN1 readouts, and to facilitate assays for exon inclusion (see, e.g., Hua, et al. “Enhancement of SMN2 Exon 7 inclusion by Antisense Oligonucleotides Targeting the Exon,” PLoS

The gRNAs encompass the scaffold sequence and the spacer sequences as provided herein, or as determined based on the knowledge of the skilled practitioner and as would be understood to the skilled practitioner in the art as suitable for use in disruption of splicing. (See, e.g., Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1).

For targeted mutations, and some mutations in Intron 6, a lenti-HEK line containing a limited region of an SMN2 polynucleotide is used as a screening tool to evaluate editing efficiencies. Exon inclusion is measured as a follow-up. Guide sequences are evaluated in combination with various of the base editors provided herein to identify combinations that are associated with base editing efficiencies of greater than 30%.

Guides associated with high base editing efficiencies (e.g., efficiencies greater than 30%) are validated in human fibroblasts, including primary fibroblasts, from patients with spinal muscular atrophy. The base editors are introduced to the cells using electroporation. If many guides are screened, some base editors are pre-screened in SHSY5Y cells. Top validated hits are functionally assessed (e.g., quantification of exon inclusion by qPCR, and/or assessment of SMN protein level restoration by ELISA or Western Blott).

Following functional assessment, top hits are evaluated using in vivo and in vitro modeling in patient-derived cells. Top hits are evaluated in mouse/rat models of SMA. Top hits are also evaluated for off-target binding using ONE-SEQ, Digenome-Seq, and/or rhAMPSeq.

Various delivery strategies are evaluated, including lipid nanoparticles. Delivery strategies are assessed in rats. The base editor systems are delivered by intrathecal injection into the cisterna magna, as well as by lumbar puncture. In vivo dose-ranges are evaluated, durability of the edits is evaluated, biodistribution is determined, and safety data is gathered (e.g., minimal effective dose, optimal effective dose, maximum tolerance dose).

Efficacy of base editing is evaluated in transgenic mouse models of SMA as well as in patient iPSC-derived neurons. Editing and exon inclusion is evaluated by intrathecal and/or intracerebroventricular delivery to the mice. The patient-derived iPSCs are differentiated to motor neurons and other cell types. The base editor systems are delivered to the differentiated iPSCs by lipid nanoparticles. Editing and exon inclusion, as well as restoration of SMN polypeptide expression, is evaluated in the differentiated iPSCs.

Delivery and editing are assessed in non-human primates. Dose-range finding is conducted in the non-human primates and durability of the edits and biodistribution are determined, and safety data is gathered (e.g., minimal effective dose, optimal effective dose, and maximum tolerated dose).

### Example 3. Developing Base Editor Systems

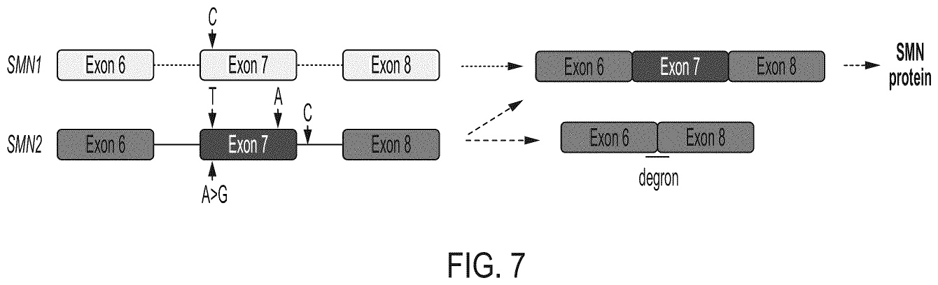

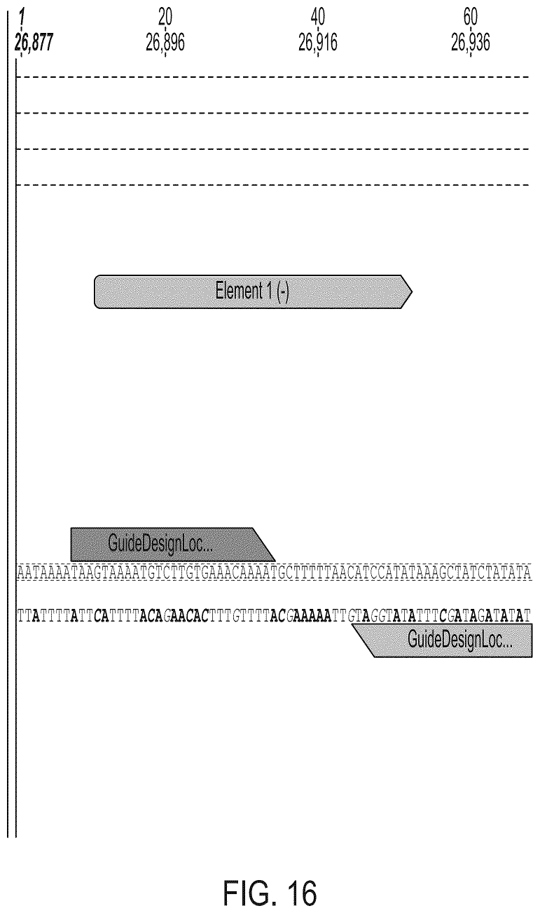

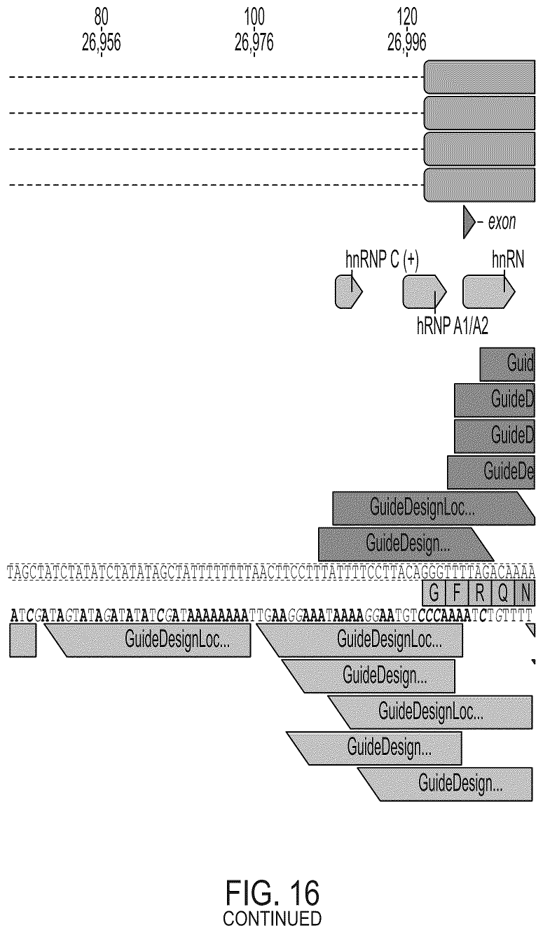

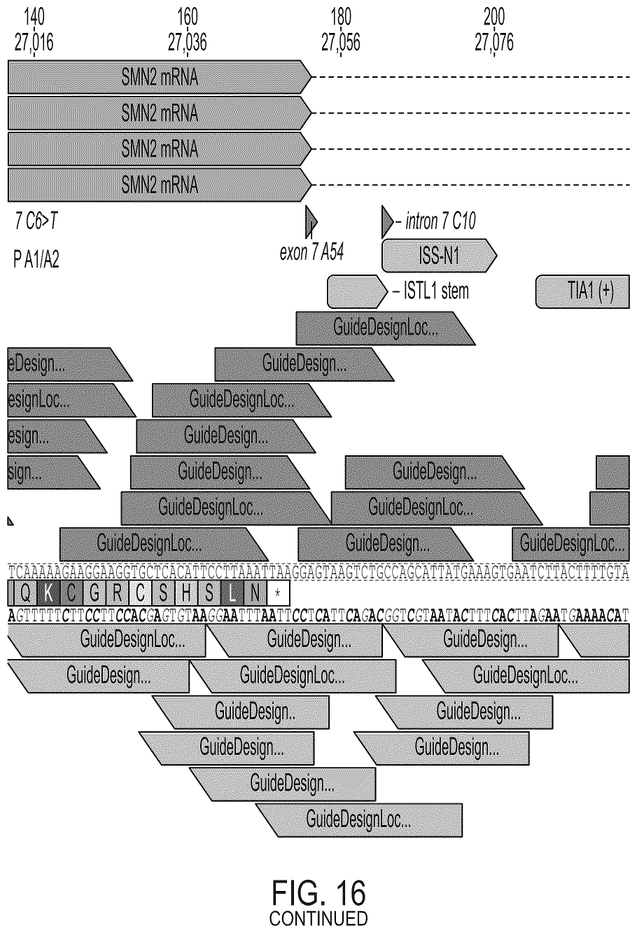

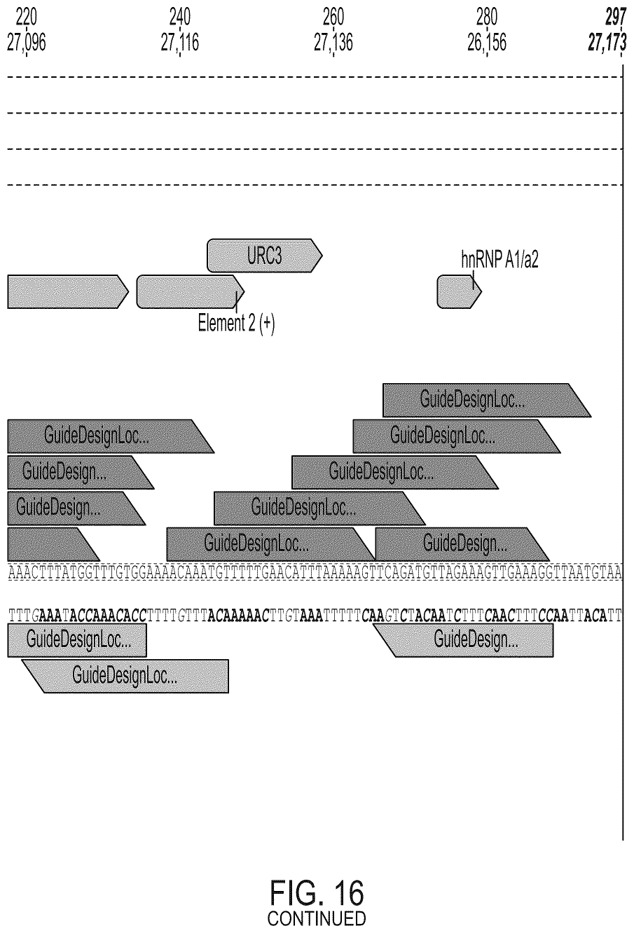

Experiments were undertaken to develop base editor systems suitable for use in introducing nucleobase alterations (e.g., C4→T or A→G) to an SMN2 polynucleotide. In particular, guide polynucleotides were designed for use in targeting base editors to deaminate nucleobases in an SMN2 polynucleotide to increase levels of incorporation of Exon 7 into spliced SMN2 transcripts expressed from the SMN2 polynucleotide (see, FIG. 16 and Tables 2B and 2C). In particular, guides polynucleotides were designed to introduce one of the following alterations to the SMN2 polynucleotide, among others: deamination of nucleobase C10 or C11 of Intron 7 of SMN2; deamination of the adenosine complementary to nucleobase T6 in Exon 7 of SMN2; deamination of nucleobase A54 of Exon 7 of SMN2 (see FIG. 7).

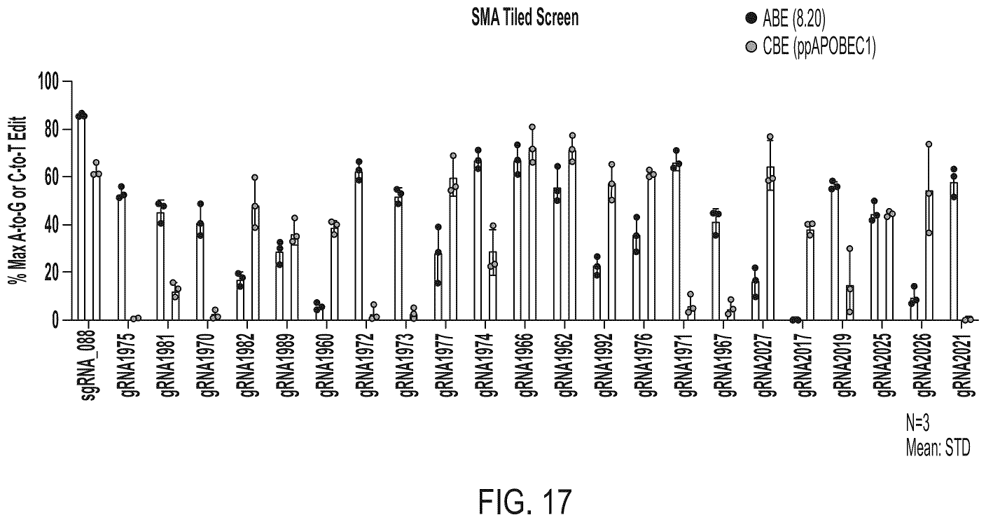

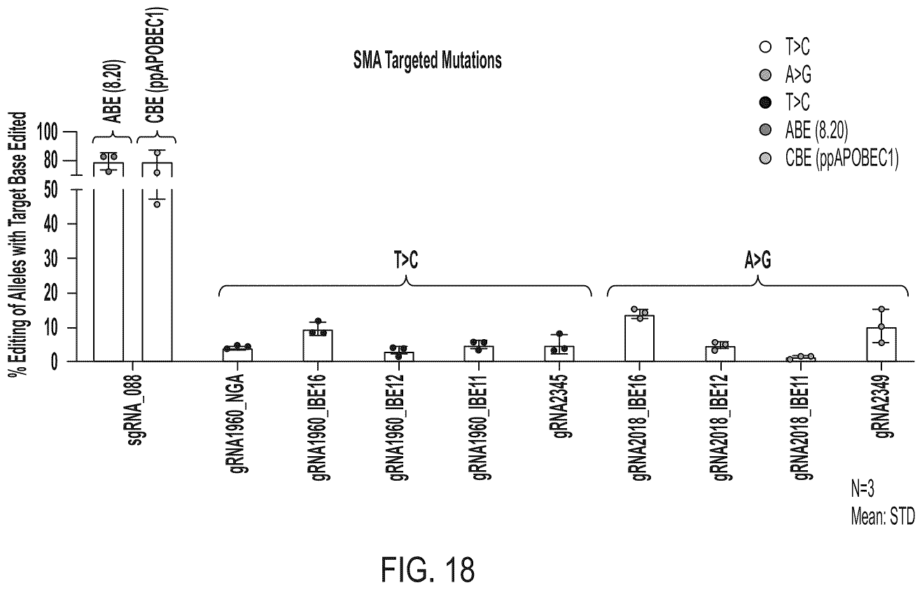

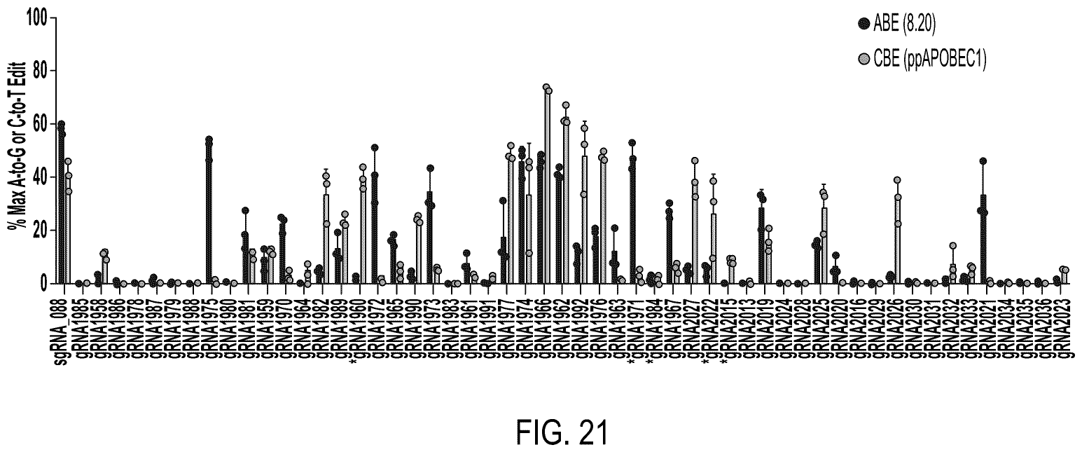

In an initial screen to identify effective base editor systems comprising the guide polynucleotides, HepG2 cells were contacted with base editor systems containing the guides listed along the x-axes of FIGS. 17, 18, and 21 and the adenosine base editor ABE8.20, the cytidine base editor ppAPOBEC1, or one of the inlaid base editors (IBEs) IBE16, IBE12, IBE11 indicated on the x-axes of FIGS. 17, 18, and 21. The inlaid base editors each contained a TadA*8.20 deaminase domain. Further details relating to the base editors used in combination with the guide polynucleotides listed in FIG. 17 are provide in Table 21 below. The HepG2 cells were transfected with mRNA encoding the base editor and a guide polynucleotide using lipofectamine MessangerMAX. Base editing efficiencies for editing of SMN2 were evaluated in triplicate. A number of the base editor systems achieved base editing efficiencies of greater than 40%. About 30 of the base editor systems evaluated had base editing efficiencies of over 25% in the HepG2 cells. The screen allowed for the identification of base editor systems effective in altering a nucleobase in SMN2. The inlaid base editors (IBEs) were effective at facilitating base editing of SMN2 in the cells.

TABLE 21
Details relating to base editor systems of FIG. 17. See Tables 2B and 2C for guide sequences.
For the ABEs listed, 8.20 indicates TadA*8.20, and the final letters indicate the PAM sequence
recognized by the indicated Cas9. For the CBEs listed, ppAPOBEC1 indicates the cytidine
deaminase, and the final letters indicate the PAM sequence recognized by the indicated Cas9.
“Sa” indicates “Staplylococcus aureus,” “Sp” indicates “Streptococcus pyogenes.”

| | Name for | Name for | | |
|---|---|---|---|---|
| | mRNA | mRNA | | |
| Guide | encoding | encoding | | |
| Name | the ABE | the CBE | ABE | CBE |
| gRNA1975 | MRNA2001 | MRNA1895 | ABE_8.20_SaCas9_NNGRRT | CBE_ppAPOBEC1_SaCas9_NNGRRT |
| gRNA1981 | MRNA2005 | MRNA1899 | ABE_8.20_SaCas9_NNNRRT | CBE_ppAPOBEC1_SaCas9_NNNRRT |
| gRNA1970 | MRNA1907 | MRNA1986 | ABE_8.20_SpCas9_NGG | CBE_ppAPOBEC1_SpCas9_NGG |
| gRNA1982 | MRNA2005 | MRNA1899 | ABE_8.20_SaCas9_NNNRRT | CBE_ppAPOBEC1_SaCas9_NNNRRT |
| gRNA1989 | MRNA2005 | MRNA1899 | ABE_8.20_SaCas9_NNNRRT | CBE_ppAPOBEC1_SaCas9_NNNRRT |
| gRNA1960 | MRNA1998 | MRNA1891 | ABE_8.20_SpCas9_NGA | CBE_ppAPOBEC1_SpCas9_NGA |
| gRNA1972 | MRNA1907 | MRNA1986 | ABE_8.20_SpCas9_NGG | CBE_ppAPOBEC1_SpCas9_NGG |
| gRNA1973 | MRNA1907 | MRNA1986 | ABE_8.20_SpCas9_NGG | CBE_ppAPOBEC1_SpCas9_NGG |
| gRNA1977 | MRNA2001 | MRNA1895 | ABE_8.20_SaCas9_NNGRRT | CBE_ppAPOBEC1_SaCas9_NNGRRT |
| gRNA1974 | MRNA1907 | MRNA1986 | ABE_8.20_SpCas9_NGG | CBE_ppAPOBEC1_SpCas9_NGG |
| gRNA1966 | MRNA1998 | MRNA1891 | ABE_8.20_SpCas9_NGA | CBE_ppAPOBEC1_SpCas9_NGA |
| gRNA1962 | MRNA1998 | MRNA1891 | ABE_8.20_SpCas9_NGA | CBE_ppAPOBEC1_SpCas9_NGA |
| gRNA1992 | MRNA2005 | MRNA1899 | ABE_8.20_SaCas9_NNNRRT | CBE_ppAPOBEC1_SaCas9_NNNRRT |
| gRNA1976 | MRNA2001 | MRNA1895 | ABE_8.20_SaCas9_NNGRRT | CBE_ppAPOBEC1_SaCas9_NNGRRT |
| gRNA1971 | MRNA1907 | MRNA1986 | ABE_8.20_SpCas9_NGG | CBE_ppAPOBEC1_SpCas9_NGG |
| gRNA1967 | MRNA1998 | MRNA1891 | ABE_8.20_SpCas9_NGA | CBE_ppAPOBEC1_SpCas9_NGA |
| gRNA2027 | MRNA2005 | MRNA1899 | ABE_8.20 SaCas9_NNNRRT | CBE_ppAPOBEC1_SaCas9_NNNRRT |
| gRNA2019 | MRNA1907 | MRNA1986 | ABE_8.20_SpCas9_NGG | CBE_ppAPOBEC1_SpCas9_NGG |
| gRNA2025 | MRNA2005 | MRNA1899 | ABE_8.20_SaCas9_NNNRRT | CBE_ppAPOBEC1_SaCas9_NNNRRT |
| gRNA2026 | MRNA2005 | MRNA1899 | ABE_8.20_SaCas9_NNNRRT | CBE_ppAPOBEC1_SaCas9_NNNRRT |
| gRNA2021 | MRNA1907 | MRNA1986 | ABE_8.20_SpCas9_NGG | CBE_ppAPOBEC1_SpCas9_NGG |

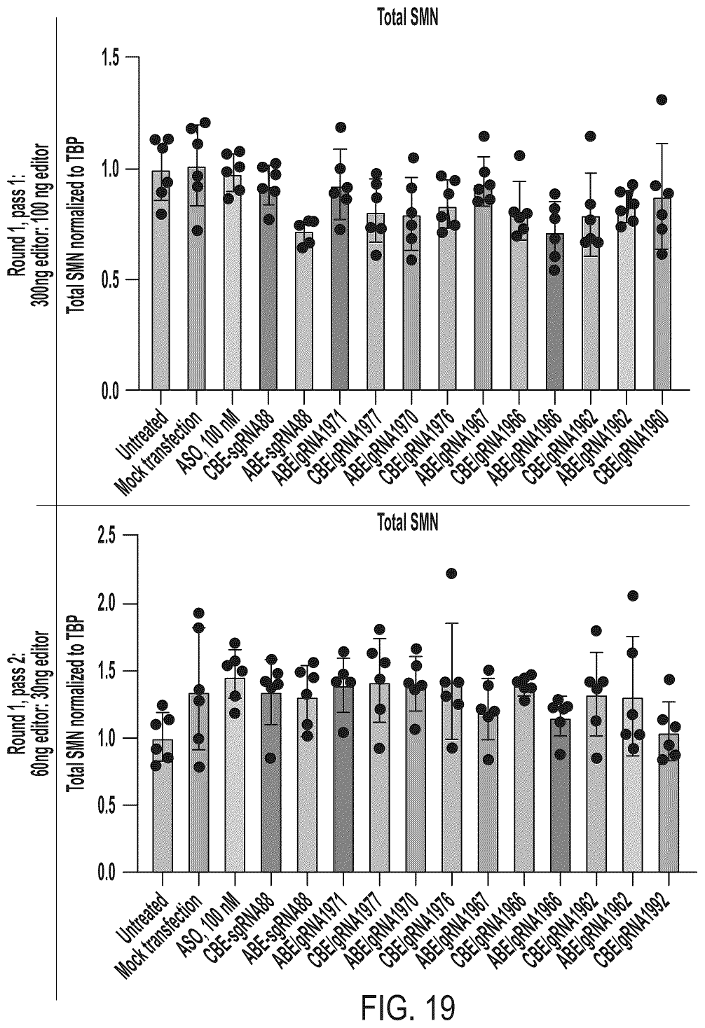

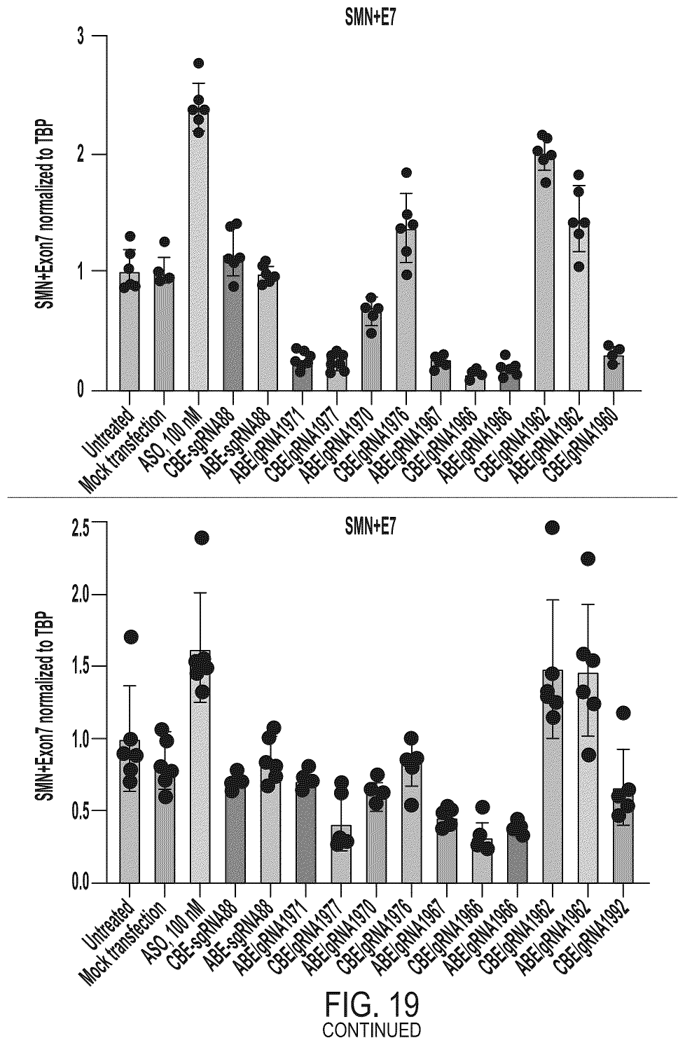

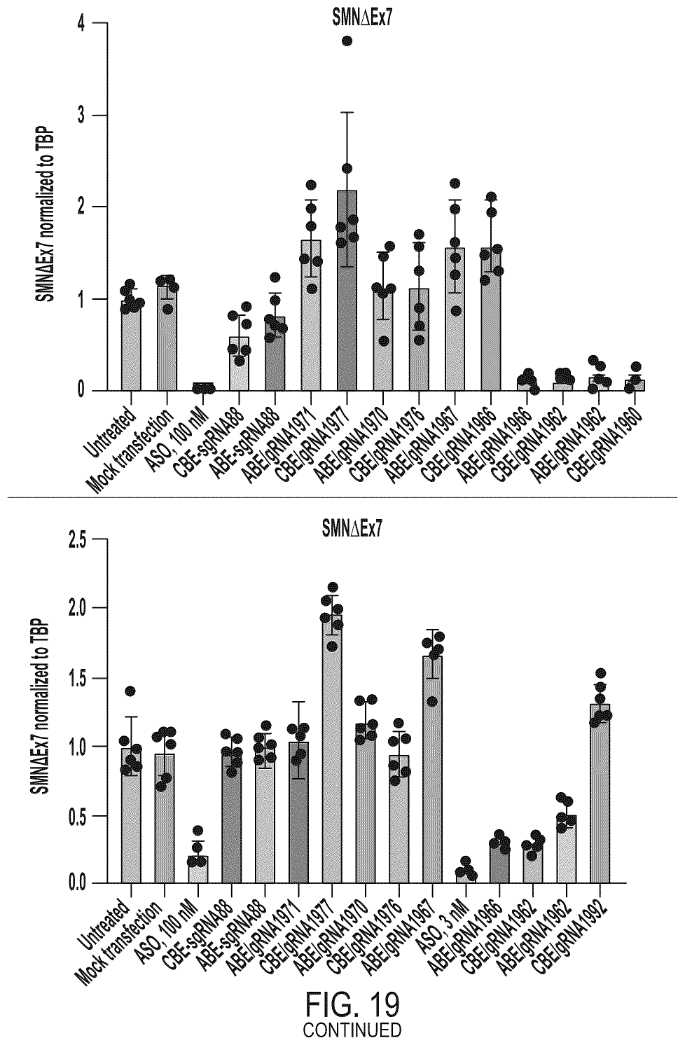

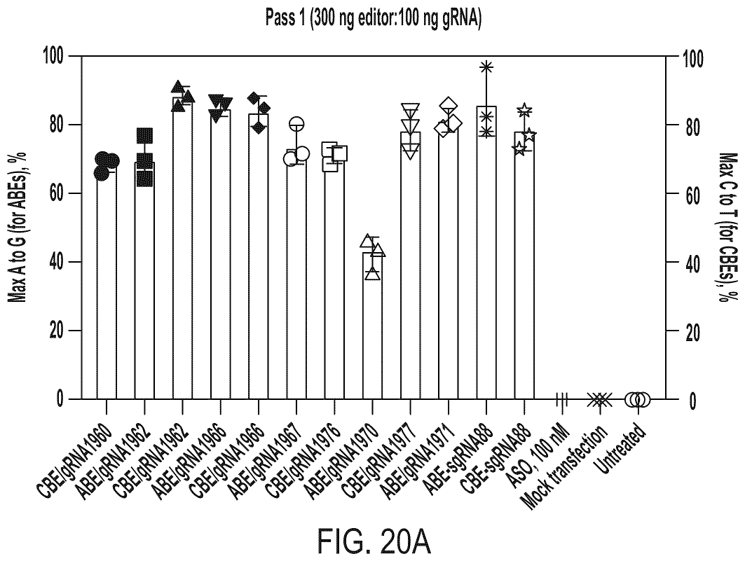

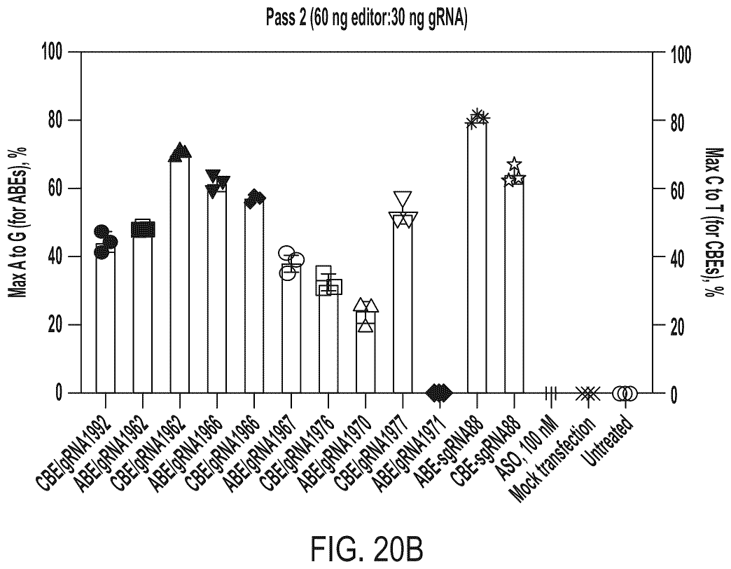

Next, having established which guide-base editor combinations were effective in carrying out base editing in the HepG2 cells, screens were conducted to determine the effect of nucleobase alterations introduced to an SMN2 polynucleotide in spinal muscular atrophy (SMA) fibroblast cells (GM03813 cells) on the incorporation of Exon 7 into spliced transcripts expressed form the SMN2 polynucleotide. The GM03813 cells were homozygous for a deletion of Exons 7 and 8 of SMN1 and contained 3 copies of the SMN2 gene. As a first step in undertaking the screens, a screen was undertaken using two different doses of the base editor system. At one dose, cells were contacted with 300 ng of mRNA encoding a base editor and 100 ng of a guide polynucleotide (see FIGS. 20A and 19). At another dose, cells were contacted with 60 ng of mRNA encoding the base editor and 30 ng of the guide polynucleotide (see FIGS. 20B and 19). It was determined that the dose containing 60 ng of mRNA encoding the base editor and 30 ng of the guide polynucleotide resulted in better editing efficiencies and cell survival rates post-transduction than the higher dose. Therefore, all subsequent screens using the SMA fibroblast cells were undertaken using the lower dose of the base editor system.

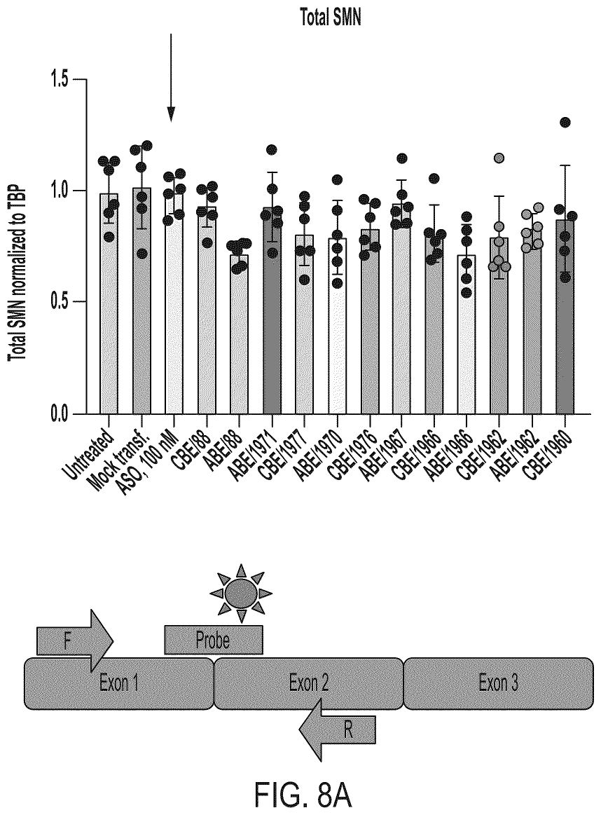

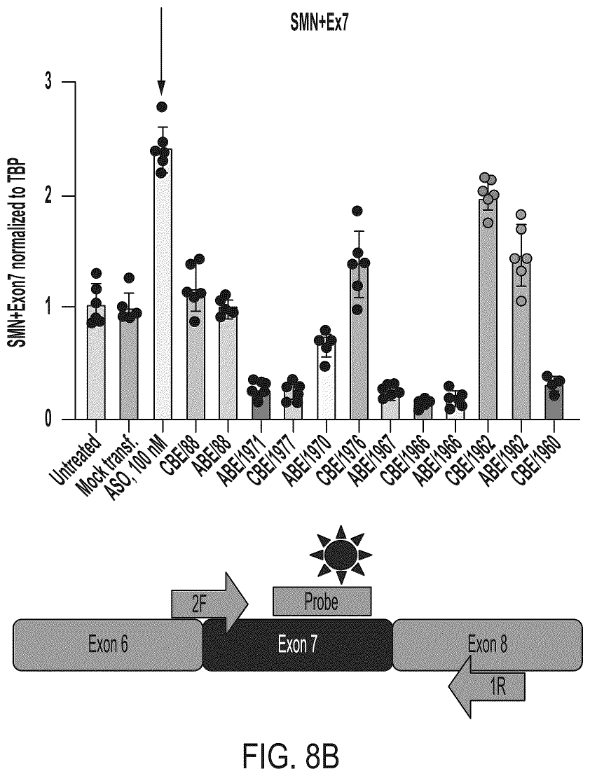

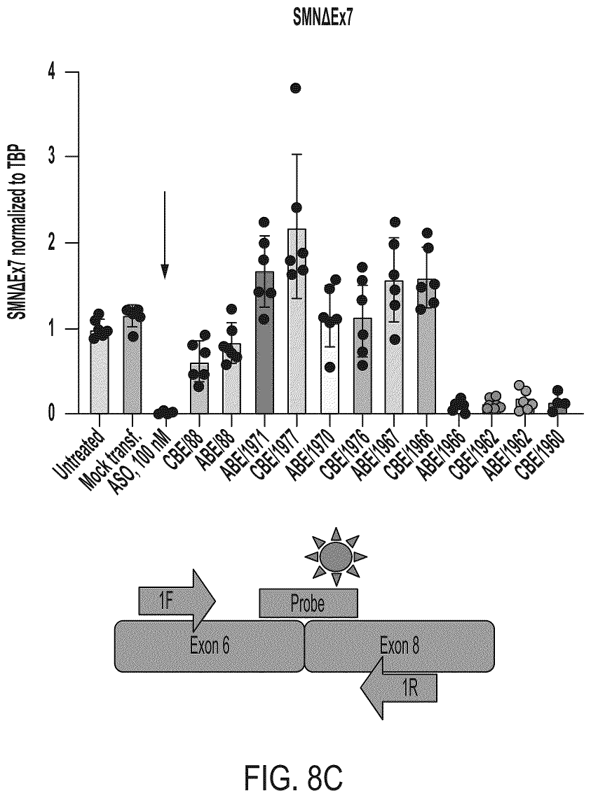

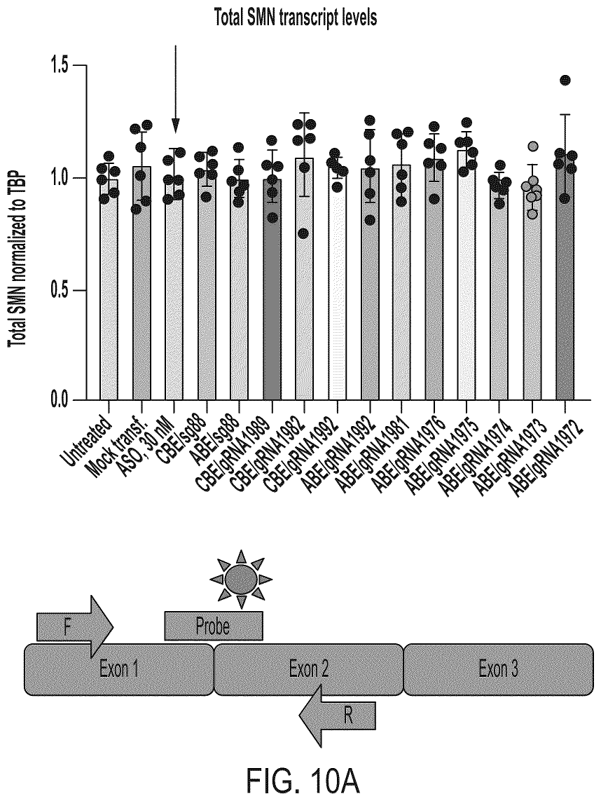

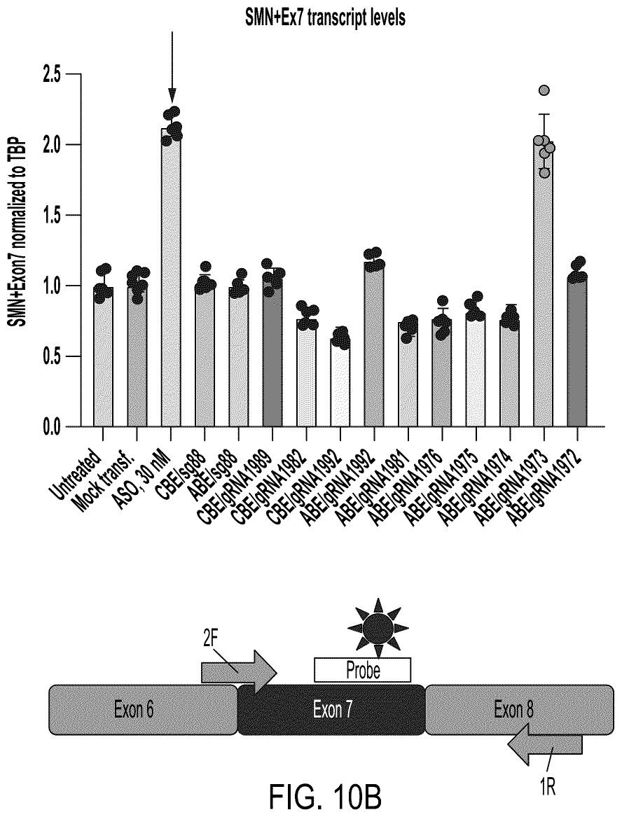

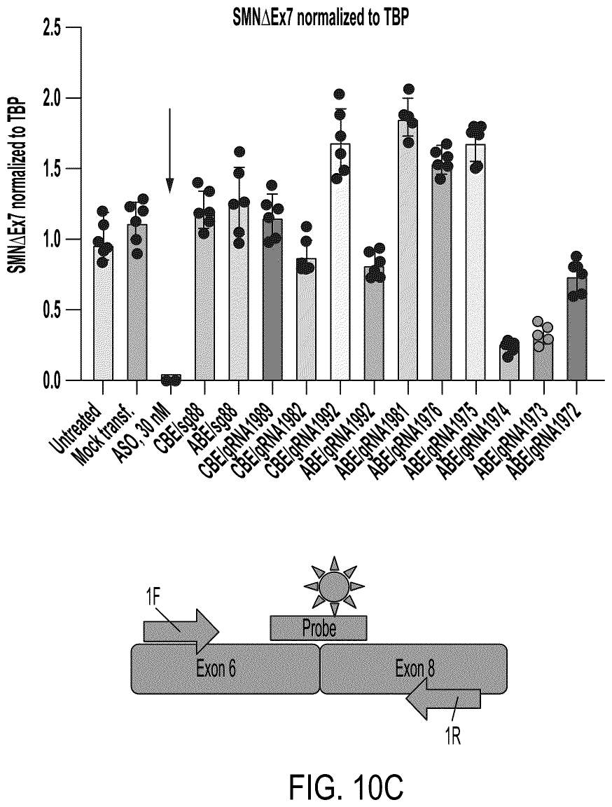

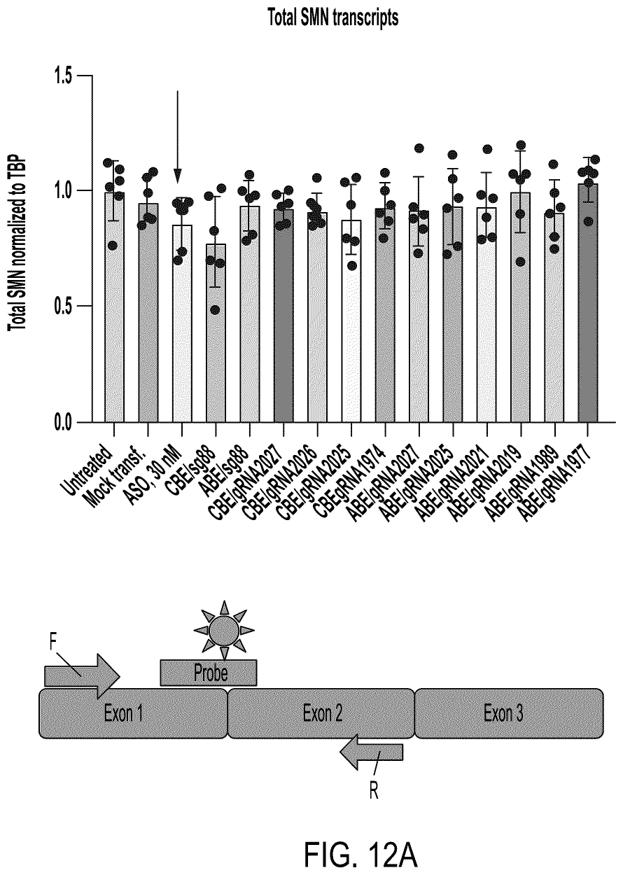

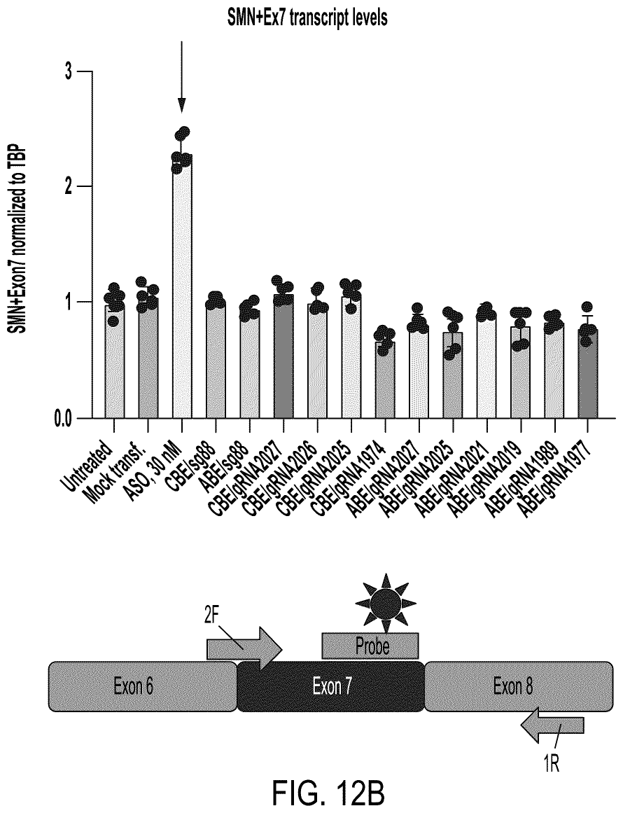

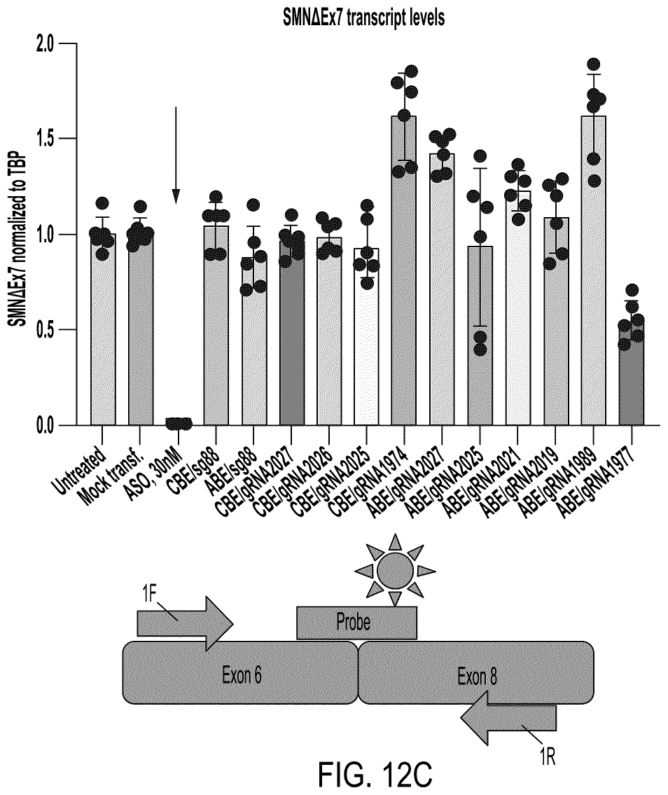

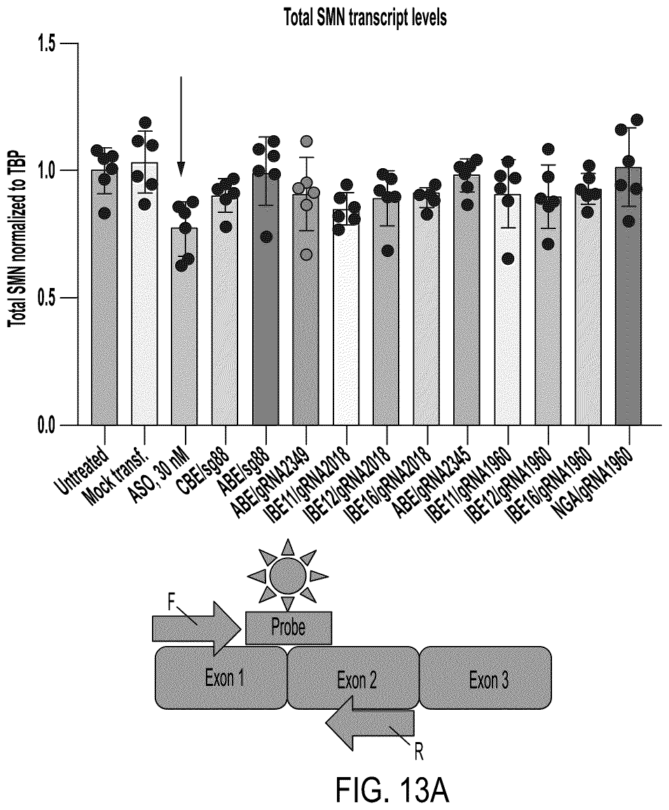

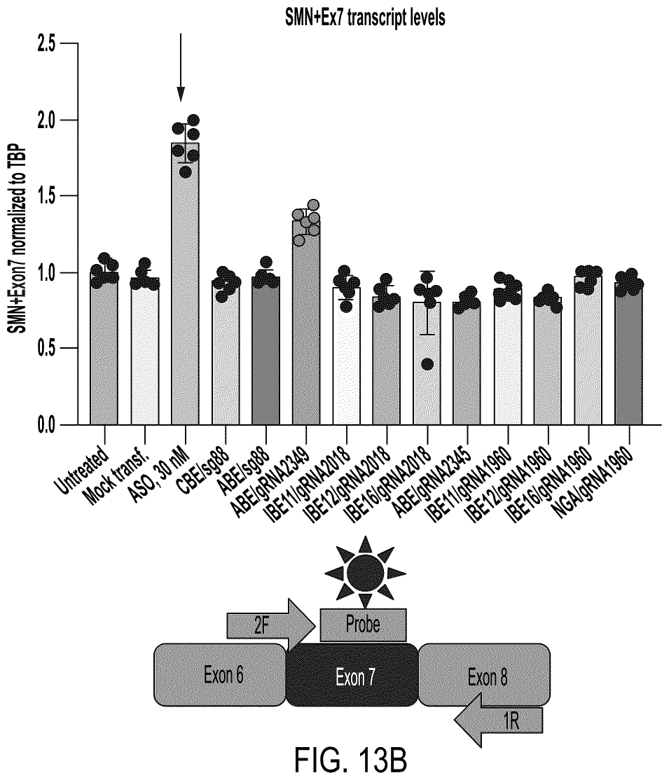

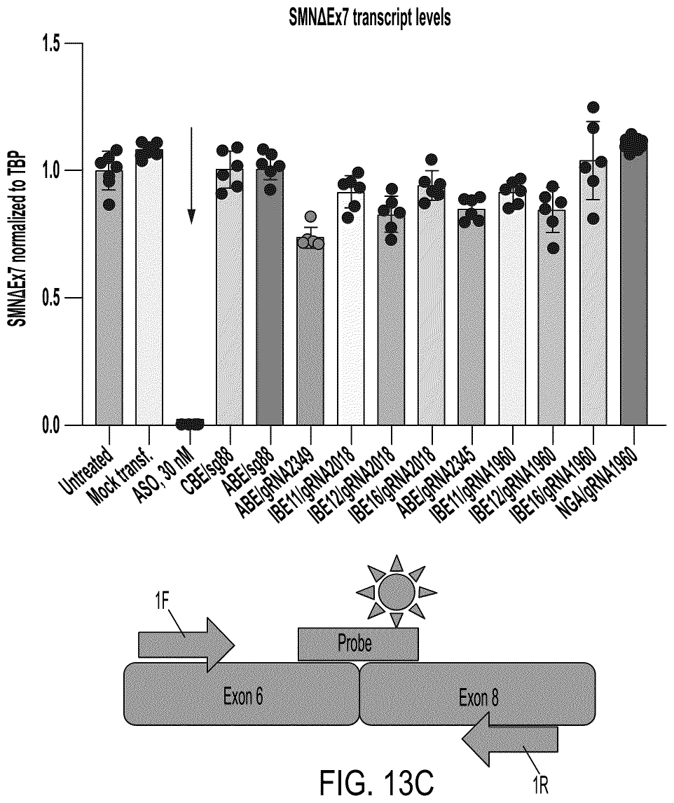

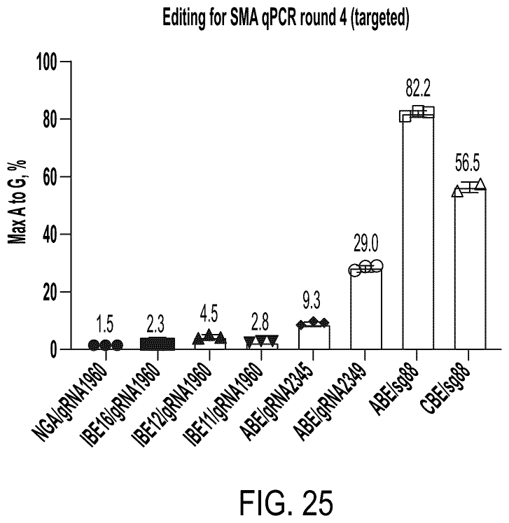

Having established an optimized dose of the base editor system to use in the screens, further screens were conducted to evaluate the effect of base editing using the systems on incorporation of Exon 7 into spliced transcripts expressed from the SMN2 polynucleotide (FIGS. 8A-8C, 10A-10C, 12A-12C, and 13A-13C). The following base editor systems were screened: ABE+gRNA1971; CBE+gRNA1977; ABE+gRNA1970; CBE+gRNA1976; ABE+gRNA1967; CBE+gRNA1966; ABE+gRNA1966; CBE+gRNA1962; ABE+gRNA1962; CBE+gRNA1960 (FIGS. 8A-8C); CBE+gRNA1989; CBE+gRNA1982; CBE+gRNA1992; ABE+gRNA1992; ABE+gRNA1981; ABE+gRNA1976; ABE+gRNA1975; ABE+gRNA1974; ABE+gRNA1973; ABE+gRNA1972 (FIGS. 10A-10C); CBE+gRNA2027; CBE+gRNA2026; CBE+gRNA2025; CBE+gRNA1974; ABE+gRNA2027; ABE+gRNA2025; ABE+gRNA2021; ABE+gRNA2019; ABE+gRNA1989; ABE+gRNA1977 (FIGS. 12A-12C); ABE+gRNA2349; IBE11+gRNA2018; IBE12+gRNA2018; IBE16+gRNA2018; ABE+gRNA2345; IBE11+gRNA1960; IBE12+gRNA1960; IBE16+gRNA1960; NGA+gRNA1960 (FIGS. 13A-13C). See Table 22 for further details relating to the base editor systems. The base editors IBE11, IBE12, and IBE16 each contained a TadA*8.20 adenosine deaminase domain. SMA patient fibroblast cells (GM03813 cells) were transfected with 60 ng of mRNA encoding the base editor and 30 ng of the guide polynucleotide for each base editor system. Following transfection, total SMN transcript levels, levels of SMN transcripts containing Exon 7, and levels of SMN transcripts lacking Exon 7 were measured using qRT-PCR. Base editing efficiencies were also measured for the following base editing systems (see FIG. 25): NGA+gRNA1960; IBE16+gRNA1960; IBE12+gRNA1960; IBE11+gRNA1960; ABE+gRNA2345; and ABE+gRNA2349. The following base editing systems were effective at introducing alterations to SMN2 that were associated with increased levels of spliced SMN2 transcripts containing Exon 7 and reduced levels of spliced transcripts lacking Exon 7: CBE+gRNA1962; ABE+gRNA1962; ABE+gRNA1973; ABE+gRNA2349.

TABLE 22
Details relating to base editor systems of FIGS. 8A to 13C. Each base
editor is named using the following convention: [Name of Type of Base
Editor]_[Deaminase Domain]_[nucleic acid programmable DNA
binding protein domain]_[PAM sequence], where “Sa” indicates
“Staplylococcus aureus,” “Sp” indicates “Streptococcus pyogenes,” and
“8.20” indicates “TadA*8.20.” See Tables 2B and 2C for guide sequences.

| | Name for | |
|---|---|---|
| | mRNA | |
| Type of Base Editor | Encoding the | |
| Used and Guide Name | Base Editor | Base Editor |
| CBE/88 | mRNA2923 | CBE_ppAPOBEC1_SpCas9_NGG |
| ABE/88 | mRNA2926 | ABE_8.20_SpCas9_NGG |
| ABE/1971 | mRNA2926 | ABE_8.20_SpCas9_NGG |
| CBE/1977 | mRNA2745 | CBE_ppAPOBEC1_SaCas9_NNGRRT |
| ABE/1970 | mRNA2926 | ABE_8.20_SpCas9_NGG |
| CBE/1976 | mRNA2889 | ABE_8.20_SaCas9_NNGRRT |
| ABE/1967 | mRNA2928 | ABE_8.20_SpCas9_NGA |
| CBE/1966 | mRNA2744 | CBE_ppAPOBEC1_SpCas9_NGA |
| ABE/1966 | mRNA2928 | ABE_8.20_SpCas9_NGA |
| CBE/1962 | mRNA2744 | CBE_ppAPOBEC1_SpCas9_NGA |
| ABE/1962 | mRNA2928 | ABE_8.20_SpCas9_NGA |
| CBE/1960 | mRNA2744 | CBE_ppAPOBEC1_SpCas9_NGA |
| ABE/gRNA1972 | mRNA2926 | ABE_8.20_SpCas9_NGG |
| ABE/gRNA1973 | mRNA2926 | ABE_8.20_SpCas9_NGG |
| ABE/gRNA1974 | mRNA2926 | ABE_8.20_SpCas9_NGG |
| ABE/gRNA1975 | mRNA2889 | ABE_8.20_SaCas9_NNGRRT |
| ABE/gRNA1976 | mRNA2889 | ABE_8.20_SaCas9_NNGRRT |
| ABE/gRNA1981 | mRNA2892 | ABE_8.20_SaCas9_NNNRRT |
| ABE/gRNA1992 | mRNA2892 | ABE_8.20_SaCas9_NNNRRT |
| CBE/gRNA1992 | mRNA2225 | CBE_ppAPOBEC1_SaCas9_NNNRRT |
| CBE/gRNA1982 | mRNA2225 | CBE_ppAPOBEC1_SaCas9_NNNRRT |
| CBE/gRNA1989 | mRNA2225 | CBE_ppAPOBEC1_SaCas9_NNNRRT |
| ABE/gRNA1977 | mRNA2889 | ABE_8.20_SaCas9_NNGRRT |
| ABE/gRNA1989 | mRNA2892 | ABE_8.20_SaCas9_NNNRRT |
| ABE/gRNA2019 | mRNA2926 | ABE_8.20_SpCas9_NGG |
| ABE/gRNA2021 | mRNA2926 | ABE_8.20_SpCas9_NGG |
| ABE/gRNA2025 | mRNA2892 | ABE_8.20_SaCas9_NNNRRT |
| ABE/gRNA2027 | mRNA2892 | ABE_8.20_SaCas9_NNNRRT |
| CBE/gRNA1974 | mRNA2923 | CBE_ppAPOBEC1_SpCas9_NGG |
| CBE/gRNA2025 | mRNA2225 | CBE_ppAPOBEC1_SaCas9_NNNRRT |
| CBE/gRNA2026 | mRNA2225 | CBE_ppAPOBEC1_SaCas9_NNNRRT |
| CBE/gRNA2027 | mRNA2225 | CBE_ppAPOBEC1_SaCas9_NNNRRT |
| NGA/gRNA1960 | mRNA2928 | ABE_8.20_SpCas9_NGA |
| IBE16/gRNA1960 | mRNA3165 | IBE16_ABE_8.20_SpCas9_NGA |
| | | (SEQ ID NO: 637) |
| IBE12/gRNA1960 | mRNA3166 | IBE12_ABE_8.20_SpCas9_NGA |
| | | (SEQ ID NO: 635) |
| IBE11/gRNA1960 | mRNA3168 | IBE11_ABE_8.20_SpCas9_NGA |
| | | (SEQ ID NO: 633) |
| ABE/gRNA2345 | mRNA2866 | ABE_8.20_SpCas9_NRTH |
| ABE/gRNA2349 | mRNA2866 | ABE_8.20_SpCas9_NRTH |

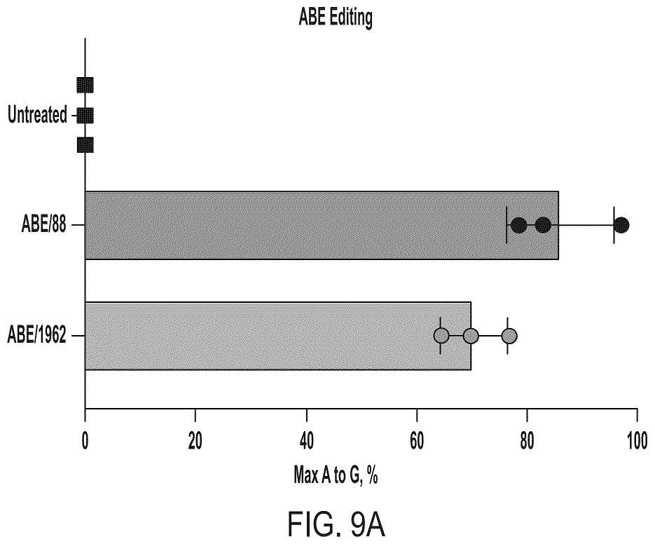

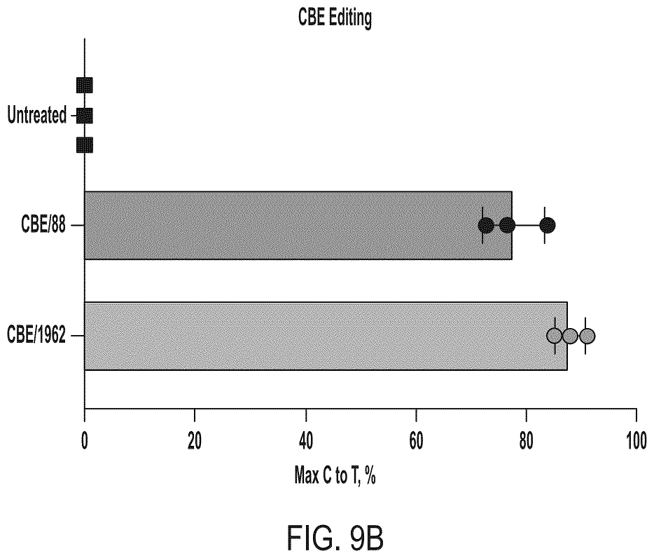

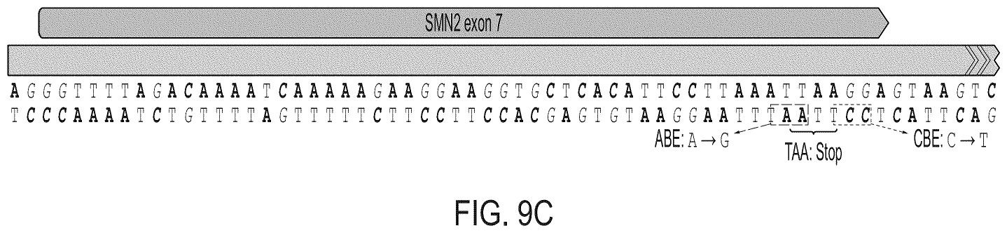

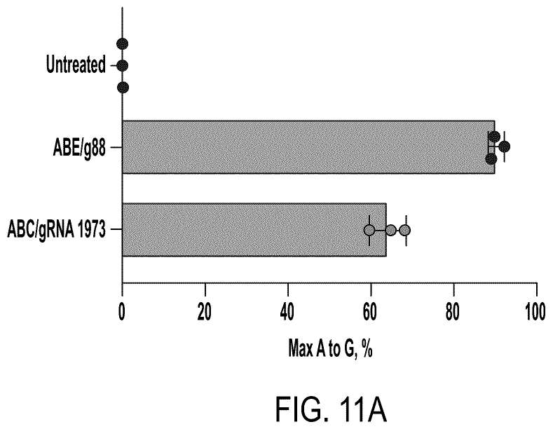

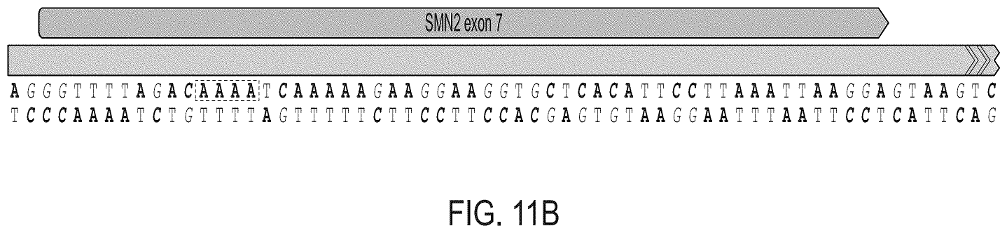

Further experiments were undertaken to evaluate base editing efficiencies in SMA patient fibroblast cells (GM03813 cells) for the following base editor systems that performed well in the above screens: ABE+gRNA1962 (FIG. 9A), CBE+gRNA1962 (FIG. 9B), and ABE+gRNA1973 (FIG. 11A). The base editor system ABE+gRNA1962, which targeted the two As boxed in FIG. 9C for deamination, had a maximum A to G percent base editing efficiency of over 60%. The base editor system CBE+gRNA1962, which targeted the two Cs boxed in FIG. 9C for deamination, had a maximum C to T percent base editing efficiency of over 8000. The base editor system ABE+gRNA1973, which targeted the four As boxed in FIG. 11B, had a maximum A to G percent base editing efficiency of over 60%.

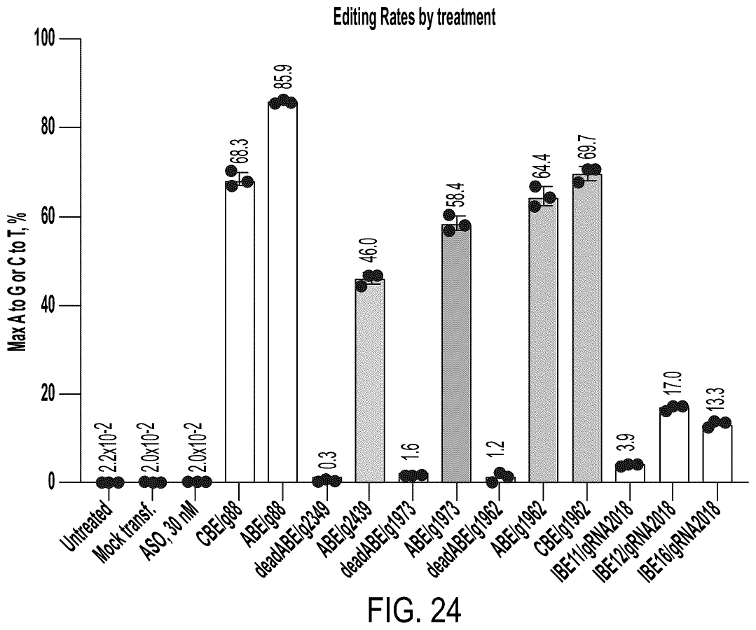

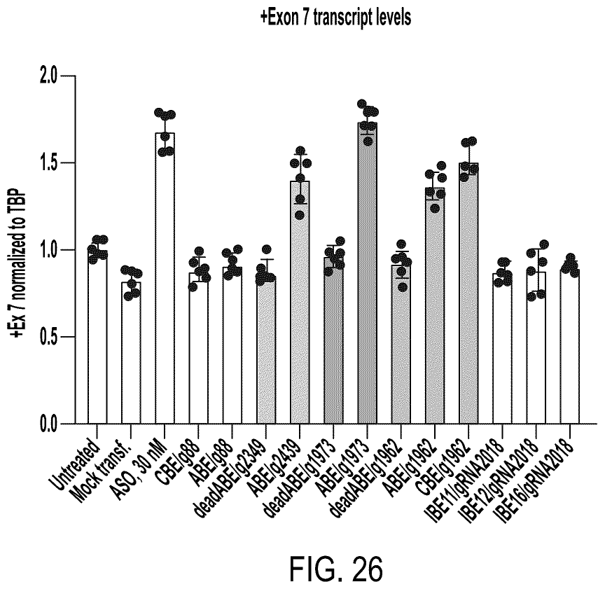

An experiment was undertaken to confirm that splicing changes observed following base editing of the SMA patient fibroblast cells were associated with base editing and not the result of binding of the base editor system SMN2. SMA patient fibroblast cells (GM03813 cells) were transduced with mRNA encoding the base editors indicate along the x-axis of FIGS. 24 and 26 in combination with the indicated guide polynucleotides. See Table 23 for further details relating to the base editor systems of FIG. 24. Five (5) days following transduction, levels of spliced SMN2 transcripts containing Exon 7 were measured. By comparing levels of spliced SMN2 transcripts containing Exon 7 in cells base edited using a catalytically active base editor or a corresponding base editor variant with low-to-no base editing activity (i.e., dead), it was confirmed that the observed changes in transcript levels in the base edited cells was associated with base editing and not simply with binding of a base editor to a target nucleotide without any deamination.

TABLE 23
Details relating to base editor systems of FIG. 24. Each base
editor is named using the following convention: [Name of Type of Base
Editor]_[Deaminase Domain]_[nucleic acid programmable DNA
binding protein domain]_[PAM sequence], where “Sa” indicates
“Staplylococcus aureus,” “Sp” indicates “Streptococcus pyogenes,” and
“8.20” indicates “TadA*8.20.” See Tables 2B and 2C for guide sequences.

| | Name for | |
|---|---|---|
| | mRNA | |
| Type of Base Editor | encoding the | |
| Used and Guide Name | Base Editor | Base Editor |
| IBE16/gRNA2018 | mRNA3167 | IBE16_ABE_8.20_SpCas9_NGC (SEQ ID NO: 643) |
| IBE12/gRNA2018 | mRNA3170 | IBE12_ABE_8.20_SpCas9_NGC (SEQ ID NO: 641) |
| IBE11/gRNA2018 | mRNA2959 | IBE11_ABE_8.20_SpCas9_NGC (SEQ ID NO: 639) |
| CBE/g1962 | mRNA2744 | CBE_ppAPOBEC1_SpCas9_NGA |
| ABE/g1962 | mRNA2928 | ABE_8.20_SpCas9_NGA |
| deadABE/g1962 | mRNA2971 | dead_ABE_dABE8.20_E59Q_SpCas9_NGA |
| ABE/g1973 | mRNA2926 | ABE_8.20_SpCas9_NGG |
| deadABE/g1973 | mRNA2970 | dead_ABE_dABE8.20_E59Q_SpCas9_NGG |
| ABE/g2439 | mRNA2866 | ABE_8.20_SpCas9_NRTH |
| deadABE/g2349 | mRNA2975 | dead_ABE_dABE8.20_E59Q_SpCas9_NRTH |

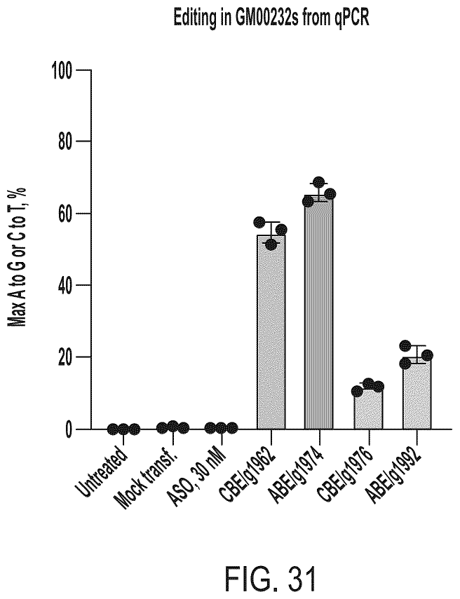

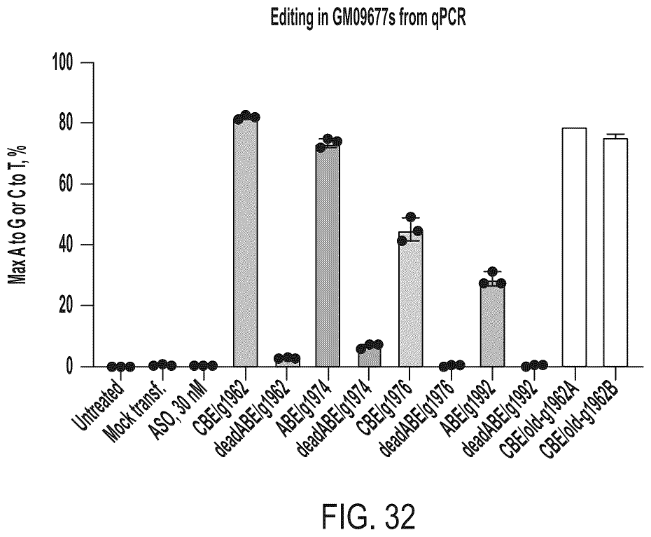

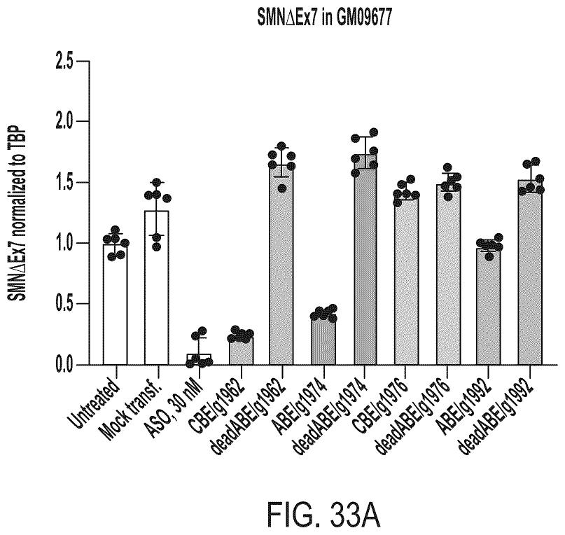

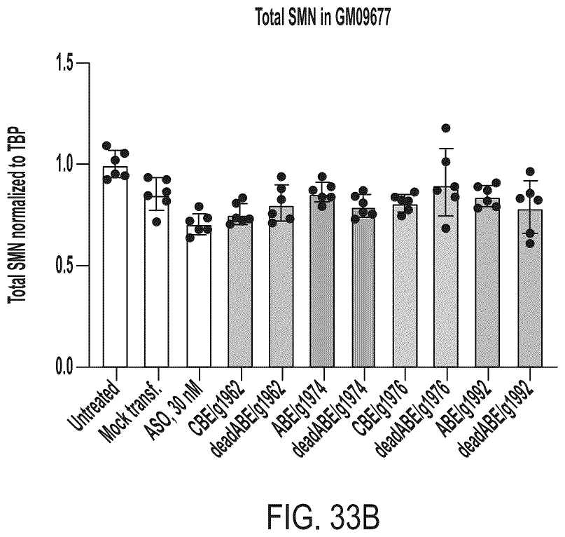

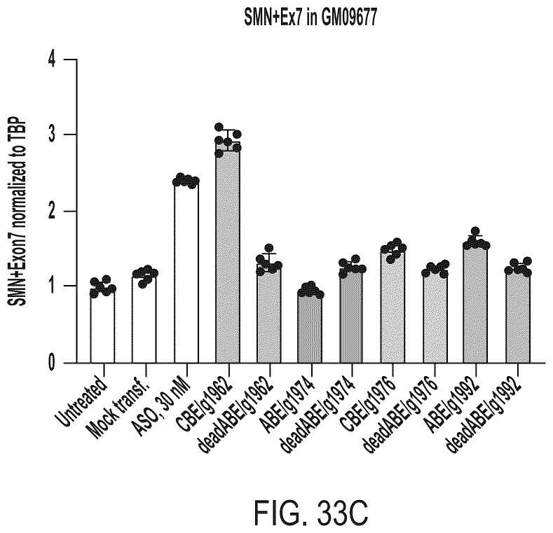

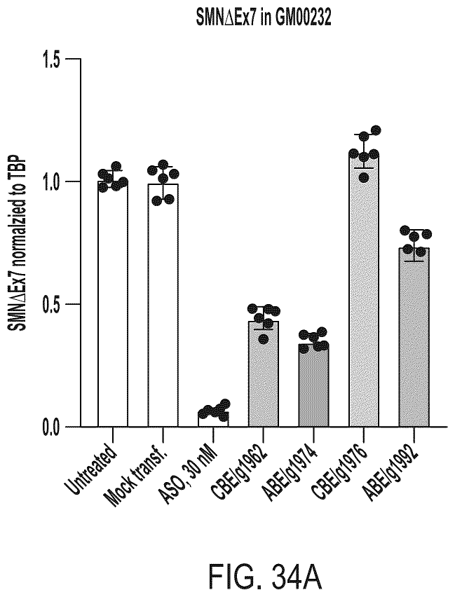

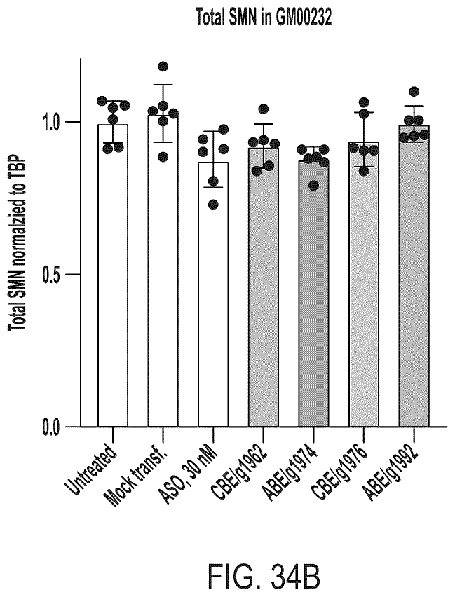

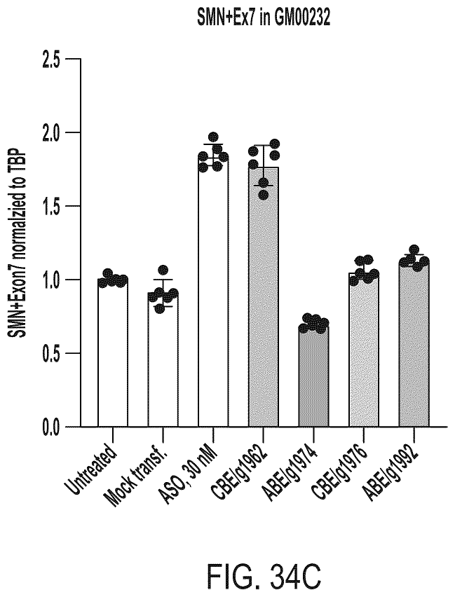

Experiments were next undertaken to evaluate base editor systems identified as described above in the SMA patient fibroblast cell lines GM00232 and GM09677. GM00232 cells were chosen because this patient fibroblast line was homozygous for deletion of exons 7 and 8 in the SMN1 gene, contained 2 copies of the SMN2 gene, and were of a different genetic background than the GM03813 cells, which contained 3 copies of the SMN2 gene. GM09677 cells were chosen because they too were homozygous for deletion of exons 7 and 8 in the SMN1 gene, had the same number of SMN2 copies as GM03813 cells, and had a different genetic background than the GM03813 cells. The effect of base editing in the two SMA fibroblast cell lines on incorporation of Exon 7 into spliced transcripts expressed from the SMN2 polynucleotide was evaluated (FIGS. 31, 32, 33A-33C, and 34A-34C). The following base editor systems were evaluated: CBE (encoded by mRNA2744)+gRNA1962; CBE (encoded by mRNA2745)+gRNA1976; ABE (encoded by mRNA2926)+gRNA1974; and ABE (encoded by mRNA2892)+gRNA1992 (see mRNA sequences and corresponding amino acid sequences provided in Table 23.1 below). As negative controls, the following systems were evaluated, which lacked base editing activity: deadCBE (encoded by mRNA2971)+gRNA1962; deadCBE (encoded by mRNA2972)+gRNA1976; deadABE (encoded by mRNA2970)+gRNA1974; and deadABE (encoded by mRNA2973)+gRNA1992 (see mRNA sequences and corresponding amino acid sequences provided in Table 23.1 below). The SMA patient fibroblast cells were transfected with 60 ng of mRNA encoding the base editor and 30 ng of the guide polynucleotide for each base editor system. Following transfection, total SMN transcript levels, levels of SMN transcripts containing Exon 7, and levels of SMN transcripts lacking Exon 7 were measured using qRT-PCR. In the GM09677 cells, dead base editors were evaluated side-by-side with active editors to confirm that the splicing changes (exon 7 inclusion+exclusion levels) measured were not due to guide/base editor binding or steric hindrance, and to confirm that the changes likely resulted only from base editing. All of the base editor systems evaluated performed similarly in all three SMA patient fibroblast cell lines (GM03813, GM00232, and GM00967), including rank order and relative magnitude of change.

TABLE 23.1
Sequences encoding base editors.

| Sequence | |
|---|---|
| Description | Sequence |
| Nucleotide | TCTAGAGTTTAAACATTTAAATCTGCAGATCCCAATGGCGCGCCGAGCTTGGCT |
| sequence | CGAGCATGGTCATAGCTGTTTCCTGTGTGAAATTGTTATCCGCTCACAATTCCA |
| of a | CACAACATACGAGCCGGAAGCATAAAGTGTAAAGCCTGGGGTGCCTAATGAGTG |
| plasmid | AGCTAACTCACATTAATTGCGTTGCGCTCACTGCCCGCTTTCCAGTCGGGAAAC |
| encoding | CTGTCGTGCCAGCTGCATTAATGAATCGGCCAACGCGCGGGGAGAGGCGGTTTG |
| mRNA2744 | CGTATTGGGCACTCTTGCGCTTCCTCGCTCACTGACTCGCTGCGCTCGGTCGTT |
| | CGGCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGGCC |
| | AGGAACCGTAAAAAGGCCGCGTTGCTGGCGTTTTTCCATAGGCTCCGCCCCCCT |
| | GACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTT |
| | CCGACCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTACGGGG |
| | TCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCA |
| | ATCTAAAGTATATATGAGTAAACTTGGTCTGACAGTTAGAAAAACTCATCGAGC |
| | ATCAAATGAAACTGCAATTTATTCATATCAGGATTATCAATACCATATTTTTGA |
| | AAAAGCCGTTTCTGTAATGAAGGAGAAAACTCACCGAGGCAGTTCCATAGGATG |
| | GCAAGATCCTGGTATCGGTCTGCGATTCCGACTCGTCCAACATCAATACAACCT |
| | ATTAATTTCCCCTCGTCAAAAATAAGGTTATCAAGTGAGAAATCACCATGAGTG |
| | ACGACTGAATCCGGTGAGAATGGCAAAAGTTTATGCATTTCTTTCCAGACTTGT |
| | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGACCTCTGAGAAGGGCCCTAG |
| | CACAGGCGACCCCACCCTGCGGCGGAGAATCGAGAGCTGGGAGTTCGACGTGTT |
| | CTACGACCCTAGAGAACTGAGAAAGGAAACCTGCCTGCTGTACGAGATCAAGTG |
| | GGGCATGAGCAGAAAGATCTGGCGGAGCTCTGGCAAGAACACCACCAACCACGT |
| | GGAAGTGAATTTCATCAAGAAGTTCACCAGCGAGAGAAGGTTCCACAGCAGCAT |
| | CAGCTGCAGCATCACCTGGTTCCTGAGCTGGTCCCCTTGCTGGGAATGCAGCCA |
| | GGCCATCAGAGAGTTCCTGAGCCAACACCCCGGAGTGACACTGGTGATCTACGT |
| | GGCCAGACTGTTCTGGCACATGGACCAGAGAAACAGACAGGGCCTGAGAGATCT |
| | GGTCAACAGCGGCGTGACTATCCAGATCATGCGGGCCAGCGAGTACTACCACTG |
| | TTGGCGGAACTTCGTGAACTACCCCCCCGGCGATGAGGCCCACTGGCCTCAGTA |
| | CCCTCCTCTGTGGATGATGCTGTACGCCCTGGAACTGCACTGCATCATCCTGTC |
| | TCTGCCTCCATGTCTGAAGATCTCTAGAAGATGGCAGAACCACCTGGCCTTCTT |
| | CAGACTGCACCTGCAGAATTGCCACTACCAGACCATCCCCCCCCACATCCTGCT |
| | GGCTACAGGCCTGATCCACCCTTCTGTGACCTGGAGAAGCGGAGGATCTAGCGG |
| | CGGCTCTAGCGGATCTGAGACACCTGGCACAAGCGAGTCTGCCACACCTGAGAG |
| | TAGCGGCGGATCTTCTGGCGGCTCCGACAAGAAGTACTCTATCGGACTGGCCAT |
| | CGGCACCAACTCTGTTGGATGGGCCGTGATCACCGACGAGTACAAGGTGCCCAG |
| | CAAGAAATTCAAGGTGCTGGGCAACACCGACCGGCACAGCATCAAGAAGAATCT |
| | GATCGGCGCCCTGCTGTTCGACTCTGGCGAAACAGCCGAAGCCACCAGACTGAA |
| | GAGAACCGCCAGGCGGAGATACACCCGGCGGAAGAACCGGATCTGCTACCTGCA |
| | AGAGATCTTCAGCAACGAGATGGCCAAGGTGGACGACAGCTTCTTCCACAGACT |
| | GGAAGAGTCCTTCCTGGTGGAAGAGGACAAGAAGCACGAGCGGCACCCCATCTT |
| | CGGCAACATCGTGGATGAGGTGGCCTACCACGAGAAGTACCCCACCATCTACCA |
| | CCTGAGAAAGAAACTGGTGGACAGCACCGACAAGGCCGACCTGAGACTGATCTA |
| | CCTGGCTCTGGCCCACATGATCAAGTTCCGGGGCCACTTTCTGATCGAGGGCGA |
| | TCTGAACCCCGACAACAGCGACGTGGACAAGCTGTTCATCCAGCTGGTGCAGAC |
| | CTACAACCAGCTGTTCGAGGAAAACCCCATCAACGCCTCTGGCGTGGACGCCAA |
| | GGCTATCCTGTCTGCCAGACTGAGCAAGAGCAGAAGGCTGGAAAACCTGATCGC |
| | CCAGCTGCCTGGCGAGAAGAAGAATGGCCTGTTCGGCAACCTGATTGCCCTGAG |
| | CCTGGGACTGACCCCTAACTTCAAGAGCAACTTCGACCTGGCCGAGGATGCCAA |
| | ACTGCAGCTGAGCAAGGACACCTACGACGACGACCTGGACAATCTGCTGGCCCA |
| | GATCGGCGATCAGTACGCCGACTTGTTTCTGGCCGCCAAGAACCTGTCCGACGC |
| | CATCCTGCTGAGCGATATCCTGAGAGTGAACACCGAGATCACAAAGGCCCCTCT |
| | GAGCGCCTCTATGATCAAGAGATACGACGAGCACCACCAGGATCTGACCCTGCT |
| | GAAGGCCCTCGTTAGACAGCAGCTGCCAGAGAAGTACAAAGAGATTTTCTTCGA |
| | TCAGTCCAAGAACGGCTACGCCGGCTACATTGATGGCGGAGCCAGCCAAGAGGA |
| | ATTCTACAAGTTCATCAAGCCCATCCTGGAAAAGATGGACGGCACCGAGGAACT |
| | GCTGGTCAAGCTGAACAGAGAGGACCTGCTGCGGAAGCAGCGGACCTTCGACAA |
| | TGGCTCTATCCCTCACCAGATCCACCTGGGAGAGCTGCACGCCATTCTGCGGAG |
| | ACAAGAGGACTTTTACCCATTCCTGAAGGACAACCGGGAAAAGATCGAGAAGAT |
| | CCTGACCTTCAGGATCCCCTACTACGTGGGACCACTGGCCAGAGGCAATAGCAG |
| | ATTCGCCTGGATGACCAGAAAGAGCGAGGAAACCATCACACCCTGGAACTTCGA |
| | GGAAGTGGTGGACAAGGGCGCCAGCGCTCAGTCCTTCATCGAGCGGATGACCAA |
| | CTTCGATAAGAACCTGCCTAACGAGAAGGTGCTGCCCAAGCACTCCCTGCTGTA |
| | TGAGTACTTCACCGTGTACAACGAGCTGACCAAAGTGAAATACGTGACCGAGGG |
| | AATGAGAAAGCCCGCCTTTCTGAGCGGCGAGCAGAAAAAGGCCATTGTGGATCT |
| | GCTGTTCAAGACCAACCGGAAAGTGACCGTGAAGCAGCTGAAAGAGGACTACTT |
| | CAAGAAAATCGAGTGCTTCGACAGCGTGGAAATCAGCGGCGTGGAAGATCGGTT |
| | CAATGCCAGCCTGGGCACATACCACGACCTGCTGAAAATTATCAAGGACAAGGA |
| | CTTCCTGGACAACGAAGAGAACGAGGACATTCTCGAGGACATCGTGCTGACCCT |
| | GACACTGTTTGAGGACAGAGAGATGATCGAGGAACGGCTGAAAACATACGCCCA |
| | CCTGTTCGACGACAAAGTGATGAAGCAACTGAAGCGGAGGCGGTACACAGGCTG |
| | GGGCAGACTGTCTCGGAAGCTGATCAACGGCATCCGGGATAAGCAGTCCGGCAA |
| | GACAATCCTGGATTTCCTGAAGTCCGACGGCTTCGCCAACAGAAACTTCATGCA |
| | GCTGATCCACGACGACAGCCTGACCTTTAAAGAGGACATCCAGAAAGCCCAGGT |
| | GTCCGGCCAAGGCGATTCTCTGCACGAGCACATTGCCAACCTGGCCGGATCTCC |
| | CGCCATTAAGAAGGGCATCCTGCAGACAGTGAAGGTGGTGGACGAGCTTGTGAA |
| | AGTGATGGGCAGACACAAGCCCGAGAACATCGTGATCGAAATGGCCAGAGAGAA |
| | CCAGACCACACAGAAGGGCCAGAAGAACAGCCGCGAGAGAATGAAGCGGATCGA |
| | AGAGGGCATCAAAGAGCTGGGCAGCCAGATCCTGAAAGAACACCCCGTGGAAAA |
| | CACCCAGCTGCAGAACGAGAAGCTGTACCTGTACTACCTGCAGAATGGACGGGA |
| | TATGTACGTGGACCAAGAGCTGGACATCAACCGGCTGAGCGACTACGATGTGGA |
| | CCATATCGTGCCCCAGAGCTTTCTGAAGGACGACTCCATCGATAACAAGGTCCT |
| | GACCAGAAGCGACAAGAACCGGGGCAAGAGCGATAACGTGCCCTCCGAAGAGGT |
| | GGTCAAGAAGATGAAGAACTACTGGCGACAGCTGCTGAACGCCAAGCTGATTAC |
| | CCAGCGGAAGTTCGATAACCTGACCAAGGCCGAGAGAGGCGGCCTGAGCGAACT |
| | TGATAAGGCCGGCTTCATTAAGCGGCAGCTGGTGGAAACCCGGCAGATCACCAA |
| | ACACGTGGCACAGATTCTGGACTCCCGGATGAACACTAAGTACGACGAGAATGA |
| | CAAGCTGATCCGGGAAGTGAAAGTCATCACCCTGAAGTCTAAGCTGGTGTCCGA |
| | TTTCCGGAAGGATTTCCAGTTCTACAAAGTGCGGGAAATCAACAACTACCATCA |
| | CGCCCACGACGCCTACCTGAATGCCGTTGTTGGAACAGCCCTGATCAAGAAGTA |
| | TCCCAAGCTGGAAAGCGAGTTCGTGTACGGCGACTACAAGGTGTACGACGTGCG |
| | GAAGATGATCGCCAAGAGCGAACAAGAGATCGGCAAGGCTACCGCCAAGTACTT |
| | TTTCTACAGCAACATCATGAACTTTTTCAAGACAGAGATCACCCTGGCCAACGG |
| | CGAGATCCGGAAAAGACCCCTGATCGAGACAAACGGCGAAACCGGGGAGATCGT |
| | GTGGGATAAGGGCAGAGATTTTGCCACAGTGCGGAAAGTGCTGAGCATGCCCCA |
| | AGTGAATATCGTGAAGAAAACCGAGGTGCAGACAGGCGGCTTCAGCAAAGAGTC |
| | TATCCTGCCTAAGCGGAACAGCGATAAGCTGATCGCCAGAAAGAAGGACTGGGA |
| | CCCCAAGAAATACGGCGGCTTCGTGTCTCCTACCGTGGCCTATTCTGTGCTGGT |
| | GGTGGCCAAAGTGGAAAAGGGCAAGTCCAAAAAGCTCAAGAGCGTGAAAGAGCT |
| | GCTGGGGATCACCATCATGGAAAGAAGCAGCTTTGAGAAGAACCCGATCGACTT |
| | TCTGGAAGCCAAGGGCTACAAAGAAGTCAAGAAGGACCTCATCATCAAGCTCCC |
| | CAAGTACAGCCTGTTCGAGCTGGAAAATGGCCGGAAGCGGATGCTGGCCTCTGC |
| | TAGAGAACTGCAGAAGGGAAACGAGCTGGCCCTGCCTAGCAAATATGTGAACTT |
| | CCTGTACCTGGCCAGCCACTATGAGAAGCTGAAGGGCAGCCCCGAGGACAATGA |
| | GCAAAAGCAGCTGTTTGTGGAACAGCACAAGCACTACCTGGACGAGATCATCGA |
| | GCAGATCAGCGAGTTCTCCAAGAGAGTGATCCTGGCCGACGCTAACCTGGATAA |
| | GGTGCTGTCTGCCTATAACAAGCACCGGGACAAGCCTATCAGAGAGCAGGCCGA |
| | GAATATCATCCACCTGTTTACCCTGACCAACCTGGGAGCCCCTGCCGCCTTCAA |
| | GTACTTCGACACCACCATCGACCGGAAGCAGTACCGGTCCACCAAAGAGGTGCT |
| | GGACGCCACACTGATCCACCAGTCTATCACCGGCCTGTACGAAACCCGGATCGA |
| | CCTGTCTCAGCTCGGCGGCGATTCTGGTGGTTCTGGCGGAAGTGGCGGATCCAC |
| | CAATCTGAGCGACATCATCGAAAAAGAGACAGGCAAGCAGCTCGTGATCCAAGA |
| | ATCCATCCTGATGCTGCCTGAAGAGGTTGAGGAAGTGATCGGCAACAAGCCTGA |
| | GTCCGACATCCTGGTGCACACCGCCTACGATGAGAGCACCGATGAGAACGTCAT |
| | GCTGCTGACAAGCGACGCCCCTGAGTACAAGCCTTGGGCTCTCGTGATTCAGGA |
| | CAGCAATGGGGAGAACAAGATCAAGATGCTGAGCGGAGGTAGCGGAGGCAGTGG |
| | CGGAAGCACAAACCTGTCTGATATCATTGAAAAAGAAACCGGGAAGCAACTGGT |
| | CATTCAAGAGTCCATTCTCATGCTCCCGGAAGAAGTCGAGGAAGTCATTGGAAA |
| | CAAACCCGAGAGCGATATTCTGGTCCACACAGCCTATGACGAGTCTACAGACGA |
| | AAACGTGATGCTCCTGACCTCTGACGCTCCCGAGTATAAGCCCTGGGCACTTGT |
| | TATCCAGGACTCTAACGGGGAAAACAAAATCAAAATGTTGTCCGGCGGCAGCAA |
| | GCGGACAGCCGATGGATCTGAGTTCGAGAGCCCCAAGAAGAAACGGAAGGTGTA |
| | ATAGTGAGCGGCCGCTTAATTAAGCTGCCTTCTGCGGGGCTTGCCTTCTGGCCA |
| | AGCCCTTCTTCTCTCCCTTGCACCTGTACCTCTTGGTCTTTGAATAAAGCCTGA |
| | GTAGGAAG (SEQ ID NO: 645) |
| Amino acid | MTSEKGPSTGDPTLRRRIESWEFDVFYDPRELRKETCLLYEIKWGMSRKIWRSS |
| sequence | GKNTTNHVEVNFIKKFTSERRFHSSISCSITWFLSWSPCWECSQAIREFLSQHP |
| of the | GVTLVIYVARLFWHMDQRNRQGLRDLVNSGVTIQIMRASEYYHCWRNFVNYPPG |
| polypeptide | DEAHWPQYPPLWMMLYALELHCIILSLPPCLKISRRWQNHLAFFRLHLQNCHYQ |
| encoded | TIPPHILLATGLIHPSVTWRSGGSSGGSSGSETPGTSESATPESSGGSSGGSDK |
| by | KYSIGLAIGTNSVGWAVITDEYKVPSKKFKVLGNTDRHSIKKNLIGALLFDSGE |
| mRNA2744 | TAEATRLKRTARRRYTRRKNRICYLQEIFSNEMAKVDDSFFHRLEESFLVEEDK |
| | KHERHPIFGNIVDEVAYHEKYPTIYHLRKKLVDSTDKADLRLIYLALAHMIKFR |
| | GHFLIEGDLNPDNSDVDKLFIQLVQTYNQLFEENPINASGVDAKAILSARLSKS |
| | RRLENLIAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMIKRYDE |
| | HHQDLTLLKALVRQQLPEKYKEIFFDQSKNGYAGYIDGGASQEEFYKFIKPILE |
| | KMDGTEELLVKLNREDLLRKQRTFDNGSIPHQIHLGELHAILRRQEDFYPFLKD |
| | NREKIEKILTFRIPYYVGPLARGNSRFAWMTRKSEETITPWNFEEVVDKGASAQ |
| | SFIERMTNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLGTYHDL |
| | LKIIKDKDFLDNEENEDILEDIVLTLTLFEDREMIEERLKTYAHLFDDKVMKQL |
| | KRRRYTGWGRLSRKLINGIRDKQSGKTILDFLKSDGFANRNFMQLIHDDSLTFK |
| | EDIQKAQVSGQGDSLHEHIANLAGSPAIKKGILQTVKVVDELVKVMGRHKPENI |
| | VIEMARENQTTQKGQKNSRERMKRIEEGIKELGSQILKEHPVENTQLQNEKLYL |
| | YYLQNGRDMYVDQELDINRLSDYDVDHIVPQSFLKDDSIDNKVLTRSDKNRGKS |
| | DNVPSEEVVKKMKNYWRQLLNAKLITQRKFDNLTKAERGGLSELDKAGFIKRQL |
| | VETRQITKHVAQILDSRMNTKYDENDKLIREVKVITLKSKLVSDFRKDFQFYKV |
| | REINNYHHAHDAYLNAVVGTALIKKYPKLESEFVYGDYKVYDVRKMIAKSEQEI |
| | GKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFVSP |
| | TVAYSVLVVAKVEKGKSKKLKSVKELLGITIMERSSFEKNPIDFLEAKGYKEVK |
| | KDLIIKLPKYSLFELENGRKRMLASARELQKGNELALPSKYVNFLYLASHYEKL |
| | KGSPEDNEQKQLFVEQHKHYLDEIIEQISEFSKRVILADANLDKVLSAYNKHRD |
| | KPIREQAENIIHLFTLTNLGAPAAFKYFDTTIDRKQYRSTKEVLDATLIHQSIT |
| | GLYETRIDLSQLGGDSGGSGGSGGSTNLSDIIEKETGKQLVIQESILMLPEEVE |
| | EVIGNKPESDILVHTAYDESTDENVMLLTSDAPEYKPWALVIQDSNGENKIKML |
| | SGGSGGSGGSTNLSDIIEKETGKQLVIQESILMLPEEVEEVIGNKPESDILVHT |
| | AYDESTDENVMLLTSDAPEYKPWALVIQDSNGENKIKMLSGGSKRTADGSEFES |
| | PKKKRKV (SEQ ID NO: 646) |
| Nucleotide | ATCAAATGAAACTGCAATTTATTCATATCAGGATTATCAATACCATATTTTTGA |
| sequence | AAAAGCCGTTTCTGTAATGAAGGAGAAAACTCACCGAGGCAGTTCCATAGGATG |
| of a | GCAAGATCCTGGTATCGGTCTGCGATTCCGACTCGTCCAACATCAATACAACCT |
| plasmid | ATTAATTTCCCCTCGTCAAAAATAAGGTTATCAAGTGAGAAATCACCATGAGTG |
| encoding | ACGACTGAATCCGGTGAGAATGGCAAAAGTTTATGCATTTCTTTCCAGACTTGT |
| mRNA2745 | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGACCTCTGAGAAGGGCCCTAG |
| | CACAGGCGACCCCACCCTGCGGCGGAGAATCGAGAGCTGGGAGTTCGACGTGTT |
| | CTACGACCCTAGAGAACTGAGAAAGGAAACCTGCCTGCTGTACGAGATCAAGTG |
| | GGGCATGAGCAGAAAGATCTGGCGGAGCTCTGGCAAGAACACCACCAACCACGT |
| | GGAAGTGAATTTCATCAAGAAGTTCACCAGCGAGAGAAGGTTCCACAGCAGCAT |
| | CAGCTGCAGCATCACCTGGTTCCTGAGCTGGTCCCCTTGCTGGGAATGCAGCCA |
| | GGCCATCAGAGAGTTCCTGAGCCAACACCCCGGAGTGACACTGGTGATCTACGT |
| | GGCCAGACTGTTCTGGCACATGGACCAGAGAAACAGACAGGGCCTGAGAGATCT |
| | GGTCAACAGCGGCGTGACTATCCAGATCATGCGGGCCAGCGAGTACTACCACTG |
| | TTGGCGGAACTTCGTGAACTACCCCCCCGGCGATGAGGCCCACTGGCCTCAGTA |
| | CCCTCCTCTGTGGATGATGCTGTACGCCCTGGAACTGCACTGCATCATCCTGTC |
| | TCTGCCTCCATGTCTGAAGATCTCTAGAAGATGGCAGAACCACCTGGCCTTCTT |
| | CAGACTGCACCTGCAGAATTGCCACTACCAGACCATCCCCCCCCACATCCTGCT |
| | GGCTACAGGCCTGATCCACCCTTCTGTGACCTGGAGAAGCGGAGGATCTAGCGG |
| | CGGCTCTAGCGGATCTGAGACACCTGGCACAAGCGAGTCTGCCACACCTGAGAG |
| | TAGCGGCGGATCTTCTGGCGGCTCCGGCAAGAGAAATTACATCCTCGGACTGGC |
| | CATCGGCATCACCTCTGTCGGCTACGGCATCATCGACTACGAGACACGGGACGT |
| | GATCGATGCCGGCGTGCGGCTGTTTAAAGAGGCCAACGTCGAGAACAACGAGGG |
| | CCGCAGATCTAAGAGAGGCGCCAGACGGCTGAAGAGAAGAAGAAGGCACCGGAT |
| | CCAGAGAGTGAAGAAGCTGCTGTTCGACTACAACCTGCTGACCGACCACAGCGA |
| | GCTGAGCGGCATCAATCCTTACGAGGCCAGAGTGAAGGGCCTGAGCCAGAAGCT |
| | GAGCGAGGAAGAGTTTAGCGCTGCCCTGCTGCACCTGGCCAAGAGAAGAGGCGT |
| | GCACAACGTGAACGAGGTGGAAGAGGACACCGGCAACGAGCTGTCCACCAAAGA |
| | GCAGATCAGCCGGAACAGCAAGGCCCTGGAAGAGAAATACGTGGCCGAACTGCA |
| | GCTGGAACGGCTGAAAAAGGATGGCGAAGTGCGGGGCAGCATCAACCGGTTCAA |
| | GACCAGCGACTACGTGAAAGAAGCCAAACAGCTGCTGAAGGTGCAGAAGGCCTA |
| | CCACCAGCTGGACCAGAGCTTCATCGACACCTACATCGACCTGCTGGAAACCCG |
| | GCGGACCTACTATGAAGGACCTGGCGAGGGAAGCCCCTTCGGCTGGAAGGACAT |
| | CAAAGAATGGTACGAGATGCTGATGGGCCACTGCACCTACTTTCCCGAGGAACT |
| | GCGGAGCGTGAAGTACGCCTACAACGCCGACCTGTACAACGCCCTGAACGACCT |
| | GAACAACCTCGTGATCACCCGGGACGAGAACGAGAAGCTGGAATATTACGAGAA |
| | GTTCCAAATCATCGAGAACGTGTTCAAGCAGAAGAAAAAGCCCACGCTGAAGCA |
| | GATCGCCAAAGAGATCCTGGTCAACGAGGAAGATATCAAGGGCTACAGAGTGAC |
| | CAGCACCGGCAAGCCCGAGTTCACCAACCTGAAGGTGTACCACGACATCAAGGA |
| | TATCACAGCCCGGAAAGAAATCATTGAGAATGCCGAGCTGCTCGACCAGATTGC |
| | CAAGATCCTGACCATCTACCAGAGCAGCGAGGACATCCAAGAGGAACTGACCAA |
| | TCTGAACTCCGAGCTGACCCAAGAAGAGATCGAACAGATCAGCAATCTGAAGGG |
| | GTACACCGGCACACACAACCTGAGCCTGAAGGCCATCAACCTGATCCTGGACGA |
| | GCTGTGGCACACCAACGACAACCAGATCGCTATCTTCAACCGGCTGAAGCTGGT |
| | GCCCAAGAAGGTGGACCTGTCTCAGCAGAAAGAGATTCCCACCACACTGGTGGA |
| | CGACTTCATTCTGAGCCCCGTGGTCAAGAGAAGCTTCATCCAGAGCATCAAAGT |
| | GATCAACGCCATCATCAAGAAGTACGGGCTGCCCAACGATATCATCATCGAGCT |
| | GGCCCGCGAGAAGAACTCCAAGGACGCCCAGAAAATGATCAACGAGATGCAGAA |
| | GCGGAACCGGCAGACCAACGAGCGGATCGAGGAAATCATCCGGACCACCGGCAA |
| | AGAGAACGCCAAGTACCTGATCGAGAAGATCAAGCTGCACGACATGCAAGAGGG |
| | CAAGTGCCTGTACAGCCTGGAAGCCATTCCTCTGGAAGATCTGCTGAACAATCC |
| | CTTCAACTATGAGGTGGACCACATCATCCCCAGAAGCGTGTCCTTCGACAACAG |
| | CTTCAACAACAAGGTGCTCGTGAAGCAAGAGGAAAACAGCAAGAAGGGCAACAG |
| | AACCCCATTCCAGTACCTGAGCAGCTCCGACAGCAAGATCAGCTACGAAACCTT |
| | CAAGAAGCACATCCTGAATCTGGCCAAAGGCAAGGGCAGAATCAGCAAGACCAA |
| | GAAAGAGTATCTGCTCGAGGAACGGGACATCAACAGATTCAGCGTGCAGAAAGA |
| | CTTCATCAATCGGAACCTGGTGGACACCAGATACGCCACCAGAGGCCTGATGAA |
| | TCTGCTGCGGAGCTACTTCAGAGTGAACAATCTGGACGTGAAAGTCAAGTCCAT |
| | CAACGGCGGCTTCACCAGCTTTCTGCGGCGGAAGTGGAAGTTTAAGAAAGAGCG |
| | GAACAAGGGCTATAAGCACCATGCCGAGGACGCCCTGATCATTGCCAACGCCGA |
| | TTTCATCTTCAAAGAGTGGAAGAAACTGGACAAGGCCAAAAAAGTGATGGAAAA |
| | CCAGATGTTCGAGGAAAAGCAGGCCGAGAGCATGCCCGAGATCGAGACTGAGCA |
| | AGAGTACAAAGAGATTTTCATCACGCCCCACCAGATCAAGCACATTAAGGACTT |
| | CAAGGACTACAAGTACAGCCACCGCGTGGACAAGAAGCCCAATAGAGAGCTGAT |
| | TAACGACACCCTGTACTCTACCCGGAAGGACGACAAGGGCAATACCCTGATCGT |
| | CAACAACCTGAACGGCCTGTACGACAAGGACAACGACAAGCTCAAGAAGCTGAT |
| | CAACAAGAGCCCCGAGAAACTGCTGATGTACCATCACGACCCTCAGACCTACCA |
| | GAAACTGAAGCTGATTATGGAACAGTACGGCGACGAGAAAAACCCGCTGTACAA |
| | GTACTACGAGGAAACCGGAAACTACCTGACCAAGTACTCCAAAAAGGACAATGG |
| | GCCCGTGATCAAGAAGATTAAGTATTACGGCAACAAGCTGAATGCCCACCTGGA |
| | CATCACCGACGACTACCCCAACAGCAGAAACAAGGTGGTCAAGCTGTCCCTGAA |
| | GCCTTACAGATTCGACGTGTACCTGGACAACGGCGTGTACAAGTTCGTGACCGT |
| | GAAGAACCTGGACGTCATCAAAAAAGAAAACTACTACGAAGTCAACAGCAAGTG |
| | CTATGAGGAAGCCAAGAAACTCAAGAAAATCAGCAACCAGGCCGAGTTTATCGC |
| | CTCCTTCTACAACAACGATCTGATCAAGATCAACGGGGAGCTGTATCGCGTGAT |
| | CGGCGTCAACAATGACCTGCTGAACCGGATCGAAGTGAACATGATCGACATCAC |
| | CTACCGCGAGTACCTCGAGAACATGAACGATAAGCGGCCTCCACGGATCATTAA |
| | GACAATCGCCAGCAAGACGCAGAGCATTAAGAAGTACAGCACAGACATCCTGGG |
| | GAACCTGTACGAAGTGAAGTCCAAAAAGCACCCGCAGATTATCAAGAAAGGCGG |
| | CAGCCCCAAGAAAAAAAGAAAGGTTTCCTCCGACTACAAGGACCACGATGGCGA |
| | TTATAAGGATCACGACATCGATTACAAGGACGATGACGACAAGAGCGGCGGCAG |
| | CGGTGGATCTGGCGGAAGCACAAATCTGAGCGACATCATCGAAAAAGAGACAGG |
| | CAAGCAGCTCGTGATCCAAGAGTCCATCCTGATGCTCCCTGAAGAGGTCGAGGA |
| | AGTGATCGGCAACAAACCCGAGAGCGATATCCTGGTGCACACCGCCTACGACGA |
| | GAGCACCGACGAGAATGTCATGCTGCTGACAAGCGACGCCCCTGAGTACAAGCC |
| | TTGGGCTCTCGTGATTCAGGACAGCAATGGCGAGAACAAGATCAAGATGCTGTC |
| | CGGAGGAAGCGGAGGTAGCGGAGGGTCTACCAACCTGTCCGACATTATTGAGAA |
| | AGAAACCGGGAAGCAGCTGGTCATTCAAGAAAGTATCCTCATGTTGCCCGAAGA |
| | GGTTGAAGAAGTCATTGGGAACAAGCCTGAGTCCGACATCCTCGTGCATACAGC |
| | CTATGATGAGTCCACCGATGAGAACGTGATGCTCCTCACCTCTGATGCCCCAGA |
| | GTATAAGCCCTGGGCACTTGTCATCCAGGACTCTAACGGGGAAAACAAAATCAA |
| | AATGCTCTCCGGCGGCAGCAAGCGGACAGCCGATGGATCTGAGTTCGAGAGCCC |
| | CAAGAAGAAACGGAAGGTGTAATAGTGAGCGGCCGCTTAATTAAGCTGCCTTCT |
| | GCGGGGCTTGCCTTCTGGCCAAGCCCTTCTTCTCTCCCTTGCACCTGTACCTCT |
| | TGGTCTTTGAATAAAGCCTGAGTAGGAAGTCTAGAGTTTAAACATTTAAATCTG |
| | CAGATCCCAATGGCGCGCCGAGCTTGGCTCGAGCATGGTCATAGCTGTTTCCTG |
| | TGTGAAATTGTTATCCGCTCACAATTCCACACAACATACGAGCCGGAAGCATAA |
| | AGTGTAAAGCCTGGGGTGCCTAATGAGTGAGCTAACTCACATTAATTGCGTTGC |
| | GCTCACTGCCCGCTTTCCAGTCGGGAAACCTGTCGTGCCAGCTGCATTAATGAA |
| | TCGGCCAACGCGCGGGGAGAGGCGGTTTGCGTATTGGGCACTCTTGCGCTTCCT |
| | CGCTCACTGACTCGCTGCGCTCGGTCGTTCGGCTGCGGCGAGCGGTATCAGCTC |
| | ACTCAAAGGCGGTAATACGGTTATCCACAGAATCAGGGGATAACGCAGGAAAGA |
| | ACATGTGAGCAAAAGGCCAGCAAAAGGCCAGGAACCGTAAAAAGGCCGCGTTGC |
| | TGGCGTTTTTCCATAGGCTCCGCCCCCCTGACGAGCATCACAAAAATCGACGCT |
| | CAAGTCAGAGGTGGCGAAACCCGACAGGACTATAAAGATACCAGGCGTTTCCCC |
| | CTGGAAGCTCCCTCGTGCGCTCTCCTGTTCCGACCCTGCCGCTTACCGGATACC |
| | TGTCCGCCTTTCTCCCTTCGGGAAGCGTGGCGCTTTCTCATAGCTCACGCTGTA |
| | GGTATCTCAGTTCGGTGTAGGTCGTTCGCTCCAAGCTGGGCTGTGTGCACGAAC |
| | CCCCCGTTCAGCCCGACCGCTGCGCCTTATCCGGTAACTATCGTCTTGAGTCCA |
| | ACCCGGTAAGACACGACTTATCGCCACTGGCAGCAGCCACTGGTAACAGGATTA |
| | GCAGAGCGAGGTATGTAGGCGGTGCTACAGAGTTCTTGAAGTGGTGGCCTAACT |
| | ACGGCTACACTAGAAGAACAGTATTTGGTATCTGCGCTCTGCTGAAGCCAGTTA |
| | CCTTCGGAAAAAGAGTTGGTAGCTCTTGATCCGGCAAACAAACCACCGCTGGTA |
| | GCGGTGGTTTTTTTGTTTGCAAGCAGCAGATTACGCGCAGAAAAAAAGGATCTC |
| | AAGAAGATCCTTTGATCTTTTCTACGGGGTCTGACGCTCAGTGGAACGAAAACT |
| | CACGTTAAGGGATTTTGGTCATGAGATTATCAAAAAGGATCTTCACCTAGATCC |
| | TTTTAAATTAAAAATGAAGTTTTAAATCAATCTAAAGTATATATGAGTAAACTT |
| | GGTCTGACAGTTAGAAAAACTCATCGAGC (SEQ ID NO: 647) |
| Amino acid | MTSEKGPSTGDPTLRRRIESWEFDVFYDPRELRKETCLLYEIKWGMSRKIWRSS |
| sequence | GKNTTNHVEVNFIKKFTSERRFHSSISCSITWFLSWSPCWECSQAIREFLSQHP |
| of the | GVTLVIYVARLFWHMDQRNRQGLRDLVNSGVTIQIMRASEYYHCWRNFVNYPPG |
| polypeptide | DEAHWPQYPPLWMMLYALELHCIILSLPPCLKISRRWQNHLAFFRLHLQNCHYQ |
| encoded | TIPPHILLATGLIHPSVTWRSGGSSGGSSGSETPGTSESATPESSGGSSGGSGK |
| by | RNYILGLAIGITSVGYGIIDYETRDVIDAGVRLFKEANVENNEGRRSKRGARRL |
| mRNA2745 | KRRRRHRIQRVKKLLFDYNLLTDHSELSGINPYEARVKGLSQKLSEEEFSAALL |
| | HLAKRRGVHNVNEVEEDTGNELSTKEQISRNSKALEEKYVAELQLERLKKDGEV |
| | RGSINRFKTSDYVKEAKQLLKVQKAYHQLDQSFIDTYIDLLETRRTYYEGPGEG |
| | SPFGWKDIKEWYEMLMGHCTYFPEELRSVKYAYNADLYNALNDLNNLVITRDEN |
| | EKLEYYEKFQIIENVFKQKKKPTLKQIAKEILVNEEDIKGYRVTSTGKPEFTNL |
| | KVYHDIKDITARKEIIENAELLDQIAKILTIYQSSEDIQEELTNLNSELTQEEI |
| | EQISNLKGYTGTHNLSLKAINLILDELWHTNDNQIAIFNRLKLVPKKVDLSQQK |
| | EIPTTLVDDFILSPVVKRSFIQSIKVINAIIKKYGLPNDIIIELAREKNSKDAQ |
| | KMINEMQKRNRQTNERIEEIIRTTGKENAKYLIEKIKLHDMQEGKCLYSLEAIP |
| | LEDLLNNPFNYEVDHIIPRSVSFDNSFNNKVLVKQEENSKKGNRTPFQYLSSSD |
| | SKISYETFKKHILNLAKGKGRISKTKKEYLLEERDINRFSVQKDFINRNLVDTR |
| | YATRGLMNLLRSYFRVNNLDVKVKSINGGFTSFLRRKWKFKKERNKGYKHHAED |
| | ALIIANADFIFKEWKKLDKAKKVMENQMFEEKQAESMPEIETEQEYKEIFITPH |
| | QIKHIKDFKDYKYSHRVDKKPNRELINDTLYSTRKDDKGNTLIVNNLNGLYDKD |
| | NDKLKKLINKSPEKLLMYHHDPQTYQKLKLIMEQYGDEKNPLYKYYEETGNYLT |
| | KYSKKDNGPVIKKIKYYGNKLNAHLDITDDYPNSRNKVVKLSLKPYRFDVYLDN |
| | GVYKFVTVKNLDVIKKENYYEVNSKCYEEAKKLKKISNQAEFIASFYNNDLIKI |
| | NGELYRVIGVNNDLLNRIEVNMIDITYREYLENMNDKRPPRIIKTIASKTQSIK |
| | KYSTDILGNLYEVKSKKHPQIIKKGGSPKKKRKVSSDYKDHDGDYKDHDIDYKD |
| | DDDKSGGSGGSGGSTNLSDIIEKETGKQLVIQESILMLPEEVEEVIGNKPESDI |
| | LVHTAYDESTDENVMLLTSDAPEYKPWALVIQDSNGENKIKMLSGGSGGSGGST |
| | NLSDIIEKETGKQLVIQESILMLPEEVEEVIGNKPESDILVHTAYDESTDENVM |
| | LLTSDAPEYKPWALVIQDSNGENKIKMLSGGSKRTADGSEFESPKKKRKV |
| | (SEQ ID NO: 648) |
| Nucleotide | ATCAAATGAAACTGCAATTTATTCATATCAGGATTATCAATACCATATTTTTGA |
| sequence | AAAAGCCGTTTCTGTAATGAAGGAGAAAACTCACCGAGGCAGTTCCATAGGATG |
| of a | GCAAGATCCTGGTATCGGTCTGCGATTCCGACTCGTCCAACATCAATACAACCT |
| plasmid | ATTAATTTCCCCTCGTCAAAAATAAGGTTATCAAGTGAGAAATCACCATGAGTG |
| encoding | ACGACTGAATCCGGTGAGAATGGCAAAAGTTTATGCATTTCTTTCCAGACTTGT |
| mRNA2926 | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGAGCGAGGTCGAGTTCTCTCA |
| | CGAATATTGGATGAGACACGCTCTCACCCTGGCTAAGAGAGCCAGGGACGAAAG |
| | AGAGGTGCCAGTTGGCGCTGTCCTGGTGTTGAACAATCGCGTCATCGGAGAAGG |
| | ATGGAATCGCGCCATTGGCCTGCACGATCCAACCGCACATGCCGAAATTATGGC |
| | TCTGCGGCAAGGCGGCCTCGTGATGCAAAATTACAGACTGTACGATGCTACCCT |
| | CTACTCCACCTTCGAGCCCTGTGTCATGTGTGCTGGGGCAATGATTCACTCCCG |
| | GATTGGCCGCGTGGTGTTTGGAGTGCGGAATGCCAAGACTGGCGCCGCTGGATC |
| | TCTGATGGACGTCCTGCACCATCCTGGGATGAACCACCGGGTCGAGATCACAGA |
| | GGGAATTCTGGCTGACGAGTGCGCTGCCCTGCTGTGCAGGTTCTTTAGAATGCC |
| | TAGAAGGGTGTTCAACGCCCAGAAAAAAGCTCAGAGCAGCACCGATTCCGGCGG |
| | AAGCAGCGGAGGATCTTCTGGAAGCGAAACCCCAGGCACCAGCGAGTCTGCCAC |
| | ACCAGAATCATCTGGCGGTAGCTCCGGCGGCAGCGACAAGAAGTATTCTATCGG |
| | ACTGGCCATCGGCACCAACTCTGTTGGATGGGCCGTGATCACCGACGAGTACAA |
| | GGTGCCCAGCAAGAAATTCAAGGTGCTGGGCAACACCGACAGGCACAGCATCAA |
| | GAAGAACCTGATCGGCGCACTGCTGTTCGACTCTGGCGAAACAGCCGAGGCCAC |
| | CAGACTGAAGAGAACAGCCCGCAGACGGTACACCAGAAGAAAGAACCGGATCTG |
| | CTACCTCCAAGAGATCTTCAGCAACGAGATGGCCAAGGTGGACGACAGCTTCTT |
| | CCACAGACTGGAAGAGTCCTTCCTGGTGGAAGAGGACAAGAAGCACGAGAGACA |
| | CCCCATCTTCGGCAACATCGTGGACGAGGTGGCCTACCACGAGAAGTACCCCAC |
| | CATCTACCACCTGAGAAAGAAACTGGTGGACAGCACCGACAAGGCCGACCTGAG |
| | ACTGATCTATCTGGCCCTGGCTCACATGATCAAGTTCCGGGGCCACTTCCTGAT |
| | CGAGGGCGACCTGAATCCTGACAACAGCGACGTGGACAAGCTGTTCATCCAGCT |
| | GGTGCAGACCTACAACCAGCTGTTCGAGGAAAACCCCATCAACGCCAGCGGAGT |
| | GGATGCCAAGGCCATCCTGTCTGCCAGACTGAGCAAGAGCAGACGGCTGGAAAA |
| | TCTGATCGCCCAGCTGCCTGGCGAGAAGAAGAATGGCCTGTTCGGCAACCTGAT |
| | TGCCCTGAGCCTGGGCCTGACACCTAACTTCAAGAGCAACTTCGACCTGGCCGA |
| | GGACGCCAAACTGCAGCTGAGCAAGGACACCTACGACGACGACCTGGACAATCT |
| | GCTGGCCCAGATCGGCGATCAGTACGCCGACTTGTTTCTGGCCGCCAAGAATCT |
| | GAGCGACGCCATCCTGCTGTCCGACATCCTGAGAGTGAACACCGAGATCACCAA |
| | GGCACCTCTGAGCGCCTCTATGATCAAGAGATACGACGAGCACCACCAGGATCT |
| | GACCCTGCTGAAGGCCCTCGTTAGACAGCAGCTGCCAGAGAAGTACAAAGAGAT |
| | TTTCTTCGACCAGAGCAAGAACGGCTACGCCGGCTACATTGATGGCGGAGCCAG |
| | CCAAGAGGAATTCTACAAGTTCATCAAGCCCATCCTCGAGAAGATGGACGGCAC |
| | CGAGGAACTGCTGGTCAAGCTGAACAGAGAGGACCTGCTGAGAAAGCAGAGAAC |
| | CTTCGACAACGGCAGCATCCCTCACCAGATCCACCTGGGAGAACTGCACGCCAT |
| | TCTGCGGAGACAAGAGGACTTTTACCCATTCCTGAAGGACAACCGGGAAAAGAT |
| | CGAGAAAATCCTGACCTTCAGGATCCCCTACTACGTGGGACCACTGGCCAGAGG |
| | CAATAGCAGATTCGCCTGGATGACCAGAAAGAGCGAGGAAACCATCACTCCCTG |
| | GAACTTCGAGGAAGTGGTGGACAAGGGCGCCAGCGCTCAGTCCTTCATCGAGCG |
| | GATGACCAACTTCGATAAGAACCTGCCTAACGAGAAGGTGCTGCCCAAGCACAG |
| | CCTGCTGTACGAGTACTTCACCGTGTACAACGAGCTGACCAAAGTGAAATACGT |
| | GACCGAGGGAATGAGAAAGCCCGCCTTTCTGAGCGGCGAGCAGAAAAAGGCCAT |
| | CGTGGATCTGCTGTTCAAGACCAACCGGAAAGTGACCGTGAAGCAGCTGAAAGA |
| | GGACTACTTCAAGAAAATCGAGTGCTTCGACAGCGTCGAGATCTCCGGCGTGGA |
| | AGATCGGTTCAATGCCAGCCTGGGCACATACCACGATCTGCTGAAAATTATCAA |
| | GGACAAGGACTTCCTGGACAACGAAGAGAACGAGGACATCCTTGAGGACATCGT |
| | GCTGACACTGACCCTGTTTGAGGACAGAGAGATGATCGAGGAACGGCTGAAAAC |
| | ATACGCCCACCTGTTCGACGACAAAGTGATGAAGCAACTGAAGCGGCGGAGATA |
| | CACCGGCTGGGGCAGACTGTCTCGGAAGCTGATCAACGGCATCCGGGATAAGCA |
| | GTCCGGCAAGACCATCCTGGACTTTCTGAAGTCCGACGGCTTCGCCAACAGAAA |
| | CTTCATGCAGCTGATTCACGACGACAGCCTCACCTTCAAAGAGGATATCCAGAA |
| | AGCCCAGGTGTCCGGCCAGGGCGATTCTCTGCATGAGCACATTGCCAACCTGGC |
| | CGGCTCTCCCGCCATTAAGAAAGGCATCCTGCAGACAGTGAAGGTGGTGGACGA |
| | GCTTGTGAAAGTGATGGGCAGACACAAGCCCGAGAACATCGTGATCGAAATGGC |
| | CAGAGAGAACCAGACCACACAGAAGGGACAGAAGAACAGCCGCGAGAGAATGAA |
| | GCGGATCGAAGAGGGCATCAAAGAGCTGGGCAGCCAGATCCTGAAAGAACACCC |
| | CGTGGAAAACACCCAGCTGCAGAACGAGAAGCTGTACCTGTACTACCTGCAGAA |
| | TGGACGGGATATGTACGTGGACCAAGAGCTGGACATCAACAGACTGTCCGACTA |
| | CGATGTGGACCATATCGTGCCCCAGTCTTTTCTGAAGGACGACTCCATCGACAA |
| | CAAGGTCCTGACCAGATCCGACAAGAATCGGGGCAAGAGCGACAACGTGCCCTC |
| | CGAAGAGGTGGTCAAGAAGATGAAGAACTACTGGCGACAGCTGCTGAACGCCAA |
| | GCTGATTACCCAGCGGAAGTTCGACAATCTGACCAAGGCCGAAAGAGGCGGCCT |
| | GAGCGAACTGGATAAGGCCGGCTTCATCAAGAGACAGCTGGTGGAAACCCGGCA |
| | GATCACAAAGCACGTGGCACAGATTCTGGACTCTCGGATGAACACTAAGTACGA |
| | CGAGAACGACAAACTGATCCGCGAAGTGAAAGTCATCACCCTGAAGTCCAAGCT |
| | GGTGTCCGATTTCCGGAAGGATTTCCAGTTCTACAAAGTGCGCGAGATCAACAA |
| | CTACCATCACGCCCACGACGCCTACCTGAATGCCGTTGTTGGAACAGCCCTGAT |
| | CAAAAAGTACCCTAAGCTGGAAAGCGAGTTCGTGTACGGCGACTACAAGGTGTA |
| | CGACGTGCGGAAGATGATCGCCAAGAGCGAGCAAGAGATTGGCAAGGCAACCGC |
| | CAAGTACTTCTTCTACAGCAACATCATGAACTTTTTCAAGACAGAGATCACCCT |
| | CGCCAACGGCGAGATCAGAAAGCGGCCTCTGATCGAGACAAACGGCGAAACCGG |
| | CGAGATTGTGTGGGATAAGGGCAGAGACTTTGCCACAGTGCGGAAAGTGCTGAG |
| | CATGCCCCAAGTGAATATCGTGAAGAAAACCGAGGTGCAGACAGGCGGCTTCAG |
| | CAAAGAGTCTATCCTGCCTAAGCGGAACTCCGACAAGCTGATCGCCAGAAAGAA |
| | GGACTGGGACCCCAAGAAGTACGGCGGCTTCGATTCTCCTACCGTGGCCTATAG |
| | CGTGCTGGTGGTGGCCAAAGTGGAAAAGGGCAAGTCCAAGAAACTCAAGAGCGT |
| | GAAAGAGCTGCTGGGGATCACCATCATGGAAAGAAGCAGCTTCGAGAAGAATCC |
| | GATCGATTTCCTCGAGGCCAAGGGCTACAAAGAAGTGAAAAAGGACCTGATCAT |
| | CAAGCTCCCCAAGTACTCCCTGTTCGAGCTGGAAAACGGCCGGAAGAGAATGCT |
| | GGCCTCTGCTGGCGAACTGCAGAAGGGAAACGAACTGGCCCTGCCTAGCAAATA |
| | TGTGAACTTCCTGTACCTGGCCAGCCACTATGAGAAGCTGAAGGGCAGCCCCGA |
| | GGACAATGAGCAAAAGCAGCTGTTTGTGGAACAGCACAAGCACTACCTGGACGA |
| | GATCATCGAGCAGATCAGCGAGTTTAGCAAGAGAGTGATTCTGGCCGACGCCAA |
| | TCTGGACAAAGTGCTGTCCGCCTACAACAAGCACCGGGACAAGCCTATCAGAGA |
| | GCAGGCCGAGAATATCATCCACCTGTTTACCCTGACCAACCTGGGAGCCCCTGC |
| | CGCCTTCAAGTACTTTGACACCACCATCGACCGGAAGCGGTACACCTCCACCAA |
| | AGAGGTGCTGGACGCCACTCTGATCCACCAGTCTATCACCGGCCTGTACGAGAC |
| | ACGGATCGACCTGTCTCAACTCGGAGGCGACGAAGGCGCCGATAAGAGAACCGC |
| | CGATGGCTCTGAGTTCGAGAGCCCCAAGAAAAAGCGCAAAGTGTAATAGTGAGC |
| | GGCCGCTTAATTAAGCTGCCTTCTGCGGGGCTTGCCTTCTGGCCAAGCCCTTCT |
| | TCTCTCCCTTGCACCTGTACCTCTTGGTCTTTGAATAAAGCCTGAGTAGGAAGT |
| | CTAGAGTTTAAACATTTAAATCTGCAGATCCCAATGGCGCGCCGAGCTTGGCTC |
| | GAGCATGGTCATAGCTGTTTCCTGTGTGAAATTGTTATCCGCTCACAATTCCAC |
| | ACAACATACGAGCCGGAAGCATAAAGTGTAAAGCCTGGGGTGCCTAATGAGTGA |
| | GCTAACTCACATTAATTGCGTTGCGCTCACTGCCCGCTTTCCAGTCGGGAAACC |
| | TGTCGTGCCAGCTGCATTAATGAATCGGCCAACGCGCGGGGAGAGGCGGTTTGC |
| | GTATTGGGCACTCTTGCGCTTCCTCGCTCACTGACTCGCTGCGCTCGGTCGTTC |
| | GGCTGCGGCGAGCGGTATCAGCTCACTCAAAGGCGGTAATACGGTTATCCACAG |
| | AATCAGGGGATAACGCAGGAAAGAACATGTGAGCAAAAGGCCAGCAAAAGGCCA |
| | GGAACCGTAAAAAGGCCGCGTTGCTGGCGTTTTTCCATAGGCTCCGCCCCCCTG |
| | ACGAGCATCACAAAAATCGACGCTCAAGTCAGAGGTGGCGAAACCCGACAGGAC |
| | TATAAAGATACCAGGCGTTTCCCCCTGGAAGCTCCCTCGTGCGCTCTCCTGTTC |
| | CGACCCTGCCGCTTACCGGATACCTGTCCGCCTTTCTCCCTTCGGGAAGCGTGG |
| | CGCTTTCTCATAGCTCACGCTGTAGGTATCTCAGTTCGGTGTAGGTCGTTCGCT |
| | CCAAGCTGGGCTGTGTGCACGAACCCCCCGTTCAGCCCGACCGCTGCGCCTTAT |
| | CCGGTAACTATCGTCTTGAGTCCAACCCGGTAAGACACGACTTATCGCCACTGG |
| | CAGCAGCCACTGGTAACAGGATTAGCAGAGCGAGGTATGTAGGCGGTGCTACAG |
| | AGTTCTTGAAGTGGTGGCCTAACTACGGCTACACTAGAAGAACAGTATTTGGTA |
| | TCTGCGCTCTGCTGAAGCCAGTTACCTTCGGAAAAAGAGTTGGTAGCTCTTGAT |
| | CCGGCAAACAAACCACCGCTGGTAGCGGTGGTTTTTTTGTTTGCAAGCAGCAGA |
| | TTACGCGCAGAAAAAAAGGATCTCAAGAAGATCCTTTGATCTTTTCTACGGGGT |
| | CTGACGCTCAGTGGAACGAAAACTCACGTTAAGGGATTTTGGTCATGAGATTAT |
| | CAAAAAGGATCTTCACCTAGATCCTTTTAAATTAAAAATGAAGTTTTAAATCAA |
| | TCTAAAGTATATATGAGTAAACTTGGTCTGACAGTTAGAAAAACTCATCGAGC |
| | (SEQ ID NO: 649) |
| Amino acid | MSEVEFSHEYWMRHALTLAKRARDEREVPVGAVLVLNNRVIGEGWNRAIGLHDP |
| sequence | TAHAEIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSDKKYSIGLAIGTNSVGW |
| encoded | AVITDEYKVPSKKFKVLGNTDRHSIKKNLIGALLFDSGETAEATRLKRTARRRY |
| by | TRRKNRICYLQEIFSNEMAKVDDSFFHRLEESFLVEEDKKHERHPIFGNIVDEV |
| mRNA2926 | AYHEKYPTIYHLRKKLVDSTDKADLRLIYLALAHMIKFRGHFLIEGDLNPDNSD |
| | VDKLFIQLVQTYNQLFEENPINASGVDAKAILSARLSKSRRLENLIAQLPGEKK |
| | NGLFGNLIALSLGLTPNFKSNFDLAEDAKLQLSKDTYDDDLDNLLAQIGDQYAD |
| | LFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNRE |
| | DLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPN |
| | EKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWGRLSRKL |
| | INGIRDKQSGKTILDFLKSDGFANRNFMQLIHDDSLTFKEDIQKAQVSGQGDSL |
| | HEHIANLAGSPAIKKGILQTVKVVDELVKVMGRHKPENIVIEMARENQTTQKGQ |
| | KNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVAQILD |
| | SRMNTKYDENDKLIREVKVITLKSKLVSDFRKDFQFYKVREINNYHHAHDAYLN |
| | AVVGTALIKKYPKLESEFVYGDYKVYDVRKMIAKSEQEIGKATAKYFFYSNIMN |
| | FFKTEITLANGEIRKRPLIETNGETGEIVWDKGRDFATVRKVLSMPQVNIVKKT |
| | EVQTGGFSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPKYSLFEL |
| | ENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFT |
| | LTNLGAPAAFKYFDTTIDRKRYTSTKEVLDATLIHQSITGLYETRIDLSQLGGD |
| | EGADKRTADGSEFESPKKKRKV (SEQ ID NO: 650) |
| Nucleotide | CATCAAATGAAACTGCAATTTATTCATATCAGGATTATCAATACCATATTTTTG |
| sequence | AAAAAGCCGTTTCTGTAATGAAGGAGAAAACTCACCGAGGCAGTTCCATAGGAT |
| of a | GGCAAGATCCTGGTATCGGTCTGCGATTCCGACTCGTCCAACATCAATACAACC |
| plasmid | TATTAATTTCCCCTCGTCAAAAATAAGGTTATCAAGTGAGAAATCACCATGAGT |
| encoding | GACGACTGAATCCGGTGAGAATGGCAAAAGTTTATGCATTTCTTTCCAGACTTG |
| mRNA2892 | TTCAACAGGCCAGCCATTACGCTCGTCATCAAAATCACTCGCATCAACCAAACC |
| | GTTATTCATTCGTGATTGCGCCTGAGCGAGACGAAATACGCGATCGCTGTTAAA |
| | AGGACAATTACAAACAGGAATCGAATGCAACCGGCGCAGGAACACTGCCAGCGC |
| | ATCAACAATATTTTCACCTGAATCAGGATATTCTTCTAATACCTGGAATGCTGT |
| | TTTCCCAGGGATCGCAGTGGTGAGTAACCATGCATCATCAGGAGTACGGATAAA |
| | ATGCTTGATGGTCGGAAGAGGCATAAATTCCGTCAGCCAGTTTAGTCTGACCAT |
| | CTCATCTGTAACATCATTGGCAACGCTACCTTTGCCATGTTTCAGAAACAACTC |
| | TGGCGCATCGGGCTTCCCATACAATCGATAGATTGTCGCACCTGATTGCCCGAC |
| | ATTATCGCGAGCCCATTTATACCCATATAAATCAGCATCCATGTTGGAATTTAA |
| | TCGCGGCCTAGAGCAAGACGTTTCCCGTTGAATATGGCTCATACTCTTCCTTTT |
| | TCAATATTATTGAAGCATTTATCAGGGTTATTGTCTCATGAGCGGATACATATT |
| | TGAATGTATTTAGAAAAATAAACAAATAGGGGTTCCGCGCACATTTCCCCGAAA |
| | AGTGCCACCTGACGTCTAAGAAACCATTATTATCATGACATTAACCTATAAAAA |
| | TAGGCGTATCACGAGGCCCTTTCGTCTCGCGCGTTTCGGTGATGACGGTGAAAA |
| | CCTCTGACACATGCAGCTCCCGGAGACGGTCACAGCTTGTCTGTAAGCGGATGC |
| | CGGGAGCAGACAAGCCCGTCAGGGCGCGTCAGCGGGTGTTGGCGGGTGTCGGGG |
| | CTGGCTTAACTATGCGGCATCAGAGCAGATTGTACTGAGAGTGCACCATATGCG |
| | GTGTGAAATACCGCACAGATGCGTAAGGAGAAAATACCGCATCAGGCGCCATTC |
| | GCCATTCAGGCTGCGCAACTGTTGGGAAGGGCGATCGGTGCGGGCCTCTTCGCT |
| | ATTACGCCAGCTGGCGAAAGGGGGATGTGCTGCAAGGCGATTAAGTTGGGTAAC |
| | GCCAGGGTTTTCCCAGTCACGACGTTGTAAAACGACGGCCAGTGAATTGACGCG |
| | TATTGGGATCGAGCTCGGTACCTAATACGACACACTATAAGGAAATAAGAGAGA |
| | AAAGAAGAGTAAGAAGAAATATAAGAGCCACCATGAGCGAGGTCGAGTTCTCTC |
| | ACGAATATTGGATGAGACACGCTCTCACCCTGGCTAAGAGAGCCAGGGACGAAA |
| | GAGAGGTGCCAGTTGGCGCTGTCCTGGTGTTGAACAATCGCGTCATCGGAGAAG |
| | GATGGAATCGCGCCATTGGCCTGCACGATCCAACCGCACATGCCGAAATTATGG |
| | CTCTGCGGCAAGGCGGCCTCGTGATGCAAAATTACAGACTGTACGATGCTACCC |
| | TCTACTCCACCTTCGAGCCCTGTGTCATGTGTGCTGGGGCAATGATTCACTCCC |
| | GGATTGGCCGCGTGGTGTTTGGAGTGCGGAATGCCAAGACTGGCGCCGCTGGAT |
| | CTCTGATGGACGTCCTGCACCATCCTGGGATGAACCACCGGGTCGAGATCACAG |
| | AGGGAATTCTGGCTGACGAGTGCGCTGCCCTGCTGTGCAGGTTCTTTAGAATGC |
| | CTAGAAGGGTGTTCAACGCCCAGAAAAAAGCTCAGAGCAGCACCGATTCCGGCG |
| | GAAGCAGCGGAGGATCTTCTGGAAGCGAAACCCCAGGCACCAGCGAGTCTGCCA |
| | CACCAGAATCATCTGGCGGTAGCTCCGGCGGCAGCAAGAGAAATTACATCCTCG |
| | GACTGGCCATCGGCATCACCTCTGTCGGCTACGGCATCATCGACTACGAGACAC |
| | GGGACGTGATCGATGCCGGCGTGCGGCTGTTTAAAGAGGCCAACGTCGAGAACA |
| | ACGAGGGCCGCAGATCTAAGAGAGGCGCCAGACGGCTGAAGAGAAGAAGAAGGC |
| | ACCGGATCCAGAGAGTGAAGAAGCTGCTGTTCGACTACAACCTGCTGACCGACC |
| | ACAGCGAGCTGAGCGGCATCAATCCTTACGAGGCCAGAGTGAAGGGCCTGAGCC |
| | AGAAGCTGAGCGAGGAAGAGTTTAGCGCTGCCCTGCTGCACCTGGCCAAGAGAA |
| | GAGGCGTGCACAACGTGAACGAGGTGGAAGAGGACACCGGCAACGAGCTGTCCA |
| | CCAAAGAGCAGATCAGCCGGAACAGCAAGGCCCTGGAAGAGAAATACGTGGCCG |
| | AACTGCAGCTGGAACGGCTGAAAAAGGATGGCGAAGTGCGGGGCAGCATCAACC |
| | GGTTCAAGACCAGCGACTACGTGAAAGAAGCCAAACAGCTGCTGAAGGTGCAGA |
| | AGGCCTACCACCAGCTGGACCAGAGCTTCATCGACACCTACATCGACCTGCTGG |
| | AAACCCGGCGGACCTACTATGAAGGACCTGGCGAGGGAAGCCCCTTCGGCTGGA |
| | AGGACATCAAAGAATGGTACGAGATGCTGATGGGCCACTGCACCTACTTTCCCG |
| | AGGAACTGCGGAGCGTGAAGTACGCCTACAACGCCGACCTGTACAACGCCCTGA |
| | ACGACCTGAACAACCTCGTGATCACCCGGGACGAGAACGAGAAGCTGGAATATT |
| | ACGAGAAGTTCCAAATCATCGAGAACGTGTTCAAGCAGAAGAAAAAGCCCACGC |
| | TGAAGCAGATCGCCAAAGAGATCCTGGTCAACGAGGAAGATATCAAGGGCTACA |
| | GAGTGACCAGCACCGGCAAGCCCGAGTTCACCAACCTGAAGGTGTACCACGACA |
| | TCAAGGATATCACAGCCCGGAAAGAAATCATTGAGAATGCCGAGCTGCTCGACC |
| | AGATTGCCAAGATCCTGACCATCTACCAGAGCAGCGAGGACATCCAAGAGGAAC |
| | TGACCAATCTGAACTCCGAGCTGACCCAAGAAGAGATCGAACAGATCAGCAATC |
| | TGAAGGGGTACACCGGCACACACAACCTGAGCCTGAAGGCCATCAACCTGATCC |
| | TGGACGAGCTGTGGCACACCAACGACAACCAGATCGCTATCTTCAACCGGCTGA |
| | AGCTGGTGCCCAAGAAGGTGGACCTGTCTCAGCAGAAAGAGATTCCCACCACAC |
| | TGGTGGACGACTTCATTCTGAGCCCCGTGGTCAAGAGAAGCTTCATCCAGAGCA |
| | TCAAAGTGATCAACGCCATCATCAAGAAGTACGGGCTGCCCAACGATATCATCA |
| | TCGAGCTGGCCCGCGAGAAGAACTCCAAGGACGCCCAGAAAATGATCAACGAGA |
| | TGCAGAAGCGGAACCGGCAGACCAACGAGCGGATCGAGGAAATCATCCGGACCA |
| | CCGGCAAAGAGAACGCCAAGTACCTGATCGAGAAGATCAAGCTGCACGACATGC |
| | AAGAGGGCAAGTGCCTGTACAGCCTGGAAGCCATTCCTCTGGAAGATCTGCTGA |
| | ACAATCCCTTCAACTATGAGGTGGACCACATCATCCCCAGAAGCGTGTCCTTCG |
| | ACAACAGCTTCAACAACAAGGTGCTCGTGAAGCAAGAGGAAAACAGCAAGAAGG |
| | GCAACAGAACCCCATTCCAGTACCTGAGCAGCTCCGACAGCAAGATCAGCTACG |
| | AAACCTTCAAGAAGCACATCCTGAATCTGGCCAAAGGCAAGGGCAGAATCAGCA |
| | AGACCAAGAAAGAGTATCTGCTCGAGGAACGGGACATCAACAGATTCAGCGTGC |
| | AGAAAGACTTCATCAATCGGAACCTGGTGGACACCAGATACGCCACCAGAGGCC |
| | TGATGAATCTGCTGCGGAGCTACTTCAGAGTGAACAATCTGGACGTGAAAGTCA |
| | AGTCCATCAACGGCGGCTTCACCAGCTTTCTGCGGCGGAAGTGGAAGTTTAAGA |
| | AAGAGCGGAACAAGGGCTATAAGCACCATGCCGAGGACGCCCTGATCATTGCCA |
| | ACGCCGATTTCATCTTCAAAGAGTGGAAGAAACTGGACAAGGCCAAAAAAGTGA |
| | TGGAAAACCAGATGTTCGAGGAAAAGCAGGCCGAGAGCATGCCCGAGATCGAGA |
| | CTGAGCAAGAGTACAAAGAGATTTTCATCACGCCCCACCAGATCAAGCACATTA |
| | AGGACTTCAAGGACTACAAGTACAGCCACCGCGTGGACAAGAAGCCCAACCGGA |
| | AGCTGATCAATGACACACTGTACAGCACCCGGAAGGACGACAAGGGCAATACCC |
| | TGATCGTCAACAACCTGAACGGCCTGTACGACAAGGACAACGACAAGCTCAAGA |
| | AGCTGATTAACAAGAGCCCCGAGAAGCTCCTGATGTACCATCACGACCCTCAGA |
| | CCTACCAGAAACTGAAGCTCATCATGGAACAGTACGGCGACGAGAAAAACCCGC |
| | TGTACAAGTACTACGAGGAAACCGGAAACTACCTGACCAAGTACTCCAAAAAGG |
| | ACAATGGGCCCGTGATCAAGAAGATTAAGTATTACGGCAACAAGCTGAATGCCC |
| | ACCTGGACATCACCGACGACTACCCCAACAGCAGAAACAAGGTGGTCAAGCTGT |
| | CCCTGAAGCCTTACAGATTCGACGTGTACCTGGACAACGGCGTGTACAAGTTCG |
| | TGACCGTGAAGAACCTGGACGTCATCAAAAAAGAAAACTACTACGAAGTCAACA |
| | GCAAGTGCTATGAGGAAGCCAAGAAACTCAAGAAAATCAGCAACCAGGCCGAGT |
| | TTATCGCCAGCTTCTACAAGAACGACCTCATCAAGATCAACGGGGAGCTGTATA |
| | GAGTGATCGGGGTCAACAATGACCTGCTGAACCGGATCGAAGTGAACATGATCG |
| | ACATCACCTACCGCGAGTACCTCGAGAACATGAACGATAAGCGGCCACCGCACA |
| | TCATTAAGACAATCGCCAGCAAGACGCAGAGCATTAAGAAGTACAGCACAGACA |
| | TCCTGGGGAACCTGTACGAAGTGAAGTCCAAAAAGCACCCGCAGATTATCAAGA |
| | AAGGCGGCAGCCCCAAGAAAAAAAGAAAGGTTTCCTCCGACTACAAGGACCACG |
| | ATGGCGATTATAAGGATCACGACATCGATTACAAGGACGATGACGACAAGGAAG |
| | GCGCCGATAAGAGAACCGCCGATGGCTCTGAGTTCGAGAGCCCCAAGAAAAAGC |
| | GCAAAGTGTAATAGTGAGCGGCCGCTTAATTAAGCTGCCTTCTGCGGGGCTTGC |
| | CTTCTGGCCAAGCCCTTCTTCTCTCCCTTGCACCTGTACCTCTTGGTCTTTGAA |
| | TAAAGCCTGAGTAGGAAGTCTAGAGTTTAAACATTTAAATCTGCAGATCCCAAT |
| | GGCGCGCCGAGCTTGGCTCGAGCATGGTCATAGCTGTTTCCTGTGTGAAATTGT |
| | TATCCGCTCACAATTCCACACAACATACGAGCCGGAAGCATAAAGTGTAAAGCC |
| | TGGGGTGCCTAATGAGTGAGCTAACTCACATTAATTGCGTTGCGCTCACTGCCC |
| | GCTTTCCAGTCGGGAAACCTGTCGTGCCAGCTGCATTAATGAATCGGCCAACGC |
| | GCGGGGAGAGGCGGTTTGCGTATTGGGCACTCTTGCGCTTCCTCGCTCACTGAC |
| | TCGCTGCGCTCGGTCGTTCGGCTGCGGCGAGCGGTATCAGCTCACTCAAAGGCG |
| | GTAATACGGTTATCCACAGAATCAGGGGATAACGCAGGAAAGAACATGTGAGCA |
| | AAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGG |
| | TGGCGAAACCCGACAGGACTATAAAGATACCAGGCGTTTCCCCCTGGAAGCTCC |
| | CTCGTGCGCTCTCCTGTTCCGACCCTGCCGCTTACCGGATACCTGTCCGCCTTT |
| | CTCCCTTCGGGAAGCGTGGCGCTTTCTCATAGCTCACGCTGTAGGTATCTCAGT |
| | TCGGTGTAGGTCGTTCGCTCCAAGCTGGGCTGTGTGCACGAACCCCCCGTTCAG |
| | CCCGACCGCTGCGCCTTATCCGGTAACTATCGTCTTGAGTCCAACCCGGTAAGA |
| | CACGACTTATCGCCACTGGCAGCAGCCACTGGTAACAGGATTAGCAGAGCGAGG |
| | TATGTAGGCGGTGCTACAGAGTTCTTGAAGTGGTGGCCTAACTACGGCTACACT |
| | AGAAGAACAGTATTTGGTATCTGCGCTCTGCTGAAGCCAGTTACCTTCGGAAAA |
| | AGAGTTGGTAGCTCTTGATCCGGCAAACAAACCACCGCTGGTAGCGGTGGTTTT |
| | TTTGTTTGCAAGCAGCAGATTACGCGCAGAAAAAAAGGATCTCAAGAAGATCCT |
| | TTGATCTTTTCTACGGGGTCTGACGCTCAGTGGAACGAAAACTCACGTTAAGGG |
| | ATTTTGGTCATGAGATTATCAAAAAGGATCTTCACCTAGATCCTTTTAAATTAA |
| | AAATGAAGTTTTAAATCAATCTAAAGTATATATGAGTAAACTTGGTCTGACAGT |
| | TAGAAAAACTCATCGAG (SEQ ID NO: 651) |
| Amino acid | MSEVEFSHEYWMRHALTLAKRARDEREVPVGAVLVLNNRVIGEGWNRAIGLHDP |
| sequence | TAHAEIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSKRNYILGLAIGITSVGY |
| encoded | GIIDYETRDVIDAGVRLFKEANVENNEGRRSKRGARRLKRRRRHRIQRVKKLLF |
| by | DYNLLTDHSELSGINPYEARVKGLSQKLSEEEFSAALLHLAKRRGVHNVNEVEE |
| mRNA2892 | DTGNELSTKEQISRNSKALEEKYVAELQLERLKKDGEVRGSINRFKTSDYVKEA |
| | KQLLKVQKAYHQLDQSFIDTYIDLLETRRTYYEGPGEGSPFGWKDIKEWYEMLM |
| | GHCTYFPEELRSVKYAYNADLYNALNDLNNLVITRDENEKLEYYEKFQIIENVF |
| | KQKKKPTLKQIAKEILVNEEDIKGYRVTSTGKPEFTNLKVYHDIKDITARKEII |
| | ENAELLDQIAKILTIYQSSEDIQEELTNLNSELTQEEIEQISNLKGYTGTHNLS |
| | LKAINLILDELWHTNDNQIAIFNRLKLVPKKVDLSQQKEIPTTLVDDFILSPVV |
| | KRSFIQSIKVINAIIKKYGLPNDIIIELAREKNSKDAQKMINEMQKRNRQTNER |
| | IEEIIRTTGKENAKYLIEKIKLHDMQEGKCLYSLEAIPLEDLLNNPFNYEVDHI |
| | IPRSVSFDNSFNNKVLVKQEENSKKGNRTPFQYLSSSDSKISYETFKKHILNLA |
| | KGKGRISKTKKEYLLEERDINRFSVQKDFINRNLVDTRYATRGLMNLLRSYFRV |
| | NNLDVKVKSINGGFTSFLRRKWKFKKERNKGYKHHAEDALIIANADFIFKEWKK |
| | LDKAKKVMENQMFEEKQAESMPEIETEQEYKEIFITPHQIKHIKDFKDYKYSHR |
| | VDKKPNRKLINDTLYSTRKDDKGNTLIVNNLNGLYDKDNDKLKKLINKSPEKLL |
| | MYHHDPQTYQKLKLIMEQYGDEKNPLYKYYEETGNYLTKYSKKDNGPVIKKIKY |
| | YGNKLNAHLDITDDYPNSRNKVVKLSLKPYRFDVYLDNGVYKFVTVKNLDVIKK |
| | ENYYEVNSKCYEEAKKLKKISNQAEFIASFYKNDLIKINGELYRVIGVNNDLLN |
| | RIEVNMIDITYREYLENMNDKRPPHIIKTIASKTQSIKKYSTDILGNLYEVKSK |
| | KHPQIIKKGGSPKKKRKVSSDYKDHDGDYKDHDIDYKDDDDKEGADKRTADGSE |
| | FESPKKKRKV (SEQ ID NO: 652) |
| Nucleotide | TCTAGAGTTTAAACATTTAAATCTGCAGATCCCAATGGCGCGCCGAGCTTGGCT |
| sequence | CGAGCATGGTCATAGCTGTTTCCTGTGTGAAATTGTTATCCGCTCACAATTCCA |
| of a | CACAACATACGAGCCGGAAGCATAAAGTGTAAAGCCTGGGGTGCCTAATGAGTG |
| plasmid | AGCTAACTCACATTAATTGCGTTGCGCTCACTGCCCGCTTTCCAGTCGGGAAAC |
| encoding | CTGTCGTGCCAGCTGCATTAATGAATCGGCCAACGCGCGGGGAGAGGCGGTTTG |
| mRNA2970 | CGTATTGGGCACTCTTGCGCTTCCTCGCTCACTGACTCGCTGCGCTCGGTCGTT |
| | CGGCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGGCC |
| | AGGAACCGTAAAAAGGCCGCGTTGCTGGCGTTTTTCCATAGGCTCCGCCCCCCT |
| | GACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTT |
| | CCGACCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTACGGGG |
| | TCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCA |
| | ATCTAAAGTATATATGAGTAAACTTGGTCTGACAGTTAGAAAAACTCATCGAGC |
| | ATCAAATGAAACTGCAATTTATTCATATCAGGATTATCAATACCATATTTTTGA |
| | AAAAGCCGTTTCTGTAATGAAGGAGAAAACTCACCGAGGCAGTTCCATAGGATG |
| | GCAAGATCCTGGTATCGGTCTGCGATTCCGACTCGTCCAACATCAATACAACCT |
| | ATTAATTTCCCCTCGTCAAAAATAAGGTTATCAAGTGAGAAATCACCATGAGTG |
| | ACGACTGAATCCGGTGAGAATGGCAAAAGTTTATGCATTTCTTTCCAGACTTGT |
| | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGAGCGAGGTCGAGTTCTCTCA |
| | CGAATATTGGATGAGACACGCTCTCACCCTGGCTAAGAGAGCCAGGGACGAAAG |
| | AGAGGTGCCAGTTGGCGCTGTCCTGGTGTTGAACAATCGCGTCATCGGAGAAGG |
| | ATGGAATCGCGCCATTGGCCTGCACGATCCAACCGCACATGCCCAGATTATGGC |
| | TCTGCGGCAAGGCGGCCTCGTGATGCAAAATTACAGACTGTACGATGCTACCCT |
| | CTACTCCACCTTCGAGCCCTGTGTCATGTGTGCTGGGGCAATGATTCACTCCCG |
| | GATTGGCCGCGTGGTGTTTGGAGTGCGGAATGCCAAGACTGGCGCCGCTGGATC |
| | TCTGATGGACGTCCTGCACCATCCTGGGATGAACCACCGGGTCGAGATCACAGA |
| | GGGAATTCTGGCTGACGAGTGCGCTGCCCTGCTGTGCAGGTTCTTTAGAATGCC |
| | TAGAAGGGTGTTCAACGCCCAGAAAAAAGCTCAGAGCAGCACCGATTCCGGCGG |
| | AAGCAGCGGAGGATCTTCTGGAAGCGAAACCCCAGGCACCAGCGAGTCTGCCAC |
| | ACCAGAATCATCTGGCGGTAGCTCCGGCGGCAGCGACAAGAAGTATTCTATCGG |
| | ACTGGCCATCGGCACCAACTCTGTTGGATGGGCCGTGATCACCGACGAGTACAA |
| | GGTGCCCAGCAAGAAATTCAAGGTGCTGGGCAACACCGACAGGCACAGCATCAA |
| | GAAGAACCTGATCGGCGCACTGCTGTTCGACTCTGGCGAAACAGCCGAGGCCAC |
| | CAGACTGAAGAGAACAGCCCGCAGACGGTACACCAGAAGAAAGAACCGGATCTG |
| | CTACCTCCAAGAGATCTTCAGCAACGAGATGGCCAAGGTGGACGACAGCTTCTT |
| | CCACAGACTGGAAGAGTCCTTCCTGGTGGAAGAGGACAAGAAGCACGAGAGACA |
| | CCCCATCTTCGGCAACATCGTGGACGAGGTGGCCTACCACGAGAAGTACCCCAC |
| | CATCTACCACCTGAGAAAGAAACTGGTGGACAGCACCGACAAGGCCGACCTGAG |
| | ACTGATCTATCTGGCCCTGGCTCACATGATCAAGTTCCGGGGCCACTTCCTGAT |
| | CGAGGGCGACCTGAATCCTGACAACAGCGACGTGGACAAGCTGTTCATCCAGCT |
| | GGTGCAGACCTACAACCAGCTGTTCGAGGAAAACCCCATCAACGCCAGCGGAGT |
| | GGATGCCAAGGCCATCCTGTCTGCCAGACTGAGCAAGAGCAGACGGCTGGAAAA |
| | TCTGATCGCCCAGCTGCCTGGCGAGAAGAAGAATGGCCTGTTCGGCAACCTGAT |
| | TGCCCTGAGCCTGGGCCTGACACCTAACTTCAAGAGCAACTTCGACCTGGCCGA |
| | GGACGCCAAACTGCAGCTGAGCAAGGACACCTACGACGACGACCTGGACAATCT |
| | GCTGGCCCAGATCGGCGATCAGTACGCCGACTTGTTTCTGGCCGCCAAGAATCT |
| | GAGCGACGCCATCCTGCTGTCCGACATCCTGAGAGTGAACACCGAGATCACCAA |
| | GGCACCTCTGAGCGCCTCTATGATCAAGAGATACGACGAGCACCACCAGGATCT |
| | GACCCTGCTGAAGGCCCTCGTTAGACAGCAGCTGCCAGAGAAGTACAAAGAGAT |
| | TTTCTTCGACCAGAGCAAGAACGGCTACGCCGGCTACATTGATGGCGGAGCCAG |
| | CCAAGAGGAATTCTACAAGTTCATCAAGCCCATCCTCGAGAAGATGGACGGCAC |
| | CGAGGAACTGCTGGTCAAGCTGAACAGAGAGGACCTGCTGAGAAAGCAGAGAAC |
| | CTTCGACAACGGCAGCATCCCTCACCAGATCCACCTGGGAGAACTGCACGCCAT |
| | TCTGCGGAGACAAGAGGACTTTTACCCATTCCTGAAGGACAACCGGGAAAAGAT |
| | CGAGAAAATCCTGACCTTCAGGATCCCCTACTACGTGGGACCACTGGCCAGAGG |
| | CAATAGCAGATTCGCCTGGATGACCAGAAAGAGCGAGGAAACCATCACTCCCTG |
| | GAACTTCGAGGAAGTGGTGGACAAGGGCGCCAGCGCTCAGTCCTTCATCGAGCG |
| | GATGACCAACTTCGATAAGAACCTGCCTAACGAGAAGGTGCTGCCCAAGCACAG |
| | CCTGCTGTACGAGTACTTCACCGTGTACAACGAGCTGACCAAAGTGAAATACGT |
| | GACCGAGGGAATGAGAAAGCCCGCCTTTCTGAGCGGCGAGCAGAAAAAGGCCAT |
| | CGTGGATCTGCTGTTCAAGACCAACCGGAAAGTGACCGTGAAGCAGCTGAAAGA |
| | GGACTACTTCAAGAAAATCGAGTGCTTCGACAGCGTCGAGATCTCCGGCGTGGA |
| | AGATCGGTTCAATGCCAGCCTGGGCACATACCACGATCTGCTGAAAATTATCAA |
| | GGACAAGGACTTCCTGGACAACGAAGAGAACGAGGACATCCTTGAGGACATCGT |
| | GCTGACACTGACCCTGTTTGAGGACAGAGAGATGATCGAGGAACGGCTGAAAAC |
| | ATACGCCCACCTGTTCGACGACAAAGTGATGAAGCAACTGAAGCGGCGGAGATA |
| | CACCGGCTGGGGCAGACTGTCTCGGAAGCTGATCAACGGCATCCGGGATAAGCA |
| | GTCCGGCAAGACCATCCTGGACTTTCTGAAGTCCGACGGCTTCGCCAACAGAAA |
| | CTTCATGCAGCTGATTCACGACGACAGCCTCACCTTCAAAGAGGATATCCAGAA |
| | AGCCCAGGTGTCCGGCCAGGGCGATTCTCTGCATGAGCACATTGCCAACCTGGC |
| | CGGCTCTCCCGCCATTAAGAAAGGCATCCTGCAGACAGTGAAGGTGGTGGACGA |
| | GCTTGTGAAAGTGATGGGCAGACACAAGCCCGAGAACATCGTGATCGAAATGGC |
| | CAGAGAGAACCAGACCACACAGAAGGGACAGAAGAACAGCCGCGAGAGAATGAA |
| | GCGGATCGAAGAGGGCATCAAAGAGCTGGGCAGCCAGATCCTGAAAGAACACCC |
| | CGTGGAAAACACCCAGCTGCAGAACGAGAAGCTGTACCTGTACTACCTGCAGAA |
| | TGGACGGGATATGTACGTGGACCAAGAGCTGGACATCAACAGACTGTCCGACTA |
| | CGATGTGGACCATATCGTGCCCCAGTCTTTTCTGAAGGACGACTCCATCGACAA |
| | CAAGGTCCTGACCAGATCCGACAAGAATCGGGGCAAGAGCGACAACGTGCCCTC |
| | CGAAGAGGTGGTCAAGAAGATGAAGAACTACTGGCGACAGCTGCTGAACGCCAA |
| | GCTGATTACCCAGCGGAAGTTCGACAATCTGACCAAGGCCGAAAGAGGCGGCCT |
| | GAGCGAACTGGATAAGGCCGGCTTCATCAAGAGACAGCTGGTGGAAACCCGGCA |
| | GATCACAAAGCACGTGGCACAGATTCTGGACTCTCGGATGAACACTAAGTACGA |
| | CGAGAACGACAAACTGATCCGCGAAGTGAAAGTCATCACCCTGAAGTCCAAGCT |
| | GGTGTCCGATTTCCGGAAGGATTTCCAGTTCTACAAAGTGCGCGAGATCAACAA |
| | CTACCATCACGCCCACGACGCCTACCTGAATGCCGTTGTTGGAACAGCCCTGAT |
| | CAAAAAGTACCCTAAGCTGGAAAGCGAGTTCGTGTACGGCGACTACAAGGTGTA |
| | CGACGTGCGGAAGATGATCGCCAAGAGCGAGCAAGAGATTGGCAAGGCAACCGC |
| | CAAGTACTTCTTCTACAGCAACATCATGAACTTTTTCAAGACAGAGATCACCCT |
| | CGCCAACGGCGAGATCAGAAAGCGGCCTCTGATCGAGACAAACGGCGAAACCGG |
| | CGAGATTGTGTGGGATAAGGGCAGAGACTTTGCCACAGTGCGGAAAGTGCTGAG |
| | CATGCCCCAAGTGAATATCGTGAAGAAAACCGAGGTGCAGACAGGCGGCTTCAG |
| | CAAAGAGTCTATCCTGCCTAAGCGGAACTCCGACAAGCTGATCGCCAGAAAGAA |
| | GGACTGGGACCCCAAGAAGTACGGCGGCTTCGATTCTCCTACCGTGGCCTATAG |
| | CGTGCTGGTGGTGGCCAAAGTGGAAAAGGGCAAGTCCAAGAAACTCAAGAGCGT |
| | GAAAGAGCTGCTGGGGATCACCATCATGGAAAGAAGCAGCTTCGAGAAGAATCC |
| | GATCGATTTCCTCGAGGCCAAGGGCTACAAAGAAGTGAAAAAGGACCTGATCAT |
| | CAAGCTCCCCAAGTACTCCCTGTTCGAGCTGGAAAACGGCCGGAAGAGAATGCT |
| | GGCCTCTGCTGGCGAACTGCAGAAGGGAAACGAACTGGCCCTGCCTAGCAAATA |
| | TGTGAACTTCCTGTACCTGGCCAGCCACTATGAGAAGCTGAAGGGCAGCCCCGA |
| | GGACAATGAGCAAAAGCAGCTGTTTGTGGAACAGCACAAGCACTACCTGGACGA |
| | GATCATCGAGCAGATCAGCGAGTTTAGCAAGAGAGTGATTCTGGCCGACGCCAA |
| | TCTGGACAAAGTGCTGTCCGCCTACAACAAGCACCGGGACAAGCCTATCAGAGA |
| | GCAGGCCGAGAATATCATCCACCTGTTTACCCTGACCAACCTGGGAGCCCCTGC |
| | CGCCTTCAAGTACTTTGACACCACCATCGACCGGAAGCGGTACACCTCCACCAA |
| | AGAGGTGCTGGACGCCACTCTGATCCACCAGTCTATCACCGGCCTGTACGAGAC |
| | ACGGATCGACCTGTCTCAACTCGGAGGCGACGAAGGCGCCGATAAGAGAACCGC |
| | CGATGGCTCTGAGTTCGAGAGCCCCAAGAAAAAGCGCAAAGTGTAATAGTGAGC |
| | GGCCGCTTAATTAAGCTGCCTTCTGCGGGGCTTGCCTTCTGGCCAAGCCCTTCT |
| | TCTCTCCCTTGCACCTGTACCTCTTGGTCTTTGAATAAAGCCTGAGTAGGAAG |
| | (SEQ ID NO: 653) |
| Amino acid | MSEVEFSHEYWMRHALTLAKRARDEREVPVGAVLVLNNRVIGEGWNRAIGLHDP |
| sequence | TAHAQIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSDKKYSIGLAIGTNSVGW |
| encoded | AVITDEYKVPSKKFKVLGNTDRHSIKKNLIGALLFDSGETAEATRLKRTARRRY |
| by | TRRKNRICYLQEIFSNEMAKVDDSFFHRLEESFLVEEDKKHERHPIFGNIVDEV |
| mRNA2970 | AYHEKYPTIYHLRKKLVDSTDKADLRLIYLALAHMIKFRGHFLIEGDLNPDNSD |
| | VDKLFIQLVQTYNQLFEENPINASGVDAKAILSARLSKSRRLENLIAQLPGEKK |
| | NGLFGNLIALSLGLTPNFKSNFDLAEDAKLQLSKDTYDDDLDNLLAQIGDQYAD |
| | LFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNRE |
| | DLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPN |
| | EKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWGRLSRKL |
| | INGIRDKQSGKTILDFLKSDGFANRNFMQLIHDDSLTFKEDIQKAQVSGQGDSL |
| | HEHIANLAGSPAIKKGILQTVKVVDELVKVMGRHKPENIVIEMARENQTTQKGQ |
| | KNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVAQILD |
| | SRMNTKYDENDKLIREVKVITLKSKLVSDFRKDFQFYKVREINNYHHAHDAYLN |
| | AVVGTALIKKYPKLESEFVYGDYKVYDVRKMIAKSEQEIGKATAKYFFYSNIMN |
| | FFKTEITLANGEIRKRPLIETNGETGEIVWDKGRDFATVRKVLSMPQVNIVKKT |
| | EVQTGGFSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPKYSLFEL |
| | ENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFT |
| | LTNLGAPAAFKYFDTTIDRKRYTSTKEVLDATLIHQSITGLYETRIDLSQLGGD |
| | EGADKRTADGSEFESPKKKRKV (SEQ ID NO: 654) |
| Nucleotide | TCTAGAGTTTAAACATTTAAATCTGCAGATCCCAATGGCGCGCCGAGCTTGGCT |
| sequence | CGAGCATGGTCATAGCTGTTTCCTGTGTGAAATTGTTATCCGCTCACAATTCCA |
| of a | CACAACATACGAGCCGGAAGCATAAAGTGTAAAGCCTGGGGTGCCTAATGAGTG |
| plasmid | AGCTAACTCACATTAATTGCGTTGCGCTCACTGCCCGCTTTCCAGTCGGGAAAC |
| encoding | CTGTCGTGCCAGCTGCATTAATGAATCGGCCAACGCGCGGGGAGAGGCGGTTTG |
| mRNA2971 | CGTATTGGGCACTCTTGCGCTTCCTCGCTCACTGACTCGCTGCGCTCGGTCGTT |
| | CGGCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGGCC |
| | AGGAACCGTAAAAAGGCCGCGTTGCTGGCGTTTTTCCATAGGCTCCGCCCCCCT |
| | GACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTT |
| | CCGACCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTACGGGG |
| | TCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCA |
| | ATCTAAAGTATATATGAGTAAACTTGGTCTGACAGTTAGAAAAACTCATCGAGC |
| | ATCAAATGAAACTGCAATTTATTCATATCAGGATTATCAATACCATATTTTTGA |
| | AAAAGCCGTTTCTGTAATGAAGGAGAAAACTCACCGAGGCAGTTCCATAGGATG |
| | GCAAGATCCTGGTATCGGTCTGCGATTCCGACTCGTCCAACATCAATACAACCT |
| | ATTAATTTCCCCTCGTCAAAAATAAGGTTATCAAGTGAGAAATCACCATGAGTG |
| | ACGACTGAATCCGGTGAGAATGGCAAAAGTTTATGCATTTCTTTCCAGACTTGT |
| | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGAGCGAGGTCGAGTTCTCTCA |
| | CGAATATTGGATGAGACACGCTCTCACCCTGGCTAAGAGAGCCAGGGACGAAAG |
| | AGAGGTGCCAGTTGGCGCTGTCCTGGTGTTGAACAATCGCGTCATCGGAGAAGG |
| | ATGGAATCGCGCCATTGGCCTGCACGATCCAACCGCACATGCCCAGATTATGGC |
| | TCTGCGGCAAGGCGGCCTCGTGATGCAAAATTACAGACTGTACGATGCTACCCT |
| | CTACTCCACCTTCGAGCCCTGTGTCATGTGTGCTGGGGCAATGATTCACTCCCG |
| | GATTGGCCGCGTGGTGTTTGGAGTGCGGAATGCCAAGACTGGCGCCGCTGGATC |
| | TCTGATGGACGTCCTGCACCATCCTGGGATGAACCACCGGGTCGAGATCACAGA |
| | GGGAATTCTGGCTGACGAGTGCGCTGCCCTGCTGTGCAGGTTCTTTAGAATGCC |
| | TAGAAGGGTGTTCAACGCCCAGAAAAAAGCTCAGAGCAGCACCGATTCCGGCGG |
| | AAGCAGCGGAGGATCTTCTGGAAGCGAAACCCCAGGCACCAGCGAGTCTGCCAC |
| | ACCAGAATCATCTGGCGGTAGCTCCGGCGGCAGCGACAAGAAGTACTCTATCGG |
| | ACTGGCCATCGGCACCAACTCTGTTGGATGGGCCGTGATCACCGACGAGTACAA |
| | GGTGCCCAGCAAGAAATTCAAGGTGCTGGGCAACACCGACCGGCACAGCATCAA |
| | GAAGAATCTGATCGGCGCCCTGCTGTTCGACTCTGGCGAAACAGCCGAAGCCAC |
| | CAGACTGAAGAGAACCGCCAGGCGGAGATACACCCGGCGGAAGAACCGGATCTG |
| | CTACCTGCAAGAGATCTTCAGCAACGAGATGGCCAAGGTGGACGACAGCTTCTT |
| | CCACAGACTGGAAGAGTCCTTCCTGGTGGAAGAGGACAAGAAGCACGAGCGGCA |
| | CCCCATCTTCGGCAACATCGTGGATGAGGTGGCCTACCACGAGAAGTACCCCAC |
| | CATCTACCACCTGAGAAAGAAACTGGTGGACAGCACCGACAAGGCCGACCTGAG |
| | ACTGATCTACCTGGCTCTGGCCCACATGATCAAGTTCCGGGGCCACTTTCTGAT |
| | CGAGGGCGATCTGAACCCCGACAACAGCGACGTGGACAAGCTGTTCATCCAGCT |
| | GGTGCAGACCTACAACCAGCTGTTCGAGGAAAACCCCATCAACGCCTCTGGCGT |
| | GGACGCCAAGGCTATCCTGTCTGCCAGACTGAGCAAGAGCAGAAGGCTGGAAAA |
| | CCTGATCGCCCAGCTGCCTGGCGAGAAGAAGAATGGCCTGTTCGGCAACCTGAT |
| | TGCCCTGAGCCTGGGACTGACCCCTAACTTCAAGAGCAACTTCGACCTGGCCGA |
| | GGATGCCAAACTGCAGCTGAGCAAGGACACCTACGACGACGACCTGGACAATCT |
| | GCTGGCCCAGATCGGCGATCAGTACGCCGACTTGTTTCTGGCCGCCAAGAACCT |
| | GTCCGACGCCATCCTGCTGAGCGATATCCTGAGAGTGAACACCGAGATCACAAA |
| | GGCCCCTCTGAGCGCCTCTATGATCAAGAGATACGACGAGCACCACCAGGATCT |
| | GACCCTGCTGAAGGCCCTCGTTAGACAGCAGCTGCCAGAGAAGTACAAAGAGAT |
| | TTTCTTCGATCAGTCCAAGAACGGCTACGCCGGCTACATTGATGGCGGAGCCAG |
| | CCAAGAGGAATTCTACAAGTTCATCAAGCCCATCCTGGAAAAGATGGACGGCAC |
| | CGAGGAACTGCTGGTCAAGCTGAACAGAGAGGACCTGCTGCGGAAGCAGCGGAC |
| | CTTCGACAATGGCTCTATCCCTCACCAGATCCACCTGGGAGAGCTGCACGCCAT |
| | TCTGCGGAGACAAGAGGACTTTTACCCATTCCTGAAGGACAACCGGGAAAAGAT |
| | CGAGAAGATCCTGACCTTCAGGATCCCCTACTACGTGGGACCACTGGCCAGAGG |
| | CAATAGCAGATTCGCCTGGATGACCAGAAAGAGCGAGGAAACCATCACACCCTG |
| | GAACTTCGAGGAAGTGGTGGACAAGGGCGCCAGCGCTCAGTCCTTCATCGAGCG |
| | GATGACCAACTTCGATAAGAACCTGCCTAACGAGAAGGTGCTGCCCAAGCACTC |
| | CCTGCTGTATGAGTACTTCACCGTGTACAACGAGCTGACCAAAGTGAAATACGT |
| | GACCGAGGGAATGAGAAAGCCCGCCTTTCTGAGCGGCGAGCAGAAAAAGGCCAT |
| | TGTGGATCTGCTGTTCAAGACCAACCGGAAAGTGACCGTGAAGCAGCTGAAAGA |
| | GGACTACTTCAAGAAAATCGAGTGCTTCGACAGCGTGGAAATCAGCGGCGTGGA |
| | AGATCGGTTCAATGCCAGCCTGGGCACATACCACGACCTGCTGAAAATTATCAA |
| | GGACAAGGACTTCCTGGACAACGAAGAGAACGAGGACATTCTCGAGGACATCGT |
| | GCTGACCCTGACACTGTTTGAGGACAGAGAGATGATCGAGGAACGGCTGAAAAC |
| | ATACGCCCACCTGTTCGACGACAAAGTGATGAAGCAACTGAAGCGGAGGCGGTA |
| | CACAGGCTGGGGCAGACTGTCTCGGAAGCTGATCAACGGCATCCGGGATAAGCA |
| | GTCCGGCAAGACAATCCTGGATTTCCTGAAGTCCGACGGCTTCGCCAACAGAAA |
| | CTTCATGCAGCTGATCCACGACGACAGCCTGACCTTTAAAGAGGACATCCAGAA |
| | AGCCCAGGTGTCCGGCCAAGGCGATTCTCTGCACGAGCACATTGCCAACCTGGC |
| | CGGATCTCCCGCCATTAAGAAGGGCATCCTGCAGACAGTGAAGGTGGTGGACGA |
| | GCTTGTGAAAGTGATGGGCAGACACAAGCCCGAGAACATCGTGATCGAAATGGC |
| | CAGAGAGAACCAGACCACACAGAAGGGCCAGAAGAACAGCCGCGAGAGAATGAA |
| | GCGGATCGAAGAGGGCATCAAAGAGCTGGGCAGCCAGATCCTGAAAGAACACCC |
| | CGTGGAAAACACCCAGCTGCAGAACGAGAAGCTGTACCTGTACTACCTGCAGAA |
| | TGGACGGGATATGTACGTGGACCAAGAGCTGGACATCAACCGGCTGAGCGACTA |
| | CGATGTGGACCATATCGTGCCCCAGAGCTTTCTGAAGGACGACTCCATCGATAA |
| | CAAGGTCCTGACCAGAAGCGACAAGAACCGGGGCAAGAGCGATAACGTGCCCTC |
| | CGAAGAGGTGGTCAAGAAGATGAAGAACTACTGGCGACAGCTGCTGAACGCCAA |
| | GCTGATTACCCAGCGGAAGTTCGATAACCTGACCAAGGCCGAGAGAGGCGGCCT |
| | GAGCGAACTTGATAAGGCCGGCTTCATTAAGCGGCAGCTGGTGGAAACCCGGCA |
| | GATCACCAAACACGTGGCACAGATTCTGGACTCCCGGATGAACACTAAGTACGA |
| | CGAGAATGACAAGCTGATCCGGGAAGTGAAAGTCATCACCCTGAAGTCTAAGCT |
| | GGTGTCCGATTTCCGGAAGGATTTCCAGTTCTACAAAGTGCGGGAAATCAACAA |
| | CTACCATCACGCCCACGACGCCTACCTGAATGCCGTTGTTGGAACAGCCCTGAT |
| | CAAGAAGTATCCCAAGCTGGAAAGCGAGTTCGTGTACGGCGACTACAAGGTGTA |
| | CGACGTGCGGAAGATGATCGCCAAGAGCGAACAAGAGATCGGCAAGGCTACCGC |
| | CAAGTACTTTTTCTACAGCAACATCATGAACTTTTTCAAGACAGAGATCACCCT |
| | GGCCAACGGCGAGATCCGGAAAAGACCCCTGATCGAGACAAACGGCGAAACCGG |
| | GGAGATCGTGTGGGATAAGGGCAGAGATTTTGCCACAGTGCGGAAAGTGCTGAG |
| | CATGCCCCAAGTGAATATCGTGAAGAAAACCGAGGTGCAGACAGGCGGCTTCAG |
| | CAAAGAGTCTATCCTGCCTAAGCGGAACAGCGATAAGCTGATCGCCAGAAAGAA |
| | GGACTGGGACCCCAAGAAATACGGCGGCTTCGTGTCTCCTACCGTGGCCTATTC |
| | TGTGCTGGTGGTGGCCAAAGTGGAAAAGGGCAAGTCCAAAAAGCTCAAGAGCGT |
| | GAAAGAGCTGCTGGGGATCACCATCATGGAAAGAAGCAGCTTTGAGAAGAACCC |
| | GATCGACTTTCTGGAAGCCAAGGGCTACAAAGAAGTCAAGAAGGACCTCATCAT |
| | CAAGCTCCCCAAGTACAGCCTGTTCGAGCTGGAAAATGGCCGGAAGCGGATGCT |
| | GGCCTCTGCTAGAGAACTGCAGAAGGGAAACGAGCTGGCCCTGCCTAGCAAATA |
| | TGTGAACTTCCTGTACCTGGCCAGCCACTATGAGAAGCTGAAGGGCAGCCCCGA |
| | GGACAATGAGCAAAAGCAGCTGTTTGTGGAACAGCACAAGCACTACCTGGACGA |
| | GATCATCGAGCAGATCAGCGAGTTCTCCAAGAGAGTGATCCTGGCCGACGCTAA |
| | CCTGGATAAGGTGCTGTCTGCCTATAACAAGCACCGGGACAAGCCTATCAGAGA |
| | GCAGGCCGAGAATATCATCCACCTGTTTACCCTGACCAACCTGGGAGCCCCTGC |
| | CGCCTTCAAGTACTTCGACACCACCATCGACCGGAAGCAGTACCGGTCCACCAA |
| | AGAGGTGCTGGACGCCACACTGATCCACCAGTCTATCACCGGCCTGTACGAAAC |
| | CCGGATCGACCTGTCTCAGCTCGGAGGCGACGAAGGCGCCGATAAGAGAACCGC |
| | CGATGGCTCTGAGTTCGAGAGCCCCAAGAAAAAGCGCAAAGTGTAATAGTGAGC |
| | GGCCGCTTAATTAAGCTGCCTTCTGCGGGGCTTGCCTTCTGGCCAAGCCCTTCT |
| | TCTCTCCCTTGCACCTGTACCTCTTGGTCTTTGAATAAAGCCTGAGTAGGAAG |
| | (SEQ ID NO: 655) |
| Amino acid | MSEVEFSHEYWMRHALTLAKRARDEREVPVGAVLVLNNRVIGEGWNRAIGLHDP |
| sequence | TAHAQIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSDKKYSIGLAIGTNSVGW |
| encoded | AVITDEYKVPSKKFKVLGNTDRHSIKKNLIGALLFDSGETAEATRLKRTARRRY |
| by | TRRKNRICYLQEIFSNEMAKVDDSFFHRLEESFLVEEDKKHERHPIFGNIVDEV |
| mRNA2971 | AYHEKYPTIYHLRKKLVDSTDKADLRLIYLALAHMIKFRGHFLIEGDLNPDNSD |
| | VDKLFIQLVQTYNQLFEENPINASGVDAKAILSARLSKSRRLENLIAQLPGEKK |
| | NGLFGNLIALSLGLTPNFKSNFDLAEDAKLQLSKDTYDDDLDNLLAQIGDQYAD |
| | LFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNRE |
| | DLLRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFDKNLPN |
| | EKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWGRLSRKL |
| | INGIRDKQSGKTILDFLKSDGFANRNFMQLIHDDSLTFKEDIQKAQVSGQGDSL |
| | HEHIANLAGSPAIKKGILQTVKVVDELVKVMGRHKPENIVIEMARENQTTQKGQ |
| | KNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVAQILD |
| | SRMNTKYDENDKLIREVKVITLKSKLVSDFRKDFQFYKVREINNYHHAHDAYLN |
| | AVVGTALIKKYPKLESEFVYGDYKVYDVRKMIAKSEQEIGKATAKYFFYSNIMN |
| | FFKTEITLANGEIRKRPLIETNGETGEIVWDKGRDFATVRKVLSMPQVNIVKKT |
| | EVQTGGFSKESILPKRNSDKLIARKKDWDPKKYGGFVSPTVAYSVLVVAKVEKG |
| | KSKKLKSVKELLGITIMERSSFEKNPIDFLEAKGYKEVKKDLIIKLPKYSLFEL |
| | ENGRKRMLASARELQKGNELALPSKYVNFLYLASHYEKLKGSPEDNEQKQLFVE |
| | QHKHYLDEIIEQISEFSKRVILADANLDKVLSAYNKHRDKPIREQAENIIHLFT |
| | LTNLGAPAAFKYFDTTIDRKQYRSTKEVLDATLIHQSITGLYETRIDLSQLGGD |
| | EGADKRTADGSEFESPKKKRKV (SEQ ID NO: 656) |
| Nucleotide | TCTAGAGTTTAAACATTTAAATCTGCAGATCCCAATGGCGCGCCGAGCTTGGCT |
| sequence | CGAGCATGGTCATAGCTGTTTCCTGTGTGAAATTGTTATCCGCTCACAATTCCA |
| of a | CACAACATACGAGCCGGAAGCATAAAGTGTAAAGCCTGGGGTGCCTAATGAGTG |
| plasmid | AGCTAACTCACATTAATTGCGTTGCGCTCACTGCCCGCTTTCCAGTCGGGAAAC |
| encoding | CTGTCGTGCCAGCTGCATTAATGAATCGGCCAACGCGCGGGGAGAGGCGGTTTG |
| mRNA2972 | CGTATTGGGCACTCTTGCGCTTCCTCGCTCACTGACTCGCTGCGCTCGGTCGTT |
| | CGGCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGGCC |
| | AGGAACCGTAAAAAGGCCGCGTTGCTGGCGTTTTTCCATAGGCTCCGCCCCCCT |
| | GACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTT |
| | CCGACCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTACGGGG |
| | TCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCA |
| | ATCTAAAGTATATATGAGTAAACTTGGTCTGACAGTTAGAAAAACTCATCGAGC |
| | ATCAAATGAAACTGCAATTTATTCATATCAGGATTATCAATACCATATTTTTGA |
| | AAAAGCCGTTTCTGTAATGAAGGAGAAAACTCACCGAGGCAGTTCCATAGGATG |
| | GCAAGATCCTGGTATCGGTCTGCGATTCCGACTCGTCCAACATCAATACAACCT |
| | ATTAATTTCCCCTCGTCAAAAATAAGGTTATCAAGTGAGAAATCACCATGAGTG |
| | ACGACTGAATCCGGTGAGAATGGCAAAAGTTTATGCATTTCTTTCCAGACTTGT |
| | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGAGCGAGGTCGAGTTCTCTCA |
| | CGAATATTGGATGAGACACGCTCTCACCCTGGCTAAGAGAGCCAGGGACGAAAG |
| | AGAGGTGCCAGTTGGCGCTGTCCTGGTGTTGAACAATCGCGTCATCGGAGAAGG |
| | ATGGAATCGCGCCATTGGCCTGCACGATCCAACCGCACATGCCCAGATTATGGC |
| | TCTGCGGCAAGGCGGCCTCGTGATGCAAAATTACAGACTGTACGATGCTACCCT |
| | CTACTCCACCTTCGAGCCCTGTGTCATGTGTGCTGGGGCAATGATTCACTCCCG |
| | GATTGGCCGCGTGGTGTTTGGAGTGCGGAATGCCAAGACTGGCGCCGCTGGATC |
| | TCTGATGGACGTCCTGCACCATCCTGGGATGAACCACCGGGTCGAGATCACAGA |
| | GGGAATTCTGGCTGACGAGTGCGCTGCCCTGCTGTGCAGGTTCTTTAGAATGCC |
| | TAGAAGGGTGTTCAACGCCCAGAAAAAAGCTCAGAGCAGCACCGATTCCGGCGG |
| | AAGCAGCGGAGGATCTTCTGGAAGCGAAACCCCAGGCACCAGCGAGTCTGCCAC |
| | ACCAGAATCATCTGGCGGTAGCTCCGGCGGCAGCAAGAGAAATTACATCCTGGG |
| | CCTCGCCATCGGCATCACCTCTGTCGGCTACGGCATCATCGACTACGAGACACG |
| | GGATGTGATTGATGCCGGCGTGCGGCTGTTCAAAGAGGCCAACGTCGAGAACAA |
| | CGAGGGCCGCAGATCTAAGAGGGGAGCCAGACGGCTGAAGAGAAGGCGGAGACA |
| | CAGAATCCAGAGAGTGAAGAAGCTGCTGTTCGACTACAACCTGCTGACCGACCA |
| | CAGCGAGCTGAGCGGCATCAACCCTTATGAGGCCAGAGTGAAGGGCCTGAGCCA |
| | GAAGCTGAGCGAGGAAGAGTTTAGCGCCGCACTGCTGCACCTGGCCAAGAGAAG |
| | AGGCGTGCACAACGTGAACGAAGTGGAAGAGGACACCGGCAACGAGCTGTCCAC |
| | CAAAGAGCAGATCAGCAGAAACAGCAAGGCCCTGGAAGAGAAATACGTGGCCGA |
| | ACTGCAGCTGGAACGGCTGAAAAAGGATGGCGAAGTGCGGGGCAGCATCAACCG |
| | GTTCAAGACCAGCGACTACGTGAAAGAAGCCAAACAGCTGCTGAAGGTGCAGAA |
| | GGCCTACCACCAGCTGGACCAGAGCTTCATCGACACCTACATCGACCTGCTGGA |
| | AACCCGGCGGACCTACTATGAAGGACCTGGCGAGGGAAGCCCCTTCGGCTGGAA |
| | GGACATCAAAGAATGGTACGAGATGCTGATGGGCCACTGCACCTACTTTCCCGA |
| | GGAACTGCGGAGCGTGAAGTACGCCTACAACGCCGACCTGTACAACGCCCTGAA |
| | CGACCTGAACAACCTCGTGATCACCCGGGACGAGAACGAGAAGCTGGAATATTA |
| | CGAGAAGTTCCAGATCATCGAGAACGTGTTCAAGCAGAAGAAGAAGCCCACACT |
| | GAAGCAGATCGCCAAAGAGATCCTGGTCAACGAGGAAGATATCAAGGGCTACAG |
| | AGTGACCAGCACCGGCAAGCCCGAGTTCACCAACCTGAAGGTGTACCACGACAT |
| | CAAGGATATCACAGCCCGGAAAGAGATTATTGAGAACGCCGAGCTGCTGGACCA |
| | AATCGCCAAGATCCTGACCATCTACCAGTCCTCCGAGGACATCCAAGAGGAACT |
| | GACCAATCTGAACTCCGAGCTGACCCAAGAAGAGATCGAGCAGATCTCTAATCT |
| | GAAGGGGTACACAGGCACCCACAACCTGAGCCTGAAGGCCATCAACCTGATCCT |
| | GGACGAGCTGTGGCACACCAACGACAACCAGATTGCCATCTTCAACCGGCTGAA |
| | GCTGGTGCCCAAGAAGGTGGACCTCAGCCAGCAGAAAGAAATCCCCACCACACT |
| | GGTGGACGACTTCATTCTGAGCCCCGTGGTCAAGAGAAGCTTCATCCAGAGCAT |
| | CAAAGTGATCAACGCCATCATCAAGAAGTACGGGCTGCCCAACGATATCATCAT |
| | CGAGCTGGCCCGCGAGAAGAACTCCAAGGACGCTCAGAAAATGATCAACGAGAT |
| | GCAGAAGCGGAACCGGCAGACCAACGAGCGGATCGAGGAAATCATCCGGACCAC |
| | CGGCAAAGAGAACGCCAAGTACCTGATCGAGAAGATCAAGCTGCACGACATGCA |
| | AGAGGGCAAGTGTCTGTACAGCCTGGAAGCCATTCCTCTGGAAGATCTGCTGAA |
| | CAATCCCTTCAACTACGAGGTGGACCACATCATCCCCAGAAGCGTGTCCTTCGA |
| | CAACAGCTTCAACAACAAGGTGCTCGTGAAGCAAGAGGAAAACTCCAAGAAGGG |
| | CAACAGAACCCCATTCCAGTACCTGAGCAGCTCCGACAGCAAAATCAGCTACGA |
| | AACCTTCAAGAAGCACATCCTGAATCTGGCCAAAGGCAAGGGCCGCATCAGCAA |
| | GACCAAGAAAGAATACCTGCTCGAGGAACGGGACATCAACAGATTCAGCGTGCA |
| | GAAAGACTTCATCAATCGGAACCTGGTGGACACCAGATACGCCACCAGAGGCCT |
| | GATGAATCTGCTGAGAAGCTACTTCCGCGTGAACAATCTGGACGTGAAAGTCAA |
| | GTCCATCAACGGCGGCTTCACCAGCTTTCTGCGGAGAAAGTGGAAGTTCAAGAA |
| | AGAGCGGAACAAGGGCTATAAGCACCACGCCGAGGACGCCCTGATCATTGCCAA |
| | CGCCGATTTCATCTTCAAAGAGTGGAAGAAACTGGACAAGGCCAAAAAAGTGAT |
| | GGAAAACCAGATGTTCGAGGAAAAGCAGGCCGAGAGCATGCCCGAGATCGAAAC |
| | CGAGCAAGAGTACAAAGAGATTTTCATCACGCCCCACCAGATCAAGCACATTAA |
| | GGACTTCAAGGACTACAAGTACAGCCACCGCGTGGACAAGAAGCCTAATAGAGA |
| | GCTGATTAACGACACCCTGTACAGCACCCGGAAGGACGACAAGGGCAATACCCT |
| | GATCGTCAACAACCTGAACGGCCTGTACGACAAGGACAACGACAAGCTCAAGAA |
| | GCTGATCAACAAGAGCCCCGAGAAACTGCTGATGTACCACCACGATCCTCAGAC |
| | CTACCAGAAACTGAAGCTCATCATGGAACAGTACGGCGACGAGAAGAATCCCCT |
| | GTACAAGTACTACGAGGAAACCGGGAACTACCTGACCAAGTACTCCAAAAAGGA |
| | CAATGGGCCCGTGATCAAGAAGATTAAGTATTACGGCAACAAGCTGAATGCCCA |
| | CCTGGACATCACCGACGACTACCCCAACTCCAGAAACAAGGTGGTCAAGCTGTC |
| | CCTGAAGCCTTACAGATTCGACGTGTACCTGGACAACGGCGTGTACAAGTTCGT |
| | GACCGTGAAGAACCTGGATGTGATCAAAAAAGAAAACTACTACGAAGTGAACAG |
| | CAAGTGCTATGAGGAAGCCAAGAAACTCAAGAAAATCAGCAACCAGGCCGAGTT |
| | TATCGCCTCCTTCTACAACAACGATCTGATCAAGATCAACGGGGAGCTGTATAG |
| | AGTGATTGGGGTCAACAATGACCTGCTGAACCGGATCGAAGTCAACATGATCGA |
| | CATCACCTACCGCGAGTACCTCGAGAACATGAACGACAAGAGGCCTCCACGGAT |
| | CATTAAGACAATCGCCAGCAAGACGCAGAGCATTAAGAAGTACAGCACTGACAT |
| | TCTGGGCAACCTGTACGAAGTCAAGAGCAAAAAGCACCCGCAGATTATCAAGAA |
| | AGGCGAGGGCGCCGACAAGAGAACAGCCGATGGTTCCGAGTTCGAAAGCCCCAA |
| | GAAGAAGAGGAAAGTCTAATAGTGAGCGGCCGCTTAATTAAGCTGCCTTCTGCG |
| | GGGCTTGCCTTCTGGCCAAGCCCTTCTTCTCTCCCTTGCACCTGTACCTCTTGG |
| | TCTTTGAATAAAGCCTGAGTAGGAAG (SEQ ID NO: 657) |
| Amino acid | MSEVEFSHEYWMRHALTLAKRARDEREVPVGAVLVLNNRVIGEGWNRAIGLHDP |
| sequence | TAHAQIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSKRNYILGLAIGITSVGY |
| encoded | GIIDYETRDVIDAGVRLFKEANVENNEGRRSKRGARRLKRRRRHRIQRVKKLLF |
| by | DYNLLTDHSELSGINPYEARVKGLSQKLSEEEFSAALLHLAKRRGVHNVNEVEE |
| mRNA2972 | DTGNELSTKEQISRNSKALEEKYVAELQLERLKKDGEVRGSINRFKTSDYVKEA |
| | KQLLKVQKAYHQLDQSFIDTYIDLLETRRTYYEGPGEGSPFGWKDIKEWYEMLM |
| | GHCTYFPEELRSVKYAYNADLYNALNDLNNLVITRDENEKLEYYEKFQIIENVF |
| | KQKKKPTLKQIAKEILVNEEDIKGYRVTSTGKPEFTNLKVYHDIKDITARKEII |
| | ENAELLDQIAKILTIYQSSEDIQEELTNLNSELTQEEIEQISNLKGYTGTHNLS |
| | LKAINLILDELWHTNDNQIAIFNRLKLVPKKVDLSQQKEIPTTLVDDFILSPVV |
| | KRSFIQSIKVINAIIKKYGLPNDIIIELAREKNSKDAQKMINEMQKRNRQTNER |
| | IEEIIRTTGKENAKYLIEKIKLHDMQEGKCLYSLEAIPLEDLLNNPFNYEVDHI |
| | IPRSVSFDNSFNNKVLVKQEENSKKGNRTPFQYLSSSDSKISYETFKKHILNLA |
| | KGKGRISKTKKEYLLEERDINRFSVQKDFINRNLVDTRYATRGLMNLLRSYFRV |
| | NNLDVKVKSINGGFTSFLRRKWKFKKERNKGYKHHAEDALIIANADFIFKEWKK |
| | LDKAKKVMENQMFEEKQAESMPEIETEQEYKEIFITPHQIKHIKDFKDYKYSHR |
| | VDKKPNRELINDTLYSTRKDDKGNTLIVNNLNGLYDKDNDKLKKLINKSPEKLL |
| | MYHHDPQTYQKLKLIMEQYGDEKNPLYKYYEETGNYLTKYSKKDNGPVIKKIKY |
| | YGNKLNAHLDITDDYPNSRNKVVKLSLKPYRFDVYLDNGVYKFVTVKNLDVIKK |
| | ENYYEVNSKCYEEAKKLKKISNQAEFIASFYNNDLIKINGELYRVIGVNNDLLN |
| | RIEVNMIDITYREYLENMNDKRPPRIIKTIASKTQSIKKYSTDILGNLYEVKSK |
| | KHPQIIKKGEGADKRTADGSEFESPKKKRKV (SEQ ID NO: 658) |
| Nucleotide | TCTAGAGTTTAAACATTTAAATCTGCAGATCCCAATGGCGCGCCGAGCTTGGCT |
| sequence | CGAGCATGGTCATAGCTGTTTCCTGTGTGAAATTGTTATCCGCTCACAATTCCA |
| of a | CACAACATACGAGCCGGAAGCATAAAGTGTAAAGCCTGGGGTGCCTAATGAGTG |
| plasmid | AGCTAACTCACATTAATTGCGTTGCGCTCACTGCCCGCTTTCCAGTCGGGAAAC |
| encoding | CTGTCGTGCCAGCTGCATTAATGAATCGGCCAACGCGCGGGGAGAGGCGGTTTG |
| mRNA2973 | CGTATTGGGCACTCTTGCGCTTCCTCGCTCACTGACTCGCTGCGCTCGGTCGTT |
| | CGGCTGCGGCGAGCGGTATCAGCTCACTCAAAGGCGGTAATACGGTTATCCACA |
| | GAATCAGGGGATAACGCAGGAAAGAACATGTGAGCAAAAGGCCAGCAAAAGGCC |
| | AGGAACCGTAAAAAGGCCGCGTTGCTGGCGTTTTTCCATAGGCTCCGCCCCCCT |
| | GACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTT |
| | CCGACCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGT |
| | ATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAG |
| | ATTACGCGCAGAAAAAAAGGATCTCAAGAAGATCCTTTGATCTTTTCTACGGGG |
| | TCTGACGCTCAGTGGAACGAAAACTCACGTTAAGGGATTTTGGTCATGAGATTA |
| | TCAAAAAGGATCTTCACCTAGATCCTTTTAAATTAAAAATGAAGTTTTAAATCA |
| | ATCTAAAGTATATATGAGTAAACTTGGTCTGACAGTTAGAAAAACTCATCGAGC |
| | ATCAAATGAAACTGCAATTTATTCATATCAGGATTATCAATACCATATTTTTGA |
| | AAAAGCCGTTTCTGTAATGAAGGAGAAAACTCACCGAGGCAGTTCCATAGGATG |
| | GCAAGATCCTGGTATCGGTCTGCGATTCCGACTCGTCCAACATCAATACAACCT |
| | ATTAATTTCCCCTCGTCAAAAATAAGGTTATCAAGTGAGAAATCACCATGAGTG |
| | ACGACTGAATCCGGTGAGAATGGCAAAAGTTTATGCATTTCTTTCCAGACTTGT |
| | TCAACAGGCCAGCCATTACGCTCGTCATCAAAATCACTCGCATCAACCAAACCG |
| | TTATTCATTCGTGATTGCGCCTGAGCGAGACGAAATACGCGATCGCTGTTAAAA |
| | GGACAATTACAAACAGGAATCGAATGCAACCGGCGCAGGAACACTGCCAGCGCA |
| | TCAACAATATTTTCACCTGAATCAGGATATTCTTCTAATACCTGGAATGCTGTT |
| | TTCCCAGGGATCGCAGTGGTGAGTAACCATGCATCATCAGGAGTACGGATAAAA |
| | TGCTTGATGGTCGGAAGAGGCATAAATTCCGTCAGCCAGTTTAGTCTGACCATC |
| | TCATCTGTAACATCATTGGCAACGCTACCTTTGCCATGTTTCAGAAACAACTCT |
| | GGCGCATCGGGCTTCCCATACAATCGATAGATTGTCGCACCTGATTGCCCGACA |
| | TTATCGCGAGCCCATTTATACCCATATAAATCAGCATCCATGTTGGAATTTAAT |
| | CGCGGCCTAGAGCAAGACGTTTCCCGTTGAATATGGCTCATACTCTTCCTTTTT |
| | CAATATTATTGAAGCATTTATCAGGGTTATTGTCTCATGAGCGGATACATATTT |
| | GAATGTATTTAGAAAAATAAACAAATAGGGGTTCCGCGCACATTTCCCCGAAAA |
| | GTGCCACCTGACGTCTAAGAAACCATTATTATCATGACATTAACCTATAAAAAT |
| | AGGCGTATCACGAGGCCCTTTCGTCTCGCGCGTTTCGGTGATGACGGTGAAAAC |
| | CTCTGACACATGCAGCTCCCGGAGACGGTCACAGCTTGTCTGTAAGCGGATGCC |
| | GGGAGCAGACAAGCCCGTCAGGGCGCGTCAGCGGGTGTTGGCGGGTGTCGGGGC |
| | TGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCG |
| | CCATTCAGGCTGCGCAACTGTTGGGAAGGGCGATCGGTGCGGGCCTCTTCGCTA |
| | TTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAATTGACGCGT |
| | ATTGGGATCGAGCTCGGTACCTAATACGACACACTATAAGGAAATAAGAGAGAA |
| | AAGAAGAGTAAGAAGAAATATAAGAGCCACCATGAGCGAGGTCGAGTTCTCTCA |
| | CGAATATTGGATGAGACACGCTCTCACCCTGGCTAAGAGAGCCAGGGACGAAAG |
| | AGAGGTGCCAGTTGGCGCTGTCCTGGTGTTGAACAATCGCGTCATCGGAGAAGG |
| | ATGGAATCGCGCCATTGGCCTGCACGATCCAACCGCACATGCCCAGATTATGGC |
| | TCTGCGGCAAGGCGGCCTCGTGATGCAAAATTACAGACTGTACGATGCTACCCT |
| | CTACTCCACCTTCGAGCCCTGTGTCATGTGTGCTGGGGCAATGATTCACTCCCG |
| | GATTGGCCGCGTGGTGTTTGGAGTGCGGAATGCCAAGACTGGCGCCGCTGGATC |
| | TCTGATGGACGTCCTGCACCATCCTGGGATGAACCACCGGGTCGAGATCACAGA |
| | GGGAATTCTGGCTGACGAGTGCGCTGCCCTGCTGTGCAGGTTCTTTAGAATGCC |
| | TAGAAGGGTGTTCAACGCCCAGAAAAAAGCTCAGAGCAGCACCGATTCCGGCGG |
| | AAGCAGCGGAGGATCTTCTGGAAGCGAAACCCCAGGCACCAGCGAGTCTGCCAC |
| | ACCAGAATCATCTGGCGGTAGCTCCGGCGGCAGCAAGAGAAATTACATCCTCGG |
| | ACTGGCCATCGGCATCACCTCTGTCGGCTACGGCATCATCGACTACGAGACACG |
| | GGACGTGATCGATGCCGGCGTGCGGCTGTTTAAAGAGGCCAACGTCGAGAACAA |
| | CGAGGGCCGCAGATCTAAGAGAGGCGCCAGACGGCTGAAGAGAAGAAGAAGGCA |
| | CCGGATCCAGAGAGTGAAGAAGCTGCTGTTCGACTACAACCTGCTGACCGACCA |
| | CAGCGAGCTGAGCGGCATCAATCCTTACGAGGCCAGAGTGAAGGGCCTGAGCCA |
| | GAAGCTGAGCGAGGAAGAGTTTAGCGCTGCCCTGCTGCACCTGGCCAAGAGAAG |
| | AGGCGTGCACAACGTGAACGAGGTGGAAGAGGACACCGGCAACGAGCTGTCCAC |
| | CAAAGAGCAGATCAGCCGGAACAGCAAGGCCCTGGAAGAGAAATACGTGGCCGA |
| | ACTGCAGCTGGAACGGCTGAAAAAGGATGGCGAAGTGCGGGGCAGCATCAACCG |
| | GTTCAAGACCAGCGACTACGTGAAAGAAGCCAAACAGCTGCTGAAGGTGCAGAA |
| | GGCCTACCACCAGCTGGACCAGAGCTTCATCGACACCTACATCGACCTGCTGGA |
| | AACCCGGCGGACCTACTATGAAGGACCTGGCGAGGGAAGCCCCTTCGGCTGGAA |
| | GGACATCAAAGAATGGTACGAGATGCTGATGGGCCACTGCACCTACTTTCCCGA |
| | GGAACTGCGGAGCGTGAAGTACGCCTACAACGCCGACCTGTACAACGCCCTGAA |
| | CGACCTGAACAACCTCGTGATCACCCGGGACGAGAACGAGAAGCTGGAATATTA |
| | CGAGAAGTTCCAAATCATCGAGAACGTGTTCAAGCAGAAGAAAAAGCCCACGCT |
| | GAAGCAGATCGCCAAAGAGATCCTGGTCAACGAGGAAGATATCAAGGGCTACAG |
| | AGTGACCAGCACCGGCAAGCCCGAGTTCACCAACCTGAAGGTGTACCACGACAT |
| | CAAGGATATCACAGCCCGGAAAGAAATCATTGAGAATGCCGAGCTGCTCGACCA |
| | GATTGCCAAGATCCTGACCATCTACCAGAGCAGCGAGGACATCCAAGAGGAACT |
| | GACCAATCTGAACTCCGAGCTGACCCAAGAAGAGATCGAACAGATCAGCAATCT |
| | GAAGGGGTACACCGGCACACACAACCTGAGCCTGAAGGCCATCAACCTGATCCT |
| | GGACGAGCTGTGGCACACCAACGACAACCAGATCGCTATCTTCAACCGGCTGAA |
| | GCTGGTGCCCAAGAAGGTGGACCTGTCTCAGCAGAAAGAGATTCCCACCACACT |
| | GGTGGACGACTTCATTCTGAGCCCCGTGGTCAAGAGAAGCTTCATCCAGAGCAT |
| | CAAAGTGATCAACGCCATCATCAAGAAGTACGGGCTGCCCAACGATATCATCAT |
| | CGAGCTGGCCCGCGAGAAGAACTCCAAGGACGCCCAGAAAATGATCAACGAGAT |
| | GCAGAAGCGGAACCGGCAGACCAACGAGCGGATCGAGGAAATCATCCGGACCAC |
| | CGGCAAAGAGAACGCCAAGTACCTGATCGAGAAGATCAAGCTGCACGACATGCA |
| | AGAGGGCAAGTGCCTGTACAGCCTGGAAGCCATTCCTCTGGAAGATCTGCTGAA |
| | CAATCCCTTCAACTATGAGGTGGACCACATCATCCCCAGAAGCGTGTCCTTCGA |
| | CAACAGCTTCAACAACAAGGTGCTCGTGAAGCAAGAGGAAAACAGCAAGAAGGG |
| | CAACAGAACCCCATTCCAGTACCTGAGCAGCTCCGACAGCAAGATCAGCTACGA |
| | AACCTTCAAGAAGCACATCCTGAATCTGGCCAAAGGCAAGGGCAGAATCAGCAA |
| | GACCAAGAAAGAGTATCTGCTCGAGGAACGGGACATCAACAGATTCAGCGTGCA |
| | GAAAGACTTCATCAATCGGAACCTGGTGGACACCAGATACGCCACCAGAGGCCT |
| | GATGAATCTGCTGCGGAGCTACTTCAGAGTGAACAATCTGGACGTGAAAGTCAA |
| | GTCCATCAACGGCGGCTTCACCAGCTTTCTGCGGCGGAAGTGGAAGTTTAAGAA |
| | AGAGCGGAACAAGGGCTATAAGCACCATGCCGAGGACGCCCTGATCATTGCCAA |
| | CGCCGATTTCATCTTCAAAGAGTGGAAGAAACTGGACAAGGCCAAAAAAGTGAT |
| | GGAAAACCAGATGTTCGAGGAAAAGCAGGCCGAGAGCATGCCCGAGATCGAGAC |
| | TGAGCAAGAGTACAAAGAGATTTTCATCACGCCCCACCAGATCAAGCACATTAA |
| | GGACTTCAAGGACTACAAGTACAGCCACCGCGTGGACAAGAAGCCCAACCGGAA |
| | GCTGATCAATGACACACTGTACAGCACCCGGAAGGACGACAAGGGCAATACCCT |
| | GATCGTCAACAACCTGAACGGCCTGTACGACAAGGACAACGACAAGCTCAAGAA |
| | GCTGATTAACAAGAGCCCCGAGAAGCTCCTGATGTACCATCACGACCCTCAGAC |
| | CTACCAGAAACTGAAGCTCATCATGGAACAGTACGGCGACGAGAAAAACCCGCT |
| | GTACAAGTACTACGAGGAAACCGGAAACTACCTGACCAAGTACTCCAAAAAGGA |
| | CAATGGGCCCGTGATCAAGAAGATTAAGTATTACGGCAACAAGCTGAATGCCCA |
| | CCTGGACATCACCGACGACTACCCCAACAGCAGAAACAAGGTGGTCAAGCTGTC |
| | CCTGAAGCCTTACAGATTCGACGTGTACCTGGACAACGGCGTGTACAAGTTCGT |
| | GACCGTGAAGAACCTGGACGTCATCAAAAAAGAAAACTACTACGAAGTCAACAG |
| | CAAGTGCTATGAGGAAGCCAAGAAACTCAAGAAAATCAGCAACCAGGCCGAGTT |
| | TATCGCCAGCTTCTACAAGAACGACCTCATCAAGATCAACGGGGAGCTGTATAG |
| | AGTGATCGGGGTCAACAATGACCTGCTGAACCGGATCGAAGTGAACATGATCGA |
| | CATCACCTACCGCGAGTACCTCGAGAACATGAACGATAAGCGGCCACCGCACAT |
| | CATTAAGACAATCGCCAGCAAGACGCAGAGCATTAAGAAGTACAGCACAGACAT |
| | CCTGGGGAACCTGTACGAAGTGAAGTCCAAAAAGCACCCGCAGATTATCAAGAA |
| | AGGCGGCAGCCCCAAGAAAAAAAGAAAGGTTTCCTCCGACTACAAGGACCACGA |
| | TGGCGATTATAAGGATCACGACATCGATTACAAGGACGATGACGACAAGGAAGG |
| | CGCCGATAAGAGAACCGCCGATGGCTCTGAGTTCGAGAGCCCCAAGAAAAAGCG |
| | CAAAGTGTAATAGTGAGCGGCCGCTTAATTAAGCTGCCTTCTGCGGGGCTTGCC |
| | TTCTGGCCAAGCCCTTCTTCTCTCCCTTGCACCTGTACCTCTTGGTCTTTGAAT |
| | AAAGCCTGAGTAGGAAG (SEQ ID NO: 659) |
| Amino acid | MSEVEFSHEYWMRHALTLAKRARDEREVPVGAVLVLNNRVIGEGWNRAIGLHDP |
| sequence | TAHAQIMALRQGGLVMQNYRLYDATLYSTFEPCVMCAGAMIHSRIGRVVFGVRN |
| of the | AKTGAAGSLMDVLHHPGMNHRVEITEGILADECAALLCRFFRMPRRVFNAQKKA |
| polypeptide | QSSTDSGGSSGGSSGSETPGTSESATPESSGGSSGGSKRNYILGLAIGITSVGY |
| encoded | GIIDYETRDVIDAGVRLFKEANVENNEGRRSKRGARRLKRRRRHRIQRVKKLLF |
| by | DYNLLTDHSELSGINPYEARVKGLSQKLSEEEFSAALLHLAKRRGVHNVNEVEE |
| mRNA2973 | DTGNELSTKEQISRNSKALEEKYVAELQLERLKKDGEVRGSINRFKTSDYVKEA |
| | KQLLKVQKAYHQLDQSFIDTYIDLLETRRTYYEGPGEGSPFGWKDIKEWYEMLM |
| | GHCTYFPEELRSVKYAYNADLYNALNDLNNLVITRDENEKLEYYEKFQIIENVF |
| | KQKKKPTLKQIAKEILVNEEDIKGYRVTSTGKPEFTNLKVYHDIKDITARKEII |
| | ENAELLDQIAKILTIYQSSEDIQEELTNLNSELTQEEIEQISNLKGYTGTHNLS |
| | LKAINLILDELWHTNDNQIAIFNRLKLVPKKVDLSQQKEIPTTLVDDFILSPVV |
| | KRSFIQSIKVINAIIKKYGLPNDIIIELAREKNSKDAQKMINEMQKRNRQTNER |
| | IEEIIRTTGKENAKYLIEKIKLHDMQEGKCLYSLEAIPLEDLLNNPFNYEVDHI |
| | IPRSVSFDNSFNNKVLVKQEENSKKGNRTPFQYLSSSDSKISYETFKKHILNLA |
| | KGKGRISKTKKEYLLEERDINRFSVQKDFINRNLVDTRYATRGLMNLLRSYFRV |
| | NNLDVKVKSINGGFTSFLRRKWKFKKERNKGYKHHAEDALIIANADFIFKEWKK |
| | LDKAKKVMENQMFEEKQAESMPEIETEQEYKEIFITPHQIKHIKDFKDYKYSHR |
| | VDKKPNRKLINDTLYSTRKDDKGNTLIVNNLNGLYDKDNDKLKKLINKSPEKLL |
| | MYHHDPQTYQKLKLIMEQYGDEKNPLYKYYEETGNYLTKYSKKDNGPVIKKIKY |
| | YGNKLNAHLDITDDYPNSRNKVVKLSLKPYRFDVYLDNGVYKFVTVKNLDVIKK |
| | ENYYEVNSKCYEEAKKLKKISNQAEFIASFYKNDLIKINGELYRVIGVNNDLLN |
| | RIEVNMIDITYREYLENMNDKRPPHIIKTIASKTQSIKKYSTDILGNLYEVKSK |
| | KHPQIIKKGGSPKKKRKVSSDYKDHDGDYKDHDIDYKDDDDKEGADKRTADGSE |
| | FESPKKKRKV (SEQ ID NO: 660) |

### Example 4. Impact of Base Editing on SMN Polypeptide Expression

Experiments were undertaken to evaluate the impact of base editing of SMN2 in SMA patient fibroblast cells (GM03813 cells) on SMN polypeptide expression.

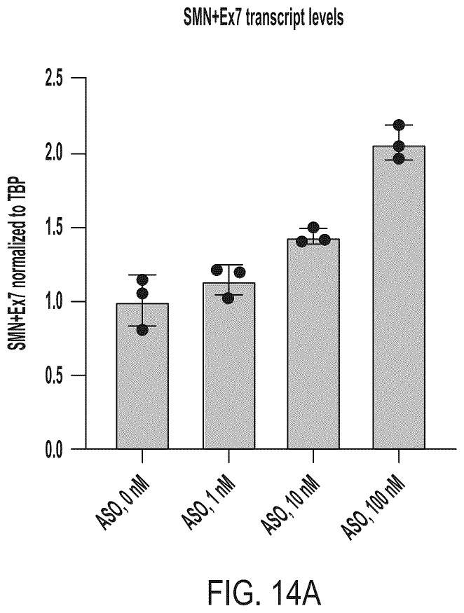

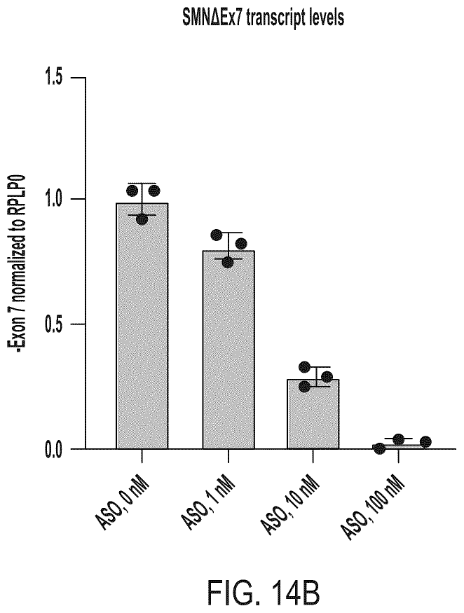

First, as a control experiment, spinal muscular atrophy (SMA) patient fibroblast cells (GM03813 cells) were transfected using Lipofectamine MessangerMAX and 0 nM, 1 nM, 10 nM, or 100 nM of the antisense oligonucleotide (ASO) nusinersen, which is known to improve levels of spliced SMN2 transcripts containing Exon 7 expressed in cells. Increasing concentrations of nusinersen was directly correlated with increased full-length SMN2 transcripts expressed in the cells (FIGS. 14A and 14B).

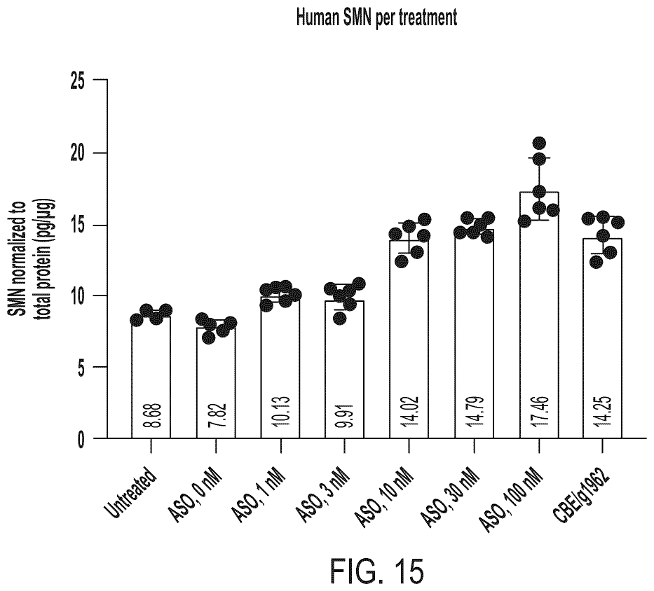

Next, an experiment was undertaken to evaluate the impact of nusinersen and base editing on SMN polypeptide levels (SMN levels) in SMA patient fibroblast cells (GM03813 cells). The base edited SMA patient fibroblast cells were transduced with mRNA encoding a cytidine base editor and the guide polynucleotide gRNA1962. SMN polypeptide levels were evaluated 5 days following transfection with the base editor system or following being transfected with nusinersen using an enzyme-linked immunosorbent assay (FIG. 15). Base edited cells had SMN polypeptide expression levels comparable to levels achieved in cells contacted with 10 nM nusinersen.

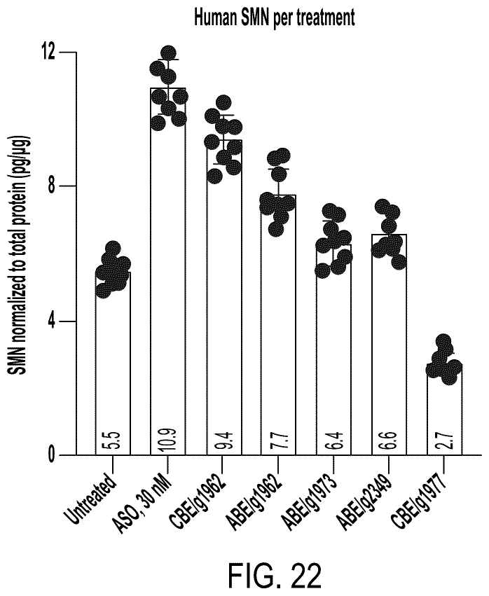

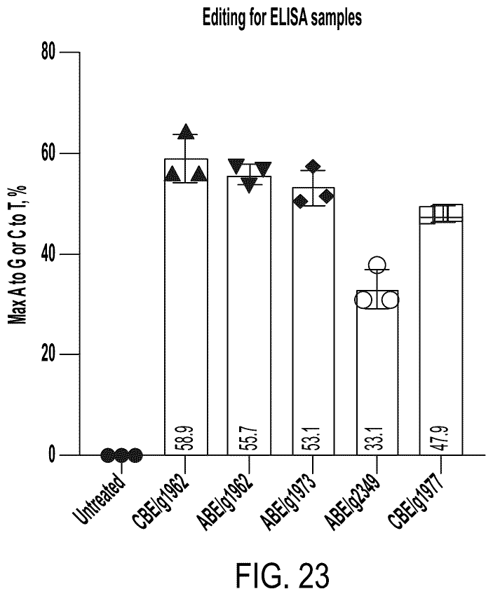

A further experiment was undertaken to evaluate the impact of base editing on SMN polypeptide levels (SMN levels) in SMA patient fibroblast cells (GM03813 cells). The base edited SMA patient fibroblast cells were prepared using the following base editor systems: CBE+gRNA1962; ABE+gRNA1962; ABE+gRNA1973; ABE+gRNA2349; and CBE+gRNA1977 (FIG. 22). Cells were transduced with mRNA encoding the base editor and the guide polynucleotide. All of the base editor systems achieved base editing efficiencies of greater than about 30% (FIG. 23). SMN polypeptide levels were evaluated 5 days following transfection with each the base editor system using an enzyme-linked immunosorbent assay (FIG. 22). Cells base edited using the base editor system CBE+gRNA1962 showed an increase in SMN polypeptide levels of about 70% relative to untreated cells, the base editor systems CBE+gRNA1962, ABE+gRNA1962, ABE+gRNA1973, and ABE+gRNA2349 were all associated with an increase in SMN polypeptide levels. Advantageously, the bases edited using the guide gRNA1962 fell outside of the coding region of SMN2.

TABLE 23
Details relating to base editor systems of FIG. 22. Each base
editor is named using the following convention: [Name of Type of Base
Editor]_[Deaminase Domain]_[nucleic acid programmable DNA
binding protein domain]_[PAM sequence], where “Sa” indicates
“Staplylococcus aureus,” “Sp” indicates “Streptococcus pyogenes,” and
“8.20” indicates “TadA*8.20.” See Tables 2B and 2C for guide sequences.

| | Name for | |
|---|---|---|
| | mRNA | |
| Type of Base Editor | encoding the | |
| Used and Guide Name | Base Editor | Base Editor |
| IBE16/gRNA2018 | mRNA3167 | IBE16_ABE_8.20_SpCas9_NGC |
| IBE12/gRNA2018 | mRNA3170 | IBE12_ABE_8.20_SpCas9_NGC |
| IBE11/gRNA2018 | mRNA2959 | IBE11_ABE_8.20_SpCas9_NGC |
| CBE/g1962 | mRNA2744 | CBE_ppAPOBEC1_SpCas9_NGA |
| ABE/g1962 | mRNA2928 | ABE_8.20_SpCas9_NGA |
| deadABE/g1962 | mRNA2971 | dead_ABE_dABE8.20_E59Q_SpCas9_NGA |
| ABE/g1973 | mRNA2926 | ABE_8.20_SpCas9_NGG |
| deadABE/g1973 | mRNA2970 | dead_ABE_dABE8.20_E59Q_SpCas9_NGG |
| ABE/g2439 | mRNA2866 | ABE_8.20_SpCas9_NRTH |
| deadABE/g2349 | mRNA2975 | dead_ABE_dABE8.20_E59Q_SpCas9_NRTH |

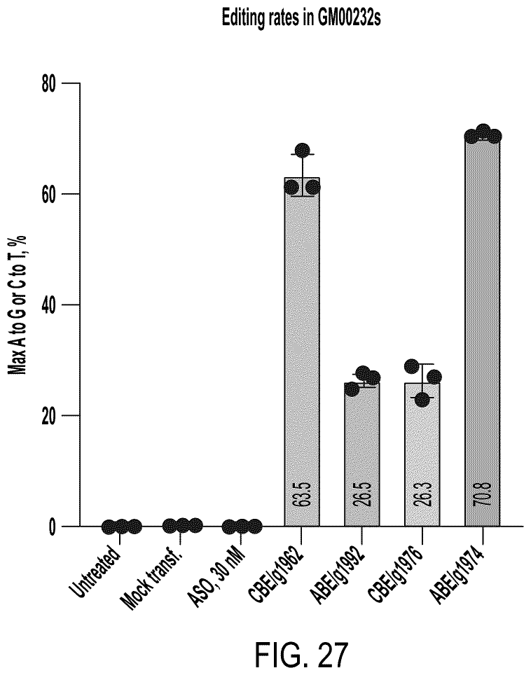

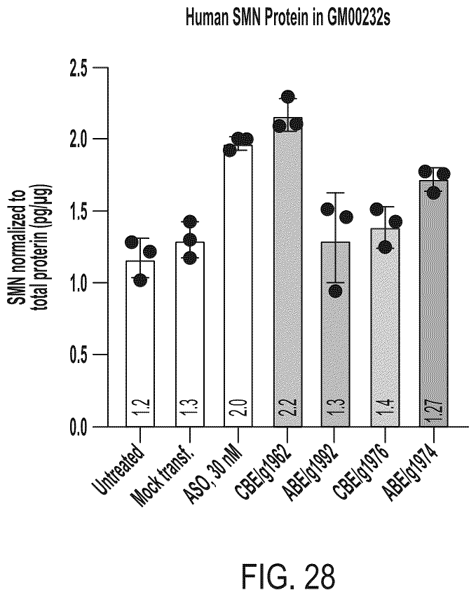

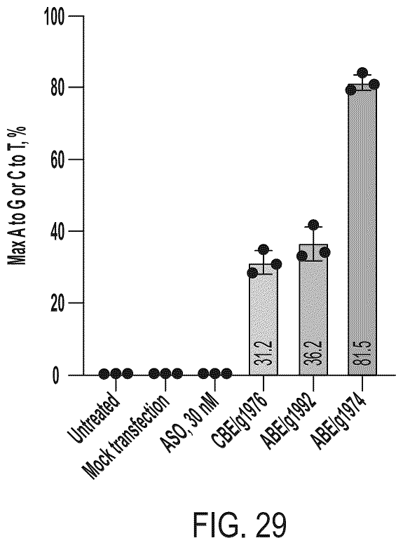

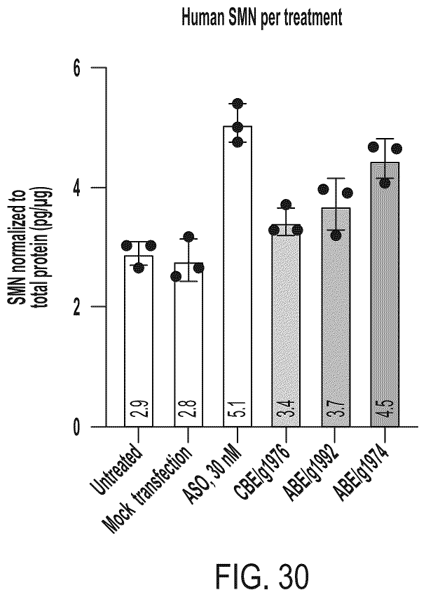

Experiments were undertaken to evaluate the impact of base editing on SMN2 polypeptide levels in GM00232 cells (FIGS. 28 and 28) and in GM03813 cells (FIGS. 29 and 30). The GM00232 cells were human fibroblast cells collected from a 7-month old while male child with muscular atrophy. The child had the following characteristics: progressive muscular atrophy; absent deep tendon reflexes; abnormal EMG; 2 copies of the SMN2 gene; and homozygous for deletion of exons 7 and 8 of the SMN1 gene. The GM03813 cells were also homozygous for deletions of exons 7 and 8 of the SMN1 gene but contained 3 copies of the SMN2 gene. The GM00232 cells were base edited using the following base editor systems: CBE (encoded by mRNA2744)+gRNA1962; ABE (encoded by mRNA2892)+gRNA1992; CBE (encoded by mRNA2745)+gRNA1976; ABE (encoded by mRNA2926)+gRNA1974. The GM03813 cells were base edited using the following base editor systems; CBE (encoded by mRNA2745)+gRNA1976; ABE (encoded by mRNA2926)+gRNA1974; ABE (encoded by mRNA2892)+gRNA1992. Cells were transduced with mRNA encoding the base editor and the guide polynucleotide. All of the base editor systems achieved base editing efficiencies of greater than about 25% (FIGS. 27 and 29). SMN polypeptide levels were evaluated 5 days following transfection with each the base editor system using an enzyme-linked immunosorbent assay (FIGS. 28 and 30). The base editor systems CBE+gRNA1976, ABE+gRNA1974, ABE+gRNA1992 were all associated with an increase in SMN polypeptide levels ranging from about 15% to about 50% in the GM03813 cells. The base editor systems CBE (encoded by mRNA2744)+gRNA1962, ABE (encoded by mRNA2892)+gRNA1992, CBE (encoded by mRNA2745)+gRNA1976, and ABE (encoded by mRNA2926)+gRNA1974 were all associated with an increase in SMN polypeptide levels ranging from about 8% to about 85% in the GM00232 cells, and in the same rank order as measured for GM03813 cells.

The following methods were or are employed in Examples 1 and 2.

### Codon-Optimization

Polynucleotide sequences encoding components of the base editor systems were codon-optimized. It has been established that Cas9 codon usage and nuclear localization sequence can dramatically alter genome editing efficiencies in eukaryotes (see e.g., Kim, S. et al., Rescue of high-specificity Cas9 variants using sgRNAs with matched 5′ nucleotides. Genome Biol 18, 218, doi:10.1186/s13059-017-1355-3 (2017); Mikami, M. et al., Comparison of CRISPR/Cas9 expression constructs for efficient targeted mutagenesis in rice. Plant Mol Biol 88, 561-572, doi:10.1007/s11103-015-0342-x (2015); Jinek, M. et al., RNA-programmed genome editing in human cells. Elife 2, e00471, doi:10.7554/eLife.00471 (2013)). The original Cas9n component of base editors contains six potential polyadenylation sites, leading to poor expression in eukaryotes (see e.g., Kim, S. et al., Rescue of high-specificity Cas9 variants using sgRNAs with matched 5′ nucleotides. Genome Biol 18, 218, doi:10.1186/s13059-017-1355-3 (2017); Komor, A. C. et al., Programmable editing of a target base in genomic DNA without double-stranded DNA cleavage. Nature 533, 420-424, doi:10.1038/nature17946 (2016); Gaudelli, N. M. et al.

Programmable base editing of A*T to G*C in genomic DNA without DNA cleavage. Nature 551, 464-471, doi:10.1038/nature24644 (2017)). Replacing this with an extensively optimized codon sequence improved base editing efficiencies (see e.g., Cong, L. et al. Multiplex genome engineering using CRISPR/Cas systems. Science 339, 819-823, doi:10.1126/science.1231143 (2013); Koblan, L. W. et al. Improving cytidine and adenine base editors by expression optimization and ancestral reconstruction. Nat Biotechnol, doi:10.1038/nbt.4172 (2018); Zafra, M. P. et al. Optimized base editors enable efficient editing in cells, organoids and mice. Nat Biotechnol, doi:10.1038/nbt.4194 (2018)).

### Cloning

All cloning was conducted via USER enzyme (New England Biolabs) cloning methods (see Geu-Flores et al., USER fusion: a rapid and efficient method for simultaneous fusion and cloning of multiple PCR products. Nucleic Acids Res 35, e55, doi:10.1093/nar/gkm106 (2007)) and templates for PCR amplification were purchased as bacterial or mammalian codon optimized gene fragments (GeneArt). Vectors were transformed into Mach T1R Competent Cells (Thermo Fisher Scientific) and maintained at −80 C for long-term storage. All primers used in this work were purchased from Integrated DNA Technologies and PCRs were carried out using either Phusion U DNA Polymerase Green MultiPlex PCR Master Mix (ThermoFisher) or Q5 Hot Start High-Fidelity 2x Master Mix (New England Biolabs). All plasmids used in this work were freshly prepared from 50 mL of Machi culture using ZymoPURE Plasmid Midiprep (Zymo Research Corporation) which involves an endotoxin removal procedure. Molecular biology grade, Hyclone water (GE Healthcare Life Sciences) was used in all assays, transfections, and PCR reactions to ensure exclusion of DNAse activity.

### Mammalian Cell Culture Conditions

The HEK293T (293T) cell line is obtained from the American Tissue Culture Collection (ATCC). Cells are cultured at 37° C. with 5% CO₂. HEK293T cells [CLBTx013, American Type Cell Culture Collection (ATCC)] are cultured in Dulbecco's modified Eagles medium plus Glutamax (10566-016, Thermo Fisher Scientific) with 10% (v/v) fetal bovine serum (A31606-02, Thermo Fisher Scientific). Cells are tested for mycoplasma after receipt from the supplier.

Hek293T Transfection and Genomic DNA (gDNA) Extraction

HEK293T cells are seeded on 48-well collagen-coated BioCoat plates (Corning) and transfected at approximately 85% confluency. Briefly, HEK293T cells are seeded onto 48-well well Poly-D-Lysine treated BioCoat plates (Corning) at a density of 35,000 cells/well and transfected 18-24 hours after plating. Cells are counted using a NucleoCounter NC-200 (Chemometec). To these cells are added 750 ng of base editor or nuclease, 250 ng of sgRNA, and 10 ng of GFP-max plasmid (Lonza) diluted to 12.5 μL total volume in Opti-MEM reduced serum media (ThermoFisher Scientific). The solution is combined with 1.5 μL of Lipofectamine 2000 (ThermoFisher) in 11 μL of Opti-MEM reduced serum media and left to rest at room temperature for 15 min. The entire 25 μL mixture is then transferred to the pre-seeded Hek293T cells and left to incubate for about 120 h. Following incubation, media is aspirated and cells are washed two times with 250 μL of 1× PBS solution (ThermoFisher Scientific) and 100 μL of freshly prepared lysis buffer is added (100 mM Tris-HCl, pH 7.0, 0.05% SDS, 25 μg/mL Proteinase K (Thermo Fisher Scientific). Transfection plates containing lysis buffer are incubated at 37° C. for 1 hour and the mixture is transferred to a 96-well PCR plate and heated at 80° C. for 30 min.

### Analysis of DNA and RNA Off-Target Editing

HEK293T cells are plated on 48-well poly-D-lysine coated plates (Corning) 16 to 20 hours before lipofection at a density of 30,000 cells per well in DMEM+Glutamax medium (Thermo Fisher Scientific) without antibiotics. 750 ng nickase, nuclease, or base editor expression plasmid DNA is combined with 250 ng of sgRNA expression plasmid DNA in 15 μl OPTIMEM+Glutamax. This is combined with 10 μl of lipid mixture, comprising 1.5 μl Lipofectamine 2000 and 8.5 μl OPTIMEM+Glutamax per well. Cells are harvested 3 days after transfection and either DNA or RNA is harvested. For DNA analysis, cells are washed once in 1×PBS, and then lysed in 100 μl QuickExtract™ Buffer (Lucigen) according to the manufacturer's instructions. For RNA harvest, the MagMAX™ mirVana™ Total RNA Isolation Kit (Thermo Fisher Scientific) is used with the KingFisher™ Flex Purification System according to the manufacturer's instructions.

Targeted RNA sequencing is performed largely as previously described (see Rees, H. A. et al., Analysis and minimization of cellular RNA editing by DNA adenine base editors. Sci Adv 5, eaax5717, doi:10.1126/sciadv.aax5717 (2019)). cDNA is prepared from the isolated RNA using the SuperScript IV One-Step RT-PCR System with EZDnase (Thermo Fisher Scientific) according to the manufacturer's instructions. The following program is used: 58° C. for 12 min; 98° C. for 2 min; followed by PCR cycles which varied by amplicon. No RT controls are run concurrently with the samples. Following the combined RT-PCR, amplicons are barcoded and sequenced using an Illumina Miseq. The first 125nt in each amplicon, beginning at the first base after the end of the forward primer in each amplicon, is aligned to a reference sequence and used for analysis of mean and maximum base editing frequencies in each amplicon.

Off-target DNA sequencing is performed using previously published primers (see Komor, A. C. et al., Programmable editing of a target base in genomic DNA without double-stranded DNA cleavage. Nature 533, 420-424, doi:10.1038/nature17946 (2016); Rees, H. A. et al., Analysis and minimization of cellular RNA editing by DNA adenine base editors. Sci Adv 5, eaax5717, doi:10.1126/sciadv.aax5717 (2019)) using a two-step PCR and barcoding method to prepare samples for sequencing using Illumina Miseq sequencers.

Deep sequencing is performed on PCR amplicons from genomic DNA or RNA harvested from duplicate transfections of 293T cells. After validating the quality of PCR product by gel electrophoresis, the PCR products are isolated by gel extraction, e.g., using the Zymoclean Gel DNA Recovery Kit (Zymo Research). Shotgun libraries are prepared without shearing. The library is quantified by qPCR and sequenced on one MiSeq Nano flowcell for 251 cycles from each end of the fragments using a MiSeq 500-cycle sequencing kit version 2. Fastq files are generated and demultiplexed with the bcl2fastq v2.17.1.14 Conversion Software (Illumina).

mRNA Production

Editors/nucleases are cloned into a plasmid encoding a dT7 promoter followed by a 5′UTR, Kozak sequence, ORF, and 3′UTR. The dT7 promoter carries an inactivating point mutation within the T7 promoter that prevents transcription from circular plasmid. This plasmid templates a PCR reaction (Q5 Hot Start 2X Master Mix), in which the forward primer corrects the SNP within the T7 promoter and the reverse primer appends a 120A tail to the 3′ UTR. The resulting PCR product is purified on a Zymo Research 25 μg DCC column and used as mRNA template in the subsequent in vitro transcription. The NEB HiScribe High-Yield Kit is used as per the instruction manual but with full substitution of N1-methyl-pseudouridine for uridine and co-transcriptional capping with CleanCap AG (Trilink). Reaction cleanup is performed by lithium chloride precipitation.

In Vitro Transcription of sgRNAs.

Linear DNA fragments containing the CMV promoter followed by the sgRNA target sequence are transcribed in vitro using the TranscriptAid T7 High Yield Transcription Kit (ThermoFisher Scientific) according to the manufacturer's instructions. sgRNA products are purified using the MEGAclear Kit (ThermoFisher Scientific) according to the manufacturer's instructions and quantified by UV absorbance.

### High-Throughput DNA Sequencing

Samples are sequenced on an Illumina MiSeq as previously described (Pattanayak, Nature Biotechnol. 31, 839-843 (2013)).

### Data Analysis

Sequencing reads are automatically demultiplexed using MiSeq Reporter (Illumina), and individual FASTQ files are analyzed with a custom Matlab. Each read is pairwise aligned to the appropriate reference sequence using the Smith-Waterman algorithm. Base calls with a Q-score below 31 are replaced with Ns and are thus excluded in calculating nucleotide frequencies. This treatment yields an expected MiSeq base-calling error rate of approximately 1 in 1,000. Aligned sequences in which the read and reference sequence contain no gaps are stored in an alignment table from which base frequencies can be tabulated for each locus. Indel frequencies are quantified with a custom Matlab script using previously described criteria (Zuris, et al., Nature Biotechnol. 33, 73-80 (2015). Sequencing reads are scanned for exact matches to two 10-bp sequences that flank both sides of a window in which indels might occur. If no exact matches are located, the read is excluded from analysis. If the length of this indel window exactly matched the reference sequence, the read is classified as not containing an indel. If the indel window was two or more bases longer or shorter than the reference sequence, then the sequencing read is classified as an insertion or deletion, respectively.

The following materials and methods were employed in Examples 3 and 4.

### Evaluation of Base Editor Systems

### GM03813 Cells

Two 48 well plates of SMA patient fibroblast cells (GM03813, GM00232, or GM09677 cells) were plated such that they would be ˜80% confluent the next day for transfection (˜11,000 cells per plate for GM00232 and GM09677).

Once ˜80% confluent, the two plates were identically transfected with 300 ng or 60 ng of the respective editor mRNA and 100 ng or 30 ng of the respective gRNA, respectively, using 1 uL of Lipofectamine MessengerMAX (LMRNA001), otherwise following the manufacturer's protocol. It was found that transfection with 60 ng of the mRNA and 30 ng of the gRNA resulted in comparatively high cell density post-transfection and good base editing efficiencies. All treatments were done in triplicate. Mock transfection controls underwent the same protocol as other samples, but no RNA was ever added. Editor/guide combinations were chosen to maximize base editing efficiencies. Base editor controls included an adenosine deaminase used in combination with the guide sgRNA88 and a cytidine deaminase used in combination with the guide sgRNA88. As a positive control, unedited cells were contacted with 100 nM or 30 nM of the antisense oligonucleotide (ASO) nusinersen. The editor/guide combinations evaluated in GM00232 and GM09577 cells were chosen due to their ability to make edits that increase Exon 7 inclusion on an RNA level and increase SMN protein in GM03813s. In some cases, active base editors were tested alongside dead base editor variants with low-to-no base editing activity.

The cells were processed 5 days post-transfection. One plate was processed using a Cells-to-CT™ 1-Step TaqMan™ Kit (A25603) and lysed using 75 uL of the included lysis buffer to cover the bottom of each well, otherwise following the manufacturer's protocol. For all lysates qRT-PCR was used to measure total levels of spliced SMN2 and/or SMN1 transcripts as described below.

The other plate was processed using QuickExtract DNA Extraction Solution (QE09050) for next-generation sequencing (NGS) submission using the manufacturer's protocol.

### Enzyme-Linked Immunosorbent Assay

One 24 well plate was seeded with about 13,000 or 15,000 GM03813 or GM00232 patient fibroblasts per well so that the following day, each well would be ˜85% confluent and ready for transfection. Alternatively, two 24 well plates were seeded with 35,000 GM03813 patient fibroblasts per well so that about 3 hours later the same day, each well would be ˜85% confluent and the cells would settle and be ready for transfection, where one plate was used for ELISA analysis and the other for NGS processing.

Once adequately confluent, the plate was transfected with 1 nM, 3 nM, 10 nM, 30 nM, or 100 nM of the antisense oligonucleotide (ASO) nusinersen or transfected with 104 ng of mRNA encoding a base editor and 52 ng of a guide polynucleotide (e.g., gRNA1962, gRNA1973, gRNA1974, gRNA1976, gRNA1992, gRNA2349, gRNA1977). The transfections were completed using 1.5 uL of Lipofectamine MessengerMAX (LMRNA001), otherwise following the manufacturer's protocol. The amount of editor mRNA and gRNA was chosen because it showed a combination of good cell density post-transfection and good editing efficiencies. The dose was scaled up from 60 ng editor mRNA:30 ng gRNA in a 48 well plate. The mock transfection control underwent the same protocol as the others, but no RNA was ever added. All treatments were done in triplicate.

The plate was lysed according to the ELISA kit's protocol and collected on day 5. Each well was pelleted, washed with PBS, pelleted again, and then lysed in 100 uL of complete cell lysis buffer. No significant cell death was seen at any point after transfection. The SMN ELISA was performed using the Enzo kit (ADI-900-209) and its respective protocol. Cells were lysed in 500 uL of complete lysis buffer. ELISA results were normalized to total protein amount using the Pierce™ Rapid Gold BCA Protein Assay Kit (A53227) and its respective protocol.

Quantitative Reverse Transcriptase Polymerase Chain Reaction (qRT-PCR)

Two 48 well plates of GM03813 cells were plated such that they would be ˜80% confluent the next day for transfection. 11,000 cells per well were plated to achieve this end.

Once ˜80% confluent, the two plates were transfected with mRNA encoding a base editor and a guide polynucleotide (gRNA) using 1 uL of Lipofectamine MessengerMAX (LMRNA001), otherwise following the manufacturer's protocol. All treatments were done in triplicate. A mock transfection control underwent the same protocol as the others, but no RNA was ever added. Various guides/editor combinations and transfection and treatment controls were included in the transfection.

The cells were processed 5 days post-transfection. One plate was processed using a Cells-to-CT™ 1-Step TaqMan™ Kit (A25603) and lysed using 75 uL of the included lysis buffer to cover the bottom of each well, otherwise following the manufacturer's protocol. The following were measured for all lysates: total levels of spliced SMN2 and/or SMN1 transcripts (ThermoFisher kit Hs00165806_m1) and levels of transcripts including or excluding Exon 7. Levels of TBP transcripts were measured for normalizing SMN2 and/or SMN1 transcript levels. The following off-the shelf assays were used: Hs00165806_m1 from ThermoFisher (for measuring total SMN transcript levels); Hs00427620_m1 from ThermoFisher (for measuring TBP transcript levels).

The following primers were used: 5′-CACATTCCAGATCTGTCTGATCG-3′ (SEQ ID NO: 620; 1R); 5′-CACCACCTCCCATATGTCCAG-3′ (SEQ ID NO: 621; 1F); 5′-TACTGGCTATTATATGGGTTTTAG-3′ (SEQ ID NO: 622; 2F). The following probe was used for detecting spliced SMN1 and/or SMN2 transcripts lacking Exon 7: 5′-/SHEX/ACTGGCTAT/ZEN/TATATGGAAATGCTGGCA/31ABkFQ/-3′ (SEQ ID NO: 623), where “SHEX” and “31ABkFQ” represent fluorescent dyes and “ZEN” represents an internal quencher. The following probe was used for detecting spliced SMN1 and/or SMN2 transcripts that included Exon 7: 5′-/56-FAM/AAGAAGGAA/ZEN/GGTGCTCACATTCCT/31ABkFQ/-3′ (SEQ ID NO: 624), where “56-FAM” and “31ABkFQ” represent fluorescent dyes and “ZEN” represents an internal quencher.

qRT-PCR was conducted according to the Cells-to-CT kit's protocol. 2 uL of lysate was used in each 20 uL reaction in a 96 well qPCR plate. The 60° C. step during amplification was shortened from 1 min to 20s. The no reverse transcriptase (-RT) control was prepared by heating the Cells-to-CT qPCR mix at 90° C. for 10 minutes. Duplicates of each treatment triplicate were analyzed. Spliced SMN1 and/or SMN2 transcript Ct results were normalized to TBP Ct results.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed:

1. A method of editing a nucleobase of a survival of motor neuron 2 (SMN2) polynucleotide, the method comprising contacting the SMN2 polynucleotide with a guide polynucleotide and a base editor comprising a fusion protein or a protein complex comprising a nucleic acid programmable DNA binding protein (napDNAbp) domain and a deaminase domain, wherein said guide polynucleotide targets said base editor to effect an alteration of the nucleobase of the SMN2 polynucleotide.

2. The method of claim 1, wherein the SMN2 polynucleotide is in a motor neuron or fibroblast, and wherein the cell is in vivo or in vitro.

3. A method of treating spinal muscular atrophy (SMA) in a subject in need thereof, the method comprising contacting a cell of the subject with a base editor comprising a fusion protein or protein complex comprising a nucleic acid programmable DNA binding protein (napDNAbp) domain and a deaminase domain, or a polynucleotide encoding the base editor, and one, two, or more guide polynucleotide(s), or polynucleotide(s) encoding the guide polynucleotide(s), that target the base editor to effect an alteration of a survival of motor neuron 2 (SMN2) polynucleotide, thereby treating SMA in the subject.

4. The method of claim 1, wherein the guide polynucleotide(s) comprises a nucleic acid sequence comprising at least 10 contiguous nucleotides of a spacer nucleic acid sequence listed in Table 2A or Table 2C.

5. The method of claim 4, wherein the guide polynucleotide comprises the nucleotide sequence ACUCCUUAAUUUAAGGAAUG (SEQ ID NO: 562; gRNA1962); GACAAAAUCAAAAAGAAGGA (SEQ ID No: 514; gRNA1973); UUAAGGAGUAAGUCUGCCAG (SEQ ID NO: 626; gRNA2349); UGCUCACAUUCCUUAAAUUA (SEQ ID NO: 574; gRNA1974); GCAGACUUACUCCUUAAUUUA (SEQ ID NO: 576; gRNA1976); or UCACAUUCCUUAAAUUAAGGA (SEQ ID NO: 592; gRNA1992).

6. The method of any one of claim 1, wherein the guide polynucleotide comprises a scaffold comprising the nucleotide sequence:
SpCas9 scaffold
(SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU;
or
SaCas9 scaffold
(SEQ ID NO: 425)
GUUUUAGUACUCUGUAAUGAAAAUUACAGAAUCUACUAAAACAAGGCAA
AAUGCCGUGUUUAUCUCGUCAACUUGUUGGCGAGAUUUU.

7. The method of claim 1, wherein the guide polynucleotide comprises the sequence
(SEQ ID NO: 503; gRNA1962)
ACUCCUUAAUUUAAGGAAUGGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 514; gRNA1973)
GACAAAAUCAAAAAGAAGGAGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 630; gRNA2349)
UUAAGGAGUAAGUCUGCCAGGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 515; gRNA1974)
UGCUCACAUUCCUUAAAUUAGUUUUAGAGCUAGAAAUAGCAAGUUAAAA
UAAGGCUAGUCCGUUAUCAACUUGAAAAAGUGGCACCGAGUCGGUGCUU
UU;
(SEQ ID NO: 517; gRNA1976)
GCAGACUUACUCCUUAAUUUAGUUUUAGUACUCUGUAAUGAAAAUUACA
GAAUCUACUAAAACAAGGCAAAAUGCCGUGUUUAUCUCGUCAACUUGUU
GGCGAGAUUUU;
or
(SEQ ID NO: 533; gRNA1992)
UCACAUUCCUUAAAUUAAGGAGUUUUAGUACUCUGUAAUGAAAAUUACA
GAAUCUACUAAAACAAGGCAAAAUGCCGUGUUUAUCUCGUCAACUUGUU
GGCGAGAUUUU.

8. The method of claim 1, wherein alteration of the nucleobase is associated with an increase in full-length polynucleotides encoding the SMN2 polypeptide transcribed from the SMN2 polynucleotide; and/or wherein alteration of a nucleobase in the SMN2 polynucleotide results in an increase in the number of transcripts transcribed from the SMN2 polynucleotide that include Exon 7.

9. The method of claim 1, wherein the altered nucleobase in the SMN2 polynucleotide is associated with an alteration in splicing; and/or wherein the alteration is associated with an increase in levels of a survival motor neuron (SMN) polypeptide in a cell.

10. The method of claim 1, wherein the alteration is selected from the group consisting of a C10 alteration in Intron 7 of the SMN2 polynucleotide, a C7 alteration in Intron 7 of the SMN2 polynucleotide, a C11 alteration in Intron 7 of the SMN2 polynucleotide, a T6C alteration in Exon 7 of the SMN2 polynucleotide, and a A54G alteration in Exon 7 of the SMN2 polynucleotide.

11. The method of claim 1, wherein the napDNAbp domain comprises a Cas9, Cas12a/Cpf1, Cas12b/C2c1, Cas12c/C2c3, Cas12d/CasY, Cas12e/CasX, Cas12g, Cas12h, Cas12i, or Cas12j/CasΦ polynucleotide or a portion thereof,
wherein the napDNAbp domain comprises a dead Cas9 (dCas9) or a Cas9 nickase (nCas9);
wherein the napDNAbp domain is a modified Staphylococcus aureus Cas9 (SaCas9), Streptococcus thermophilus 1 Cas9 (St1Cas9), a modified Streptococcus pyogenes Cas9 (SpCas9), or variants thereof,
wherein the napDNAbp domain comprises a variant of SpCas9 having an altered protospacer-adjacent motif (PAM) specificity;
wherein the cytidine deaminase domain is an APOBEC deaminase domain or a derivative thereof;
wherein the adenosine deaminase domain is TadA deaminase domain;
wherein the napDNAbp domain further comprises one or more uracil glycosylase inhibitors (UGIs); and/or
wherein the napDNAbp domain further comprises one or more nuclear localization sequences (NLS).

12. The method of claim 3, wherein contacting the cell involves administering a polynucleotide to the subject or a vector comprising the polynucleotide, wherein the polynucleotide encodes the base editor, or a component thereof, and/or the guide polynucleotide.

13. The method of claim 12, wherein the vector is a lipid nanoparticle.

14. The method of claim 13, wherein the vector is an AAV9 vector or an AAV-PHP.B vector.

15. A modified cell comprising an alteration in a nucleobase of a survival of motor neuron 2 (SMN2) polynucleotide, wherein the alteration increases expression and/or activity of the encoded SMN2 polypeptide as compared to a control cell without the alteration.

16. A base editor system comprising a fusion protein or a polynucleotide encoding the fusion protein, wherein the fusion protein comprises a nucleic acid programmable DNA binding protein (napDNAbp) domain, a deaminase domain, and a guide polynucleotide comprising a spacer sequence selected from Table 2A or Table 2C.

17. A polynucleotide encoding the base editor system of claim 16, or a component thereof.

18. A vector comprising the polynucleotide of claim 17.

19. A cell produced by the method of claim 3.

20. A kit comprising a base editor system comprising a fusion protein or a polynucleotide encoding the fusion protein, wherein the fusion protein comprises a nucleic acid programmable DNA binding protein (napDNAbp) domain, a deaminase domain, and a guide polynucleotide comprising a spacer selected from Table 2A or Table 2C.

21. A pharmaceutical composition comprising an effective amount of a base editor system comprising a fusion protein or a polynucleotide encoding the fusion protein, wherein the fusion protein comprises a nucleic acid programmable DNA binding protein (napDNAbp) domain, a deaminase domain, and a guide polynucleotide comprising a spacer selected from Table 2A or Table 2C.

22. A guide polynucleotide comprising a sequence listed in Tables 2A-2C.
